(19) United States
(12) Patent Application Publication
(10) Pub. No.: US 2026/0284214 A1
(43) Pub. Date: Sep. 24, 2026

(54) EXATECAN DERIVATIVE AND ANTIBODY-DRUG CONJUGATE THEREOF

(71) Applicant: SHANGHAI QILU PHARMACEUTICAL RESEARCH AND DEVELOPMENT CENTRE LTD., Shanghai (CN)

(72) Inventors: Junfei WANG, Shanghai (CN); Xiaoyang BI, Shanghai (CN); Wenshu HUANG, Shanghai (CN); Xinmei WANG, Shanghai (CN); Ying HUANG, Shanghai (CN); Yang YANG, Shanghai (CN); Daqing SUN, Shanghai (CN); Weikang TAO, Shanghai (CN)

(21) Appl. No.: 19/469,842

(22) PCT Filed: Mar. 29, 2024
(86) PCT No.: PCT/CN2024/084871

§ 371 Date: Sep. 26, 2025

(30) Foreign Application Priority Data
Mar. 30, 2023 (CN) 202310332433.6
Aug. 28, 2023 (CN) 202311096034.0
Mar. 21, 2024 (CN) 202410334352.4

(51) Int. Cl. A61K 47/68; A61K 31/4745; A61P 35/00; C07D 491/22; C07K 16/32

(52) U.S. Cl. CPC A61K 47/68037; A61K 31/4745; A61K 47/6855; A61K 47/6889; A61P 35/00; C07D 491/22; C07K 16/32

(57) ABSTRACT

The present invention provides a novel Exatecan derivative having biological activity and an antibody-drug conjugate thereof, and particularly relates to preparation methods for the Exatecan derivative and the antibody-drug conjugate thereof and uses of the Exatecan derivative and the antibody-drug conjugate thereof.

### CROSS-REFERENCE TO RELATED APPLICATIONS

This application is a U.S. National Stage of International Patent Application No. PCT/CN2024/084871 filed Mar. 29, 2024, which claims priority to Chinese Patent Application No. 2023103324336 filed on Mar. 30, 2023, Chinese Patent Application No. 2023110960340 filed on Aug. 28, 2023, and Chinese Patent Application No. 2024103343524 filed on Mar. 21, 2024, all of which are incorporated by reference herein.

### TECHNICAL FIELD

The present disclosure belongs to the field of medicinal chemistry, and particularly relates to preparation methods and applications of an Exatecan derivative and an antibody-drug conjugate thereof.

### BACKGROUND

Traditional chemotherapy is one of the main ways of tumor treatment. Although chemotherapy drugs mainly act on tumor cells with disordered proliferation, there is also inevitably damage to normal cells. According to a strategy of traditional chemotherapy, drugs with different action mechanisms are combined in application, so that the drugs may exert an anti-tumor effect synergistically while avoiding overlapping toxicity. The non-selectivity and high systemic toxicity of traditional chemotherapy drugs are the main reasons of limiting their application. In order to reduce the occurrence of adverse events, a targeted drug has become a research direction of anti-tumor drug. By detecting gene targets specifically expressed in tumor cells and further developing corresponding targeted drugs, the specificity of the drugs to the tumor cells can be improved, and the damage to normal cells can be reduced, so that adverse reactions can be alleviated.

An antibody-drug conjugate (ADC) links a monoclonal antibody or an antibody fragment to a cytotoxin with biological activity through a stable chemical linker compound, which makes full use of the specificity of antibody binding to surface antigens of normal cells and tumor cells and the high efficiency of cytotoxic substance, while avoiding the defects of low curative effect of the former and excessive toxic and side effects of the latter. Compared with the traditional chemotherapy drugs, antibody-drug conjugate can bind to the tumor cells more accurately and reduce an influence on the normal cells. In order to develop more efficient ADC drugs, researchers have paid more attention to cytotoxic molecules, and have strove to make greater progress in curative effect.

### SUMMARY

The present disclosure relates to a cytotoxic drug as shown by formula (D′), a stereoisomer thereof or a pharmaceutically acceptable salt thereof,

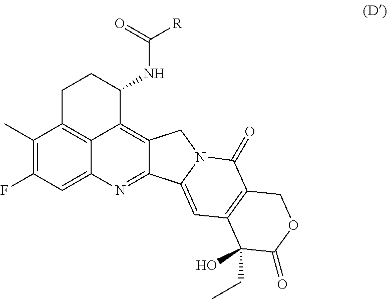

wherein, R is —NR¹R² or —OR³;
when R¹ is —CH₂CH₂OH, R² is selected from OH, CN, C₁₋₄ alkyl, C₁₋₄ alkoxy, C₂₋₄ alkenyl, C₂₋₄ alkynyl, —O—C₃₋₆ cycloalkyl, 3-6 membered heterocyclyl or C₃₋₆ cycloalkyl, and the C₁₋₄ alkyl, the C₃₋₆ cycloalkyl and the 3-6 membered heterocyclyl are optionally further substituted by 1, 2 or 3 CN or ═O; and the 3-6 membered heterocyclyl contains 1, 2 or 3 heteroatoms selected from N, O or S;
or, when R¹ is selected from H, C₁₋₄ alkyl or C₃₋₆ cycloalkyl, R² is selected from 3-6 membered heterocyclyl or —O—CH₂CH₂OH;
or, when R¹ is C₁₋₄ alkoxy, R² is selected from C₃₋₆ cycloalkyl or 3-6 membered heterocyclyl, and the C₃₋₆ cycloalkyl is optionally further substituted by one or more substituents selected from OH or NH₂; and
R³ is selected from C₃₋₆ cycloalkyl or 3-6 membered heterocyclyl, and the C₃₋₆ cycloalkyl is optionally further substituted by one or more substituents selected from OH or NH₂.

In some embodiments, in the cytotoxic drug, the stereoisomer thereof or the pharmaceutically acceptable salt thereof,
when R¹ is —CH₂CH₂OH, R² is selected from OH, CN, isopropyl, —CH₂CN, —CH₂C≡CH, —CH₂CH═CH₂, methoxy,

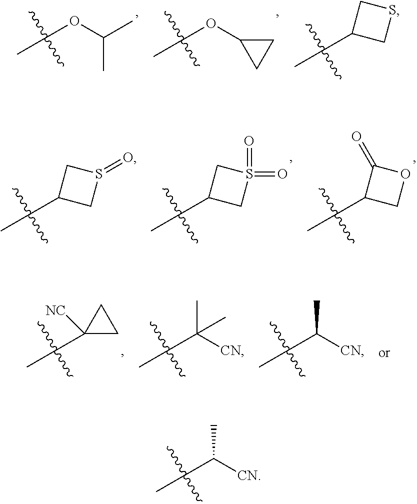

In some embodiments, in the cytotoxic drug, the stereoisomer thereof or the pharmaceutically acceptable salt thereof,
when R¹ is H, R² is selected from —O—CH₂CH₂OH or

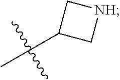 [SMILES: *C1CNC1]

or, when R¹ is selected from methyl, isopropyl or cyclopropyl, R² is —O—CH₂CH₂OH;
or, when R¹ is methoxy, R² is selected from cyclobutyl or

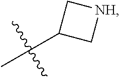 [SMILES: *C1CNC1]

and the cyclobutyl is optionally further substituted by NH₂.

In some embodiments, in the cytotoxic drug, the stereoisomer thereof or the pharmaceutically acceptable salt thereof,
R³ is selected from cyclobutyl or azacyclobutyl, and the cyclobutyl is optionally further substituted by one or more substituents selected from OH or NH₂.

In another aspect, the present disclosure relates to a cytotoxic drug (compound) as shown by formula (D′), a stereoisomer thereof or a pharmaceutically acceptable salt thereof,

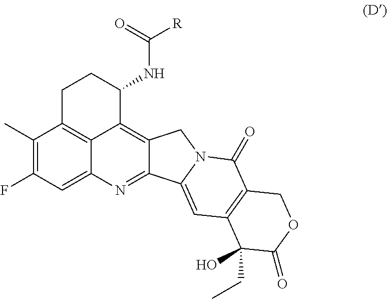

wherein, R is —NR¹R² or —OR³;
when R¹ is —CH₂CH₂OH, R² is selected from OH, CN, C₁₋₄ alkyl, C₁₋₄ alkoxy, C₂₋₄ alkenyl, C₂₋₄ alkynyl, —O—C₃₋₆ cycloalkyl, 3-6 membered heterocyclyl or C₃₋₆ cycloalkyl, and the C₁₋₄ alkyl, the C₃₋₆ cycloalkyl and the 3-6 membered heterocyclyl are optionally further substituted by 1, 2 or 3 CN or ═O; and the 3-6 membered heterocyclyl contains 1, 2 or 3 heteroatoms selected from N, O or S;
or, when R¹ is selected from H, C₁₋₄ alkyl or C₃₋₆ cycloalkyl, R² is selected from 3-6 membered heterocyclyl, —O—CH₂CH₂OH or C₃₋₆ cycloalkyl; the C₃₋₆ cycloalkyl is optionally further substituted by one or more substituents selected from OH or NH₂; and the 3-6 membered heterocyclyl contains 1, 2 or 3 heteroatoms selected from N, O or S;
or, when R¹ is C₁₋₄ alkoxy, R² is selected from C₃₋₆ cycloalkyl or 3-6 membered heterocyclyl, and the C₃₋₆ cycloalkyl is optionally further substituted by one or more substituents selected from OH or NH₂; and the 3-6 membered heterocyclyl contains 1, 2 or 3 heteroatoms selected from N, O or S;
or, R¹ and R² form an N-containing 4-6 membered heterocyclic ring together with the N atom to which R¹ and R² are connected, and the N-containing 4-6 membered heterocyclic ring is optionally further substituted by one or more substituents selected from OH or NH₂; and the N-containing 4-6 membered heterocyclic ring optionally contains 1 or 2 heteroatoms selected from O; and
R³ is selected from C₃₋₆ cycloalkyl or 3-6 membered heterocyclyl, and the C₃₋₆ cycloalkyl is optionally further substituted by one or more substituents selected from OH or NH₂; and the 3-6 membered heterocyclyl contains 1, 2 or 3 heteroatoms selected from N, O or S.

In some embodiments, in the cytotoxic drug, the stereoisomer thereof or the pharmaceutically acceptable salt thereof,
when R¹ is —CH₂CH₂OH, R² is selected from OH, CN, isopropyl, —CH₂CN, —CH₂C≡CH, —CH₂CH═CH₂, methoxy,

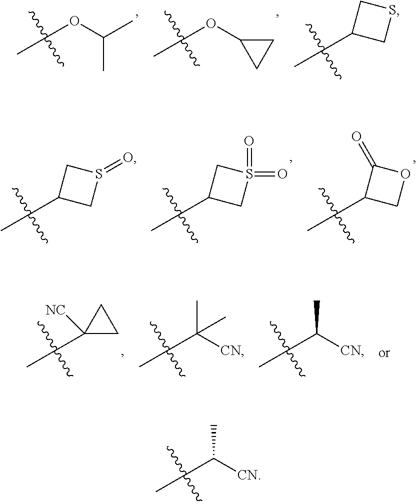

In some embodiments, in the cytotoxic drug, the stereoisomer thereof or the pharmaceutically acceptable salt thereof,
when R¹ is H, R² is selected from —O—CH₂CH₂OH,

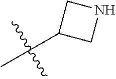 [SMILES: *C1CNC1]

or cyclobutyl, and the cyclobutyl is optionally further substituted by one or more substituents selected from OH or NH₂;
or, when R¹ is selected from methyl, isopropyl or cyclopropyl, R² is —O—CH₂CH₂OH;
or, when R¹ is methoxy, R² is selected from C₄₋₅ cycloalkyl or 4-5 membered N-containing heterocyclyl, and preferably, R² is selected from cyclobutyl or

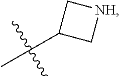 [SMILES: *C1CNC1]

and the cyclobutyl is optionally further substituted by one or more NH₂;
or, R¹ and R² form an N-containing 4-5 membered heterocyclic ring together with the N atom to which R¹ and R² are connected, and the N-containing 4-5 membered heterocyclic ring is optionally further substituted by one or more substituents selected from OH or NH₂; and the N-containing 4-5 membered heterocyclic ring optionally contains 1 or 2 heteroatoms selected from O.

In some embodiments, in the cytotoxic drug, the stereoisomer thereof or the pharmaceutically acceptable salt thereof,
R³ is selected from cyclobutyl or azacyclobutyl, and the cyclobutyl is optionally further substituted by one or more substituents selected from OH or NH₂.

In some embodiments, the cytotoxic drug, the stereoisomer thereof or the pharmaceutically acceptable salt thereof is selected from:

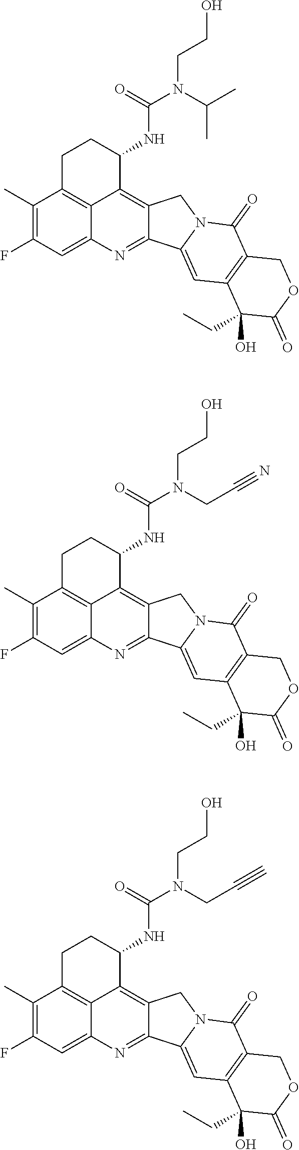 [SMILES: CC[C@@]1(O)C(=O)OCc2c1cc1n(c2=O)Cc2c-1nc1cc(F)c(C)c3c1c2[C@@H](NC(=O)N(CCO)C(C)C)CC3 ; CC[C@@]1(O)C(=O)OCc2c1cc1n(c2=O)Cc2c-1nc1cc(F)c(C)c3c1c2[C@@H](NC(=O)N(CC#N)CCO)CC3 ; C#CCN(CCO)C(=O)N[C@H]1CCc2c(C)c(F)cc3nc4c(c1c23)Cn1c-4cc2c(c1=O)COC(=O)[C@]2(O)CC]

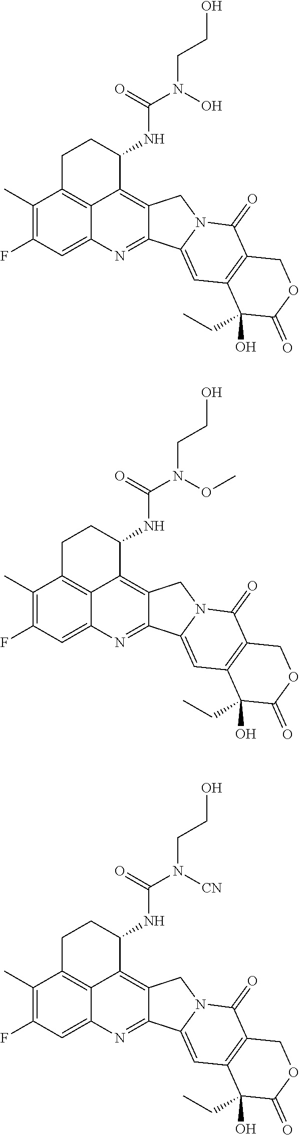 [SMILES: CC[C@@]1(O)C(=O)OCc2c1cc1n(c2=O)Cc2c-1nc1cc(F)c(C)c3c1c2[C@@H](NC(=O)N(O)CCO)CC3 ; CC[C@@]1(O)C(=O)OCc2c1cc1n(c2=O)Cc2c-1nc1cc(F)c(C)c3c1c2[C@@H](NC(=O)N(CCO)OC)CC3 ; CC[C@@]1(O)C(=O)OCc2c1cc1n(c2=O)Cc2c-1nc1cc(F)c(C)c3c1c2[C@@H](NC(=O)N(C#N)CCO)CC3]

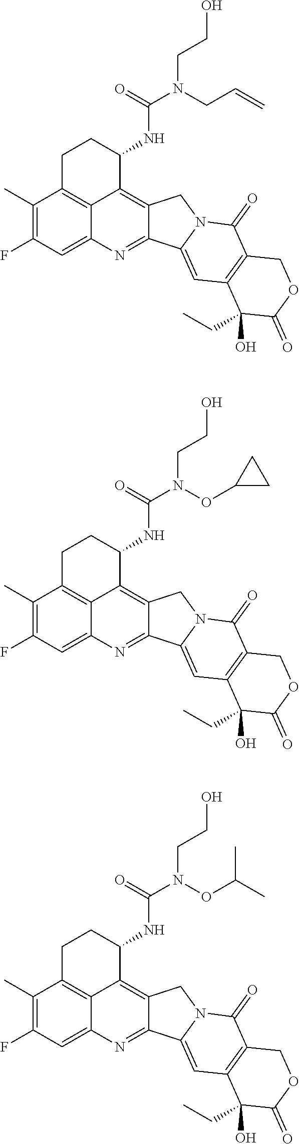 [SMILES: C=CCN(CCO)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(CCO)OC1CC1)CC3 ; CC[C@@]1(O)C(=O)OCc2c1cc1n(c2=O)Cc2c-1nc1cc(F)c(C)c3c1c2[C@@H](NC(=O)N(CCO)OC(C)C)CC3]

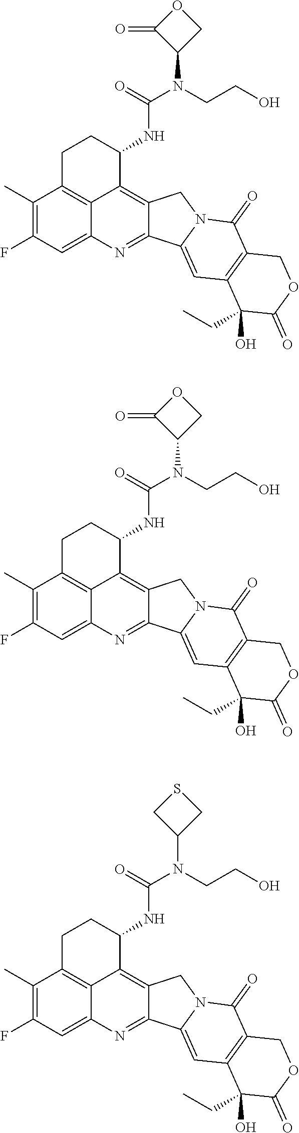 [SMILES: CC[C@@]1(O)C(=O)OCc2c1cc1n(c2=O)Cc2c-1nc1cc(F)c(C)c3c1c2[C@@H](NC(=O)N(CCO)[C@@H]1COC1=O)CC3 ; CC[C@@]1(O)C(=O)OCc2c1cc1n(c2=O)Cc2c-1nc1cc(F)c(C)c3c1c2[C@@H](NC(=O)N(CCO)[C@H]1COC1=O)CC3 ; CC[C@@]1(O)C(=O)OCc2c1cc1n(c2=O)Cc2c-1nc1cc(F)c(C)c3c1c2[C@@H](NC(=O)N(CCO)C1CSC1)CC3]

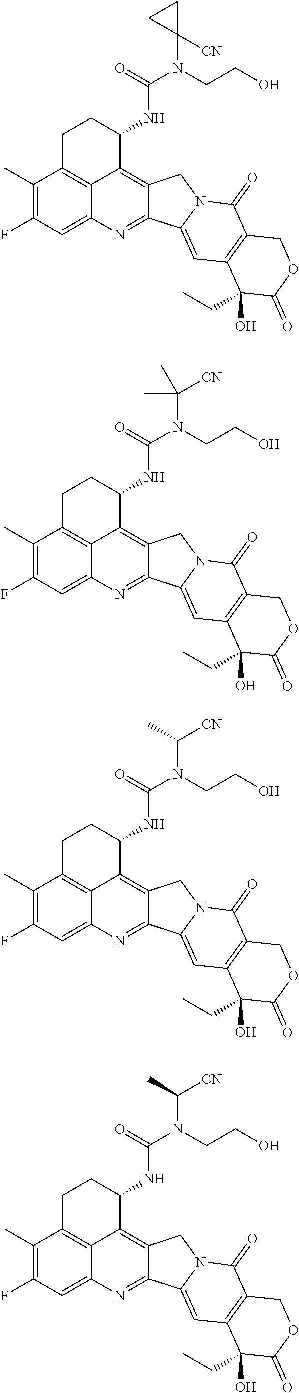 [SMILES: CC[C@@]1(O)C(=O)OCc2c1cc1n(c2=O)Cc2c-1nc1cc(F)c(C)c3c1c2[C@@H](NC(=O)N(CCO)C1(C#N)CC1)CC3 ; CC[C@@]1(O)C(=O)OCc2c1cc1n(c2=O)Cc2c-1nc1cc(F)c(C)c3c1c2[C@@H](NC(=O)N(CCO)C(C)(C)C#N)CC3 ; CC[C@@]1(O)C(=O)OCc2c1cc1n(c2=O)Cc2c-1nc1cc(F)c(C)c3c1c2[C@@H](NC(=O)N(CCO)[C@H](C)C#N)CC3 ; CC[C@@]1(O)C(=O)OCc2c1cc1n(c2=O)Cc2c-1nc1cc(F)c(C)c3c1c2[C@@H](NC(=O)N(CCO)[C@@H](C)C#N)CC3]

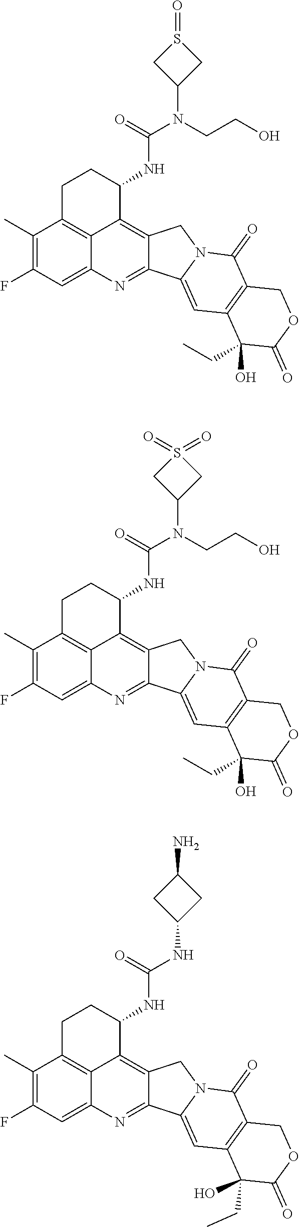 [SMILES: CC[C@@]1(O)C(=O)OCc2c1cc1n(c2=O)Cc2c-1nc1cc(F)c(C)c3c1c2[C@@H](NC(=O)N(CCO)C1CS(=O)C1)CC3 ; CC[C@@]1(O)C(=O)OCc2c1cc1n(c2=O)Cc2c-1nc1cc(F)c(C)c3c1c2[C@@H](NC(=O)N(CCO)C1CS(=O)(=O)C1)CC3 ; CC[C@@]1(O)C(=O)OCc2c1cc1n(c2=O)Cc2c-1nc1cc(F)c(C)c3c1c2[C@@H](NC(=O)N[C@H]1C[C@H](N)C1)CC3]

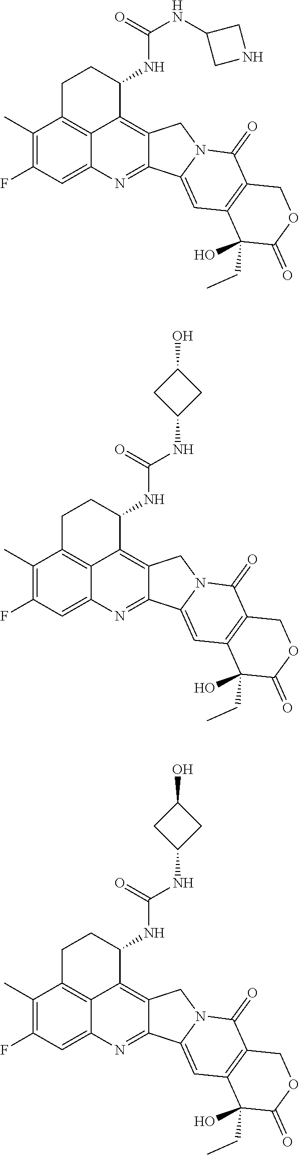 [SMILES: CC[C@@]1(O)C(=O)OCc2c1cc1n(c2=O)Cc2c-1nc1cc(F)c(C)c3c1c2[C@@H](NC(=O)NC1CNC1)CC3 ; CC[C@@]1(O)C(=O)OCc2c1cc1n(c2=O)Cc2c-1nc1cc(F)c(C)c3c1c2[C@@H](NC(=O)N[C@H]1C[C@@H](O)C1)CC3 ; CC[C@@]1(O)C(=O)OCc2c1cc1n(c2=O)Cc2c-1nc1cc(F)c(C)c3c1c2[C@@H](NC(=O)N[C@H]1C[C@H](O)C1)CC3]

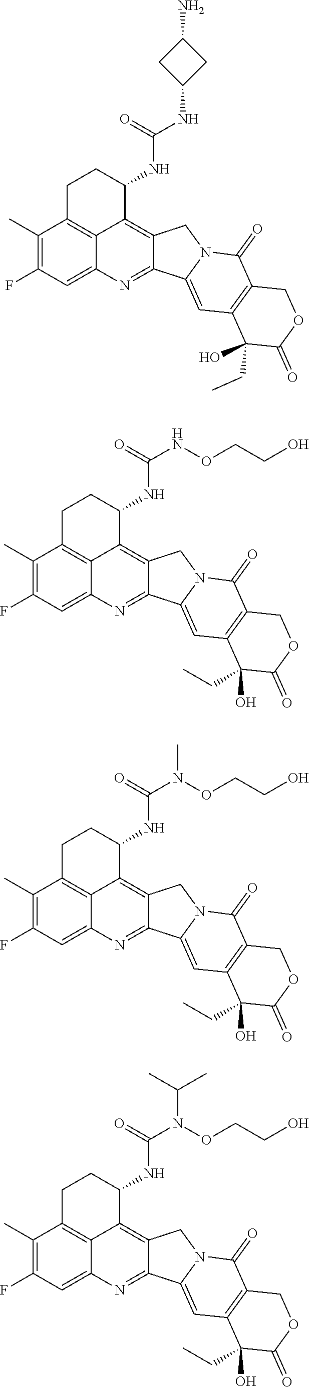 [SMILES: CC[C@@]1(O)C(=O)OCc2c1cc1n(c2=O)Cc2c-1nc1cc(F)c(C)c3c1c2[C@@H](NC(=O)N[C@H]1C[C@@H](N)C1)CC3 ; CC[C@@]1(O)C(=O)OCc2c1cc1n(c2=O)Cc2c-1nc1cc(F)c(C)c3c1c2[C@@H](NC(=O)NOCCO)CC3 ; CC[C@@]1(O)C(=O)OCc2c1cc1n(c2=O)Cc2c-1nc1cc(F)c(C)c3c1c2[C@@H](NC(=O)N(C)OCCO)CC3 ; CC[C@@]1(O)C(=O)OCc2c1cc1n(c2=O)Cc2c-1nc1cc(F)c(C)c3c1c2[C@@H](NC(=O)N(OCCO)C(C)C)CC3]

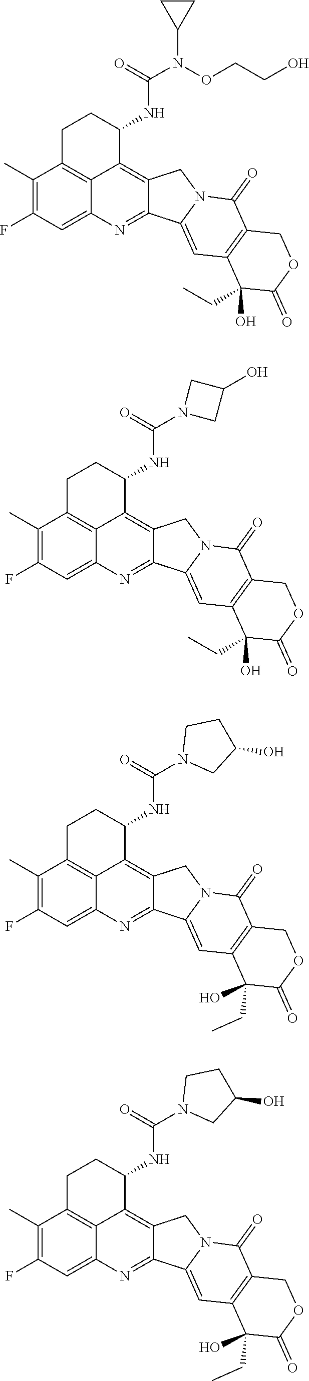

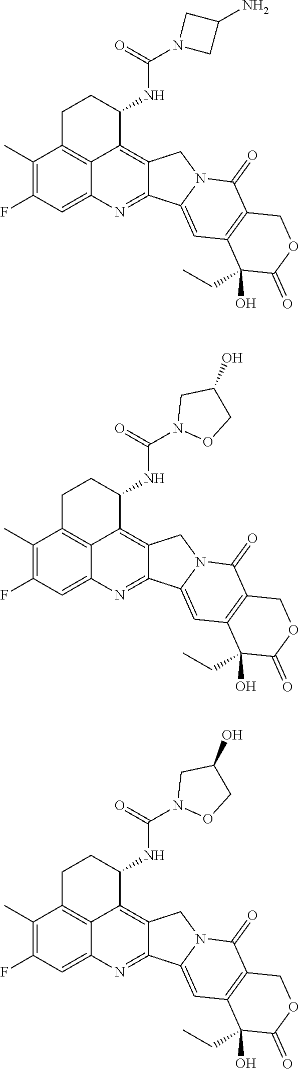 [SMILES: CC[C@@]1(O)C(=O)OCc2c1cc1n(c2=O)Cc2c-1nc1cc(F)c(C)c3c1c2[C@@H](NC(=O)N1CC(N)C1)CC3 ; CC[C@@]1(O)C(=O)OCc2c1cc1n(c2=O)Cc2c-1nc1cc(F)c(C)c3c1c2[C@@H](NC(=O)N1C[C@H](O)CO1)CC3 ; CC[C@@]1(O)C(=O)OCc2c1cc1n(c2=O)Cc2c-1nc1cc(F)c(C)c3c1c2[C@@H](NC(=O)N1C[C@@H](O)CO1)CC3]

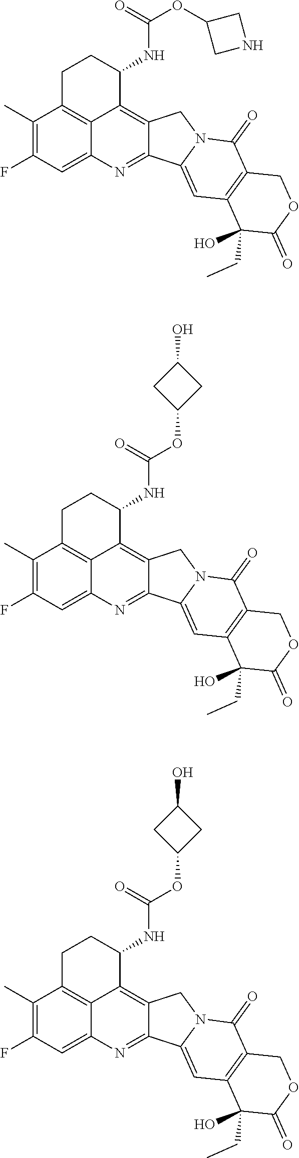 [SMILES: CC[C@@]1(O)C(=O)OCc2c1cc1n(c2=O)Cc2c-1nc1cc(F)c(C)c3c1c2[C@@H](NC(=O)OC1CNC1)CC3 ; CC[C@@]1(O)C(=O)OCc2c1cc1n(c2=O)Cc2c-1nc1cc(F)c(C)c3c1c2[C@@H](NC(=O)O[C@H]1C[C@@H](O)C1)CC3 ; CC[C@@]1(O)C(=O)OCc2c1cc1n(c2=O)Cc2c-1nc1cc(F)c(C)c3c1c2[C@@H](NC(=O)O[C@H]1C[C@H](O)C1)CC3]

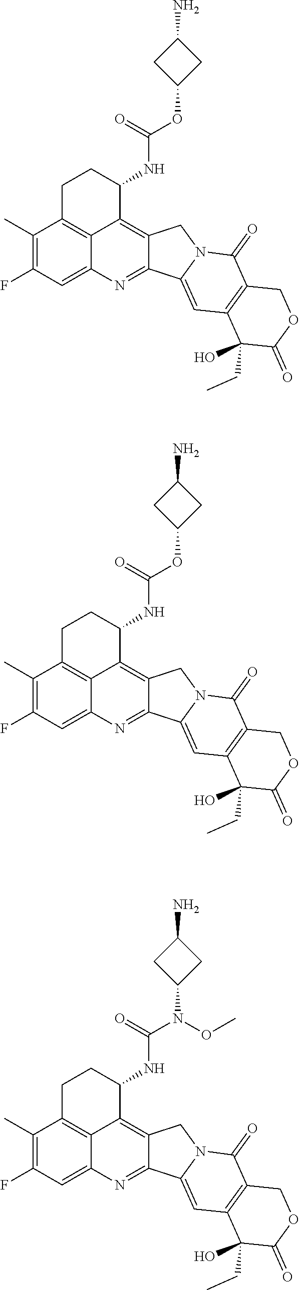 [SMILES: CC[C@@]1(O)C(=O)OCc2c1cc1n(c2=O)Cc2c-1nc1cc(F)c(C)c3c1c2[C@@H](NC(=O)O[C@H]1C[C@@H](N)C1)CC3 ; CC[C@@]1(O)C(=O)OCc2c1cc1n(c2=O)Cc2c-1nc1cc(F)c(C)c3c1c2[C@@H](NC(=O)O[C@H]1C[C@H](N)C1)CC3 ; CC[C@@]1(O)C(=O)OCc2c1cc1n(c2=O)Cc2c-1nc1cc(F)c(C)c3c1c2[C@@H](NC(=O)N(OC)[C@H]1C[C@H](N)C1)CC3]

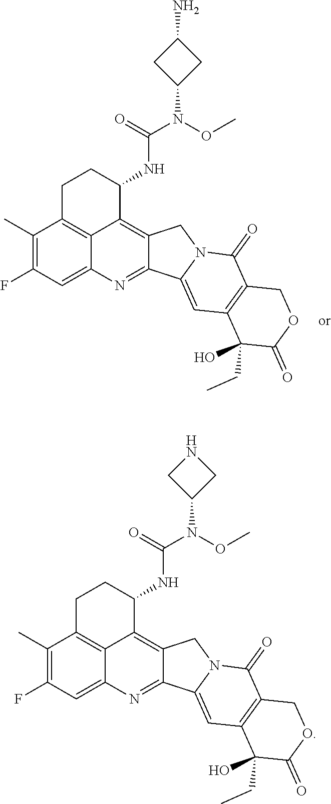

The present disclosure further provides an antibody-drug conjugate, a stereoisomer thereof or a pharmaceutically acceptable salt thereof, which is selected from a structure as shown by formula

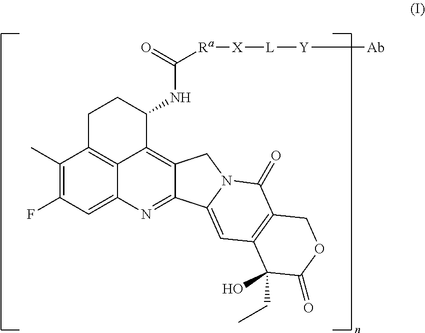

wherein, Ab is an antibody or an antigen-binding fragment;
Y is a linker part linked to the Ab;
L is a peptide residue consisting of 2 to 4 amino groups selected from phenylalanine, glycine, valine, citrulline, alanine, glutamic acid and aspartic acid;
X is a structural fragment connecting R^{a} to L;
R^{a} is a structure of R in the absence of H; or R^{a} is

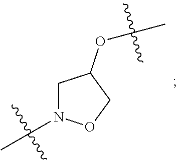 [SMILES: *OC1CON(*)C1]

and the R is as defined in formula (D′); and
n is selected from numerical values of 4-8.

In some embodiments, in the antibody-drug conjugate, the stereoisomer thereof or the pharmaceutically acceptable salt thereof, X is selected from —CH₂—NH or

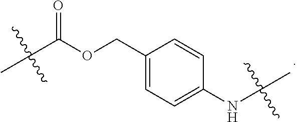 [SMILES: *Nc1ccc(COC(*)=O)cc1]

In some embodiments, in the antibody-drug conjugate, the stereoisomer thereof or the pharmaceutically acceptable salt thereof, L is selected from

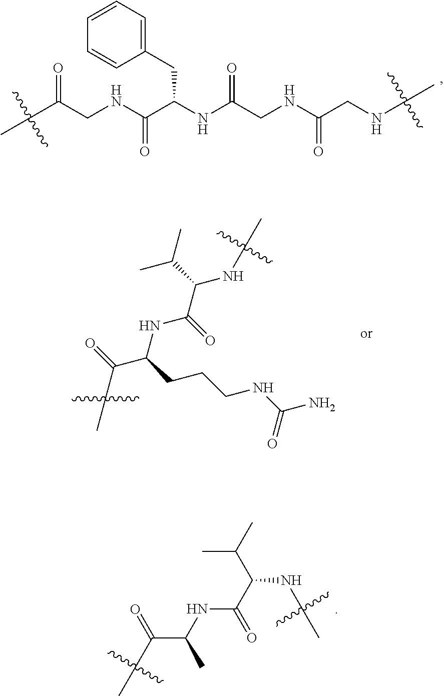 [SMILES: *NCC(=O)NCC(=O)N[C@@H](Cc1ccccc1)C(=O)NCC(*)=O ; *N[C@H](C(=O)N[C@@H](CCCNC(N)=O)C(*)=O)C(C)C ; *N[C@H](C(=O)N[C@@H](C)C(*)=O)C(C)C]

In some embodiments, in the antibody-drug conjugate, the stereoisomer thereof or the pharmaceutically acceptable salt thereof, Y is selected from —CO—Y¹—Y²—S—;
Y² is selected from 5-6 membered heteroaryl or 5-6 membered heterocyclyl;
Y¹ is selected from —(CH₂)ₘ—, —(CH₂)ₘ—NH—SO₂—, —CH₂—N⁺(CH₃)₂—(CH₂)ₘ—NH—SO₂—, —C₂₋₄ alkynylene-NH—SO₂—, —C₂₋₅ alkynylene-, —(CH₂)ₘ—O—C₂₋₄ alkynylene-, —C₃₋₅ cycloalkyl-C₂₋₄ alkynylene-, —(CH₂)ₘ-5-6 membered heterocyclyl-SO₂—, —C₆₋₁₀ aryl-SO₂—NH— and —C₆₋₁₀ aryl-NH—SO₂—; and
m is selected from 0, 1, 2, 3, 4 or 5.

In some embodiments, in the antibody-drug conjugate, the stereoisomer thereof or the pharmaceutically acceptable salt thereof, Y is selected from —CO—Y¹—Y²—S—;
Y¹ is selected from —(CH₂)₅—, —CH₂—NH—SO₂—, —(CH₂)₂—NH—SO₂—, —(CH₂)₃—NH—SO₂—, —CH₂—N⁺(CH₃)₂—(CH₂)₂—NH—SO₂—, —C≡C—CH₂—NH—SO₂—, —(CH₂)₃—C≡C—, —CH₂—O—CH₂—C≡C—,

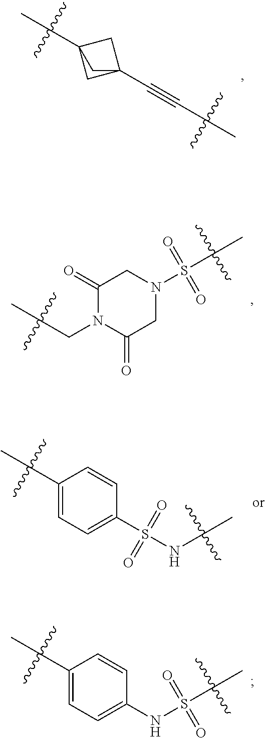 [SMILES: *C#CC12CC(*)(C1)C2 ; *CN1C(=O)CN(S(*)(=O)=O)CC1=O ; *NS(=O)(=O)c1ccc(*)cc1 ; *c1ccc(NS(*)(=O)=O)cc1]

and
Y² is selected from 5-6 membered N-containing heteroaryl or 5-6 membered N-containing heterocyclyl; and preferably, Y² is selected from

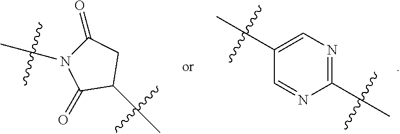 [SMILES: *C1CC(=O)N(*)C1=O ; *c1cnc(*)nc1]

In some embodiments, in the antibody-drug conjugate, the stereoisomer thereof or the pharmaceutically acceptable salt thereof, Y is selected from

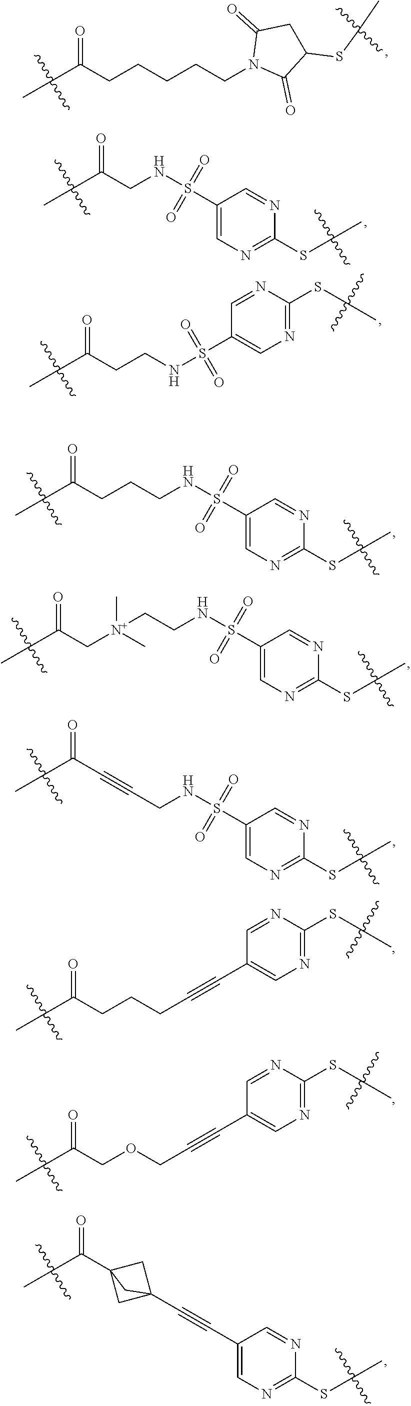

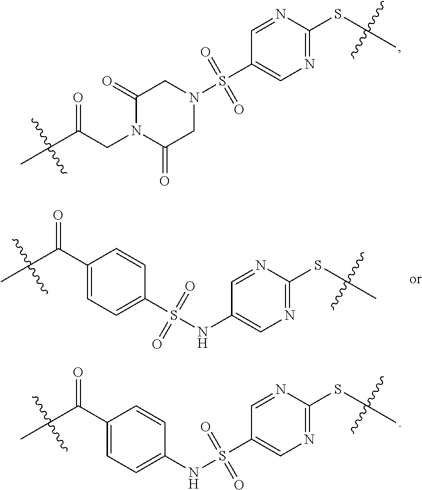 [SMILES: *Sc1ncc(S(=O)(=O)N2CC(=O)N(CC(*)=O)C(=O)C2)cn1 ; *Sc1ncc(NS(=O)(=O)c2ccc(C(*)=O)cc2)cn1 ; *Sc1ncc(S(=O)(=O)Nc2ccc(C(*)=O)cc2)cn1]

In some embodiments, in the antibody-drug conjugate, the stereoisomer thereof or the pharmaceutically acceptable salt thereof, the constitutional unit —X-L-Y— is selected from:

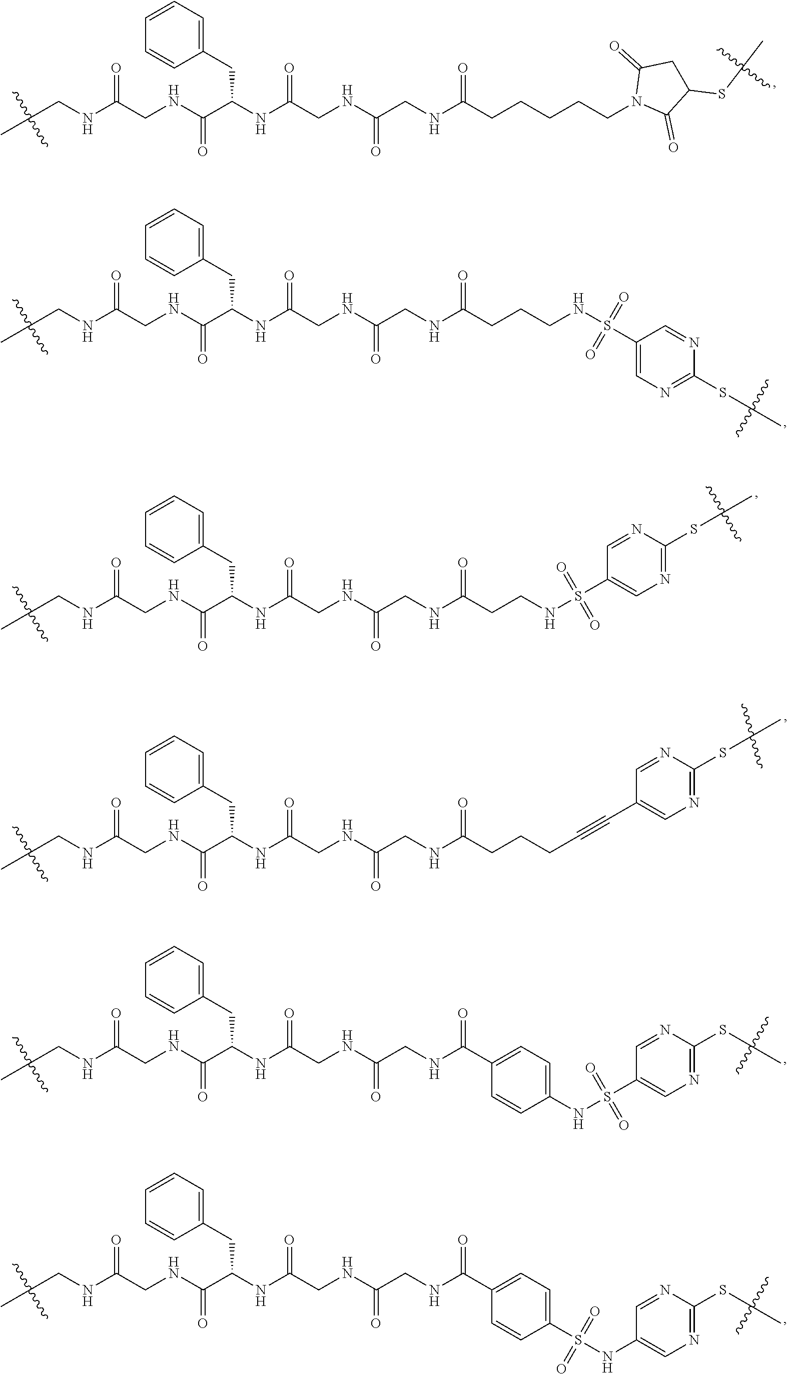

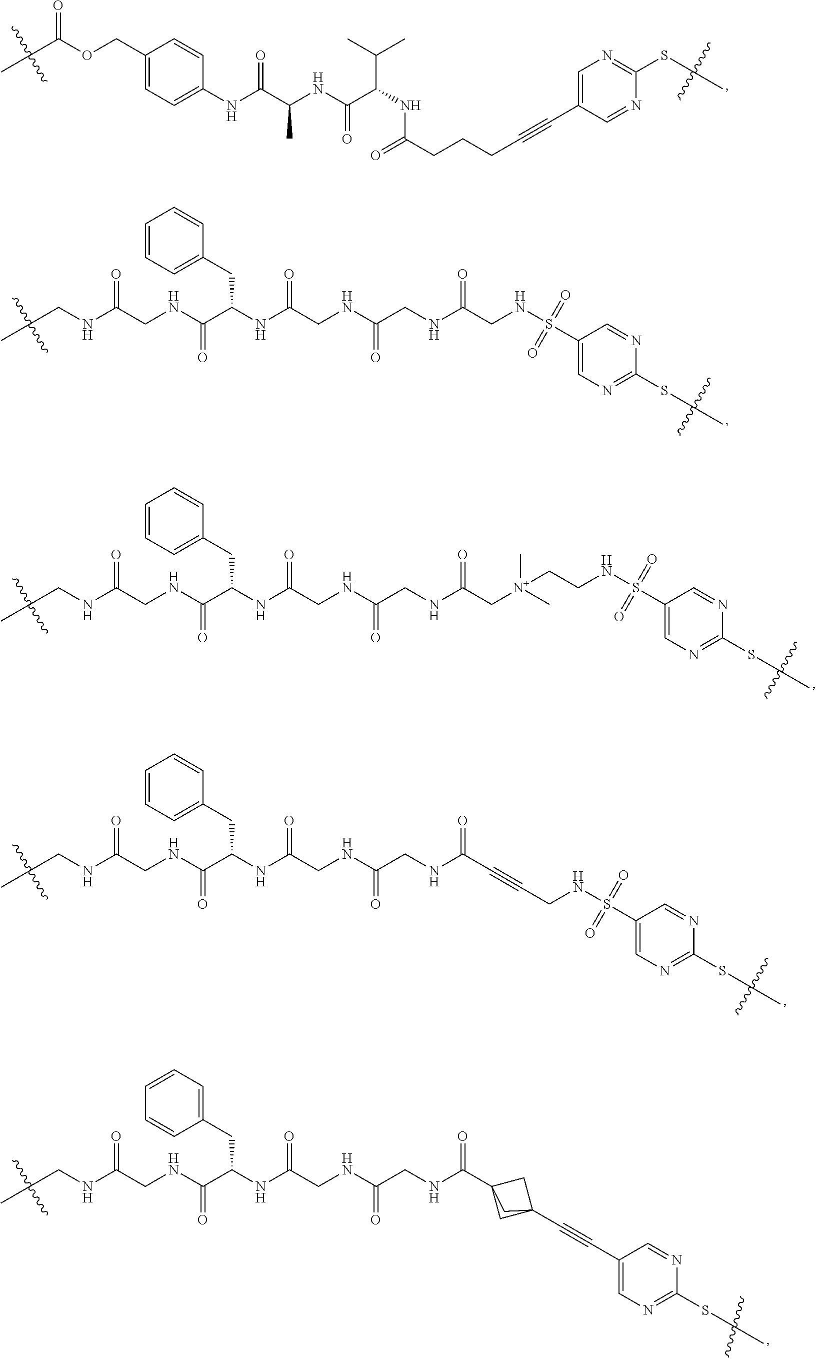

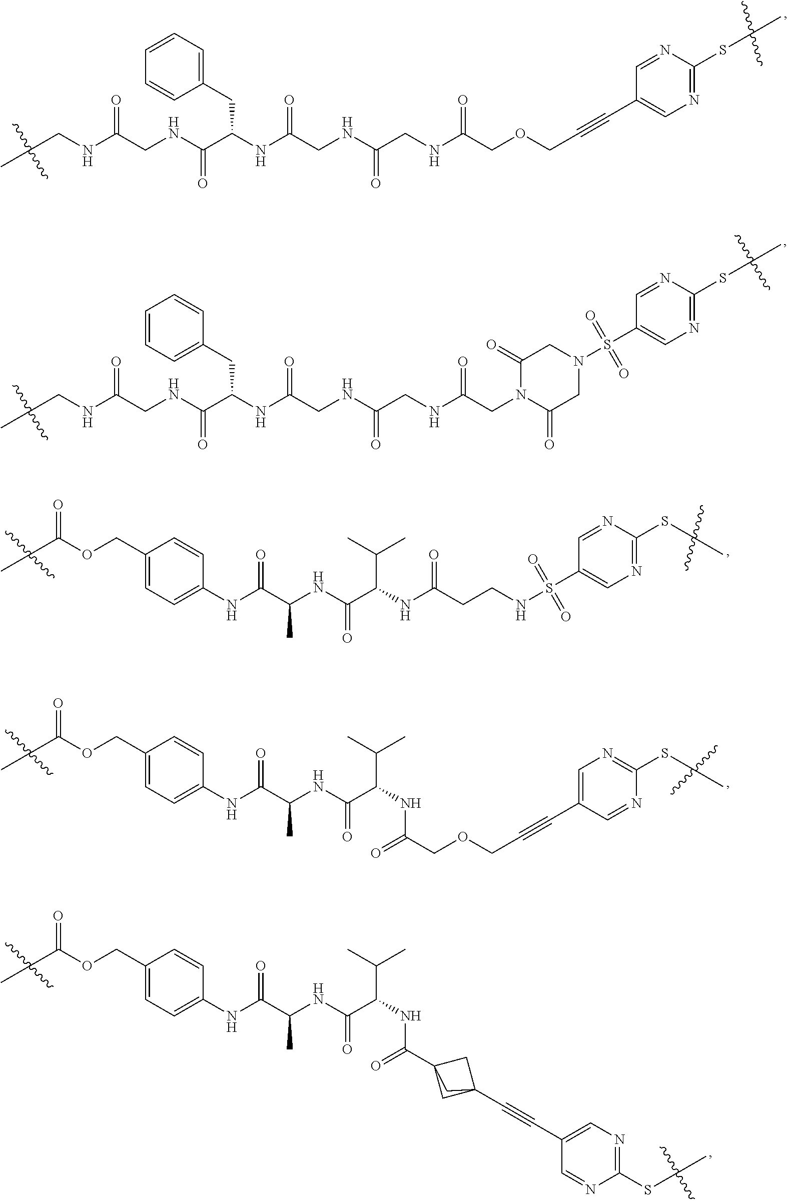

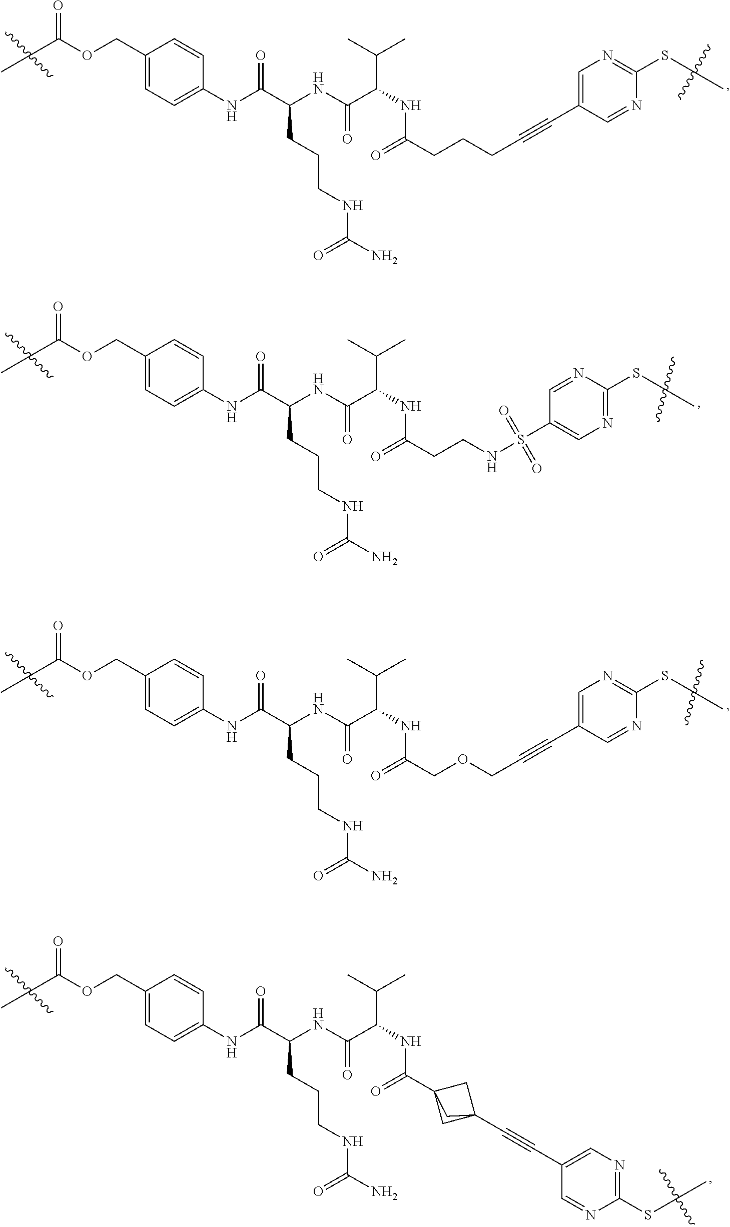

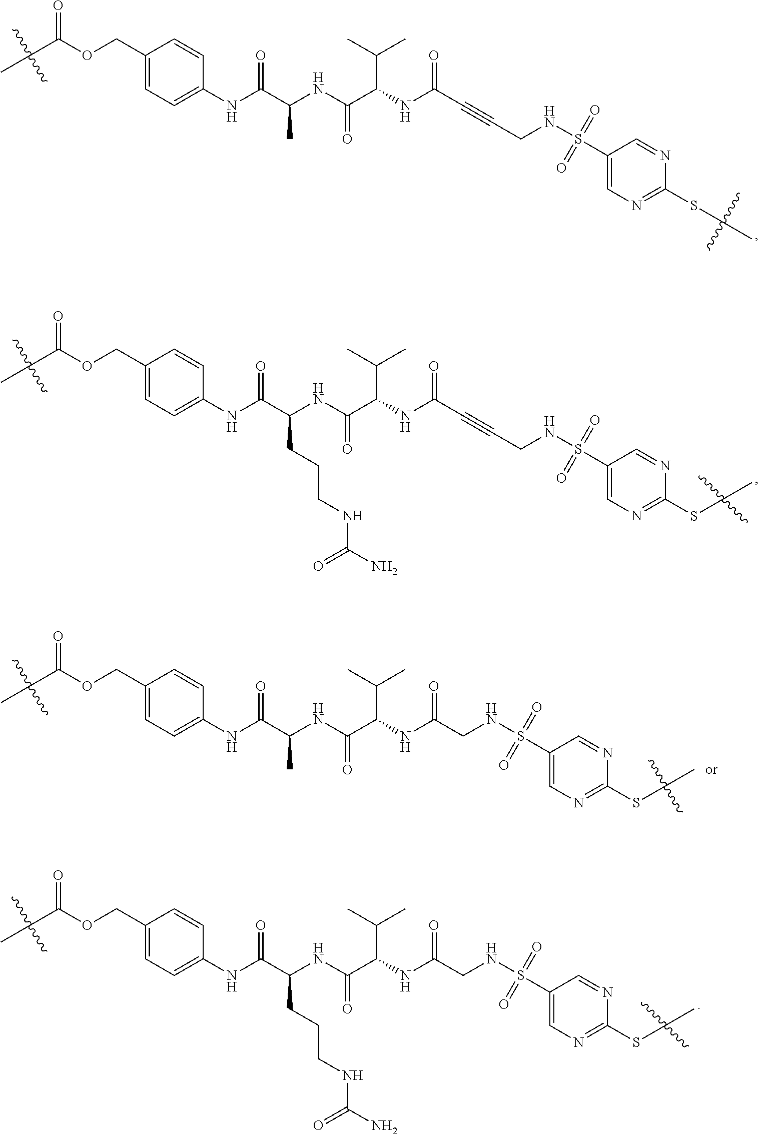

In some embodiments, in the antibody-drug conjugate, the stereoisomer thereof or the pharmaceutically acceptable salt thereof, R^{a} is —NR^{a1}R^{a2}, —OR^{a3} or

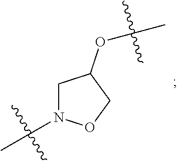 [SMILES: *OC1CON(*)C1]

when R^{a1} is connected to X, R^{a1} is —CH₂CH₂O—, R^{a2} is selected from OH, CN, C₁₋₄ alkyl, C₁₋₄ alkoxy, C₂₋₄ alkenyl, C₂₋₄ alkynyl, —O—C₃₋₆ cycloalkyl, 3-6 membered heterocyclyl or C₃₋₆ cycloalkyl, and the C₁₋₄ alkyl and the C₃₋₆ cycloalkyl are optionally further substituted by CN; and the 3-6 membered heterocyclyl contains 1, 2 or 3 heteroatoms selected from N, O or S;
when R^{a2} is connected to X, R^{a1} is selected from H, C₁₋₄ alkyl or C₃₋₆ cycloalkyl, and R^{a2} is selected from 3-6 membered N-containing heterocyclyl or —O—CH₂CH₂O—;
or, when R^{a2} is connected to X, R^{a1} is C₁₋₄ alkoxy, R^{a2} is selected from C₃₋₆ cycloalkyl or 3-6 membered N-containing heterocyclyl, and the C₃₋₆ cycloalkyl is optionally further substituted by one or more substituents selected from OH or NH₂; and
R^{a3} is selected from C₃₋₆ cycloalkyl or 3-6 membered N-containing heterocyclyl, and the C₃₋₆ cycloalkyl is optionally further substituted by one or more substituents selected from OH or NH₂.

In some embodiments, in the antibody-drug conjugate, the stereoisomer thereof or the pharmaceutically acceptable salt thereof, R^{a} is —NR^{a1}R^{a2}, and when R^{a1} is connected to X, R^{a1} is —CH₂CH₂O—, and R^{a2} is selected from OH, CN, isopropyl, —CH₂CN, —CH₂C≡CH, —CH₂CH≡CH₂, methoxy,

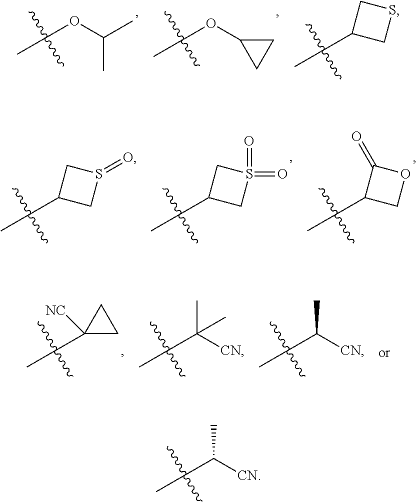

In some embodiments, in the antibody-drug conjugate, the stereoisomer thereof or the pharmaceutically acceptable salt thereof, R^{a} is —NR^{a1}R^{a2}, and when R^{a2} is connected to X:
R^{a1} is H, and R^{a2} is selected from —O—CH₂CH₂O— or

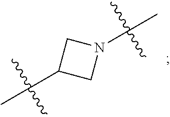 [SMILES: *C1CN(*)C1]

or, when R^{a1} is selected from methyl, isopropyl or cyclopropyl, R^{a2} is —O—CH₂CH₂O—;
or, when R^{a1} is methoxy, R^{a2} is selected from C₄₋₅ cycloalkyl or 4-5 membered N-containing heterocyclyl, and preferably, R^{a2} is selected from

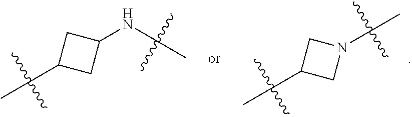 [SMILES: *NC1CC(*)C1 ; *C1CN(*)C1]

In some embodiments, in the antibody-drug conjugate, the stereoisomer thereof or the pharmaceutically acceptable salt thereof, R^{a} is —NR^{a1}R^{a2}, and when R^{a2} is connected to X:
R^{a1} is H, R^{a2} is selected from cyclobutyl, and the cyclobutyl is optionally further substituted by one or more substituents selected from OH or NH₂.

In some embodiments, in the antibody-drug conjugate, the stereoisomer thereof or the pharmaceutically acceptable salt thereof, R^{a} is —OR^{a3}, R^{a3} is selected from cyclobutyl or azacyclobutyl, and the cyclobutyl is optionally further substituted by one or more substituents selected from OH or NH₂.

In some embodiments, in the antibody-drug conjugate, the stereoisomer thereof or the pharmaceutically acceptable salt thereof, R^{a} is selected from.

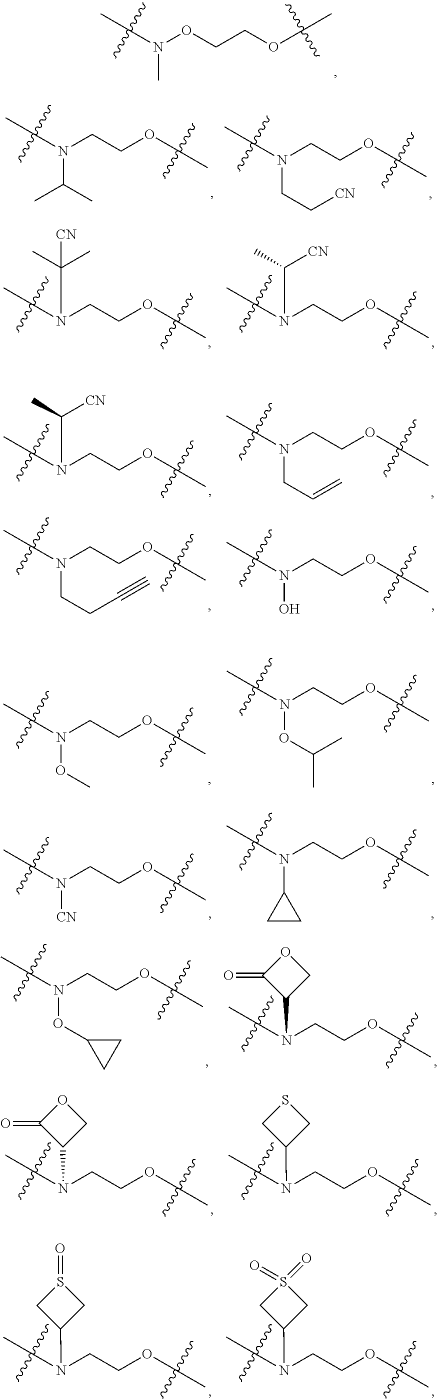

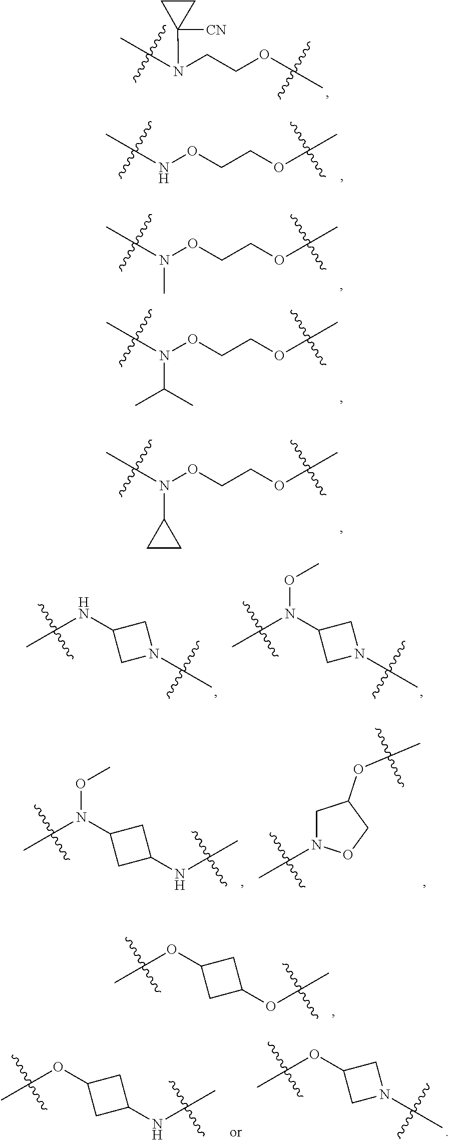

In some embodiments, R^{a} is selected from

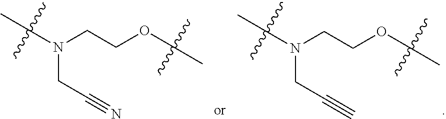

In some embodiments, the antibody-drug conjugate, the stereoisomer thereof or the pharmaceutically acceptable salt thereof is selected from:

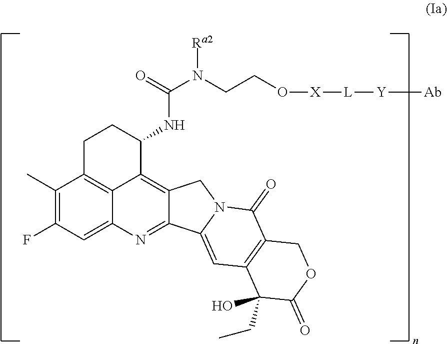

wherein, Ab, Y, L, X, R^{a2} and n are as defined in formula (I).

In another aspect, the present disclosure further provides a cytotoxic drug-linker, a stereoisomer thereof or a pharmaceutically acceptable salt thereof, wherein the cytotoxic drug-linker has a structure as shown by formula D-X-L-Y′, wherein Y′ is a structure before Y is linked to the Ab, D is a structure of a compound as shown by formula (D′) in the absence of H, X and Y are as defined in formula (I), and the compound as shown by the formula (D′) is as described above;
preferably, Y′ is selected from:

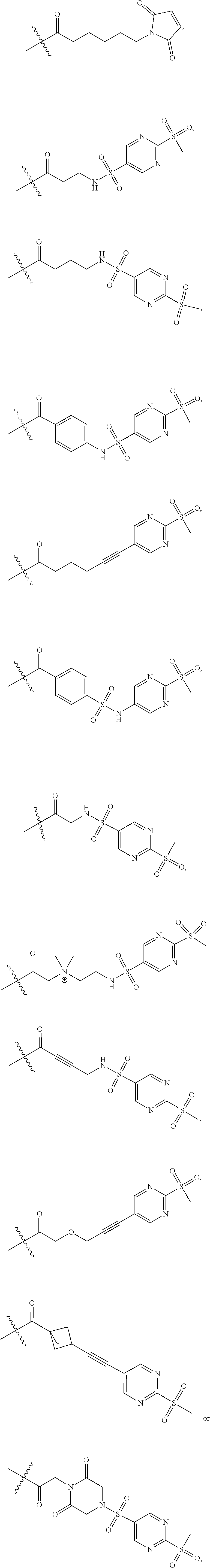

preferably, L is selected from

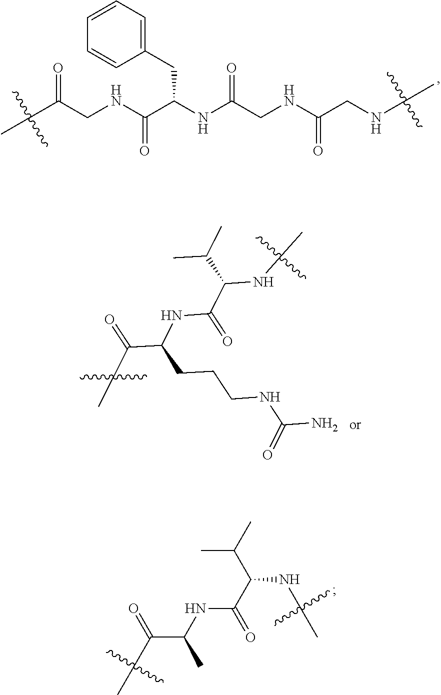 [SMILES: *NCC(=O)NCC(=O)N[C@@H](Cc1ccccc1)C(=O)NCC(*)=O ; *N[C@H](C(=O)N[C@@H](CCCNC(N)=O)C(*)=O)C(C)C ; *N[C@H](C(=O)N[C@@H](C)C(*)=O)C(C)C]

preferably, X is selected from —CH₂—NH— or

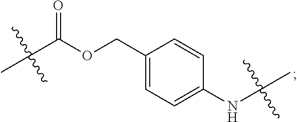 [SMILES: *Nc1ccc(COC(*)=O)cc1]

preferably, the constitutional unit —X-L-Y′ is selected from:

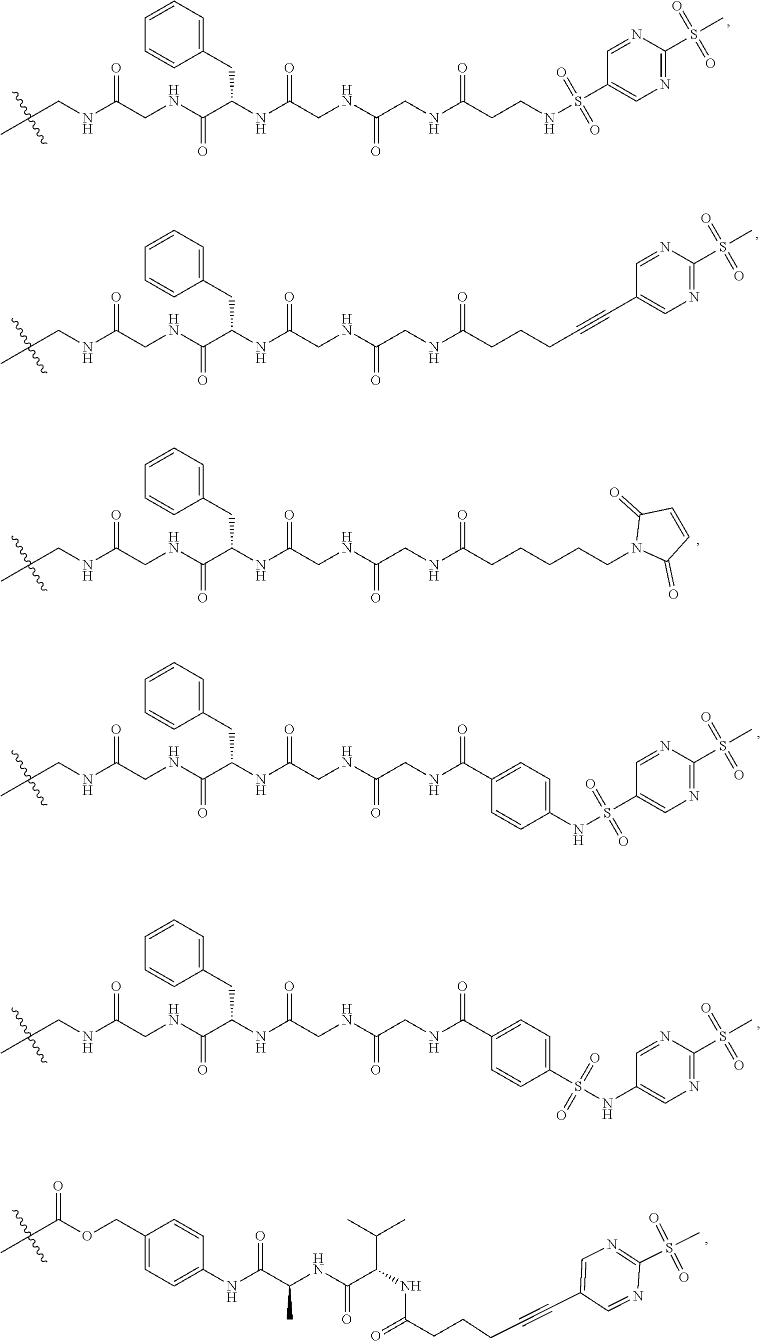

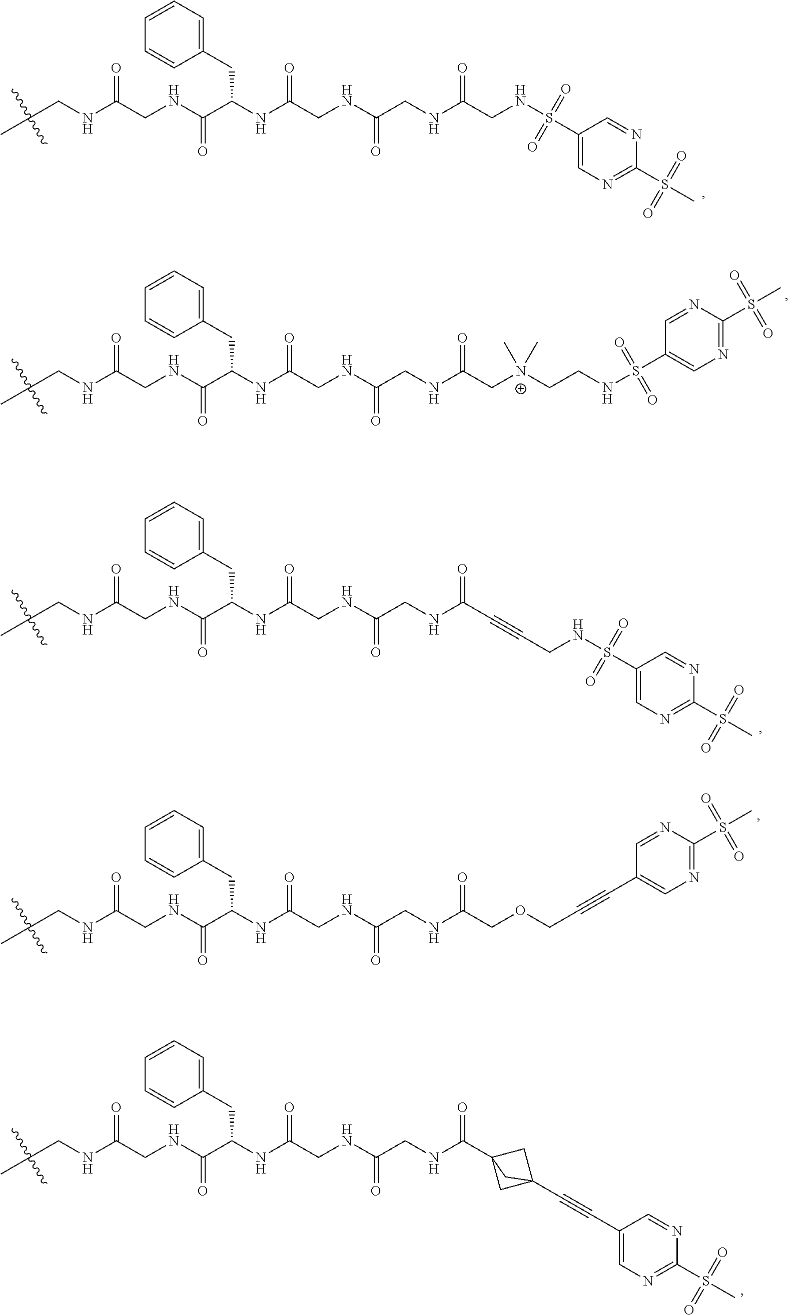 [SMILES: *CNC(=O)CNC(=O)[C@H](Cc1ccccc1)NC(=O)CNC(=O)CNC(=O)CNS(=O)(=O)c1cnc(S(C)(=O)=O)nc1 ; *CNC(=O)CNC(=O)[C@H](Cc1ccccc1)NC(=O)CNC(=O)CNC(=O)C[N+](C)(C)CCNS(=O)(=O)c1cnc(S(C)(=O)=O)nc1 ; *CNC(=O)CNC(=O)[C@H](Cc1ccccc1)NC(=O)CNC(=O)CNC(=O)C#CCNS(=O)(=O)c1cnc(S(C)(=O)=O)nc1 ; *CNC(=O)CNC(=O)[C@H](Cc1ccccc1)NC(=O)CNC(=O)CNC(=O)COCC#Cc1cnc(S(C)(=O)=O)nc1 ; *CNC(=O)CNC(=O)[C@H](Cc1ccccc1)NC(=O)CNC(=O)CNC(=O)C12CC(C#Cc3cnc(S(C)(=O)=O)nc3)(C1)C2]

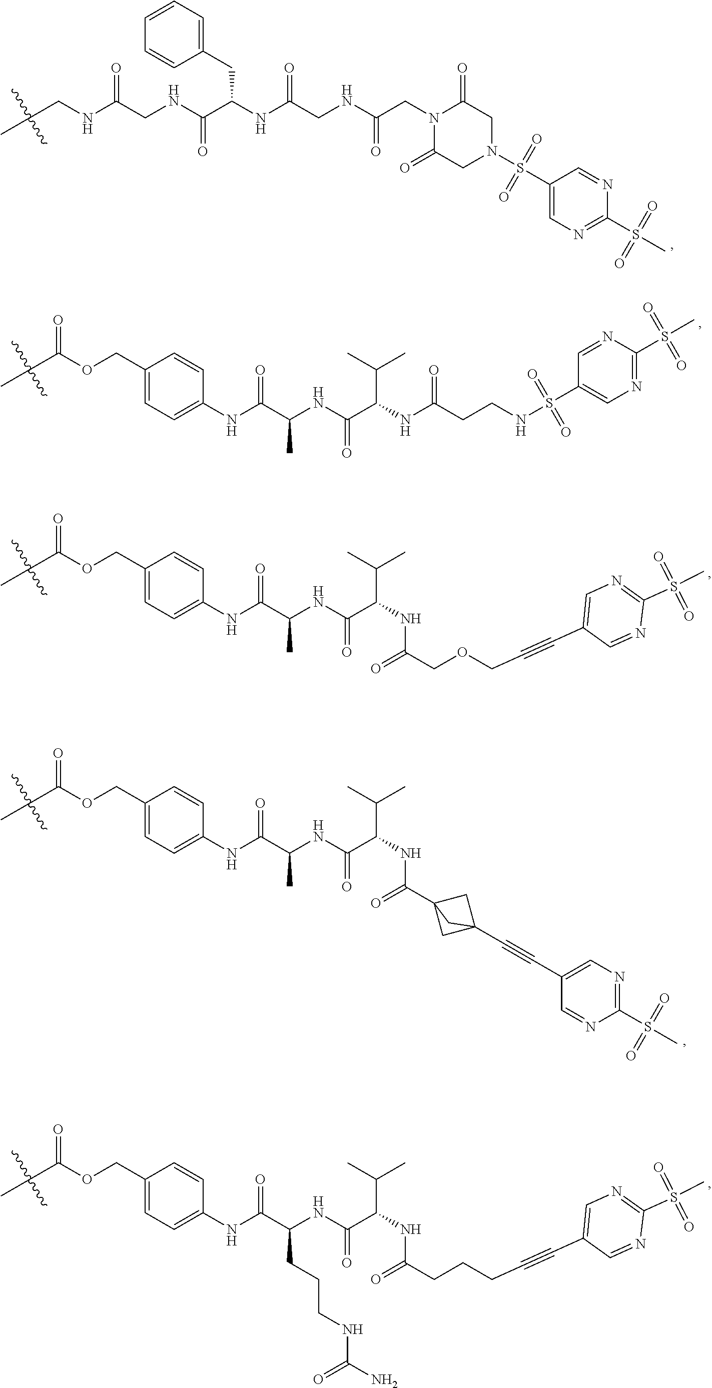 [SMILES: *CNC(=O)CNC(=O)[C@H](Cc1ccccc1)NC(=O)CNC(=O)CN1C(=O)CN(S(=O)(=O)c2cnc(S(C)(=O)=O)nc2)CC1=O ; *C(=O)OCc1ccc(NC(=O)[C@H](C)NC(=O)[C@@H](NC(=O)CCNS(=O)(=O)c2cnc(S(C)(=O)=O)nc2)C(C)C)cc1 ; *C(=O)OCc1ccc(NC(=O)[C@H](C)NC(=O)[C@@H](NC(=O)COCC#Cc2cnc(S(C)(=O)=O)nc2)C(C)C)cc1 ; *C(=O)OCc1ccc(NC(=O)[C@H](C)NC(=O)[C@@H](NC(=O)C23CC(C#Cc4cnc(S(C)(=O)=O)nc4)(C2)C3)C(C)C)cc1 ; *C(=O)OCc1ccc(NC(=O)[C@H](CCCNC(N)=O)NC(=O)[C@@H](NC(=O)CCCC#Cc2cnc(S(C)(=O)=O)nc2)C(C)C)cc1]

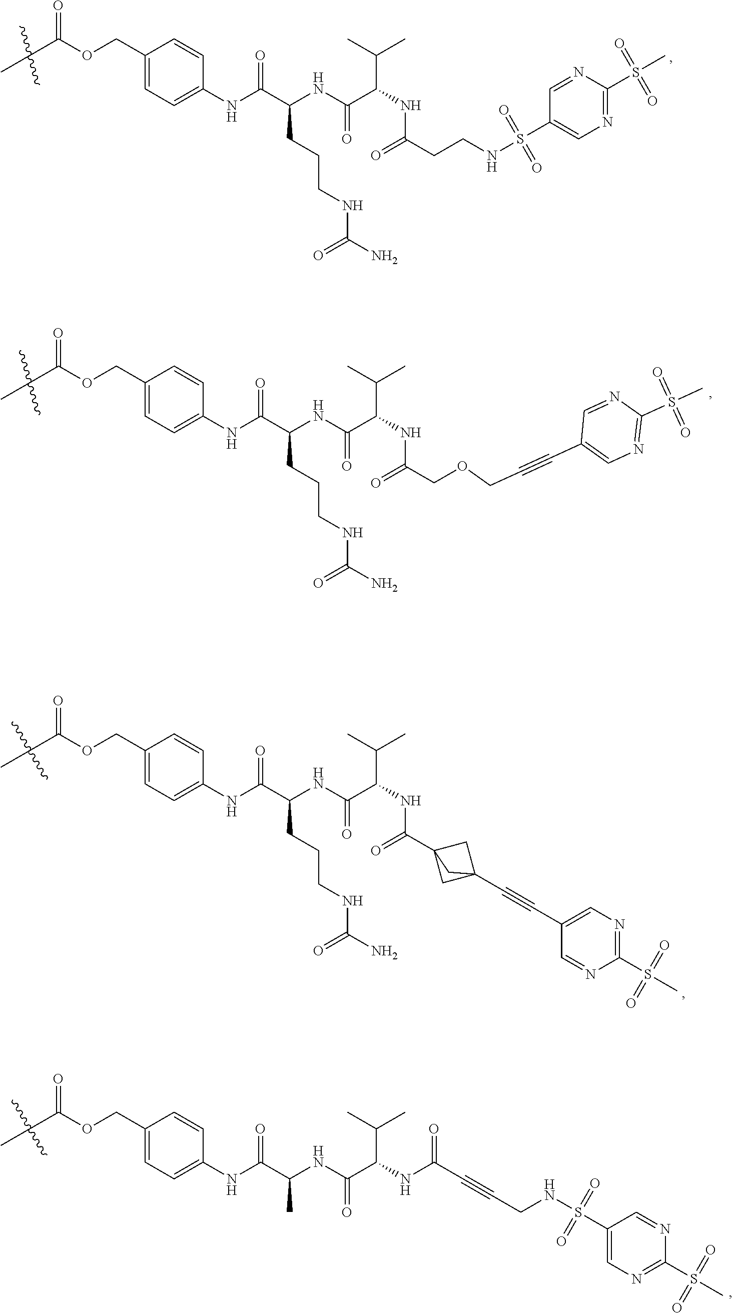 [SMILES: *C(=O)OCc1ccc(NC(=O)[C@H](CCCNC(N)=O)NC(=O)[C@@H](NC(=O)CCNS(=O)(=O)c2cnc(S(C)(=O)=O)nc2)C(C)C)cc1 ; *C(=O)OCc1ccc(NC(=O)[C@H](CCCNC(N)=O)NC(=O)[C@@H](NC(=O)COCC#Cc2cnc(S(C)(=O)=O)nc2)C(C)C)cc1 ; *C(=O)OCc1ccc(NC(=O)[C@H](CCCNC(N)=O)NC(=O)[C@@H](NC(=O)C23CC(C#Cc4cnc(S(C)(=O)=O)nc4)(C2)C3)C(C)C)cc1 ; *C(=O)OCc1ccc(NC(=O)[C@H](C)NC(=O)[C@@H](NC(=O)C#CCNS(=O)(=O)c2cnc(S(C)(=O)=O)nc2)C(C)C)cc1]

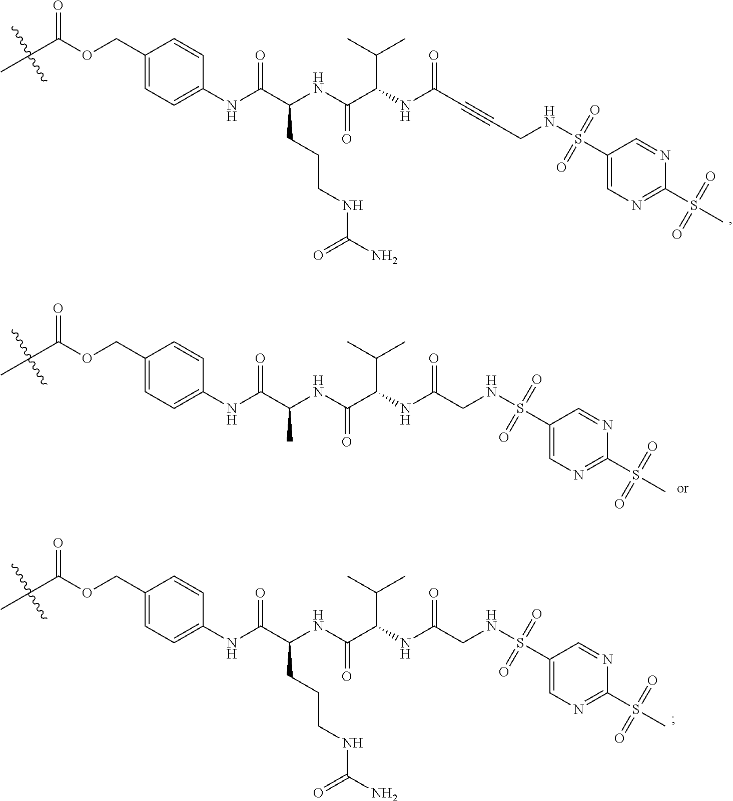 [SMILES: *C(=O)OCc1ccc(NC(=O)[C@H](CCCNC(N)=O)NC(=O)[C@@H](NC(=O)C#CCNS(=O)(=O)c2cnc(S(C)(=O)=O)nc2)C(C)C)cc1 ; *C(=O)OCc1ccc(NC(=O)[C@H](C)NC(=O)[C@@H](NC(=O)CNS(=O)(=O)c2cnc(S(C)(=O)=O)nc2)C(C)C)cc1 ; *C(=O)OCc1ccc(NC(=O)[C@H](CCCNC(N)=O)NC(=O)[C@@H](NC(=O)CNS(=O)(=O)c2cnc(S(C)(=O)=O)nc2)C(C)C)cc1]

and
preferably, D is selected from:

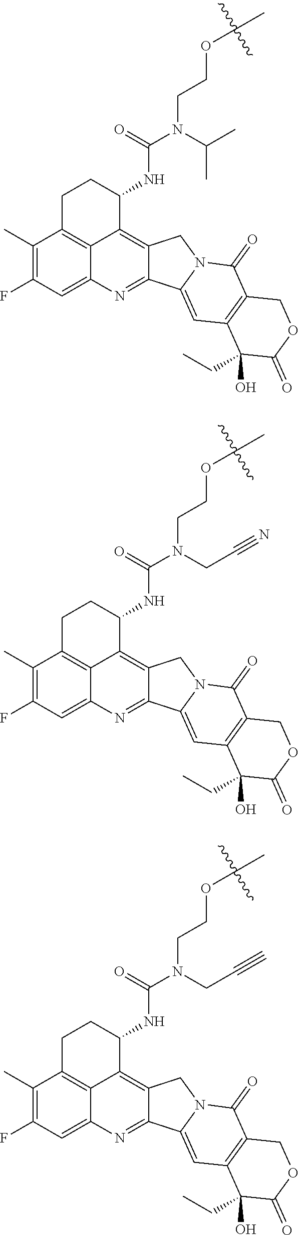 [SMILES: *OCCN(C(=O)N[C@H]1CCc2c(C)c(F)cc3nc4c(c1c23)Cn1c-4cc2c(c1=O)COC(=O)[C@]2(O)CC)C(C)C ; *OCCN(CC#N)C(=O)N[C@H]1CCc2c(C)c(F)cc3nc4c(c1c23)Cn1c-4cc2c(c1=O)COC(=O)[C@]2(O)CC ; *OCCN(CC#C)C(=O)N[C@H]1CCc2c(C)c(F)cc3nc4c(c1c23)Cn1c-4cc2c(c1=O)COC(=O)[C@]2(O)CC]

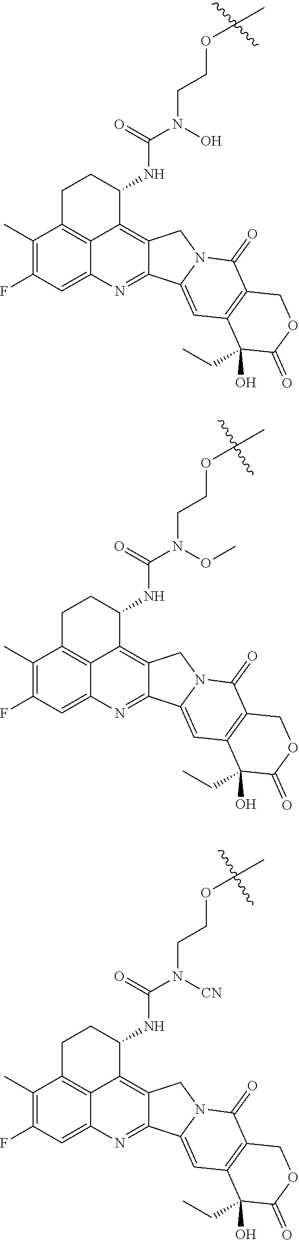 [SMILES: *OCCN(O)C(=O)N[C@H]1CCc2c(C)c(F)cc3nc4c(c1c23)Cn1c-4cc2c(c1=O)COC(=O)[C@]2(O)CC ; *OCCN(OC)C(=O)N[C@H]1CCc2c(C)c(F)cc3nc4c(c1c23)Cn1c-4cc2c(c1=O)COC(=O)[C@]2(O)CC ; *OCCN(C#N)C(=O)N[C@H]1CCc2c(C)c(F)cc3nc4c(c1c23)Cn1c-4cc2c(c1=O)COC(=O)[C@]2(O)CC]

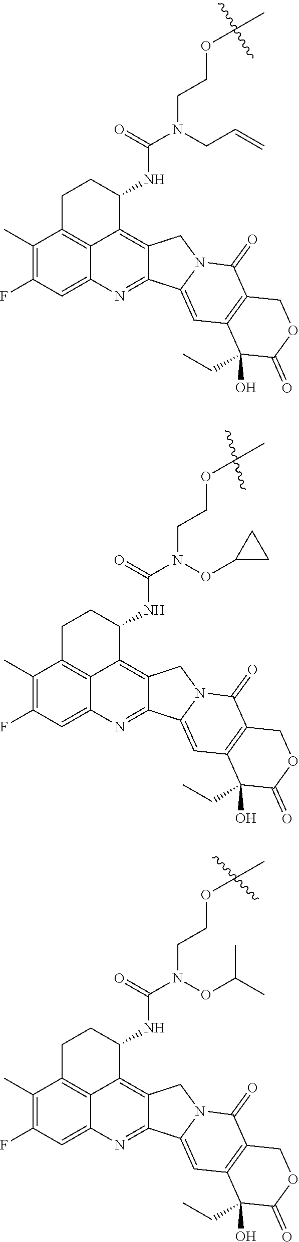 [SMILES: *OCCN(CC=C)C(=O)N[C@H]1CCc2c(C)c(F)cc3nc4c(c1c23)Cn1c-4cc2c(c1=O)COC(=O)[C@]2(O)CC ; *OCCN(OC1CC1)C(=O)N[C@H]1CCc2c(C)c(F)cc3nc4c(c1c23)Cn1c-4cc2c(c1=O)COC(=O)[C@]2(O)CC ; *OCCN(OC(C)C)C(=O)N[C@H]1CCc2c(C)c(F)cc3nc4c(c1c23)Cn1c-4cc2c(c1=O)COC(=O)[C@]2(O)CC]

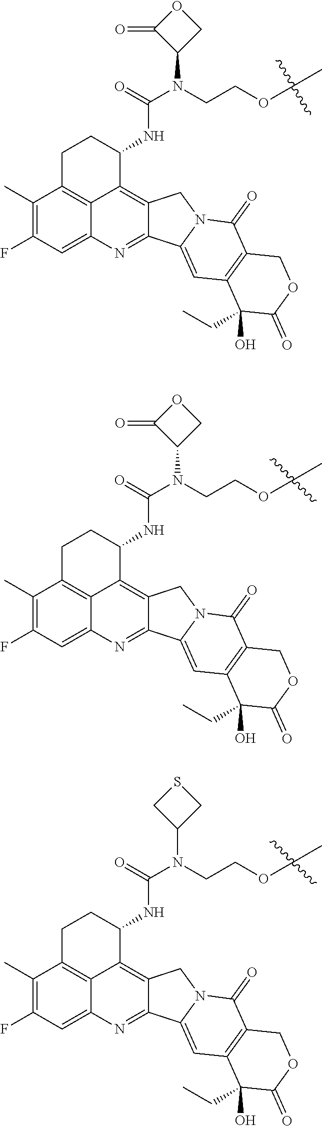 [SMILES: *OCCN(C(=O)N[C@H]1CCc2c(C)c(F)cc3nc4c(c1c23)Cn1c-4cc2c(c1=O)COC(=O)[C@]2(O)CC)[C@@H]1COC1=O ; *OCCN(C(=O)N[C@H]1CCc2c(C)c(F)cc3nc4c(c1c23)Cn1c-4cc2c(c1=O)COC(=O)[C@]2(O)CC)[C@H]1COC1=O ; *OCCN(C(=O)N[C@H]1CCc2c(C)c(F)cc3nc4c(c1c23)Cn1c-4cc2c(c1=O)COC(=O)[C@]2(O)CC)C1CSC1]

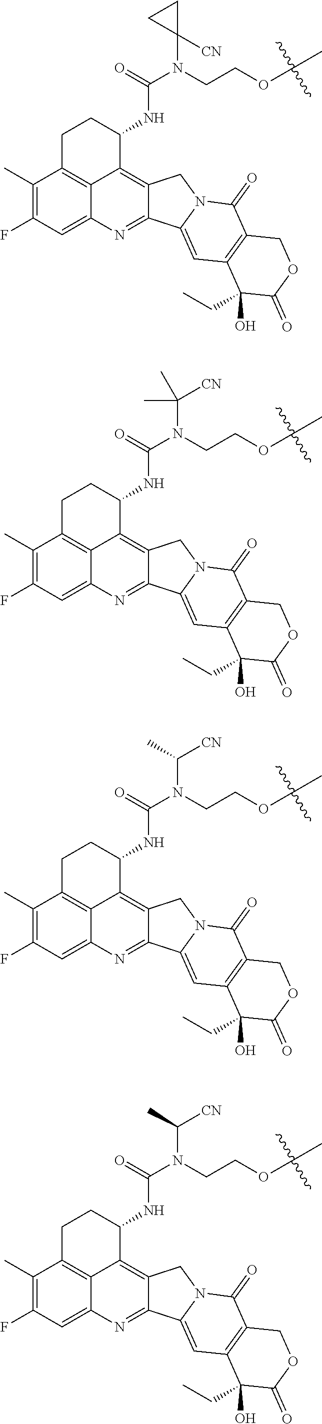 [SMILES: *OCCN(C(=O)N[C@H]1CCc2c(C)c(F)cc3nc4c(c1c23)Cn1c-4cc2c(c1=O)COC(=O)[C@]2(O)CC)C1(C#N)CC1 ; *OCCN(C(=O)N[C@H]1CCc2c(C)c(F)cc3nc4c(c1c23)Cn1c-4cc2c(c1=O)COC(=O)[C@]2(O)CC)C(C)(C)C#N ; *OCCN(C(=O)N[C@H]1CCc2c(C)c(F)cc3nc4c(c1c23)Cn1c-4cc2c(c1=O)COC(=O)[C@]2(O)CC)[C@H](C)C#N ; *OCCN(C(=O)N[C@H]1CCc2c(C)c(F)cc3nc4c(c1c23)Cn1c-4cc2c(c1=O)COC(=O)[C@]2(O)CC)[C@@H](C)C#N]

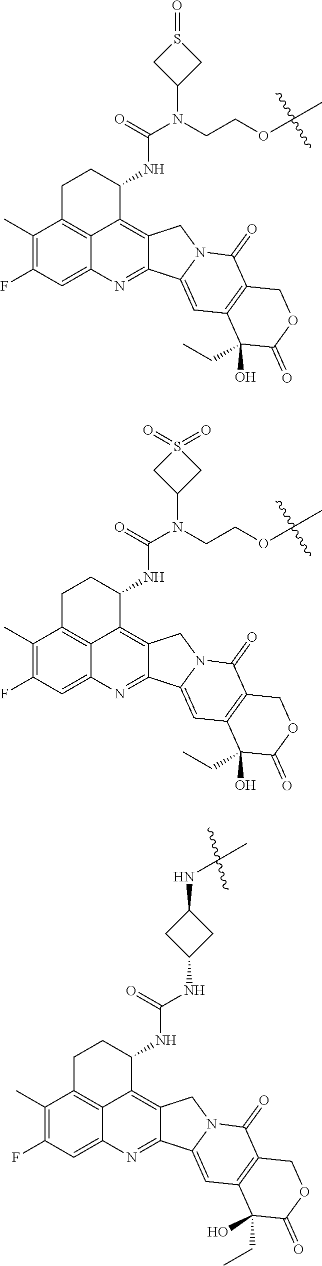 [SMILES: *OCCN(C(=O)N[C@H]1CCc2c(C)c(F)cc3nc4c(c1c23)Cn1c-4cc2c(c1=O)COC(=O)[C@]2(O)CC)C1CS(=O)C1 ; *OCCN(C(=O)N[C@H]1CCc2c(C)c(F)cc3nc4c(c1c23)Cn1c-4cc2c(c1=O)COC(=O)[C@]2(O)CC)C1CS(=O)(=O)C1 ; *N[C@H]1C[C@H](NC(=O)N[C@H]2CCc3c(C)c(F)cc4nc5c(c2c34)Cn2c-5cc3c(c2=O)COC(=O)[C@]3(O)CC)C1]

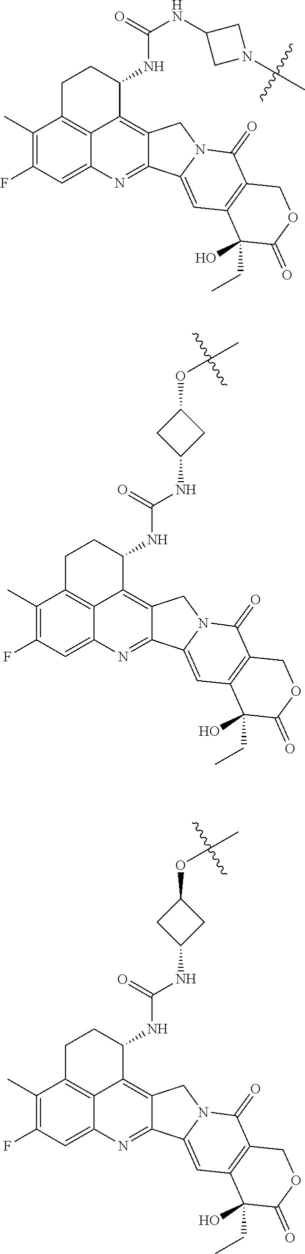 [SMILES: *N1CC(NC(=O)N[C@H]2CCc3c(C)c(F)cc4nc5c(c2c34)Cn2c-5cc3c(c2=O)COC(=O)[C@]3(O)CC)C1 ; *O[C@H]1C[C@@H](NC(=O)N[C@H]2CCc3c(C)c(F)cc4nc5c(c2c34)Cn2c-5cc3c(c2=O)COC(=O)[C@]3(O)CC)C1 ; *O[C@H]1C[C@H](NC(=O)N[C@H]2CCc3c(C)c(F)cc4nc5c(c2c34)Cn2c-5cc3c(c2=O)COC(=O)[C@]3(O)CC)C1]

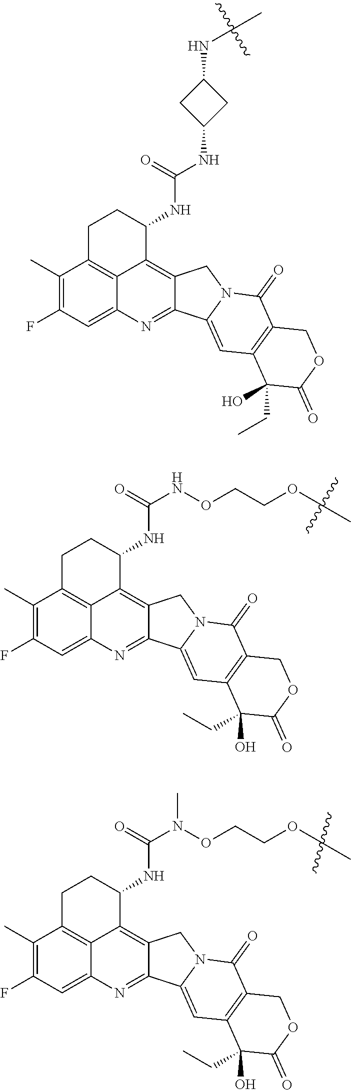 [SMILES: *N[C@H]1C[C@@H](NC(=O)N[C@H]2CCc3c(C)c(F)cc4nc5c(c2c34)Cn2c-5cc3c(c2=O)COC(=O)[C@]3(O)CC)C1 ; *OCCONC(=O)N[C@H]1CCc2c(C)c(F)cc3nc4c(c1c23)Cn1c-4cc2c(c1=O)COC(=O)[C@]2(O)CC ; *OCCON(C)C(=O)N[C@H]1CCc2c(C)c(F)cc3nc4c(c1c23)Cn1c-4cc2c(c1=O)COC(=O)[C@]2(O)CC]

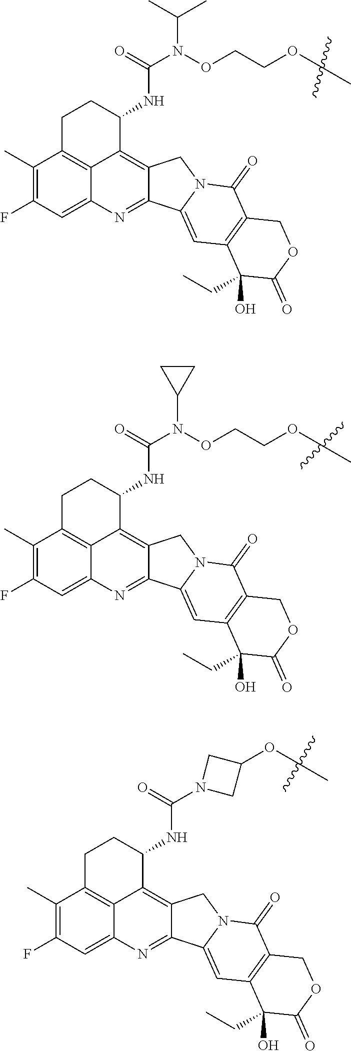 [SMILES: *OCCON(C(=O)N[C@H]1CCc2c(C)c(F)cc3nc4c(c1c23)Cn1c-4cc2c(c1=O)COC(=O)[C@]2(O)CC)C(C)C ; *OCCON(C(=O)N[C@H]1CCc2c(C)c(F)cc3nc4c(c1c23)Cn1c-4cc2c(c1=O)COC(=O)[C@]2(O)CC)C1CC1 ; *OC1CN(C(=O)N[C@H]2CCc3c(C)c(F)cc4nc5c(c2c34)Cn2c-5cc3c(c2=O)COC(=O)[C@]3(O)CC)C1]

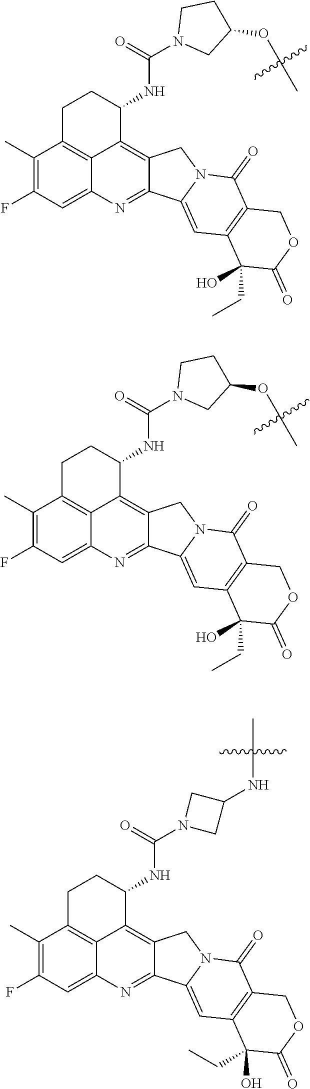

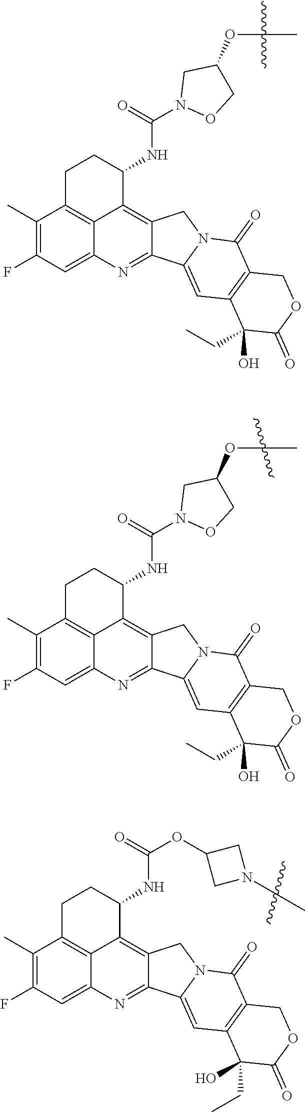 [SMILES: *O[C@@H]1CON(C(=O)N[C@H]2CCc3c(C)c(F)cc4nc5c(c2c34)Cn2c-5cc3c(c2=O)COC(=O)[C@]3(O)CC)C1 ; *O[C@H]1CON(C(=O)N[C@H]2CCc3c(C)c(F)cc4nc5c(c2c34)Cn2c-5cc3c(c2=O)COC(=O)[C@]3(O)CC)C1 ; *N1CC(OC(=O)N[C@H]2CCc3c(C)c(F)cc4nc5c(c2c34)Cn2c-5cc3c(c2=O)COC(=O)[C@]3(O)CC)C1]

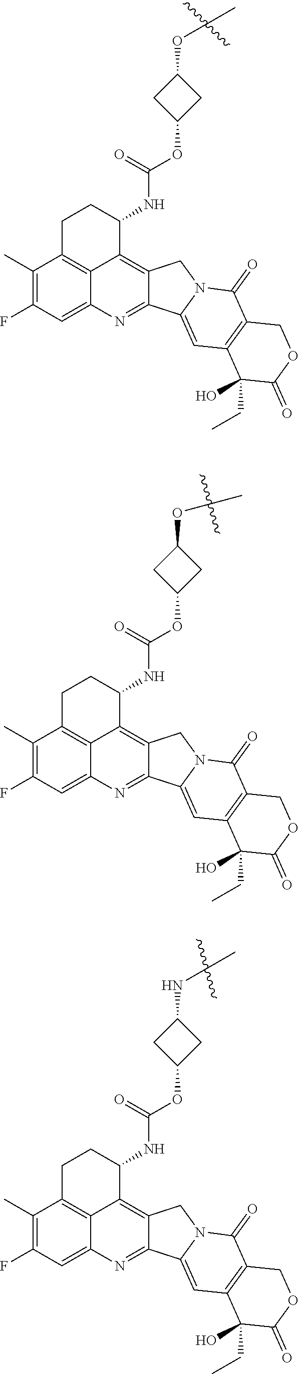 [SMILES: *O[C@H]1C[C@@H](OC(=O)N[C@H]2CCc3c(C)c(F)cc4nc5c(c2c34)Cn2c-5cc3c(c2=O)COC(=O)[C@]3(O)CC)C1 ; *O[C@H]1C[C@H](OC(=O)N[C@H]2CCc3c(C)c(F)cc4nc5c(c2c34)Cn2c-5cc3c(c2=O)COC(=O)[C@]3(O)CC)C1 ; *N[C@H]1C[C@@H](OC(=O)N[C@H]2CCc3c(C)c(F)cc4nc5c(c2c34)Cn2c-5cc3c(c2=O)COC(=O)[C@]3(O)CC)C1]

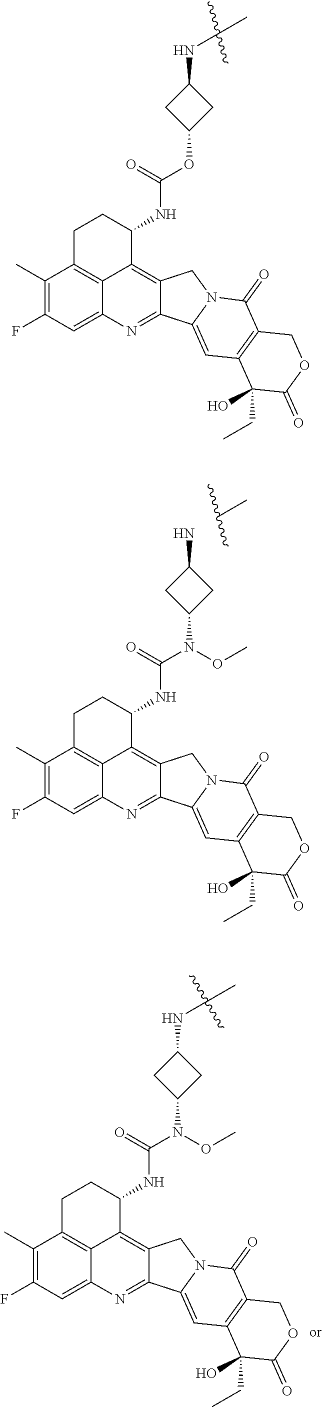

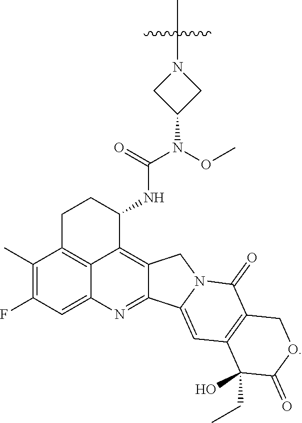

In some embodiments, the cytotoxic drug-linker, the stereoisomer thereof or the pharmaceutically acceptable salt thereof is selected from:

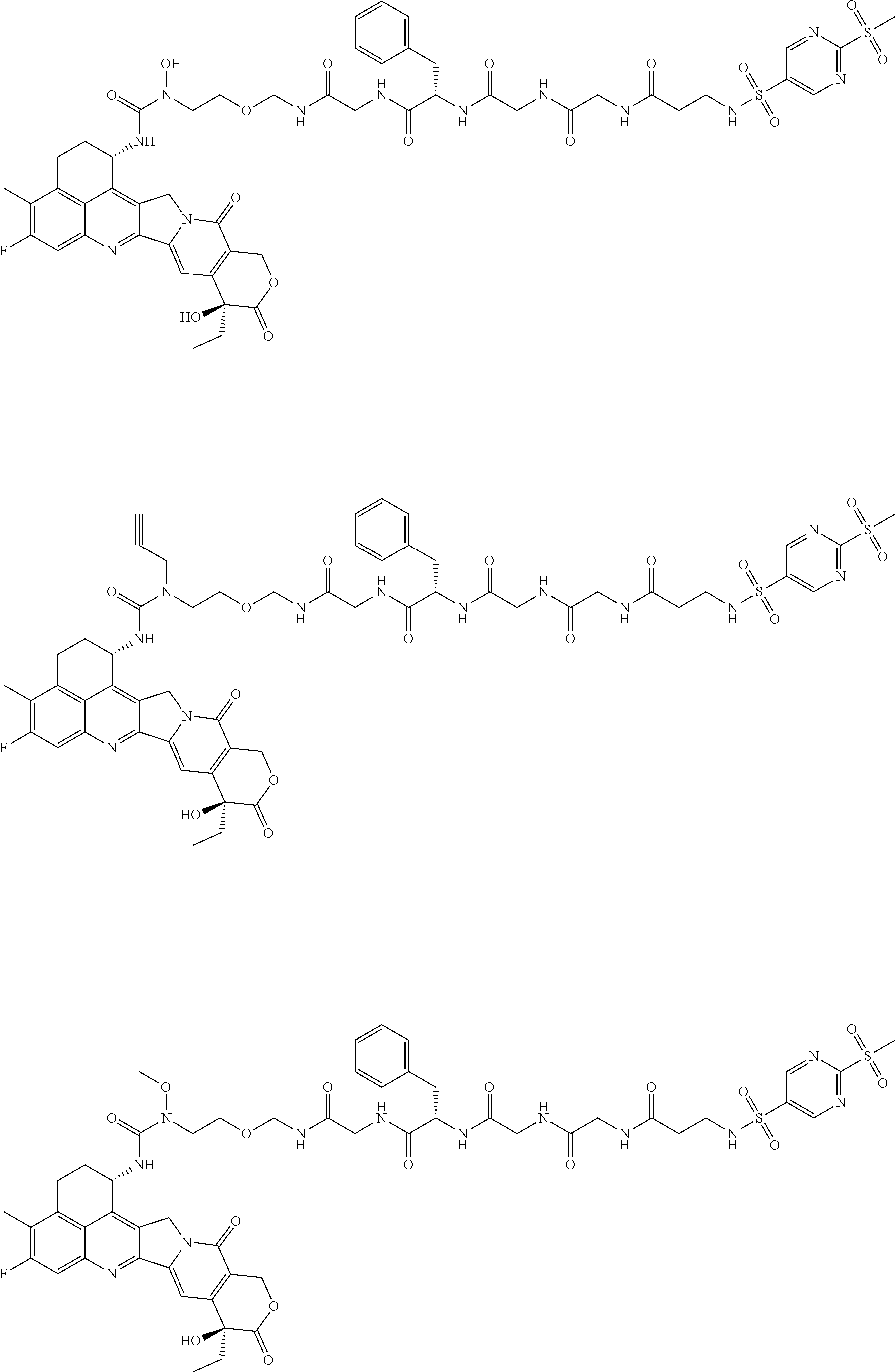 [SMILES: CC[C@@]1(O)C(=O)OCc2c1cc1n(c2=O)Cc2c-1nc1cc(F)c(C)c3c1c2[C@@H](NC(=O)N(O)CCOCNC(=O)CNC(=O)[C@H](Cc1ccccc1)NC(=O)CNC(=O)CNC(=O)CCNS(=O)(=O)c1cnc(S(C)(=O)=O)nc1)CC3 ; C#CCN(CCOCNC(=O)CNC(=O)[C@H](Cc1ccccc1)NC(=O)CNC(=O)CNC(=O)CCNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCNS(=O)(=O)c1cnc(S(C)(=O)=O)nc1)OC)CC3]

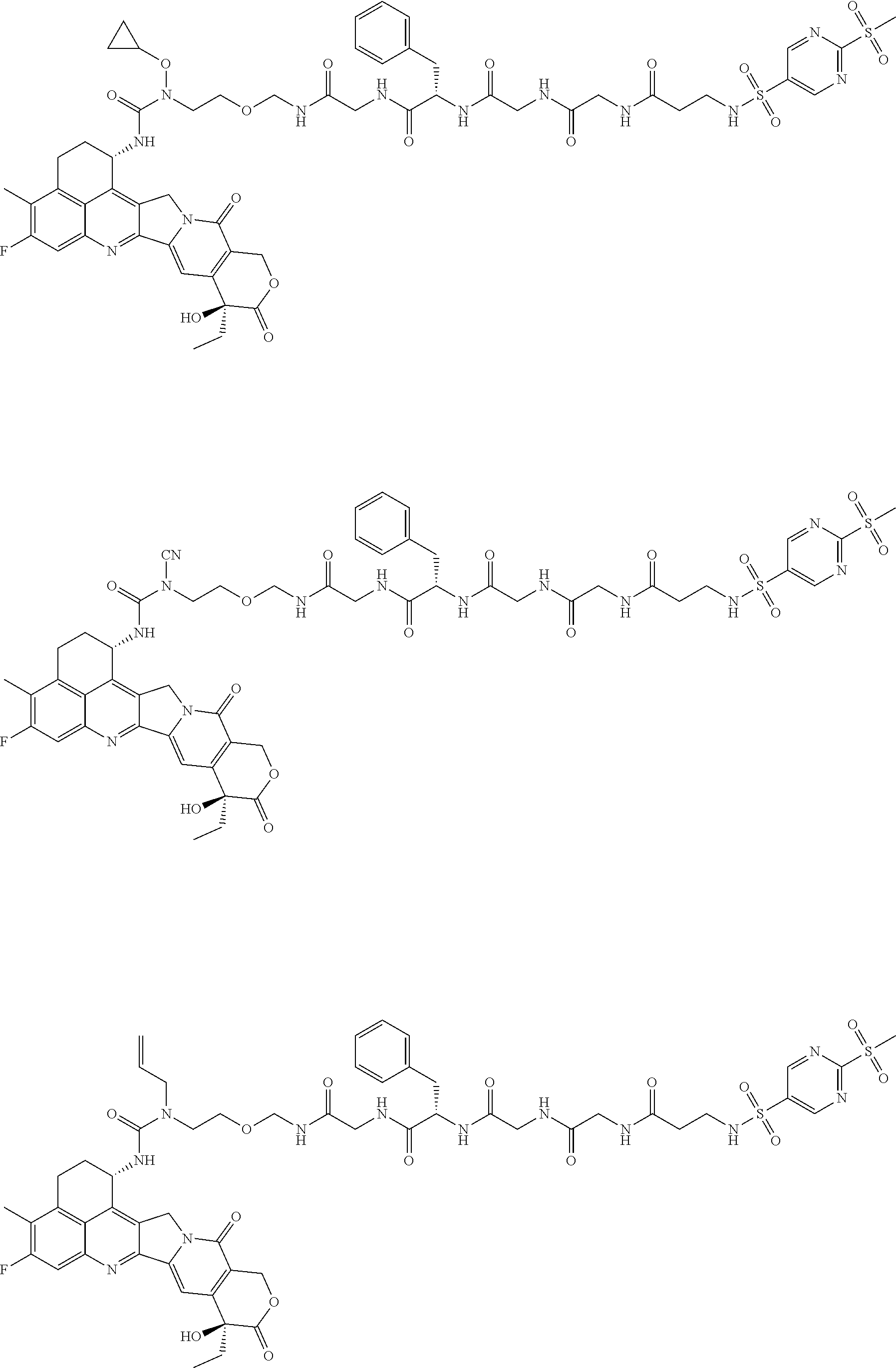 [SMILES: CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCNS(=O)(=O)c1cnc(S(C)(=O)=O)nc1)OC1CC1)CC3 ; CC[C@@]1(O)C(=O)OCc2c1cc1n(c2=O)Cc2c-1nc1cc(F)c(C)c3c1c2[C@@H](NC(=O)N(C#N)CCOCNC(=O)CNC(=O)[C@H](Cc1ccccc1)NC(=O)CNC(=O)CNC(=O)CCNS(=O)(=O)c1cnc(S(C)(=O)=O)nc1)CC3 ; C=CCN(CCOCNC(=O)CNC(=O)[C@H](Cc1ccccc1)NC(=O)CNC(=O)CNC(=O)CCNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

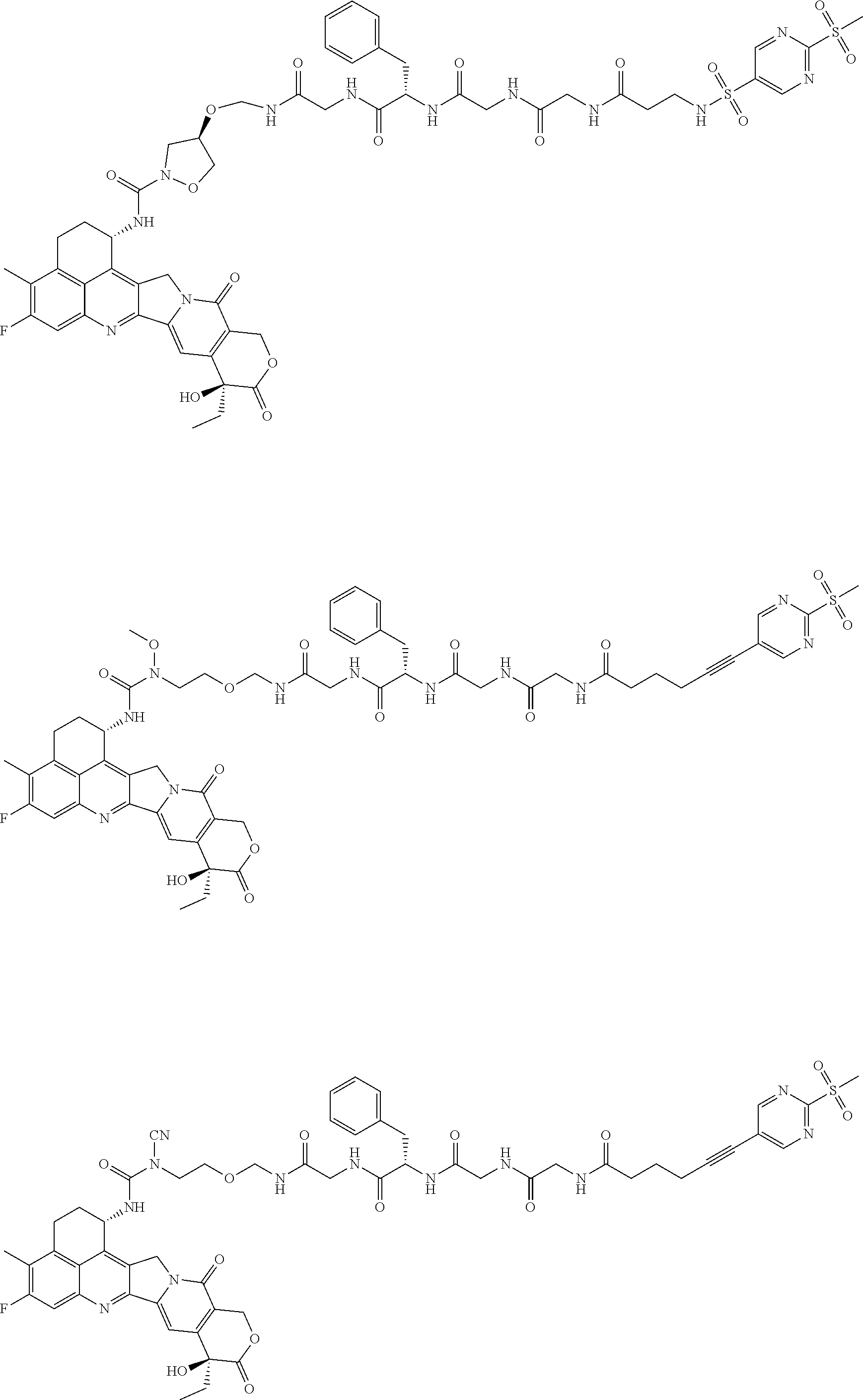 [SMILES: CC[C@@]1(O)C(=O)OCc2c1cc1n(c2=O)Cc2c-1nc1cc(F)c(C)c3c1c2[C@@H](NC(=O)N1C[C@@H](OCNC(=O)CNC(=O)[C@H](Cc2ccccc2)NC(=O)CNC(=O)CNC(=O)CCNS(=O)(=O)c2cnc(S(C)(=O)=O)nc2)CO1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCCC#Cc1cnc(S(C)(=O)=O)nc1)OC)CC3 ; CC[C@@]1(O)C(=O)OCc2c1cc1n(c2=O)Cc2c-1nc1cc(F)c(C)c3c1c2[C@@H](NC(=O)N(C#N)CCOCNC(=O)CNC(=O)[C@H](Cc1ccccc1)NC(=O)CNC(=O)CNC(=O)CCCC#Cc1cnc(S(C)(=O)=O)nc1)CC3]

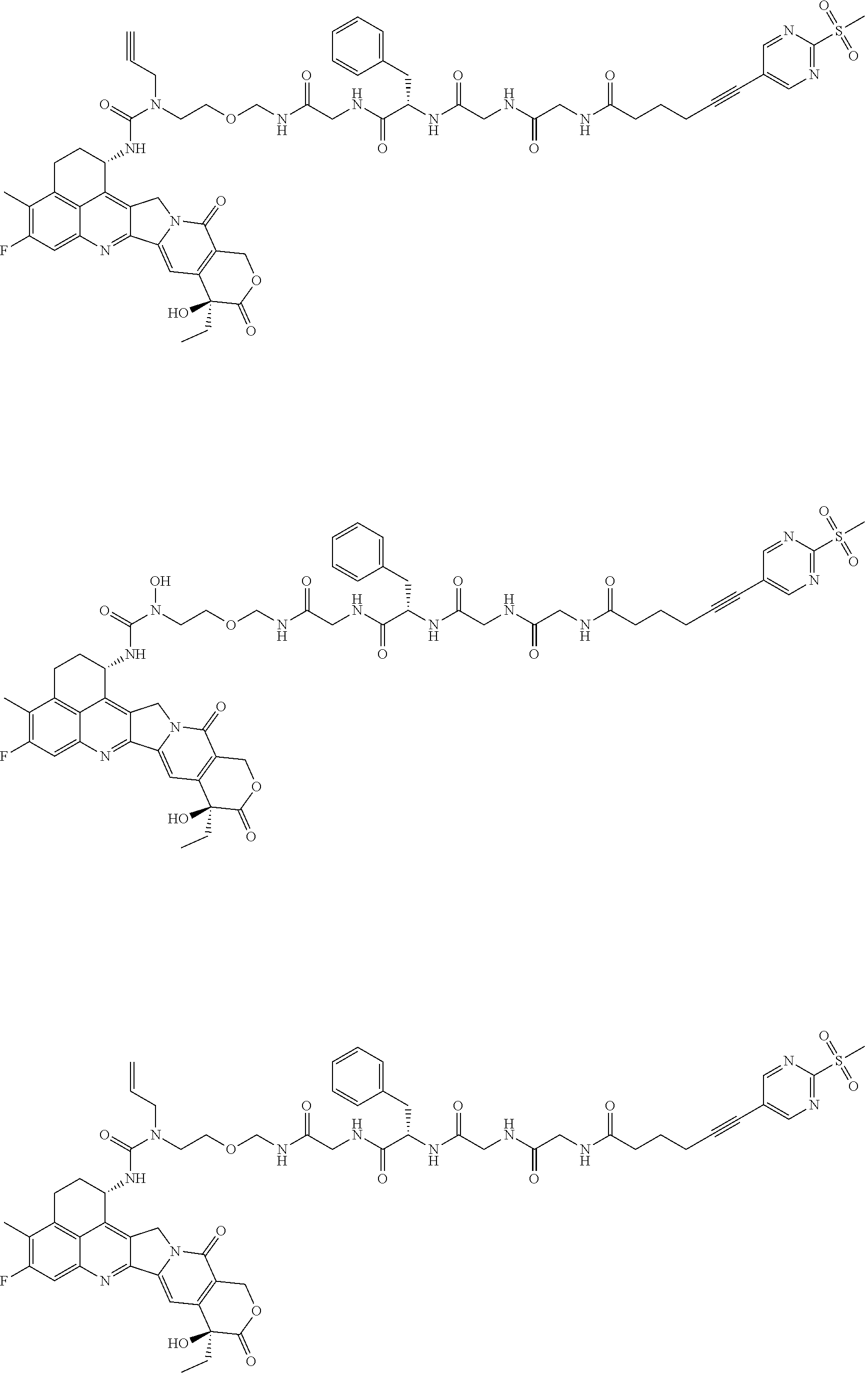 [SMILES: C#CCN(CCOCNC(=O)CNC(=O)[C@H](Cc1ccccc1)NC(=O)CNC(=O)CNC(=O)CCCC#Cc1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(O)CCOCNC(=O)CNC(=O)[C@H](Cc1ccccc1)NC(=O)CNC(=O)CNC(=O)CCCC#Cc1cnc(S(C)(=O)=O)nc1)CC3 ; C=CCN(CCOCNC(=O)CNC(=O)[C@H](Cc1ccccc1)NC(=O)CNC(=O)CNC(=O)CCCC#Cc1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

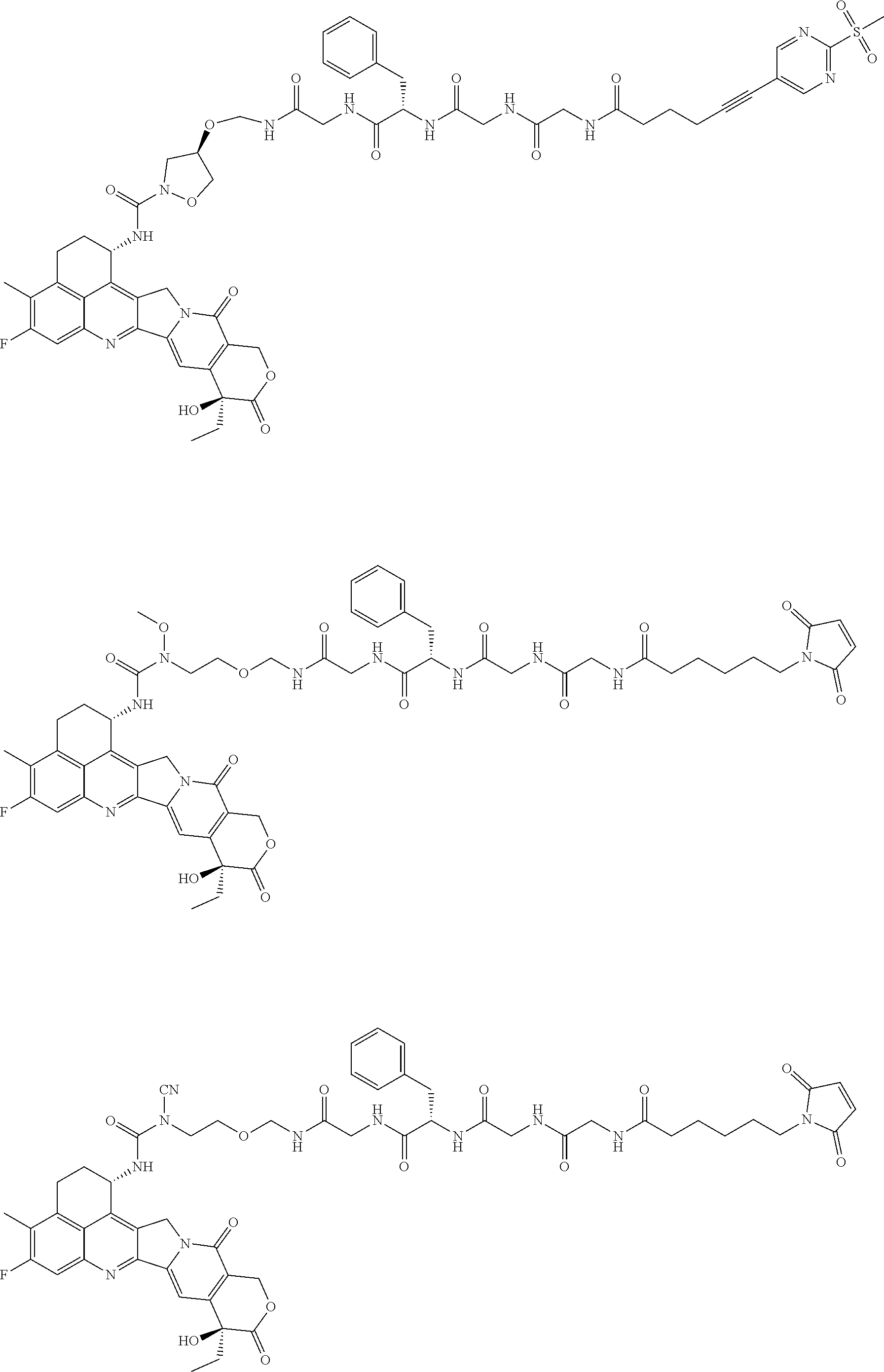 [SMILES: CC[C@@]1(O)C(=O)OCc2c1cc1n(c2=O)Cc2c-1nc1cc(F)c(C)c3c1c2[C@@H](NC(=O)N1C[C@@H](OCNC(=O)CNC(=O)[C@H](Cc2ccccc2)NC(=O)CNC(=O)CNC(=O)CCCC#Cc2cnc(S(C)(=O)=O)nc2)CO1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCCCCN1C(=O)C=CC1=O)OC)CC3 ; CC[C@@]1(O)C(=O)OCc2c1cc1n(c2=O)Cc2c-1nc1cc(F)c(C)c3c1c2[C@@H](NC(=O)N(C#N)CCOCNC(=O)CNC(=O)[C@H](Cc1ccccc1)NC(=O)CNC(=O)CNC(=O)CCCCCN1C(=O)C=CC1=O)CC3]

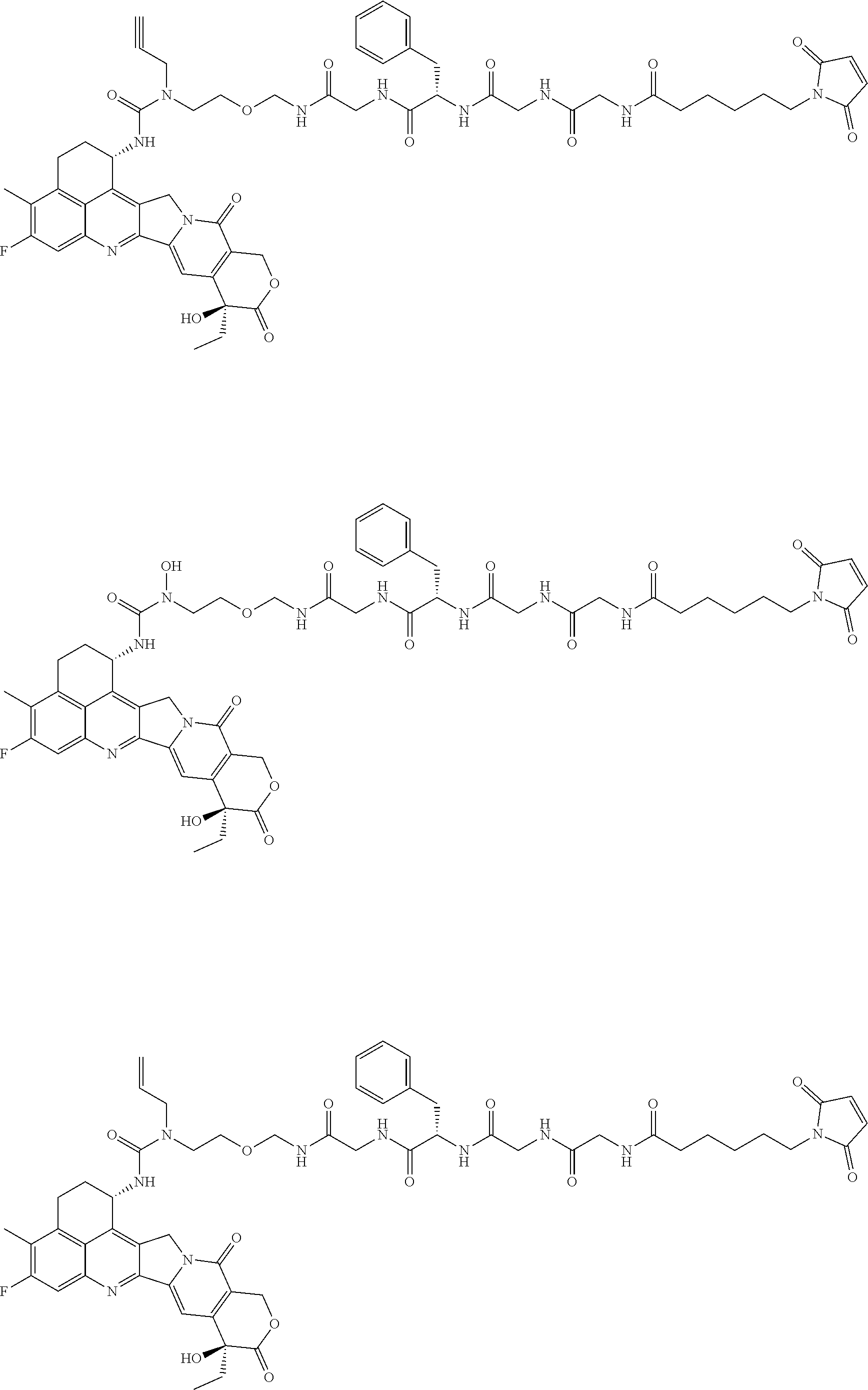 [SMILES: C#CCN(CCOCNC(=O)CNC(=O)[C@H](Cc1ccccc1)NC(=O)CNC(=O)CNC(=O)CCCCCN1C(=O)C=CC1=O)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(O)CCOCNC(=O)CNC(=O)[C@H](Cc1ccccc1)NC(=O)CNC(=O)CNC(=O)CCCCCN1C(=O)C=CC1=O)CC3 ; C=CCN(CCOCNC(=O)CNC(=O)[C@H](Cc1ccccc1)NC(=O)CNC(=O)CNC(=O)CCCCCN1C(=O)C=CC1=O)C(=O)N[C@H]1CCc2c(C)c(F)cc3nc4c(c1c23)Cn1c-4cc2c(c1=O)COC(=O)[C@]2(O)CC]

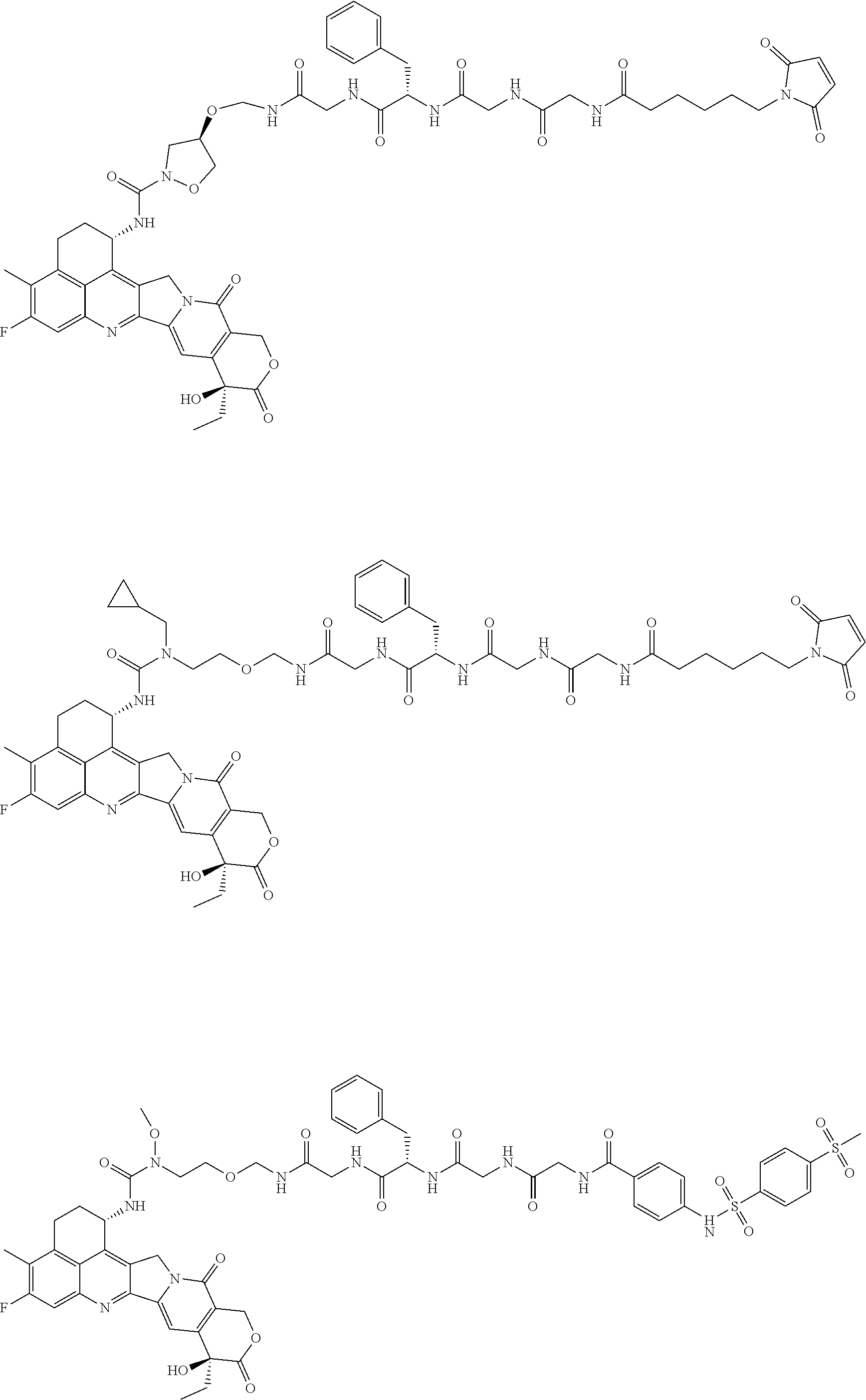 [SMILES: CC[C@@]1(O)C(=O)OCc2c1cc1n(c2=O)Cc2c-1nc1cc(F)c(C)c3c1c2[C@@H](NC(=O)N1C[C@@H](OCNC(=O)CNC(=O)[C@H](Cc2ccccc2)NC(=O)CNC(=O)CNC(=O)CCCCCN2C(=O)C=CC2=O)CO1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCCCCN1C(=O)C=CC1=O)CC1CC1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)c1ccc(NS(=O)(=O)c2ccc(S(C)(=O)=O)cc2)cc1)OC)CC3]

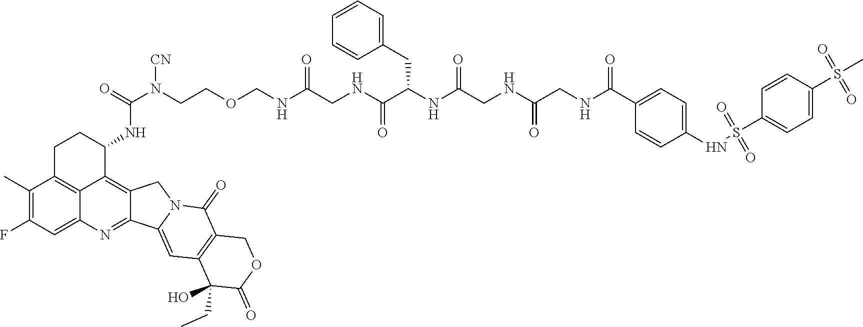 [SMILES: CC[C@@]1(O)C(=O)OCc2c1cc1n(c2=O)Cc2c-1nc1cc(F)c(C)c3c1c2[C@@H](NC(=O)N(C#N)CCOCNC(=O)CNC(=O)[C@H](Cc1ccccc1)NC(=O)CNC(=O)CNC(=O)c1ccc(NS(=O)(=O)c2ccc(S(C)(=O)=O)cc2)cc1)CC3]

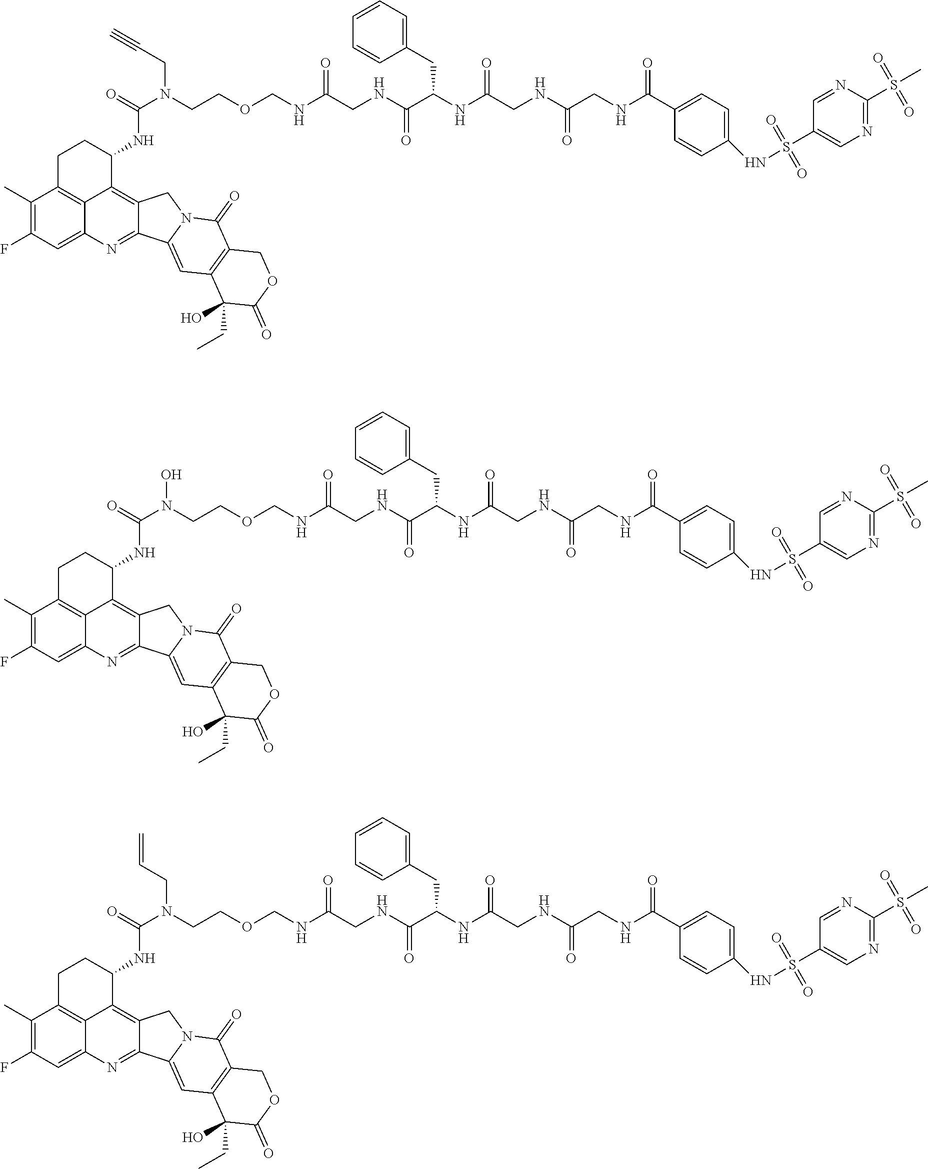

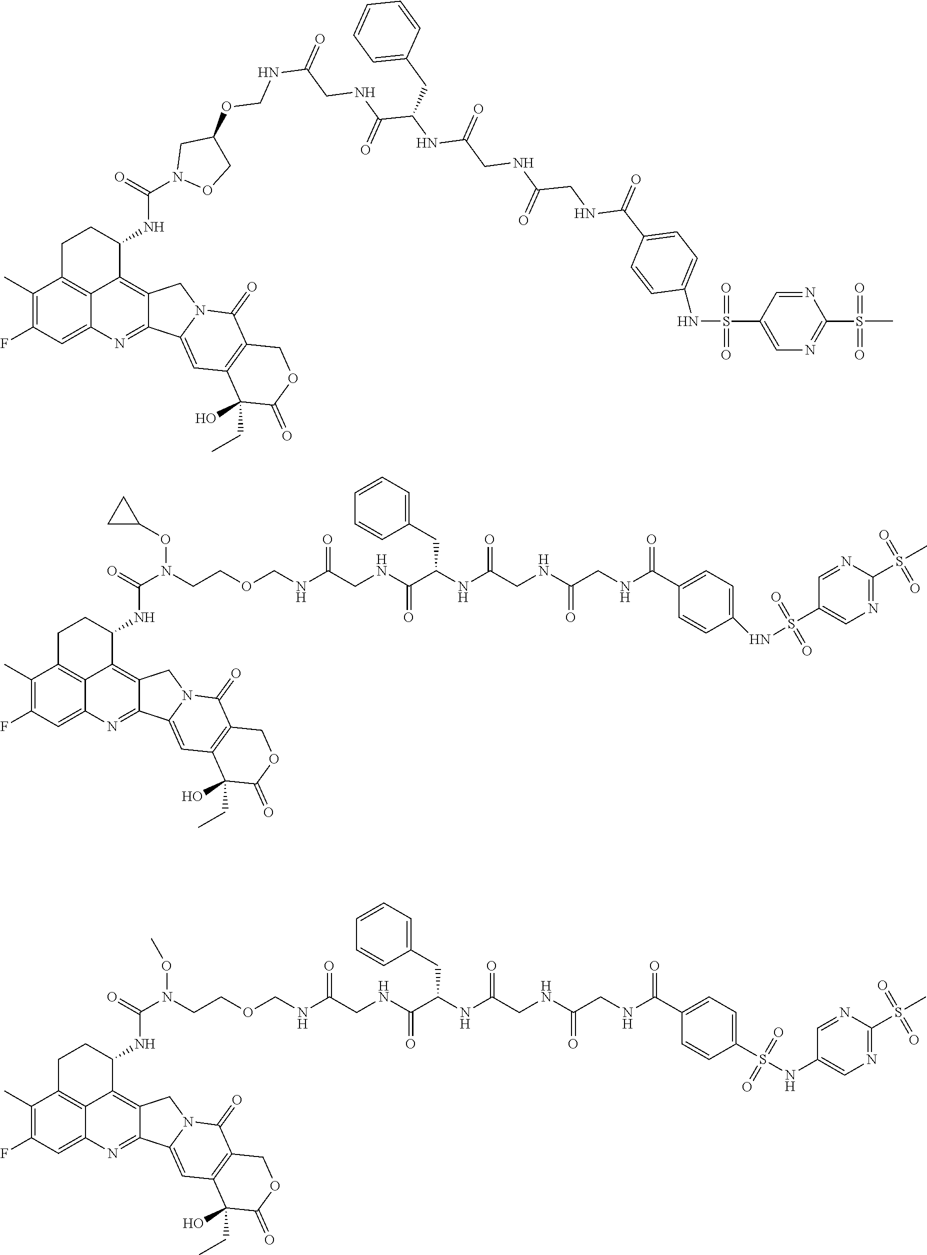

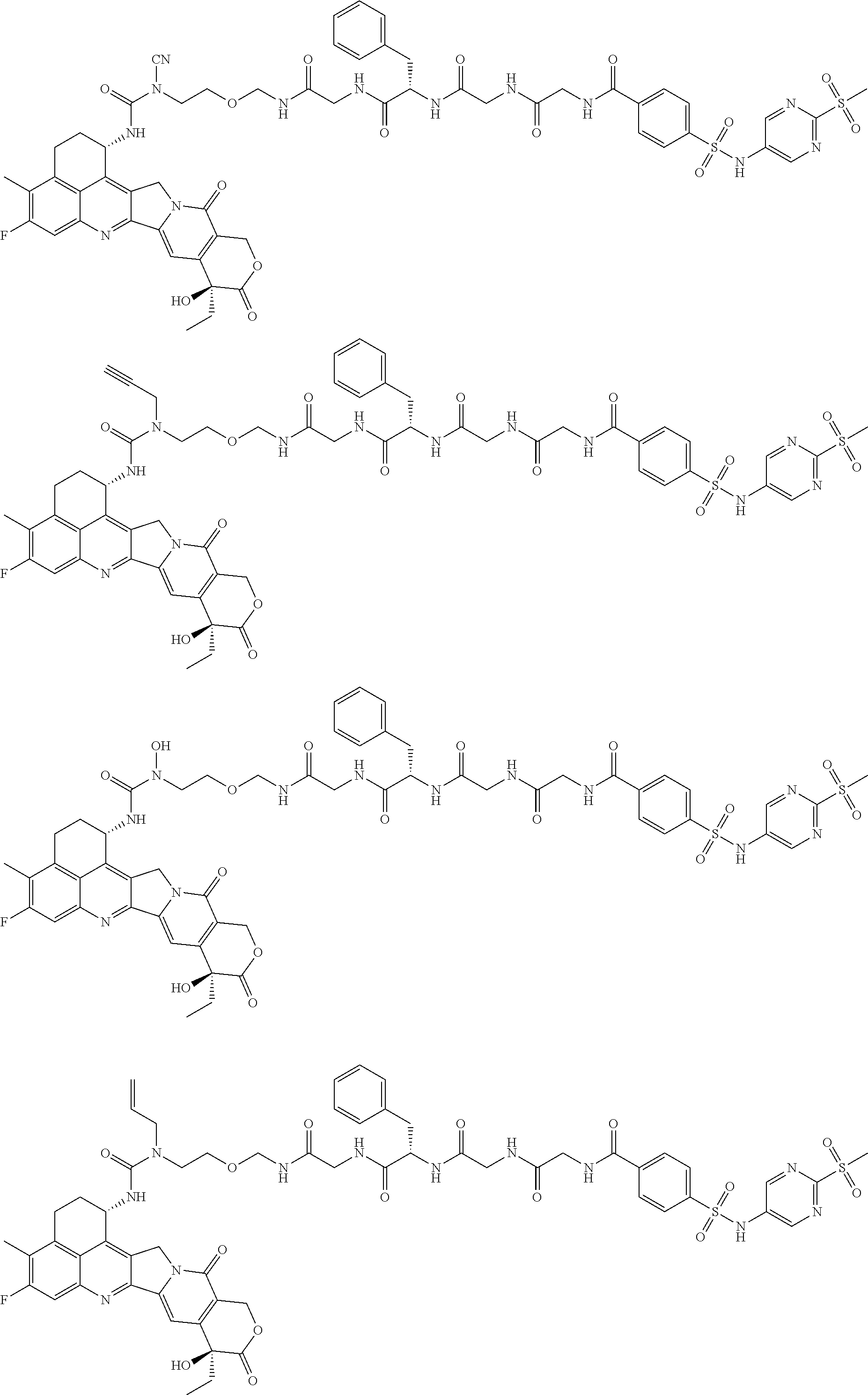

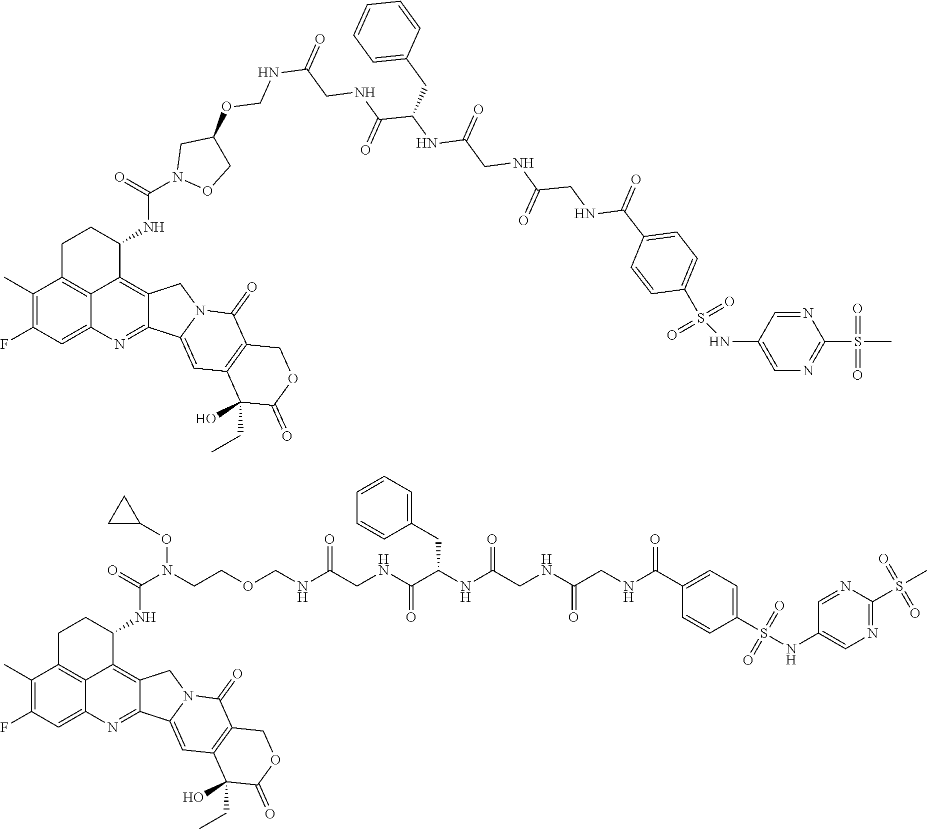

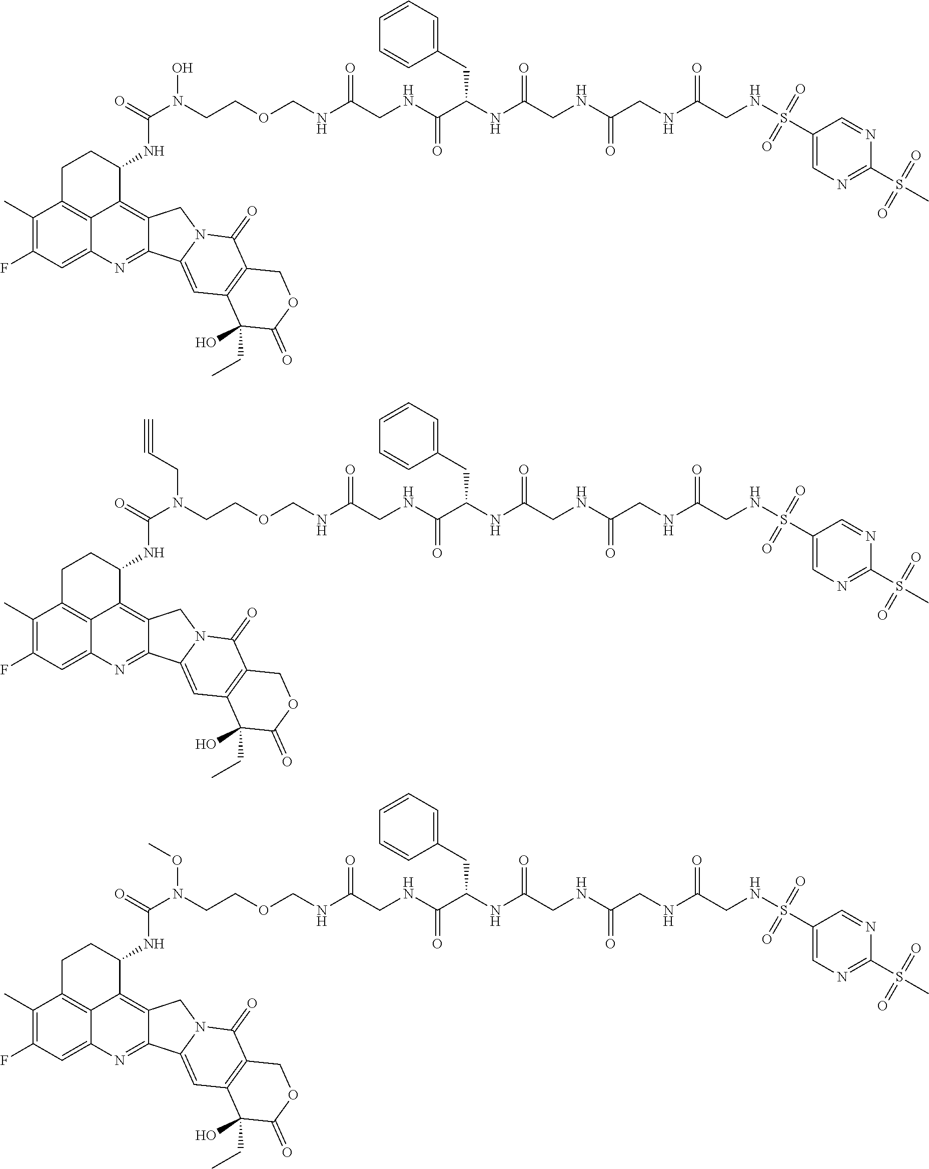

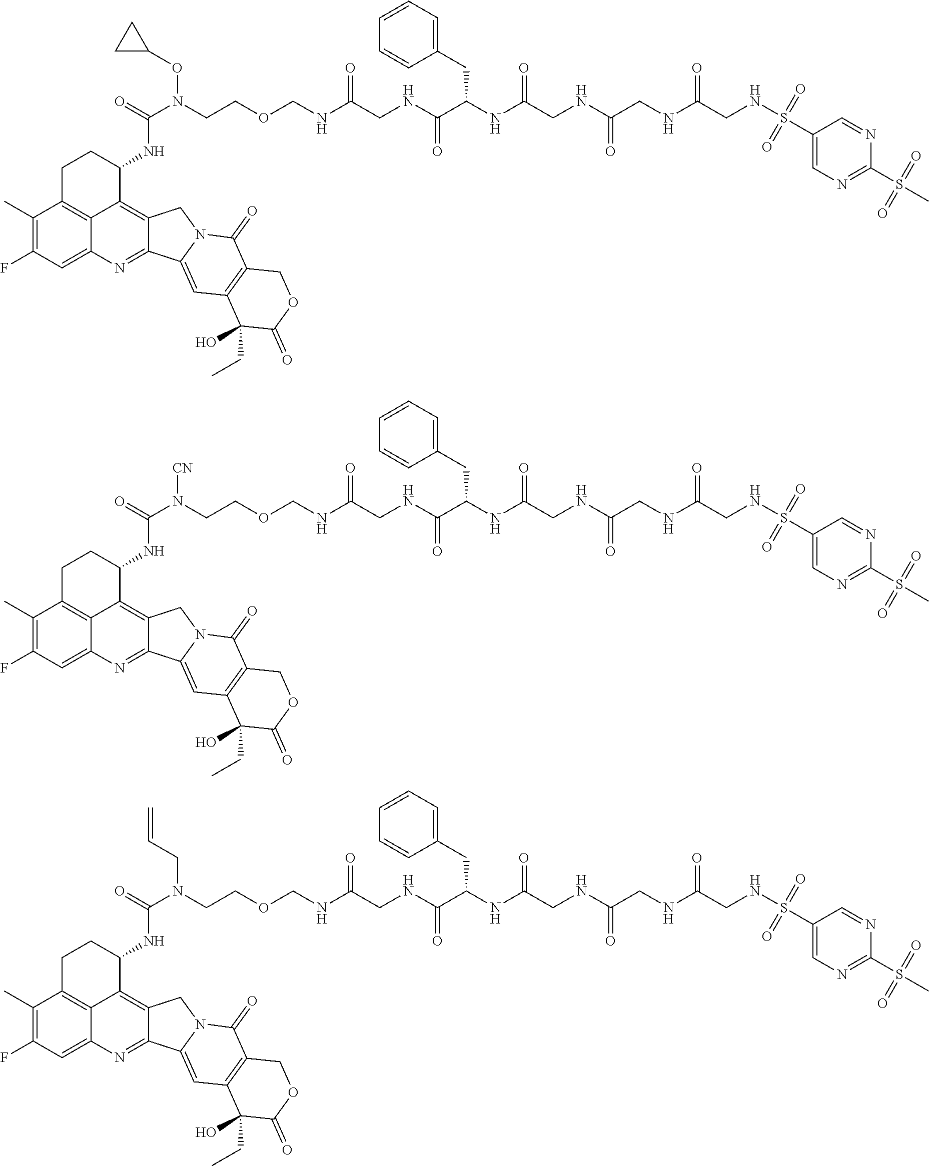

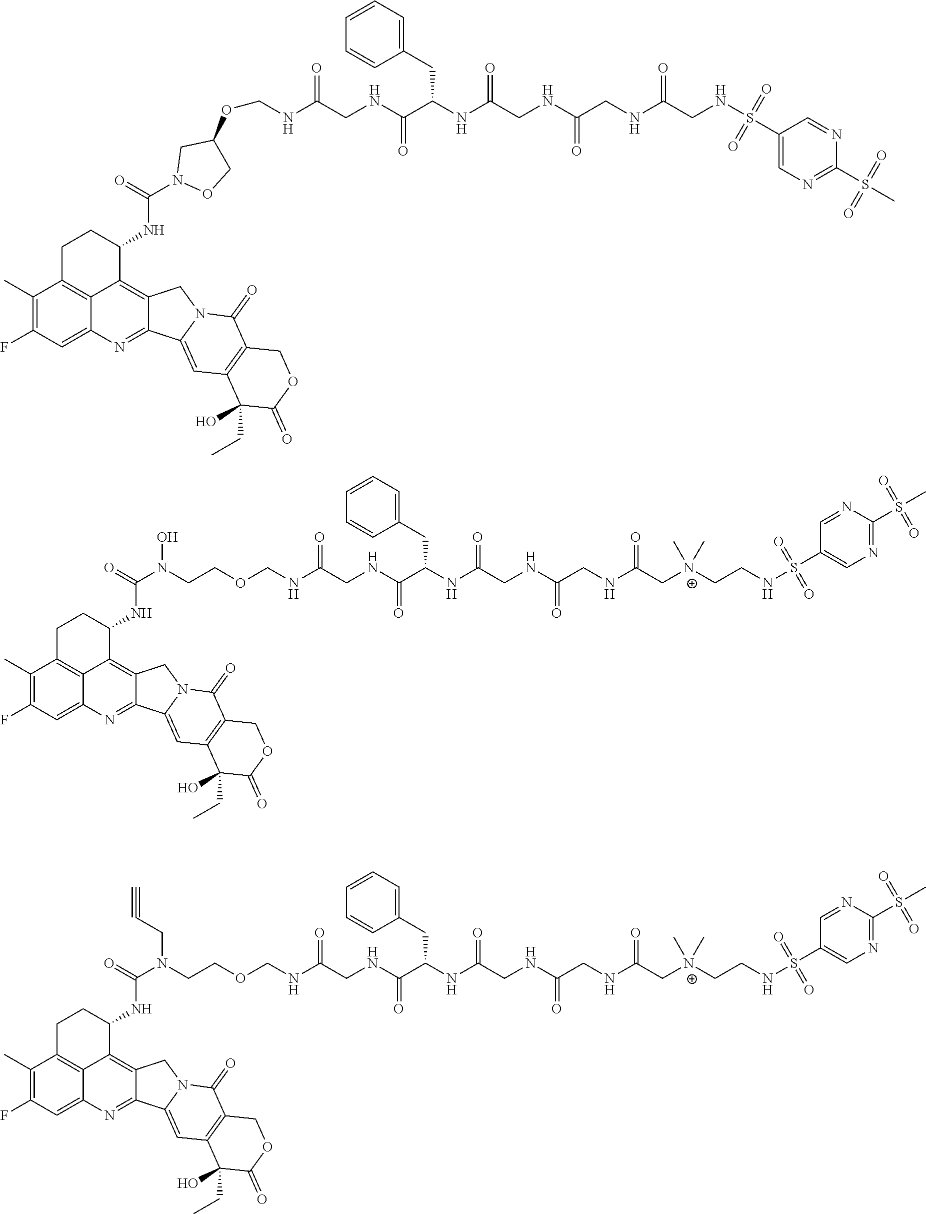

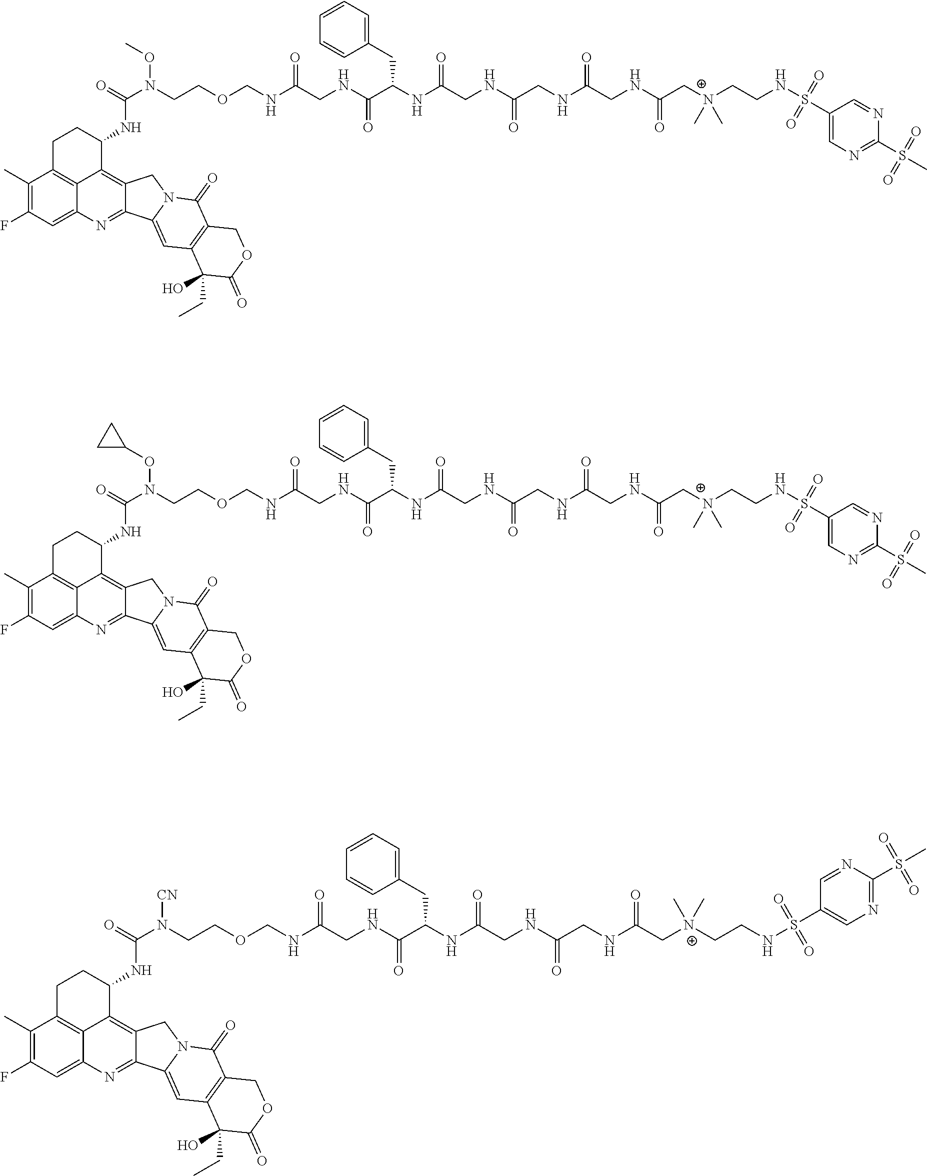

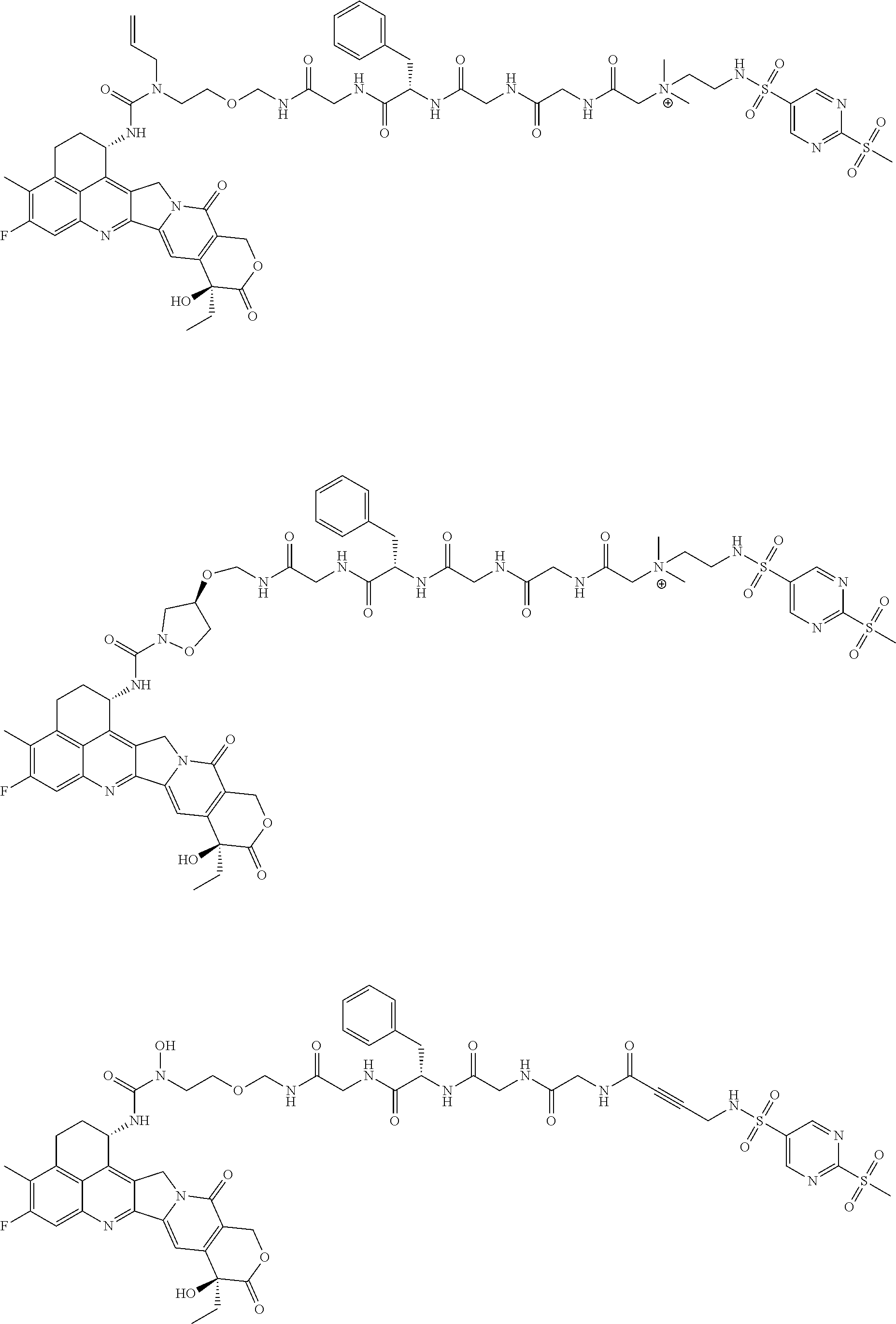

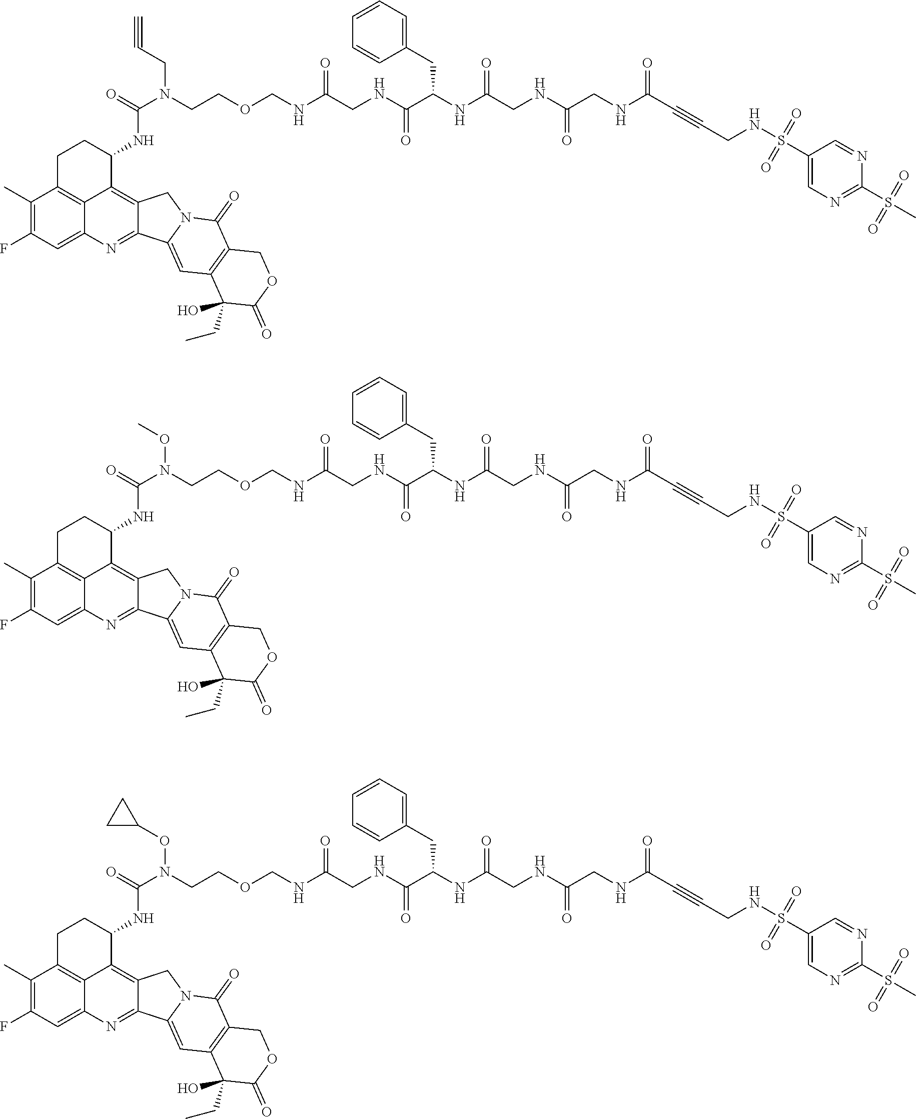

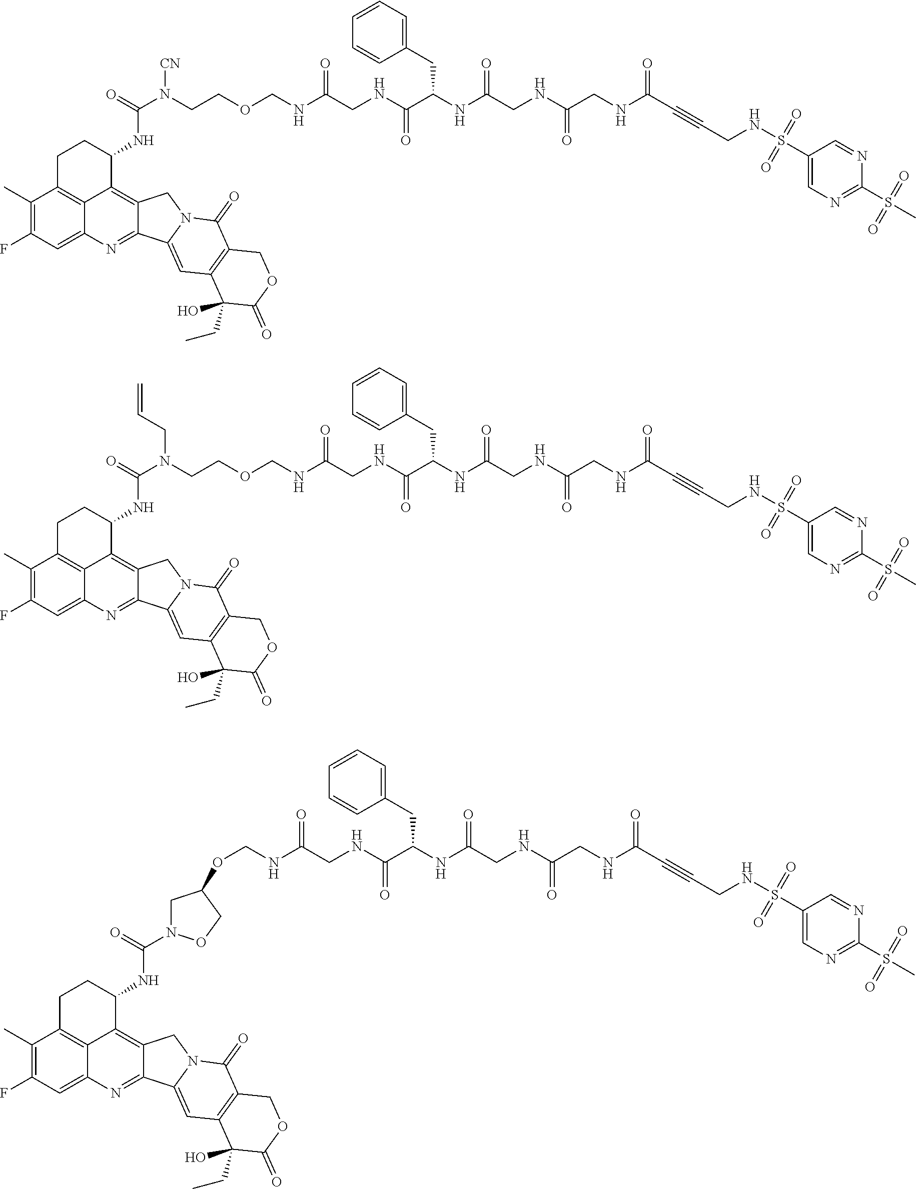

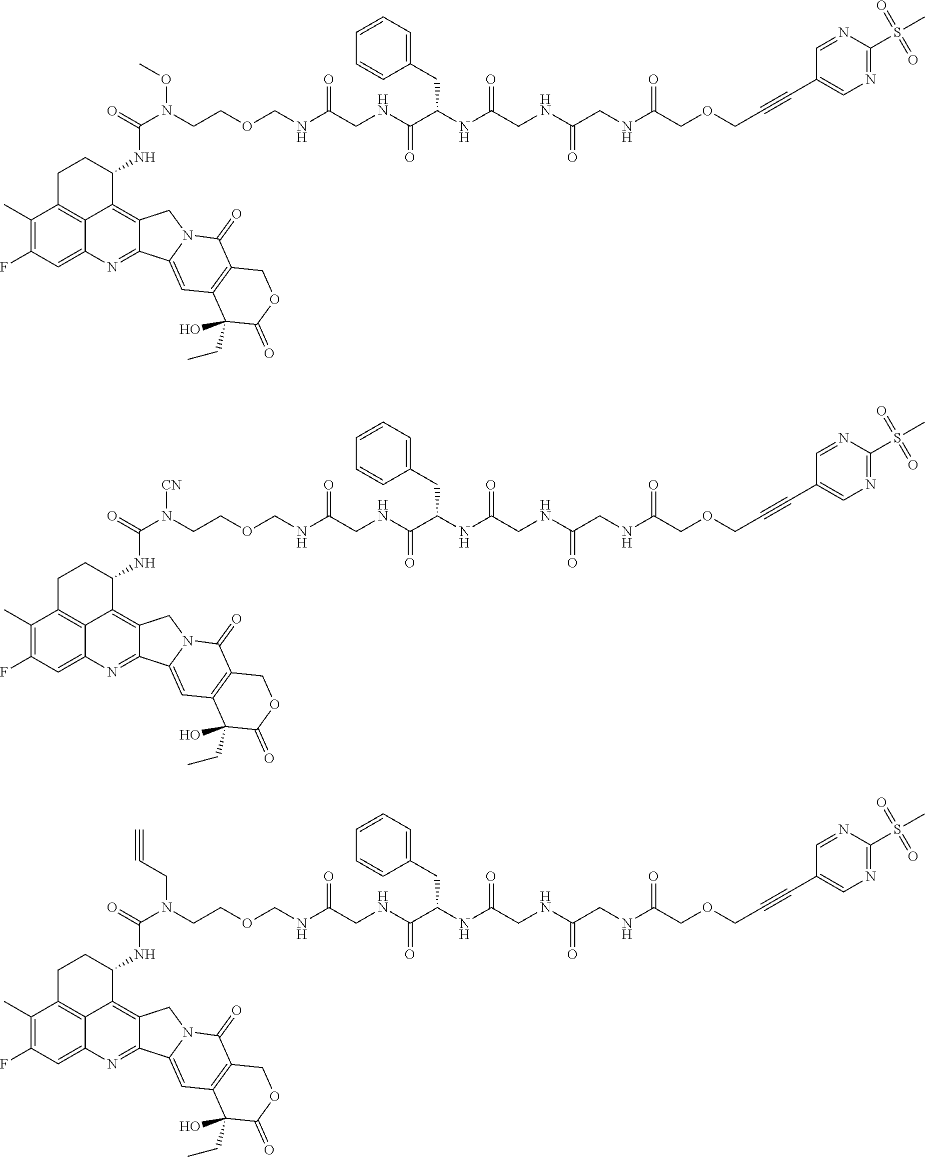

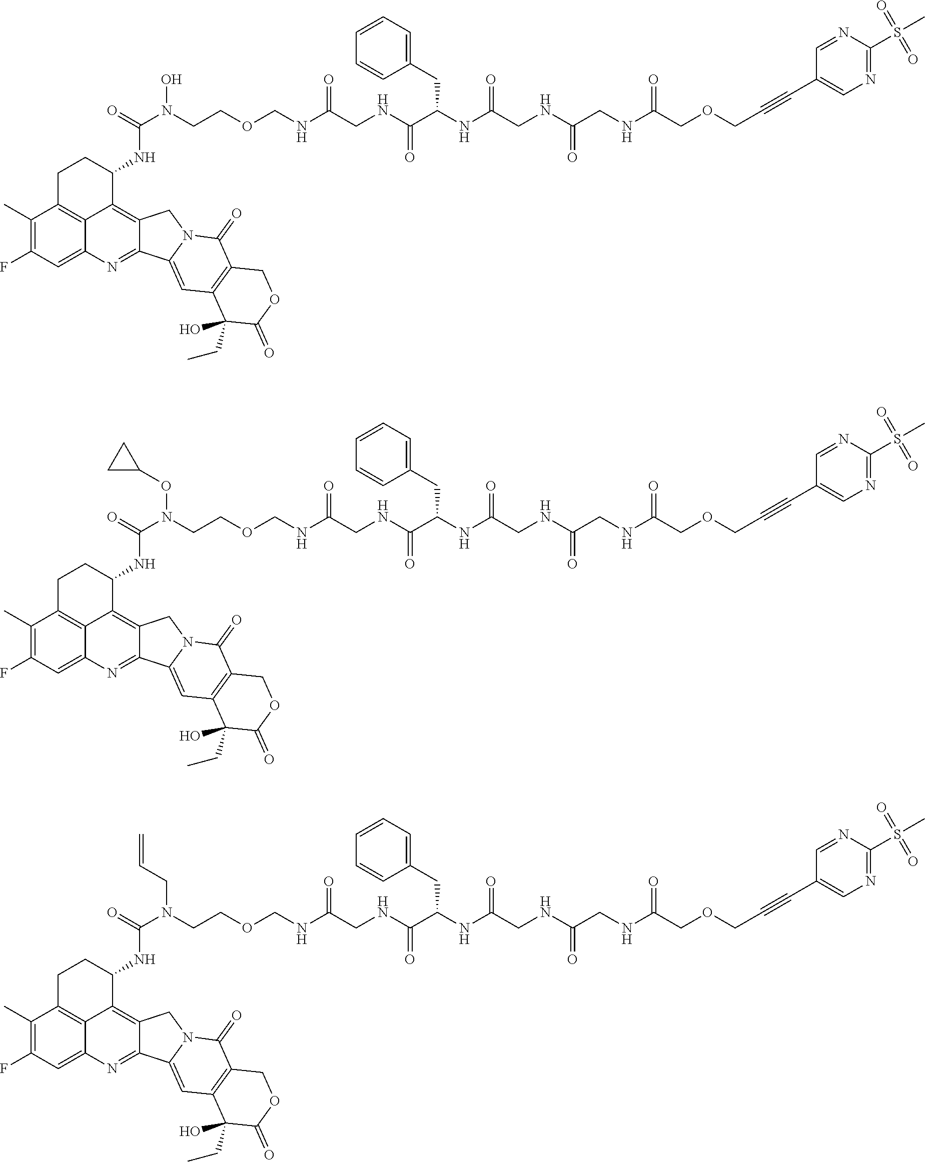

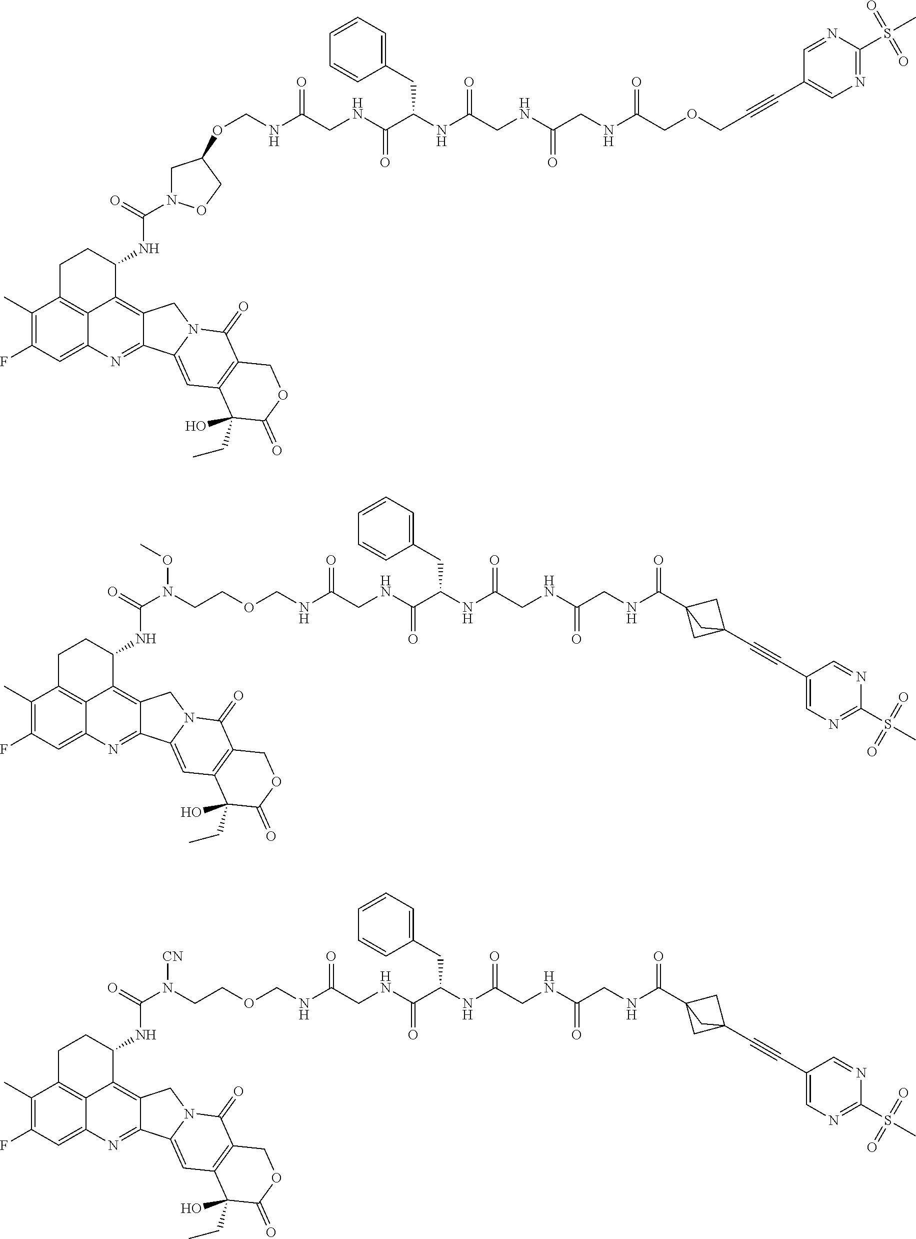

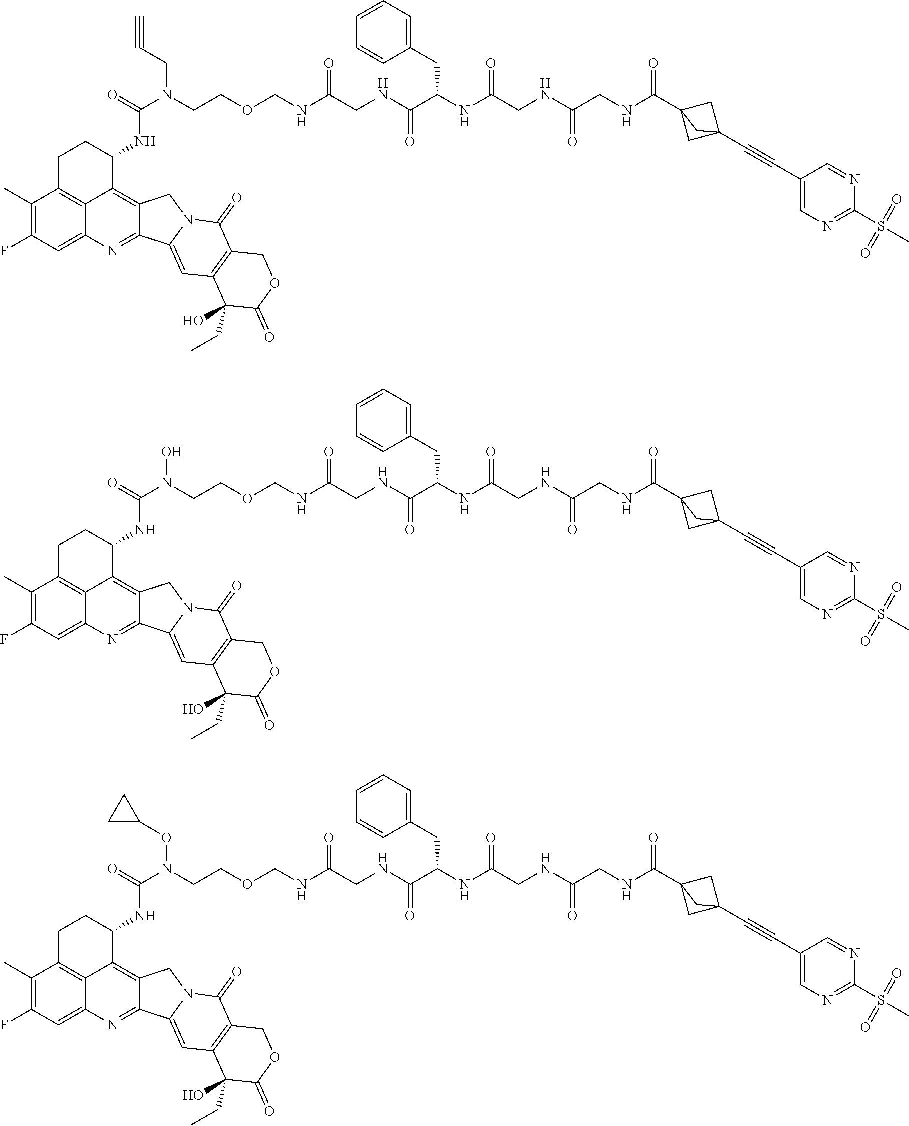

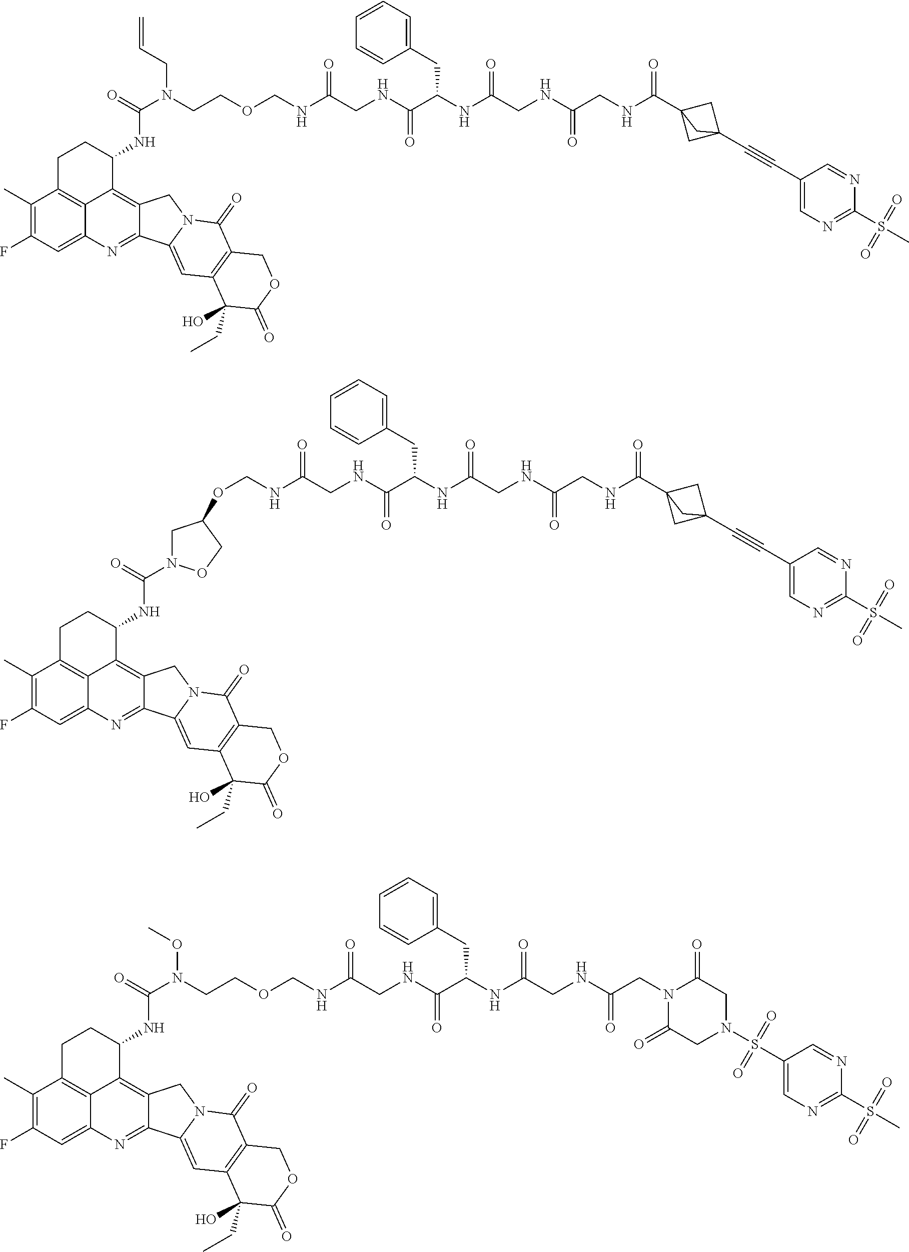

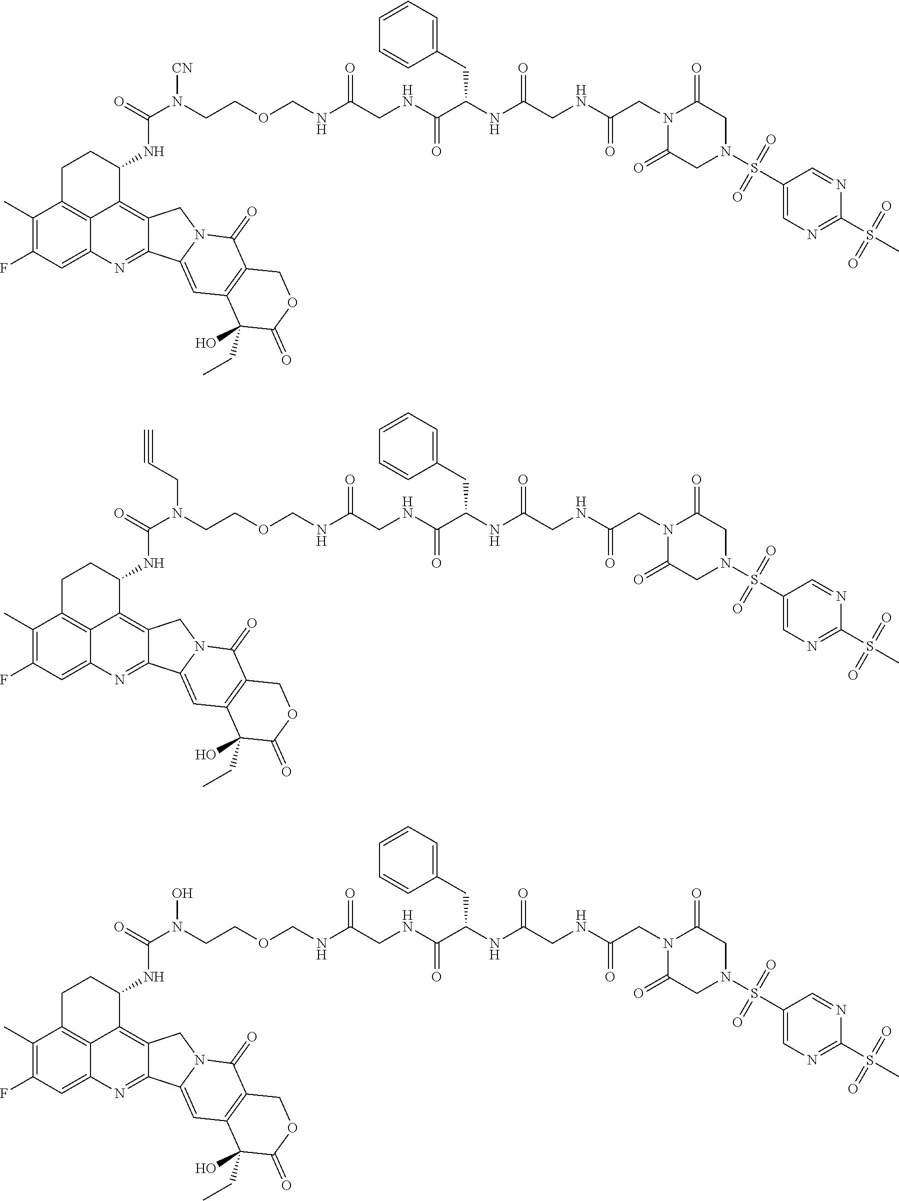

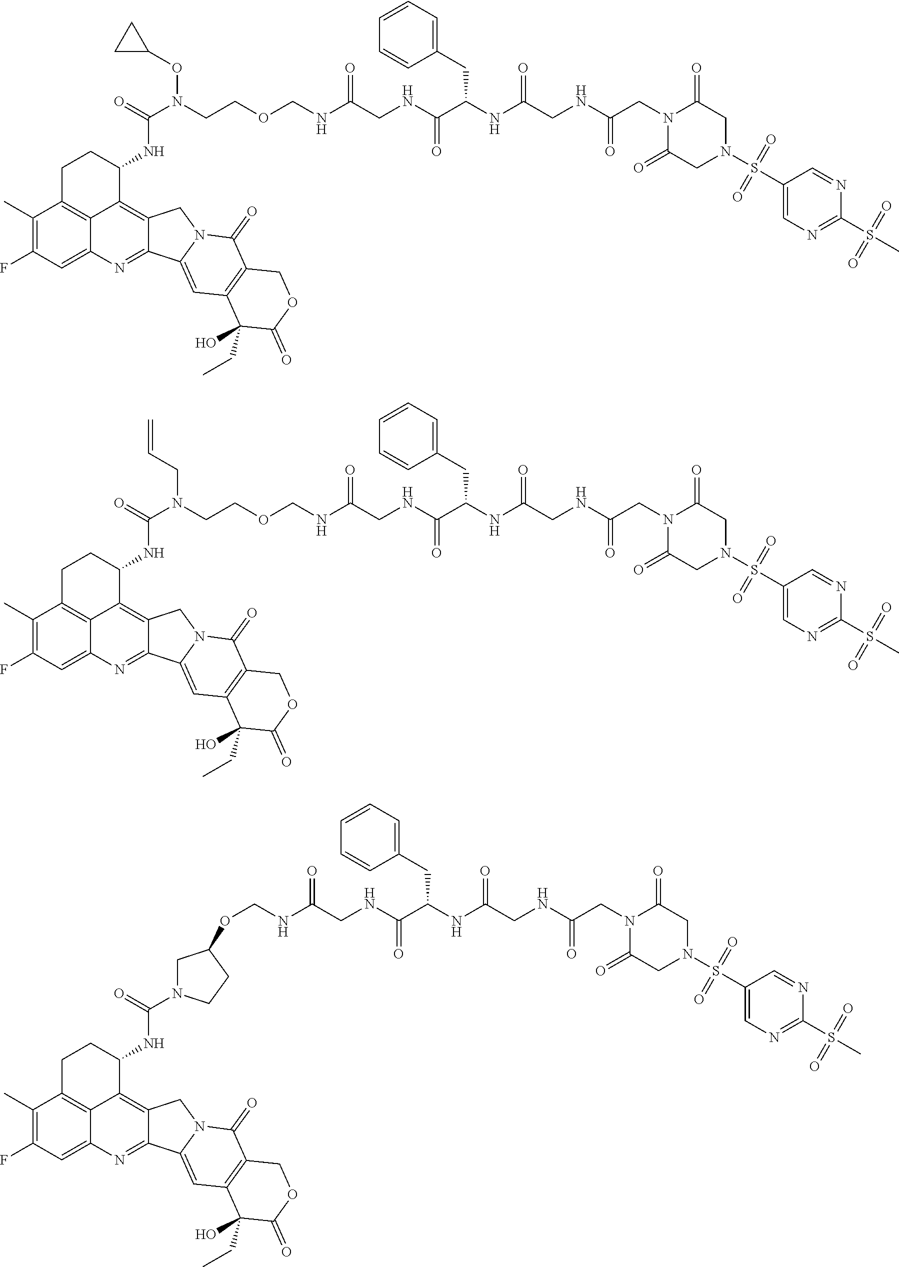

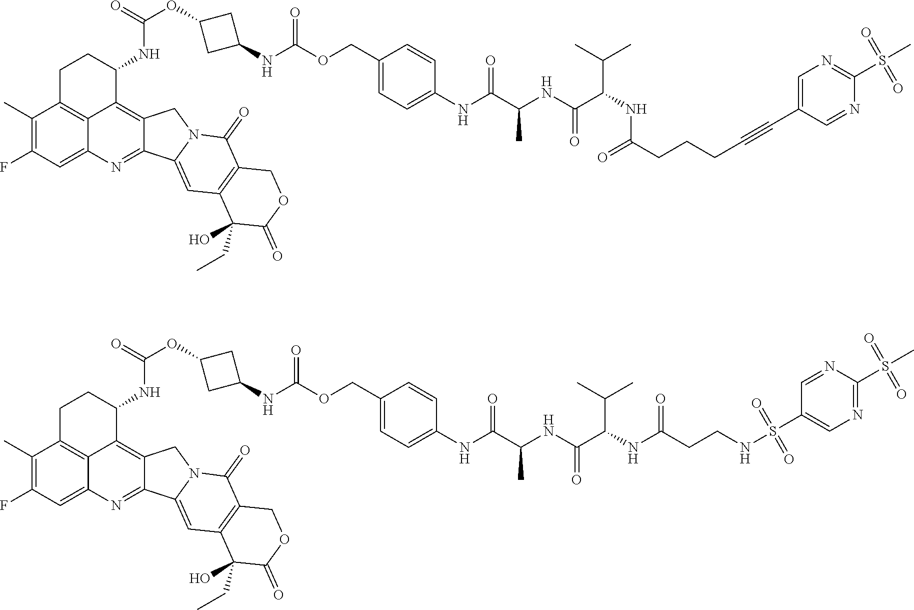

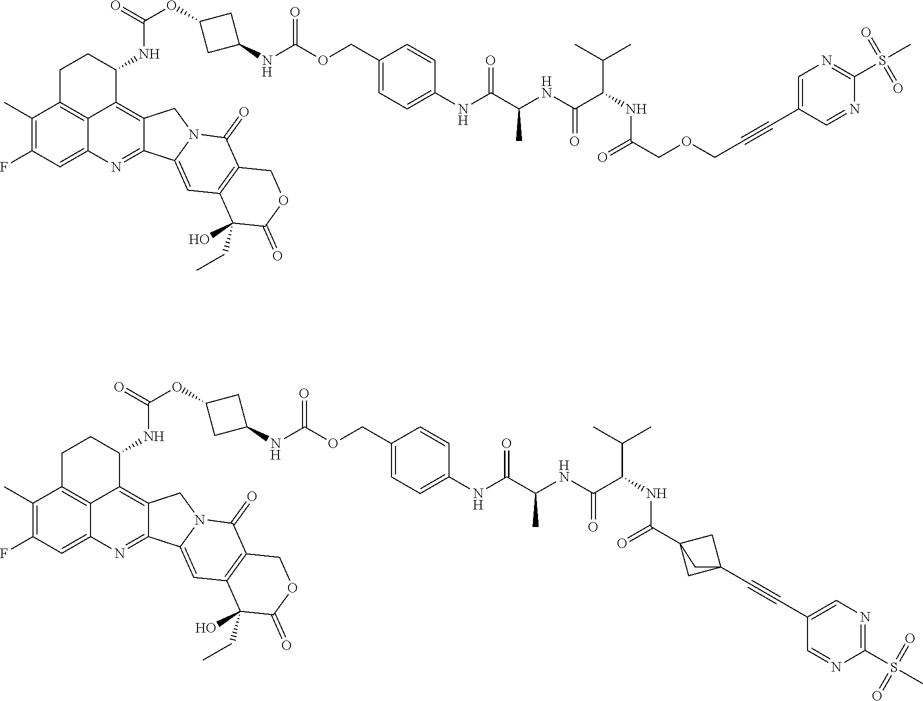

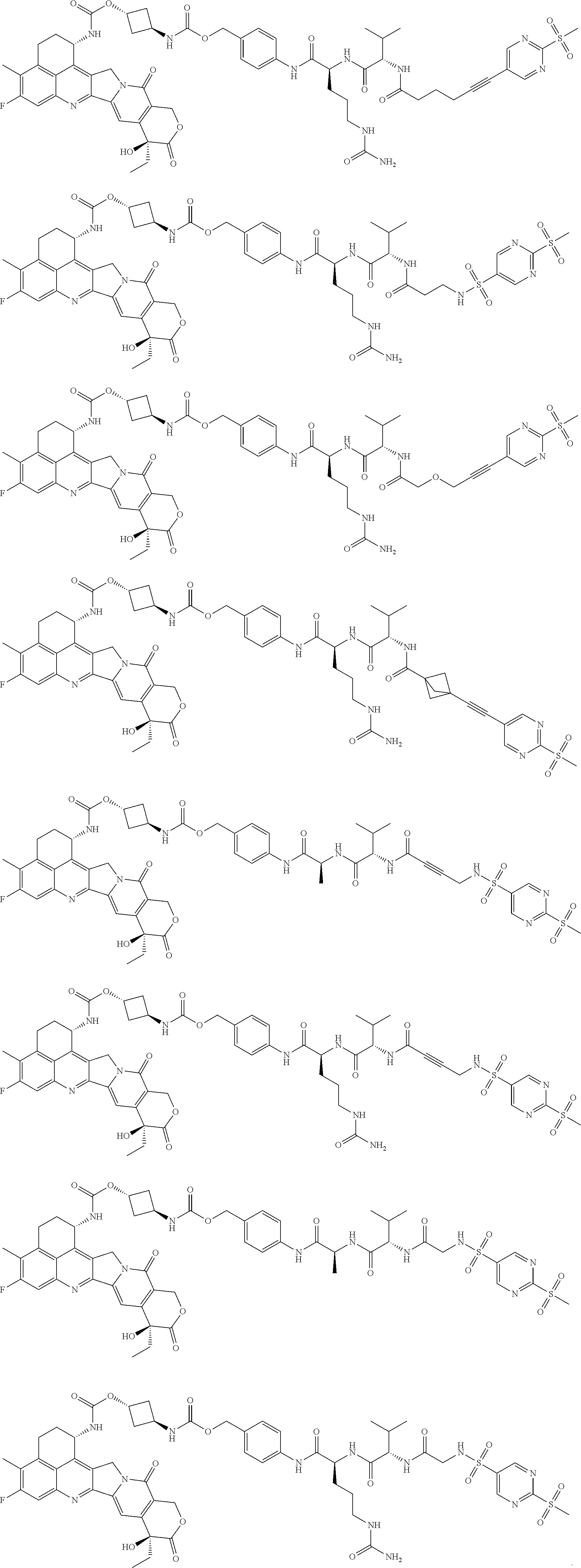

In some embodiments, the antibody-drug conjugate is selected from:

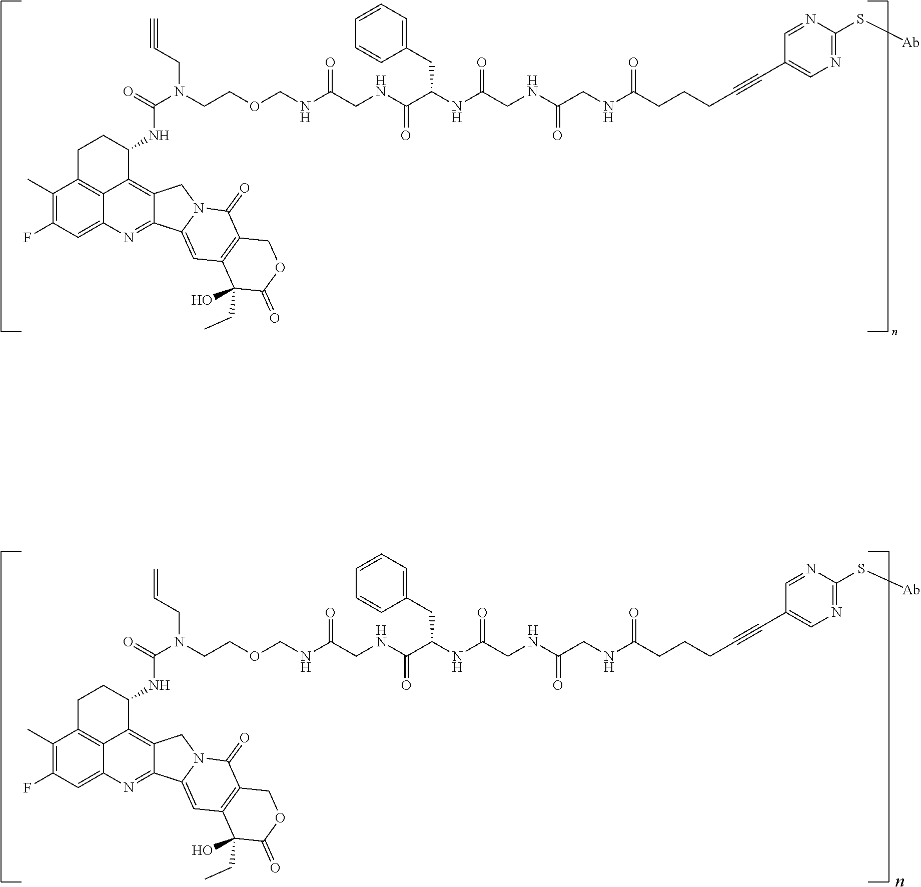

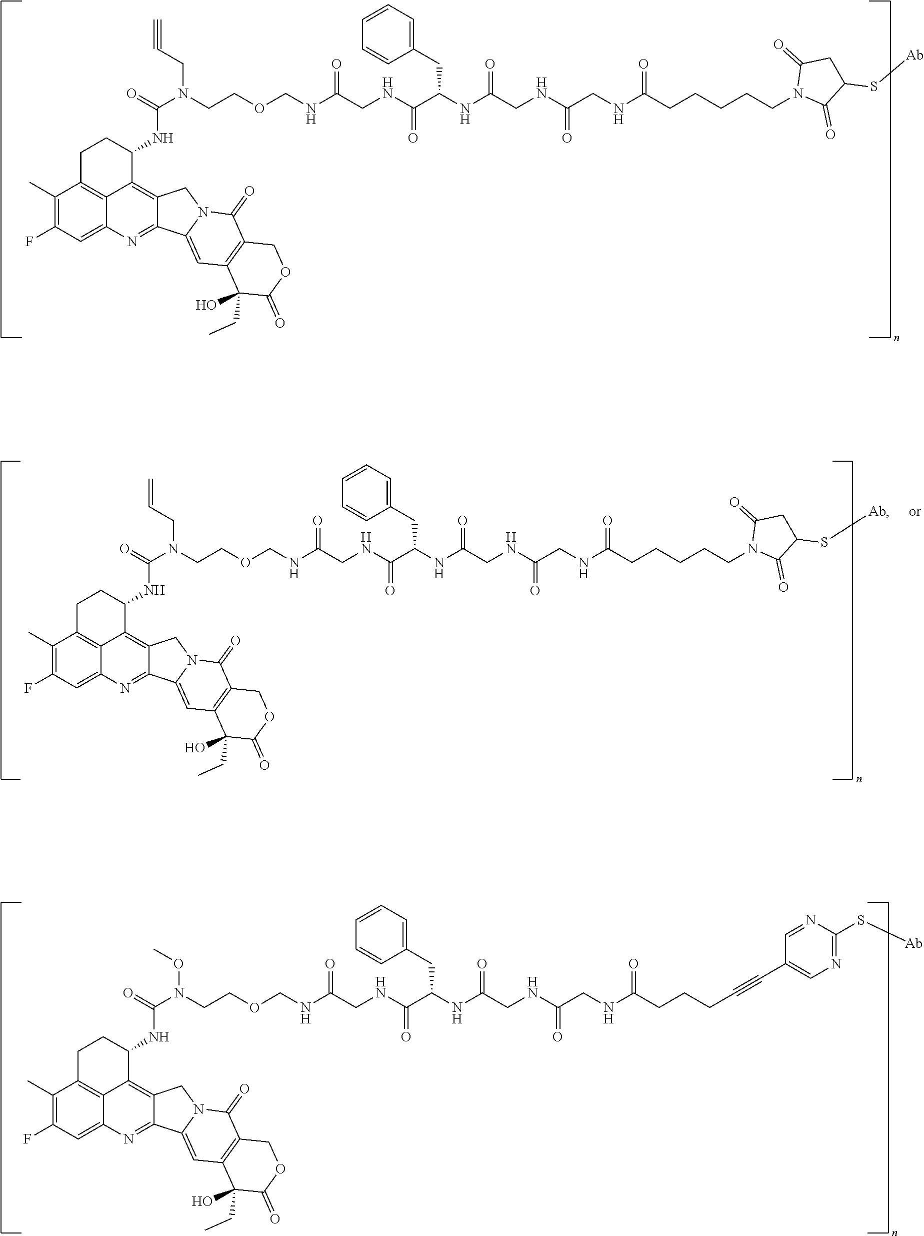

wherein, the Ab and n are as defined in formula (I).

In some embodiments, in the antibody-drug conjugate, the Ab is an anti-Her2 antibody; preferably, the Ab is Trastuzumab or Pertuzumab; and further preferably, the Ab is Pertuzumab.

In some embodiments, the antibody-drug conjugate as shown by general formula (I) is selected from an antibody-drug conjugate as shown by general formula (II):

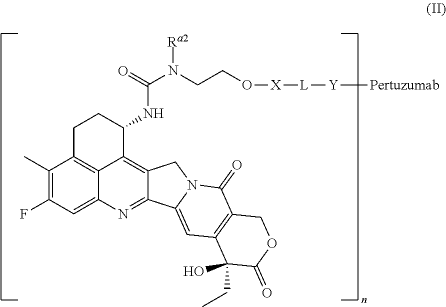

wherein, R^{a2} is selected from

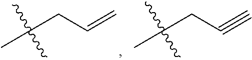

or —OCH₃;
—X-L-Y— is selected from

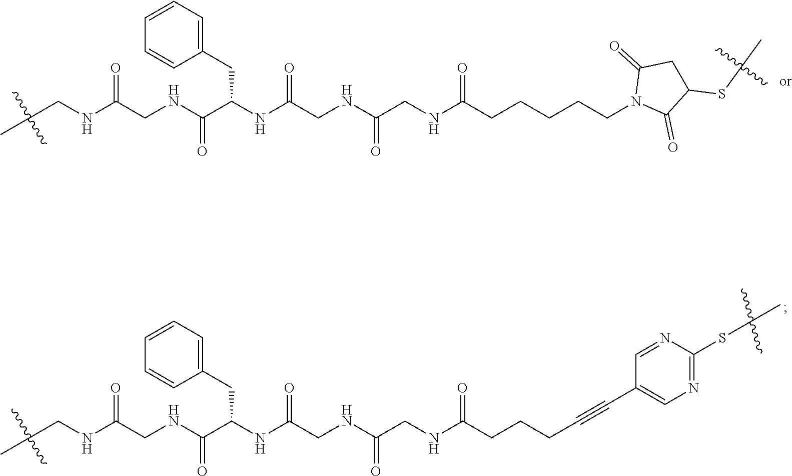 [SMILES: *CNC(=O)CNC(=O)[C@H](Cc1ccccc1)NC(=O)CNC(=O)CNC(=O)CCCCCN1C(=O)CC(S*)C1=O ; *CNC(=O)CNC(=O)[C@H](Cc1ccccc1)NC(=O)CNC(=O)CNC(=O)CCCC#Cc1cnc(S*)nc1]

and
n is selected from numerical values of 4-8; preferably, n is selected from numerical values of 6-8; and further preferably, n is selected from numerical values of 7-8.

In some embodiments, the antibody-drug conjugate is selected from:

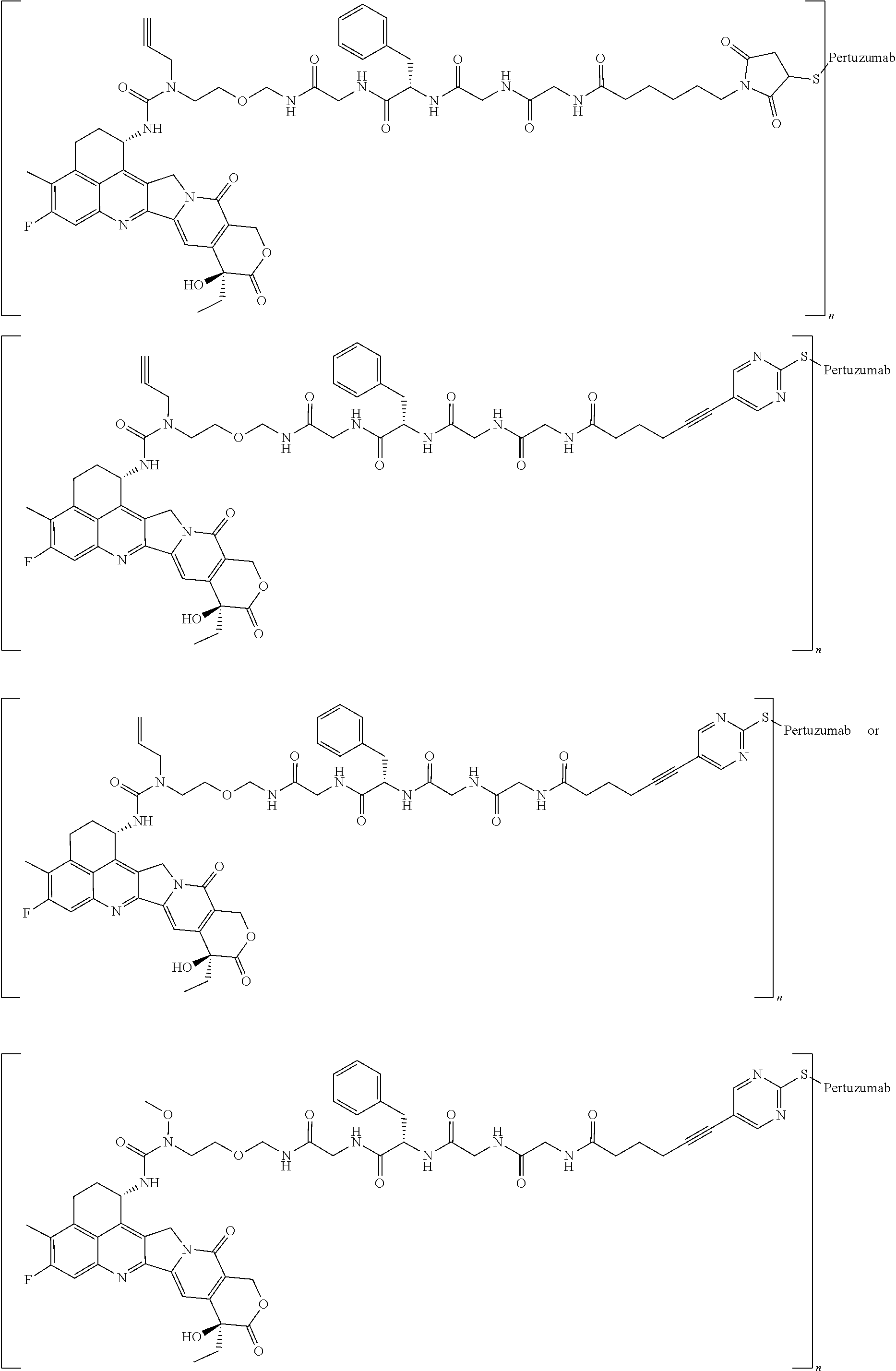

wherein, n is selected from numerical values of 6-8; and preferably, n is selected from numerical values of 7-8.

In another aspect, the present disclosure further provides a pharmaceutical composition, which comprises the antibody-drug conjugate as shown by formula (I), formula (Ia) or formula (II), the stereoisomer thereof or the pharmaceutically acceptable salt thereof, and a pharmaceutically acceptable carrier.

In some embodiments, in the pharmaceutical composition, a content of the antibody-drug conjugate is selected from 0.1 mg-1000 mg.

In some embodiments, in the pharmaceutical composition, a content of the antibody-drug conjugate is 1%-95%.

In some embodiments, in the pharmaceutical composition, the pharmaceutically acceptable carrier comprises one or more of a filler, a disintegrant, an adhesive, a glidant and a lubricant.

In another aspect, the present disclosure provides use of the antibody-drug conjugate as shown by formula (I), formula (Ia) or formula (II), the cytotoxic drug as shown by formula (D′) or the cytotoxic drug-linker, the stereoisomer thereof or the pharmaceutically acceptable salt thereof, or the pharmaceutical composition in the preparation of a medicament for treating or preventing cancer.

In another aspect, the present disclosure provides the antibody-drug conjugate as shown by formula (I), formula (Ia) or formula (II), the cytotoxic drug as shown by the formula (D′) or the cytotoxic drug-linker, the stereoisomer thereof or the pharmaceutically acceptable salt thereof, or the pharmaceutical composition for use as a medicament.

In another aspect, the present disclosure provides the antibody-drug conjugate as shown by formula (I), formula (Ia) or formula (II), the cytotoxic drug as shown by formula (D′) or the cytotoxic drug-linker, the stereoisomer thereof or the pharmaceutically acceptable salt thereof, or the pharmaceutical composition for use in the treatment or prevention of cancer.

In another aspect, the present disclosure provides a method for treating or preventing cancer, which comprises administering a therapeutically or prophylactically effective amount of the antibody-drug conjugate as shown by formula (I), formula (Ia) or formula (II), the cytotoxic drug as shown by formula (D′) or the cytotoxic drug-linker, the stereoisomer thereof or the pharmaceutically acceptable salt thereof, or the pharmaceutical composition to a subject.

In some embodiments, the cancer is selected from a Her2 target-mediated cancer.

In another aspect, the present disclosure further provides a preparation method for an antibody-drug conjugate, wherein, after treating Ab under reducing conditions, the cytotoxic drug-linker is linked to the Ab through a thioether bond to form the antibody-drug conjugate.

In some embodiments, the Ab is an anti-Her2 antibody; preferably, the Ab is Trastuzumab or Pertuzumab; and further preferably, the Ab is Pertuzumab.

In another aspect, the present disclosure further provides compounds as shown by the following formulae:

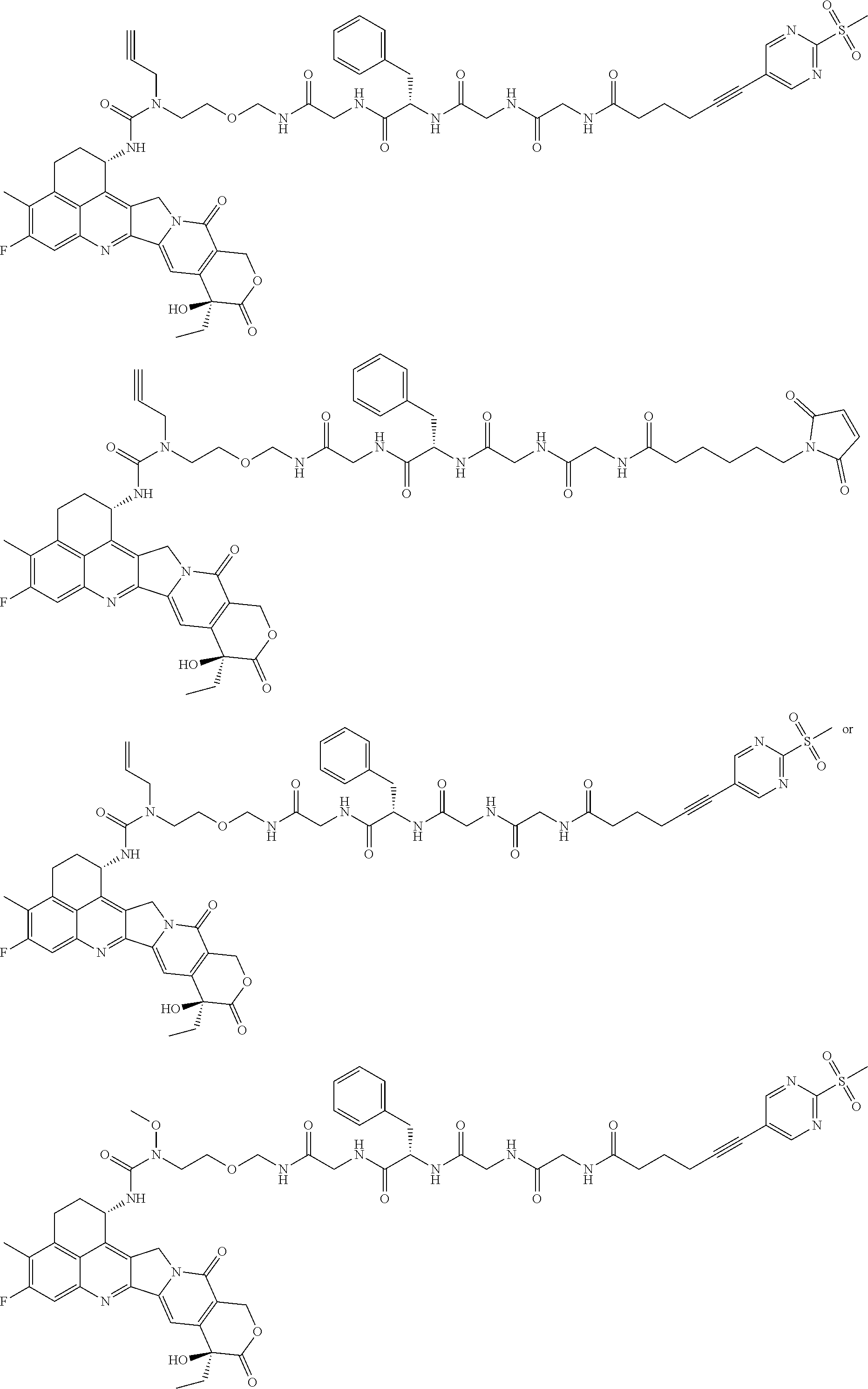

In another aspect, the present disclosure further provides the following compounds:

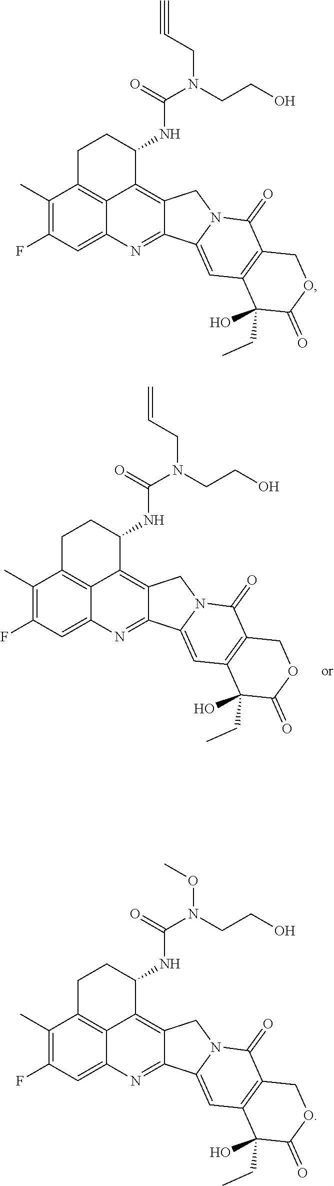 [SMILES: C#CCN(CCO)C(=O)N[C@H]1CCc2c(C)c(F)cc3nc4c(c1c23)Cn1c-4cc2c(c1=O)COC(=O)[C@]2(O)CC ; C=CCN(CCO)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(CCO)OC)CC3]

In some embodiments, provided a reuse of the following compound in the preparation of an antibody-drug conjugate, the compound is selected from:

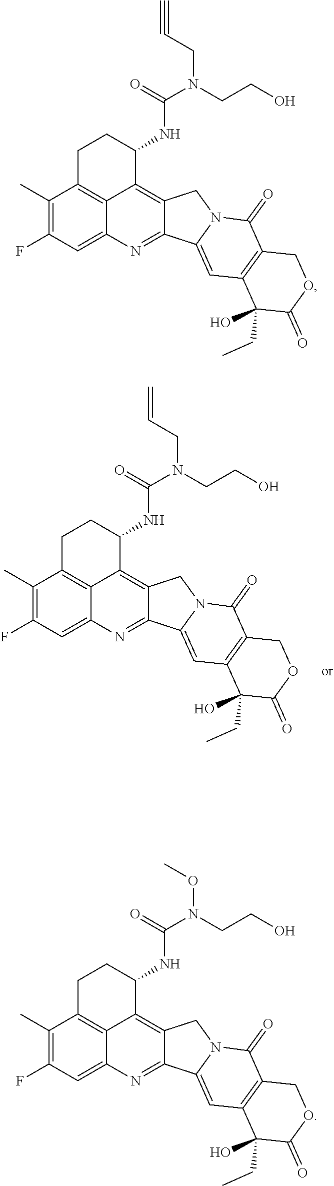 [SMILES: C#CCN(CCO)C(=O)N[C@H]1CCc2c(C)c(F)cc3nc4c(c1c23)Cn1c-4cc2c(c1=O)COC(=O)[C@]2(O)CC ; C=CCN(CCO)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(CCO)OC)CC3]

### Explanations of Terms

Unless otherwise specified, the following terms and phrases used herein are intended to have the following meanings. Without a special definition, a specific term or phrase should not be regarded as being uncertain or unclear, but should be understood according to an ordinary meaning.

The term “cytotoxic drug” or “drug” refers to a small-molecular compound with a strong destructive effect on normal tumor cell growth in tumor cells. In principle, the cytotoxic drug can kill the tumor cells at a high enough concentration, but due to the lack of specificity, the cytotoxic drug may also lead to normal cell apoptosis while killing the tumor cells, thus leading to serious side effects.

The term “antibody-drug conjugate” refers to linking an antibody to the cytotoxic drug with biological activity through a stable linker.

The term “antibody” refers to an immunoglobulin, which is a tetrapeptide chain structure composed of two identical heavy chains or two identical light chains linked through an interchain disulfide bond.

The term “antigen-binding fragment” refers to one or more fragments of the antibody, which retain an ability to specifically bind to an antigen.

The term “linker part” refers to a chemical structure fragment or bond linked to Ab.

The term “linker” refers to a chemical structure fragment linking the cytotoxic drug to Ab. In the embodiments of the present disclosure, the linker is —Y-L-X—, the cytotoxic drug is linked to an X-terminal, and the Ab is linked to a Y-terminal.

It should be noted that, in formula (Ia),

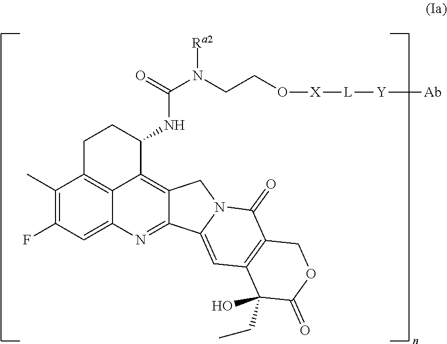

Y is selected from —CO—Y¹—Y²—S—, wherein S in Y is an S contained in the antibody itself. It can be understood by those skilled in the art that Y² is linked to sulfhydryl contained in the Ab (such as the antibody) itself after opening the disulfide bond (for example, the disulfide bond may be opened by reducing the disulfide bond with a reducing agent TCEP to generate sulfhydryl SH), that is, S between Y² and the Ab is not an additionally externally linked sulfur atom.

For example, in

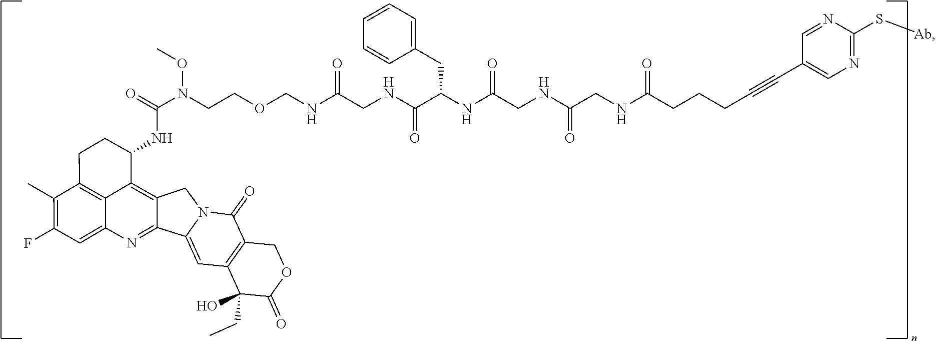

—S— is not an additionally externally linked sulfur atom, but —S— formed by linking the sulfhydryl contained in the Ab itself after opening the disulfide bond to Y², such as a right terminal of

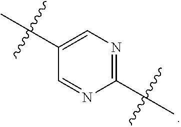 [SMILES: *c1cnc(*)nc1]

For example, in

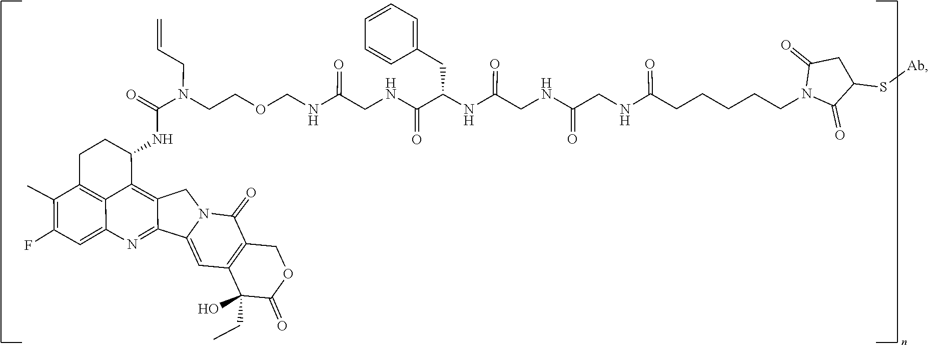

—S— is not an additionally externally linked sulfur atom, but —S— formed by linking the sulfhydryl contained in the Ab itself after opening the disulfide bond to Y², such as a right terminal of

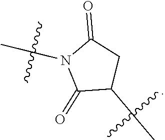 [SMILES: *C1CC(=O)N(*)C1=O]

In the present application, a positive control Dxd is purchased from MedChemexpress (MCE). Unless otherwise specified, “ring” refers to a saturated, partially saturated or unsaturated monocyclic ring or polycyclic ring.

Unless otherwise specified, the term “heterocyclyl” refers to a substituted or unsubstituted saturated or unsaturated non-aromatic ring containing 1-3 heteroatoms selected from N, O or S, and the heterocyclyl may further be substituted by ═O. The “heterocyclyl” in the present disclosure refers to a non-aromatic cyclic group which contains at least one heteroatom as a ring atom and is derived by removing one hydrogen atom; the “heterocyclyl” comprises saturated or partially saturated monocyclic heterocyclyl and polycyclic heterocyclyl. The heterocyclyl is independent of the linking position (i.e., can be bonded via a carbon atom or a heteroatom). The polycyclic heterocyclyl comprises fused heterocyclyl, spiroheterocyclyl and bridged heterocyclyl.

The “fused heterocyclyl” refers to a ring structure formed by two or more rings sharing two adjacent ring atoms with each other, and at least one ring is a heterocyclic ring. The fused heterocyclyl comprises a ring structure fused by monocyclic heterocyclyl with monocyclic heterocyclyl or cycloalkyl or aryl or heteroaryl, and further comprises a ring structure fused by heteroaryl with cycloalkyl or monocyclic heterocyclyl.

The “spiroheterocyclyl” refers to a ring structure formed by two or more rings sharing one ring atom with each other, and at least one ring is a heterocyclic ring.

The “bridged heterocyclyl” refers to a ring structure formed by two or more rings sharing non-adjacent ring atoms with each other, and at least one ring is a heterocyclic ring.

The heterocyclyl in the present disclosure is preferably 3-10 membered monocyclic heterocyclyl, 5-10 membered fused heterocyclyl, 5-10 membered spiroheterocyclyl or 5-10 membered bridged heterocyclyl; and further, the heterocyclyl is preferably 3-6 membered monocyclic heterocyclyl, 8-10 membered fused heterocyclyl, 8-10 membered spiroheterocyclyl or 7-9 membered bridged heterocyclyl; and further preferably 4-6 membered heterocyclyl.

Examples of “3-10 membered heterocyclyl” comprise, but are not limited to, tetrahydropyrrolyl, piperidinyl, piperazinyl, morpholinyl, tetrahydrofuranyl, tetrahydrothienyl, tetrahydropyranyl, 1,3-dioxolane, 1,4-dioxane,

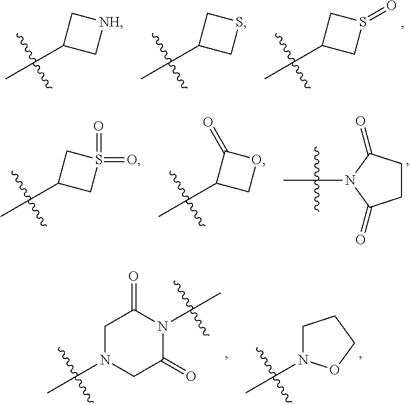

and the like.

Unless otherwise specified, the term “aryl” refers to unsaturated, usually aromatic hydrocarbyl, which may be a monocyclic ring or a plurality of rings fused together. Examples of aryl comprise, but are not limited to, phenyl and naphthyl.

Unless otherwise specified, the term “heteroaryl” refers to stable monocyclic or polycyclic aromatic hydrocarbon, preferably containing carbon atoms and 1, 2, 3 or 4 ring heteroatoms independently selected from N, O and S. The heteroaryl is preferably 5-12 membered heteroaryl, and more preferably 5-6 membered heteroaryl. Examples of heteroaryl comprise, but are not limited to, pyrrolyl, pyrazolyl, imidazolyl, pyrazinyl, isoxazolyl, thiazolyl, furyl, thienyl, pyrimidinyl and pyridyl.

Unless otherwise specified, “cycloalkyl” refers to saturated monocyclic or polycyclic hydrocarbyl. The cycloalkyl is preferably C₃₋₈ cycloalkyl, more preferably C₃₋₆ cycloalkyl, and further preferably C₅₋₆ cycloalkyl. Examples of cycloalkyl comprise, but are not limited to, cyclopropyl, cyclobutyl, cyclopentyl, cyclohexyl, cycloheptyl, cyclooctyl and

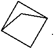

Unless otherwise specified, the term “alkyl” is used to denote straight or branched saturated hydrocarbyl. The alkyl is preferably C₁₋₆ alkyl, and more preferably C₁₋₄ alkyl. Examples of alkyl comprise, but are not limited to, methyl, ethyl, propyl, isopropyl, butyl, isobutyl, tert-butyl, pentyl, isopentyl, neopentyl, n-hexyl, and the like.

Unless otherwise specified, the term “halogen” refers to a fluorine, chlorine, bromine or iodine atom.

Unless otherwise specified, the term “alkoxy” is used to denote-O-alkyl. The alkoxy is preferably C₁₋₆ alkoxy, and more preferably C₁₋₄ alkoxy. Examples of alkoxy comprise, but are not limited to, methoxy, ethoxy, n-propoxy, isopropoxy, n-butoxy, sec-butoxy, tert-butoxy, n-pentyloxy, n-hexyloxy, and the like.

The “alkenyl” in the present disclosure refers to a group derived from straight or branched olefin (containing at least one double bond) in the absence of one hydrogen atom, comprising “C₂₋₆ alkenyl”, “C₂₋₅ alkenyl”, “C₂₋₄ alkenyl” and “C₂₋₃ alkenyl”. Specific examples of alkenyl comprise, but are not limited to: —CH═CH₂, —CH═CHCH₃, —C(CH₂)═CH₂, —CH═CHCH₂CH₃, —CH₂CH═CHCH₃, and the like.

The “alkynyl” in the present disclosure refers to a group derived from straight or branched alkyne (containing at least one triple bond) in the absence of one hydrogen atom, comprising “C₂₋₅ alkynyl”, “C₂₋₄ alkynyl” and “C₂₋₃ alkynyl”. Specific examples of alkynyl comprise, but are not limited to: —C≡CH, —C≡CHCH₃, CH≡CHCH₂—, CH≡C—C≡C—, and the like.

The term “pharmaceutically acceptable” refers to those compounds, materials, compositions and/or dosage forms, which are suitable for use in contact with human and animal tissues without excessive toxicity, irritation, allergic reaction or other problems or complications within a reasonable medical judgment range, and are commensurate with a reasonable benefit/risk ratio.

The term “pharmaceutically acceptable salt” refers to a derivative prepared from the compound of the present disclosure with a relatively nontoxic acid or base. These salts may be prepared during the synthesis, separation and purification of the compound, or prepared by allowing a free form of purified compound used alone to react with suitable acids or bases. When the compound contains relatively acidic functional groups, the compound reacts with alkali metal, alkaline earth metal hydroxide or organic amine to obtain a base addition salt, comprising cations based on alkali metal and alkaline earth metal. When the compound contains relatively basic functional groups, the compound reacts with an organic acid or an inorganic acid to obtain an acid addition salt.

The compound of the present disclosure has an isomer, such as a cis-trans isomer, an enantiomer, a diastereomer, a racemic mixture thereof and other mixtures, and all these mixtures are within the scope of the present disclosure.

The term “enantiomer” refers to stereoisomers in a mirror-image relation.

The term “diastereomer” refers to a stereoisomer that has two or more chiral centers, and molecules in a non-mirror-image relation.

The term “cis-trans isomer” refers to a configuration in which a double bond of a molecule or a single bond of ring-forming carbon atom cannot rotate freely.

Unless otherwise specified, an absolute configuration of a stereocenter is represented by a wedge-shaped solid-line bond [symbol] and a wedge-shaped dashed-line bond

[symbol]

The stereoisomer of the compound of the present disclosure may be prepared by chiral synthesis or a chiral reagent or other conventional techniques. For example, an enantiomer of some compound of the present disclosure may be prepared by an asymmetric catalysis technology or a chiral auxiliary derivation technology. Alternatively, a compound with a single stereo configuration is obtained from a mixture by a chiral resolution technology. Alternatively, the compound is directly prepared from a chiral starting material. The separation of optically pure compound in the present disclosure is usually completed by preparative chromatography, wherein a chiral chromatographic column is adopted to achieve the purpose of separating a chiral compound.

The term “pharmaceutically acceptable carrier” refers to a medium generally acceptable in the art for the delivery of a biologically active pharmaceutical agent to an animal, particularly a mammal. Depending on a method of administration and a nature of dosage form, the pharmaceutically acceptable carrier comprises an adjuvant, an excipient or a vehicle, such as a diluent, a preservative, a filler, a flow regulator, a disintegrating agent, a wetting agent, an emulsifier, a suspending agent, a sweetener, a flavoring agent, a fragrant agent, an antimicrobial, an antifungal, a lubricant and a dispersant. The pharmaceutically acceptable carrier is formulated depending on a large number of factors within the knowledge of those of ordinary skills in the art. The factors comprise, but are not limited to: the type and nature of an active agent formulated, a subject to which a composition containing the agent is to be administered, an intended route of administration of the composition, and a target therapeutic indication. The pharmaceutically acceptable carrier comprises both aqueous and non-aqueous media and a variety of solid and semi-solid dosage forms. Such a carrier comprises, in addition to the active agent, many different ingredients and additives, and additional ingredients included in the formulation for a variety of reasons (such as stabilizing the active agent and serving as an adhesive) are well known to those of ordinary skills in the art. The term “excipient” generally refers to a carrier, diluent, and/or medium required to formulate an effective pharmaceutical composition.

The term “prophylactically or therapeutically effective amount” refers to an adequate amount of the compound of the present disclosure or the pharmaceutically acceptable salt thereof which is suitable for the treatment of a disorder with a reasonable effect/risk ratio of any medical treatment and/or prevention. However, it should be understood that a total daily dosage of the compound as shown by the formula (I) of the present disclosure or the pharmaceutically acceptable salt thereof and the composition shall be determined by an attending physician within a reliable medical judgment range. For any particular patient, a specific therapeutically effective dosage level must be determined based on multiple factors, comprising a disorder to be treated and a severity of the disorder; the activity of the specific compound used; the specific composition used; the age, weight, general health status, gender, and diet of a patient; a specific administration time, an administration route, and an excretion rate of the specific compound used; a duration of treatment; drugs used in combination or simultaneously with the specific compound used; and similar factors commonly known in the medical field. For example, the practice in the art is to start with a dose of a compound below a level required to achieve a desired therapeutic effect and gradually increase the dose until the desired effect is achieved.

In the embodiments of the present disclosure, the name of the title compound is transformed through the structure of the compound with the help of Chemdraw. If the name of the compound is inconsistent with the structure of the compound, the name may be determined by synthesizing relevant information and a reaction route; and if the name cannot be confirmed by other methods, a given structural formula of the compound shall prevail. A preparation method for some compounds of the present disclosure cites a preparation method for similar compounds mentioned above. Those skilled in the art should know that, when using or referring to the cited preparation method, a feed ratio, a reaction solvent, a reaction temperature, and the like of a reactant may be appropriately adjusted according to different reactants.

The compound of the present disclosure may be prepared by various synthetic methods known to those skilled in the art, comprising the specific embodiments listed below, the embodiments formed by combining them with other chemical synthesis methods, and the equivalent alternatives known to those skilled in the art. The preferred embodiments comprise, but are not limited to, the examples of the present disclosure.

### Summary of Experimental Instruments

The structure of the compound of the present disclosure is determined by nuclear magnetic resonance (NMR) or/and liquid chromatography-mass spectrometry (LC-MS), or ultra-high-performance liquid chromatography-mass spectrometry (UPLC-MS). An NMR chemical shift (8) is given in parts per million (ppm). The NMR is performed by a Bruker Neo 400M or Bruker Ascend 400 nuclear magnetic instrument with solvents comprising deuterated dimethyl sulfoxide (DMSO-d₆), deuterated methanol (CD₃OD) and deuterated chloroform (CDCl₃), and heavy water (D₂O), and an internal standard is tetramethylsilane (TMS).

The liquid chromatography-mass spectrometry LC-MS is performed by an Agilent 1260-6125B single quadrupole mass spectrometer with a Welch Biomate column (C18, 2.7 μm. 4.6×50 mm) or a Waters H-Class SQD2 mass spectrometer with a Welch Ultimate column (XB—C18, 1.8 μm. 2.1×50 mm) (an ion source is electrospray ionization).

The ultra-high-performance liquid chromatography-mass spectrometry UPLC-MS is performed by a Water UPLC H-class SQD mass spectrometer (an ion source is electrospray ionization).

The HPLC is performed by Waters e2695-2998 or Waters ARC and Agilent 1260 or Agilent Poroshell HPH high-performance liquid chromatography.

The preparative HPLC is performed by Waters 2555-2489 (10 μm, ODS 250 cm×5 cm) or GILSON Trilution LC with a Welch XB—C18 column (5 μm, 21.2×150 mm).

A thin layer chromatography silica gel plate is a GF254 silica gel plate from Yantai Jiangyou Silica gel Development Co., Ltd. or a GF254 silica gel plate from Rushan Sanpont New Materials Co., Ltd., TLC adopts a specification of 0.15 mm-0.20 mm, preparative TLC adopts 20×20 cm, and a silica gel of 200-300 meshes from Yucheng Chemical is used as a carrier for column chromatography.

Starting materials in Embodiments of the present disclosure are known and commercially available, or may be synthesized by or according to a method known in the art. Unless otherwise specified, all reactions of the present disclosure are carried out under continuous magnetic stirring in a dry nitrogen or argon atmosphere, with the solvent being a dry solvent and the reaction temperature in degrees Celsius or ° C.

### DESCRIPTION OF THE DRAWINGS

FIG. 1 is a change trend chart of DAR value detection of each group of ADC in human plasma;

FIG. 2 is a tumor growth trend chart of each group of animals in a JIMT-1 human breast cancer model at a dosage of 3 mg/kg;

FIG. 3 is a tumor growth trend chart of each group of animals in the JIMT-1 human breast cancer model at a dosage of 10 mg/kg; and

FIG. 4 is a weight change trend chart of each group of animals in the JIMT-1 human breast cancer model.

### EXAMPLES

### Example 1

### Preparation of 1-(cyanomethyl)-3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinoline-1-yl)-1-(2-hydroxyethyl)urea (Compound 1)

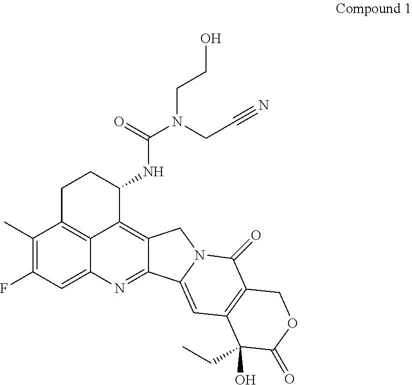 [SMILES: CC[C@@]1(O)C(=O)OCc2c1cc1n(c2=O)Cc2c-1nc1cc(F)c(C)c3c1c2[C@@H](NC(=O)N(CC#N)CCO)CC3]

### Reaction Route:

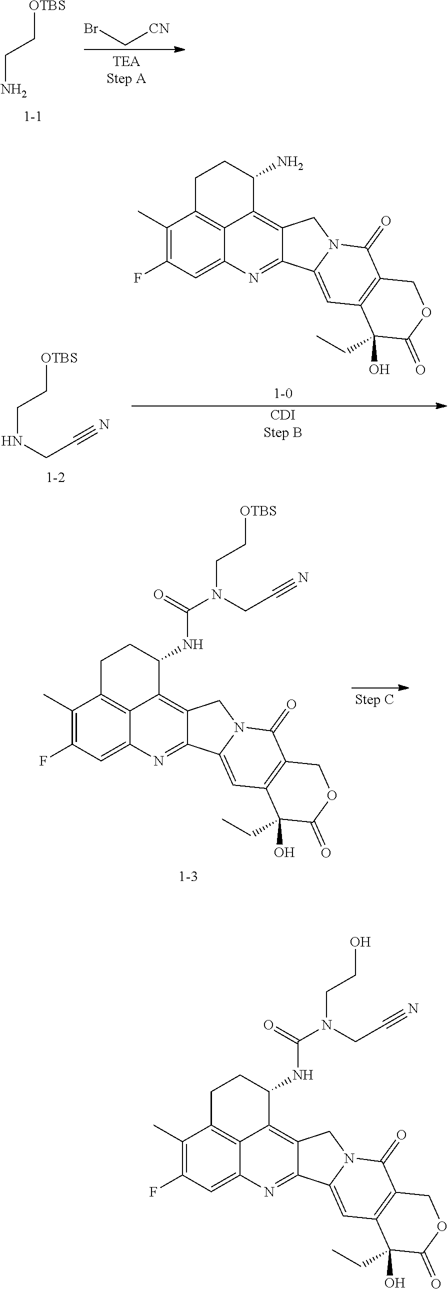

### Operation Steps:

Step A: Compound 1-1 (1.2 g, 6.85 mmol) was dissolved in tetrahydrofuran (34 mL) at room temperature. Subsequently, triethylamine (1.4 g, 13.70 mmol) and bromoacetonitrile (0.82 g, 6.85 mmol) were dropwise added to the above solution sequentially. Then, the reaction system was stirred at 40° C. for 16 hours.

After LCMS showed that the raw materials disappeared, water (15 mL) was added to the reaction solution for quenching, the mixed solution was extracted with ethyl acetate (15 mL×3 times), and organic phases were combined and washed with saturated saline solution (20 mL×2 times), then was dried with anhydrous sodium sulfate, filtered and concentrated under a reduced pressure. The obtained residue was purified by silica gel column chromatography to obtain a Compound 1-2 (350.0 mg, yield 23.8%).

MS (ESI) M/Z: 215.4 [M+H]⁺.

Step B: Compound 1-0 (70.0 mg, 0.16 mmol) was dissolved in dry N,N-dimethylformamide (1.6 mL) at 0° C. Subsequently, N,N′-carbonyldiimidazole (39.0 mg, 0.24 mmol) was added to the reaction system and the obtained mixture was stirred for 0.5 hour. Then, Compound 1-2 (51.0 mg, 0.24 mmol) was dissolved in N,N-dimethylformamide (0.2 mL), the obtained system was slowly dropwise added into the above reaction solution, and continuously stirred for 0.5 hour.

After LCMS showed that the raw materials disappeared, water (20 mL) was added to the reaction solution for quenching, the mixed solution was extracted with ethyl acetate (8 mL×3 times), and organic phases were combined and washed with saturated saline solution (15 mL×2 times), then was dried with anhydrous sodium sulfate, filtered and concentrated under a reduced pressure. The obtained residue was purified by silica gel column chromatography to obtain Compound 1-3 (75.0 mg, yield 69.4%).

MS (ESI) M: 676.3 [M+H]⁺.

Step C: Compound 1-3 (65.0 mg, 0.10 mmol) was dissolved in methanol (0.9 mL) at room temperature. Then, 1% TFA aqueous solution (1.8 mL) was slowly dropwise added to the reaction system and the obtained mixture was continuously stirred for 7 hours.

After LCMS showed that the raw materials disappeared, the reaction solution was filtered, and the obtained solid was purified by preparative high-performance liquid chromatography to obtain the title Compound 1 (12.0 mg, yield 22.2%).

MS (ESI) M: 562.3 [M+H]⁺.

¹H NMR (400 MHz, DMSO-d₆) δ 7.77 (d, J=11.2 Hz, 1H), 7.30 (s, 1H), 7.25 (d, J=8.0 Hz, 1H), 6.52 (s, 1H), 5.47-5.35 (m, 3H), 5.25 (d, J=19.2 Hz, 1H), 5.18 (d, J=18.8 Hz, 1H), 5.00 (t, J=4.6 Hz, 1H), 4.45 (d, J=17.6 Hz, 1H), 4.37 (d, J=17.6 Hz, 1H), 3.53 (q, J=5.2 Hz, 2H), 3.44-3.34 (m, 2H), 3.15 (t, J=6.0 Hz, 2H), 2.39 (s, 3H), 2.27-2.08 (m, 2H), 1.94-1.78 (m, 2H), 0.87 (t, J=7.4 Hz, 3H).

### Example 2

### Preparation of 3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-(2-hrdyoxyethyl-1-)prop-2-yn-1-yl)urea (Compound 2)

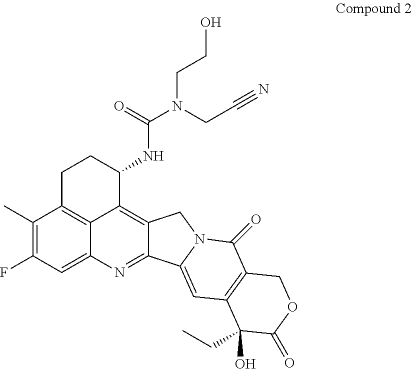 [SMILES: CC[C@@]1(O)C(=O)OCc2c1cc1n(c2=O)Cc2c-1nc1cc(F)c(C)c3c1c2[C@@H](NC(=O)N(CC#N)CCO)CC3]

### Reaction Route:

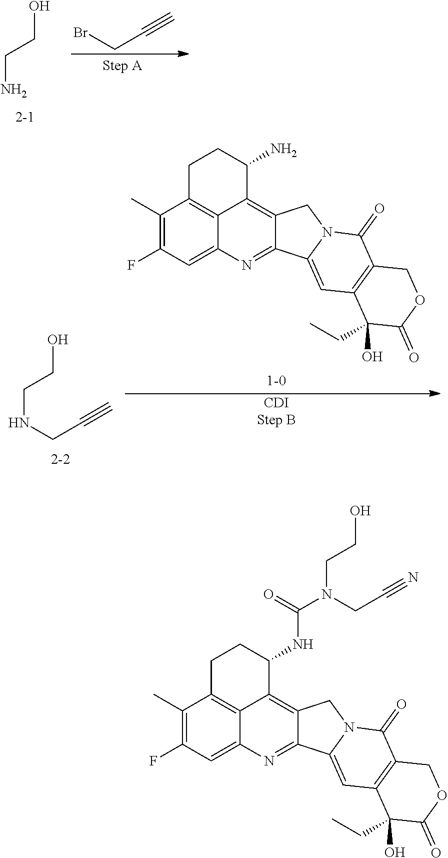

### Operation Steps:

Step A: Compound 2-1 (2.0 g, 32.73 mmol) was dissolved in dichloromethane (33 mL) at room temperature. Subsequently, 3-propargyl bromide (1.0 g, 8.18 mmol). Then, the reaction system was continuously stirred for 2 hours.

After LCMS showed that the raw materials disappeared, t dichloromethane (20 mL) was added to the reaction solution for dilution, and the mixed solution was washed with water (20 mL×3 times), then dried with anhydrous sodium sulfate, filtered, and concentrated under a reduced pressure. The obtained residue was purified by silica gel column chromatography to obtain Compound 2-2 (265.0 mg, yield 32.7%).

MS (ESI) M/Z: 100.2 [M+H]⁺.

Step B: Compound 1-0 (80.0 mg, 0.18 mmol) was dissolved in dry N,N-dimethylformamide (1.0 mL) at 0° C. Subsequently, N,N′-carbonyldiimidazole (44.0 mg, 0.27 mmol) was added to the reaction solution and the mixture was stirred for 0.5 hours. Then, Compound 2-2 (27.0 mg, 0.27 mmol) was dissolved in N,N-dimethylformamide (0.2 mL), slowly dropwise added into the above reaction solution, and continuously stirred for 0.5 hours.

After LCMS showed that the raw materials disappeared, water (10 mL) and ethyl acetate (15 mL) were added to the reaction solution for quenching, solid was precipitated, and filtered, the obtained filter cake was pulped with methanol (10 mL) and filtered, then the filter cake was washed with methanol (10 mL×2 times), and solid was collected and concentrated under a reduced pressure to obtain the title Compound 2 (14.7 mg, yield 14.3%).

MS (ESI) M: 561.2 [M+H]⁺.

¹H NMR (400 MHz, DMSO-d₆) δ 7.75 (d, J=10.8 Hz, 1H), 7.30 (s, 1H), 7.04 (d, J=8.0 Hz, 1H), 6.51 (s, 1H), 5.49-5.31 (m, 3H), 5.21 (s, 2H), 4.92 (t, J=4.8 Hz, 1H), 4.21 (dd, J=18.0, 2.0 Hz, 1H), 4.13 (dd, J=17.8, 2.2 Hz, 1H), 3.55 (q, J=5.2 Hz, 2H), 3.37 (t, J=5.4 Hz, 2H), 3.23-3.03 (m, 3H), 2.38 (s, 3H), 2.25-2.08 (m, 2H), 1.95-1.77 (m, 2H), 0.87 (t, J=7.4 Hz, 3H).

### Example 3

### Preparation of 1-allyl-3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-(2-hydroxyethyl)urea (Compound 3)

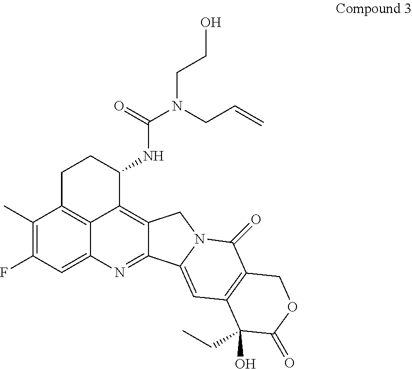 [SMILES: C=CCN(CCO)C(=O)N[C@H]1CCc2c(C)c(F)cc3nc4c(c1c23)Cn1c-4cc2c(c1=O)COC(=O)[C@]2(O)CC]

### Reaction Route:

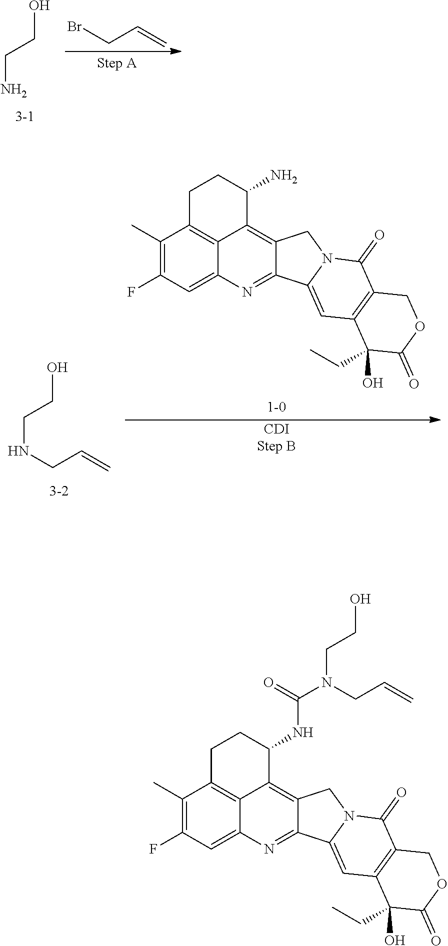

### Operation Steps:

Step A: Compound 3-1 (2.0 g, 32.73 mmol) was dissolved in tetrahydrofuran (33 mL) at 0° C. Subsequently, triethylamine (826.0 mg, 8.18 mmol) and 3-bromopropylene (990.0 mg, 8.18 mmol) were slowly dropwise added to the above solution sequentially. Then, the reaction system was continuously stirred at room temperature for 2 hours.

After LCMS showed that the raw materials disappeared, the reaction solution was concentrated under a reduced pressure. The obtained residue was purified by silica gel column chromatography to obtain Compound 3-2 (240.0 mg, yield 29.1%).

MS (ESI) M/Z: 102.3 [M+H]⁺.

Step B: Compound 1-0 (40.0 mg, 0.09 mmol) was dissolved in dry N,N-dimethylformamide (1.0 mL) at 0° C. Subsequently, N,N′-carbonyldiimidazole (23.0 mg, 0.14 mmol) was added to the above solution and stirred for 0.5 hours. Then, Compound 3-2 (18.0 mg, 0.18 mmol) was added and the obtained system was continuously stirred for 0.5 hours.

After LCMS showed that the raw materials disappeared, water (15 mL) was added to the reaction solution for quenching, the mixed solution was extracted with ethyl acetate (10 mL×3 times), and organic phases were combined and washed with saturated saline solution (20 mL×2 times). Then, the solution was dried with anhydrous sodium sulfate, filtered and concentrated under a reduced pressure. The obtained residue was purified by preparative high-performance liquid chromatography to obtain the title Compound 3 (6.8 mg, yield 13.5%).

MS (ESI) M: 563.3 [M+H]⁺.

¹H NMR (400 MHz, DMSO-d₆) δ 7.74 (d, J=11.2 Hz, 1H), 7.30 (s, 1H), 6.89 (d, J=8.0 Hz, 1H), 6.51 (s, 1H), 5.90-5.75 (m, 1H), 5.48-5.37 (m, 3H), 5.26-5.09 (m, 4H), 4.88 (t, J=4.8 Hz, 1H), 4.01 (dd, J=16.4, 5.2 Hz, 1H), 3.90 (dd, J=16.4, 5.6 Hz, 1H), 3.51 (q, J=5.6 Hz, 2H), 3.30-3.21 (m, 2H), 3.20-3.03 (m, 2H), 2.37 (s, 3H), 2.17-2.12 (m, 2H), 1.93-1.78 (m, 2H), 0.88 (t, J=7.2 Hz, 3H).

### Example 4

### Preparation of (S)—N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-4-hydroxyisoxazolidine-2-carboxamide (Compound 4)

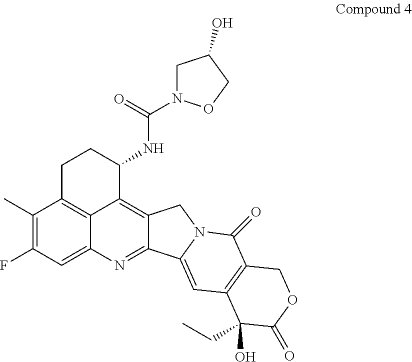 [SMILES: CC[C@@]1(O)C(=O)OCc2c1cc1n(c2=O)Cc2c-1nc1cc(F)c(C)c3c1c2[C@@H](NC(=O)N1C[C@H](O)CO1)CC3]

### Reaction Route:

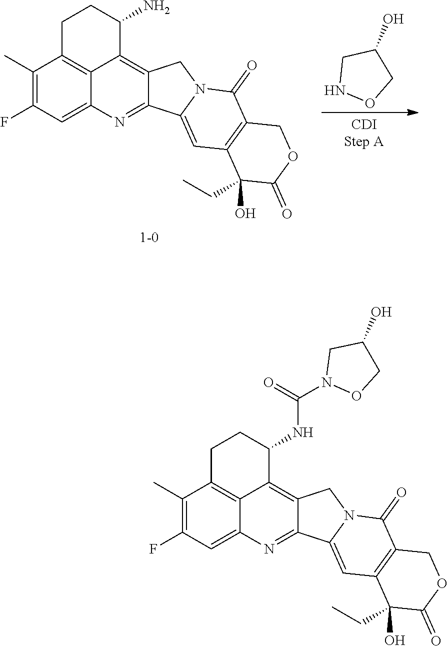 [SMILES: CC[C@@]1(O)C(=O)OCc2c1cc1n(c2=O)Cc2c-1nc1cc(F)c(C)c3c1c2[C@@H](N)CC3 ; O[C@H]1CNOC1 ; CC[C@@]1(O)C(=O)OCc2c1cc1n(c2=O)Cc2c-1nc1cc(F)c(C)c3c1c2[C@@H](NC(=O)N1C[C@H](O)CO1)CC3]

### Operation Steps:

Step B: Compound 1-0 (43.5 mg, 0.10 mmol) was dissolved in N,N-dimethylformamide solution (1.0 mL) at 0° C. Subsequently, the above solution was cooled to 0° C., N,N′-carbonyldiimidazole (32.4 mg, 0.20 mmol) was added, and the obtained system was stirred for 30 minutes. Then, (S)-isoxazolin-4-ol hydrochloride (25.0 mg, 0.20 mmol) and N,N-diisopropylethylamine (77.4 mg, 0.60 mmol) were added, heated to 50° C., and continuously stirred for 3 hours.

After LCMS showed that the raw materials disappeared, the reaction solution was directly purified by preparative high-performance liquid chromatography to obtain the title Compound 4 (25.6 mg, yield 46.5%).

MS (ESI) M: 551.2 [M+H]⁺.

¹H NMR (400 MHz, DMSO-d₆) δ 8.02 (d, J=8.8 Hz, 1H), 7.76 (d, J=10.8 Hz, 1H), 7.30 (s, 1H), 6.51 (s, 1H), 5.42 (s, 2H), 5.38-5.29 (m, 2H), 5.24 (d, J=18.8 Hz, 1H), 5.14 (d, J=18.8 Hz, 1H), 4.72-4.65 (m, 1H), 3.94 (dd, J=8.6, 5.0 Hz, 1H), 3.81-3.72 (m, 2H), 3.52 (dd, J=11.2, 1.6 Hz, 1H), 3.29-3.210 (m, 1H), 3.13-3.02 (m, 1H), 2.38 (s, 3H), 2.23-2.12 (m, 2H), 1.93-1.79 (m, 2H), 0.87 (t, J=7.2 Hz, 3H).

### Example 5

### Preparation of (R)—N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-4-hydroxyisoxazoline-2-carboxamide (Compound 5)

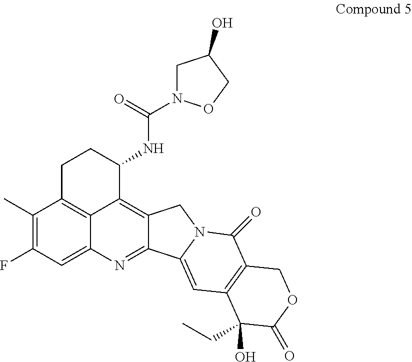 [SMILES: CC[C@@]1(O)C(=O)OCc2c1cc1n(c2=O)Cc2c-1nc1cc(F)c(C)c3c1c2[C@@H](NC(=O)N1C[C@@H](O)CO1)CC3]

### Reaction Route:

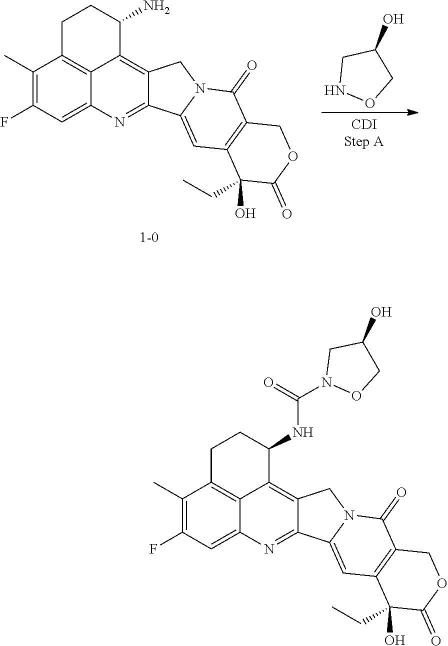 [SMILES: CC[C@@]1(O)C(=O)OCc2c1cc1n(c2=O)Cc2c-1nc1cc(F)c(C)c3c1c2[C@@H](N)CC3 ; O[C@@H]1CNOC1 ; CC[C@@]1(O)C(=O)OCc2c1cc1n(c2=O)Cc2c-1nc1cc(F)c(C)c3c1c2[C@H](NC(=O)N1C[C@@H](O)CO1)CC3]

### Operation Steps:

Step B: Compound 1-0 (43.5 mg, 0.10 mmol) was dissolved in N,N-dimethylformamide solution (1.0 mL) at 0° C. Subsequently, the above solution was cooled to 0° C., N,N′-carbonyldiimidazole (32.4 mg, 0.20 mmol) was added, and the obtained system was stirred for 30 minutes. Then, (R)-isoxazoline-4-ol hydrochloride (25.0 mg, 0.2 mmol) and N,N-diisopropylethylamine (77.4 mg, 0.6 mmol) were added, the system was heated to 50° C., and continuously stirred for 3 hours.

After LCMS showed that the raw materials disappeared, the reaction solution was directly purified by preparative high-performance liquid chromatography to obtain the title Compound 5 (25.6 mg, yield 46.5%).

MS (ESI) M: 551.2 [M+H]⁺.

¹H NMR (400 MHz, DMSO-d₆) δ 8.04 (d, J=8.8 Hz, 1H), 7.77 (d, J=10.8 Hz, 1H), 7.32 (s, 1H), 6.51 (s, 1H), 5.42 (s, 2H), 5.39-5.32 (m, 1H), 5.31-5.27 (m, 2H), 5.21 (d, J=18.8 Hz, 1H), 4.63 (s, 1H), 3.88-3.76 (m, 2H), 3.68-3.58 (m, 2H), 3.26-3.17 (m, 1H), 3.16-3.05 (m, 1H), 2.39 (s, 3H), 2.123-2.12 (m, 2H), 1.95-1.78 (m, 2H), 0.88 (t, J=7.4 Hz, 3H).

### Example 6

### Preparation of 1-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-(2-hydroxyethoxy)urea (Compound 6)

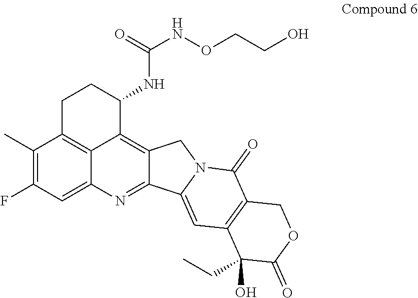 [SMILES: CC[C@@]1(O)C(=O)OCc2c1cc1n(c2=O)Cc2c-1nc1cc(F)c(C)c3c1c2[C@@H](NC(=O)NOCCO)CC3]

### Reaction Route:

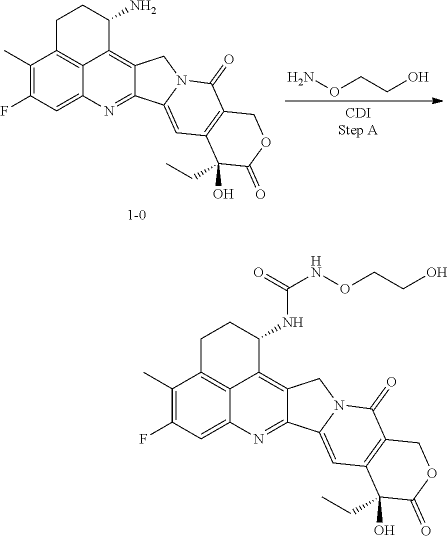 [SMILES: CC[C@@]1(O)C(=O)OCc2c1cc1n(c2=O)Cc2c-1nc1cc(F)c(C)c3c1c2[C@@H](N)CC3 ; NOCCO ; CC[C@@]1(O)C(=O)OCc2c1cc1n(c2=O)Cc2c-1nc1cc(F)c(C)c3c1c2[C@@H](NC(=O)NOCCO)CC3]

### Operation Steps:

Step A: Compound 1-0 (65.5 mg, 0.15 mmol) was dissolved in N,N-dimethylformamide solution (3.0 mL) at 0° C. Subsequently, the above solution was cooled to 0° C., N,N-diisopropylethylamine (73.0 mg, 0.45 mmol) and N,N′-carbonyldiimidazole (58.2 mg, 0.45 mmol) were added, and the obtained system was stirred for 30 minutes. Finally, 2-aminooxy ethanol (17.3 mg, 0.22 mmol) was added, and continuously stirred at room temperature for 3 hours.

After LCMS showed that the raw materials disappeared, the reaction solution was directly purified by preparative high-performance liquid chromatography to obtain the title Compound 6 (13.5 mg, yield 16.7%).

MS (ESI) M: 504.2 [M+H]⁺.

¹H NMR (400 MHz, DMSO-d₆) δ 9.46 (s, 1H), 7.78 (d, J=11.0 Hz, 1H), 7.58 (d, J=9.0 Hz, 1H), 7.31 (s, 1H), 6.51 (s, 1H), 5.50-5.42 (m, 1H), 5.42 (s, 2H), 5.33-5.17 (m, 2H), 4.81 (t, J=5.2 Hz, 1H), 3.76 (t, J=4.7 Hz, 2H), 3.57-3.46 (m, 2H), 3.31-3.19 (m, 1H), 3.18-3.07 (m, 1H), 2.39 (s, 3H), 2.25-2.12 (m, 2H), 1.94-1.80 (m, 2H), 0.88 (t, J=7.3 Hz, 3H).

### Example 7

### Preparation of 3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-hydroxy-1-(2-hydroxyethyl)urea (Compound 7)

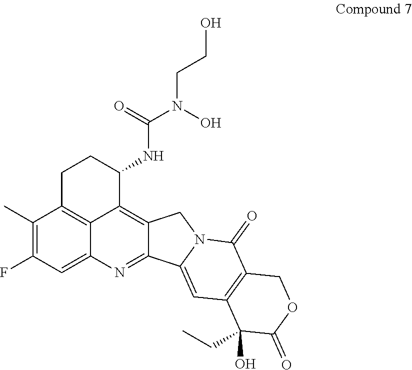 [SMILES: CC[C@@]1(O)C(=O)OCc2c1cc1n(c2=O)Cc2c-1nc1cc(F)c(C)c3c1c2[C@@H](NC(=O)N(O)CCO)CC3]

### Reaction Route:

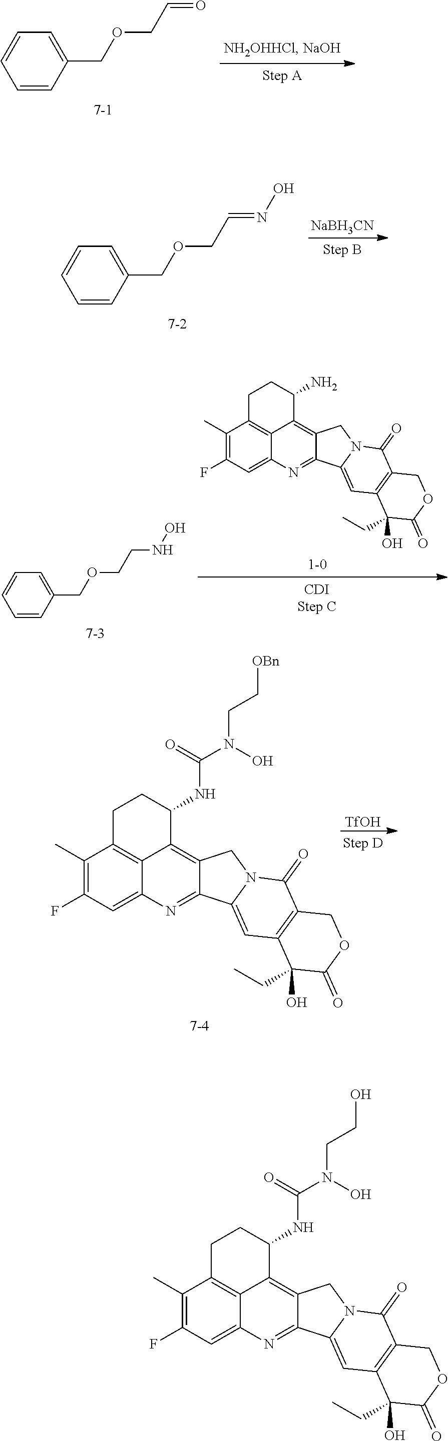

### Operation Steps:

Step A: Compound 7-1 (500.0 mg, 3.33 mmol) was added into a 25 ml vial, dissolved with water (5.0 mL), then hydroxylamine hydrochloride (694.0 mg, 9.99 mmol) was added, subsequently sodium hydroxide (466.0 mg, 11.66 mmol) was added, and the obtained system was stirred at 100° C. for 1 hour under nitrogen atmosphere.

After LCMS showed that the reaction was completed, the reaction solution was directly purified by preparative high-performance liquid chromatography, under the following purification conditions: chromatographic column: YMC-Actus Triart C18 20.0 mm×150 mm; mobile phases: water (containing 0.1% formic acid) and acetonitrile; flow rate: 20 mL/min; gradient: increase of acetonitrile from 10% to 40% within 10 minutes; and detection wavelength: 254 nm. The product was collected, and freeze-dried under a reduced pressure to obtain Compound 7-2 (340.0 mg, yield 61.8%).

MS (ESI) M: 166.1 [M+H]⁺.

Step B: Compound 7-2 (200.0 mg, 1.21 mmol) was added into a 25 ml vial, dissolved with acetic acid (2.0 mL), then sodium cyanoborohydride (152.0 mg, 2.42 mmol) was added, and the obtained system was stirred at room temperature for 1 hour under nitrogen atmosphere.

After LCMS showed that the reaction was completed, the reaction solution was directly purified by preparative high-performance liquid chromatography to obtain Compound 7-3 (120.0 mg, yield 59.3%).

MS (ESI) M: 168.1 [M+H]⁺.

Step C: Compound 1-0 (80.0 mg, 0.18 mmol) was added into a 25 ml single-necked flask, dissolved with N,N-dimethylformamide (1.0 mL), N,N′-carbonyldiimidazole (45.0 mg, 0.27 mmol) and triethylamine (54.0 mg, 0.54 mmol) were added sequentially, and stirred at room temperature for 30 minutes under nitrogen atmosphere. Then, Compound 7-3 (29.0 mg, 0.18 mmol) was added and stirred at room temperature for 1 hour under nitrogen atmosphere.

After LCMS showed that the reaction was completed, the reaction solution was directly purified by preparative high-performance liquid chromatography to obtain Compound 7-4 (60.0 mg, yield 51.9%).

MS (ESI) M: 629.3 [M+H]⁺.

¹H NMR (400 MHz, DMSO-d₆) δ 9.49 (s, 1H), 7.76 (d, J=10.9 Hz, 1H), 7.63 (d, J=9.0 Hz, 1H), 7.40-7.20 (m, 5H), 5.39 (d, J=17.7 Hz, 3H), 5.29 (d, J=19.4 Hz, 1H), 5.13 (d, J=19.0 Hz, 1H), 4.51 (s, 2H), 3.78-3.63 (m, 6H), 3.27-3.05 (m, 2H), 2.38 (s, 3H), 2.16 (d, J=6.2 Hz, 2H), 1.95-1.78 (m, 2H), 0.86 (t, J=7.4 Hz, 3H).

Step D: Compound 7-4 (40.0 mg, 0.064 mmol) was added into a 25 ml single-necked flask, dissolved with dichloromethane (1.0 mL), trifluoromethanesulfonic acid (0.1 mL) was added, and the obtained system was stirred at room temperature for 3 hours under nitrogen atmosphere.

After LCMS showed that the reaction was completed, the reaction solution was directly purified by preparative high-performance liquid chromatography to obtain the title Compound 7 (15.0 mg, yield 34.3%).

MS (ESI) M: 539.2 [M+H]⁺.

¹H NMR (400 MHz, DMSO-d₆) δ 9.49 (s, 1H), 7.76 (d, J=10.9 Hz, 1H), 7.63 (d, J=9.0 Hz, 1H), 7.40-7.20 (m, 5H), 5.39 (d, J=17.7 Hz, 3H), 5.29 (d, J=19.4 Hz, 1H), 5.13 (d, J=19.0 Hz, 1H), 4.51 (s, 2H), 3.78-3.63 (m, 6H), 3.27-3.05 (m, 2H), 2.38 (s, 3H), 2.16 (d, J=6.2 Hz, 2H), 1.95-1.78 (m, 2H), 0.86 (t, J=7.4 Hz, 3H).

### Example 8

### Preparation of 3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-(2-hydroxyethyl)-1-methoxyurea (Compound 8)

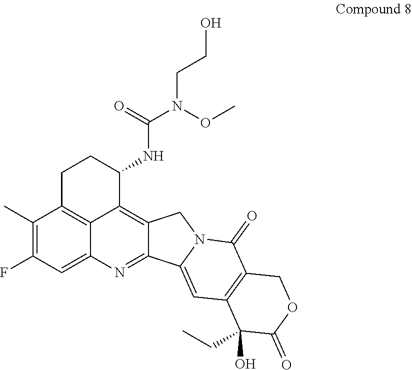 [SMILES: CC[C@@]1(O)C(=O)OCc2c1cc1n(c2=O)Cc2c-1nc1cc(F)c(C)c3c1c2[C@@H](NC(=O)N(CCO)OC)CC3]

### Reaction Route:

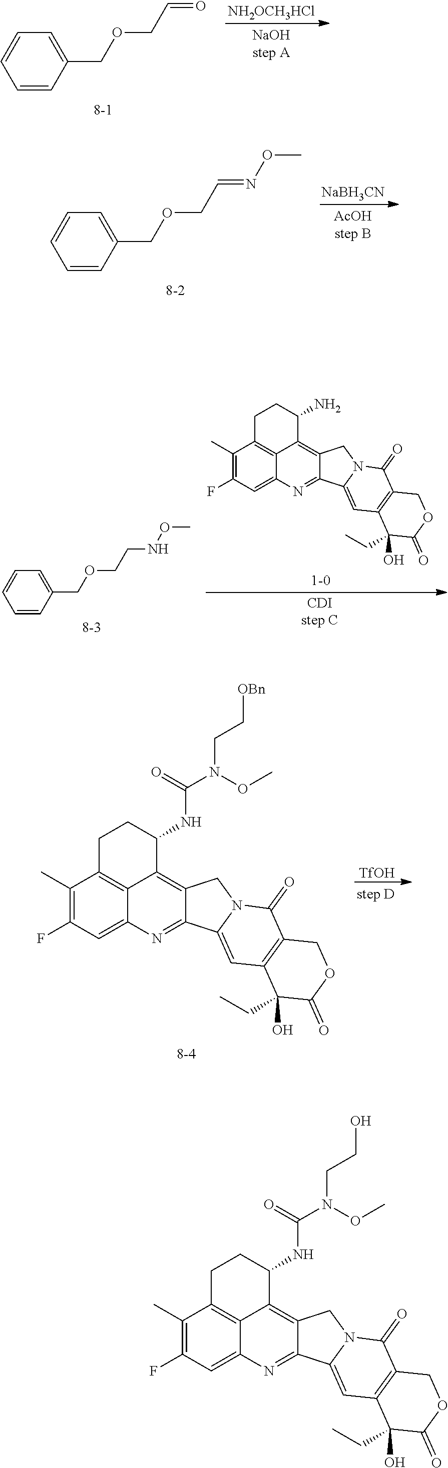

### Operation Steps:

Step A: Compound 8-1 (500.0 mg, 3.33 mmol) was dissolved with water (20.0 mL), methoxyamine hydrochloride (306.0 mg, 3.66 mmol) was added, and then sodium hydroxide (400.0 mg, 9.99 mmol) was added to react at 100° C. for 1 hour under oil bath.

After LCMS showed that the reaction was completed, the mixed solution was concentrated, diluted with methanol (20.0 mL), and filtered, and the filtrate was directly purified by preparative high-performance liquid chromatography to obtain Compound 8-2 (200.0 mg, off-white solid, yield 33.5%).

MS (ESI) M: 180.1 [M+H]⁺.

Step B: Compound 8-2 (140.0 mg, 0.782 mmol) was dissolved with AcOH (20 mL), and then NaBH₃CN (148 mg, 2.35 mmol) was added to react at room temperature for 1 hour.

After LCMS showed that the reaction was completed, the mixed solution was directly purified by preparative high-performance liquid chromatography to obtain Compound 8-3 (120.0 mg, yield 85.1%).

MS (ESI) M: 182.3 [M+H]⁺.

Step C: Compound 1-0 (20.0 mg, 0.04 mmol) was dissolved with ultra-dry N,N-dimethylformamide (1.0 mL), N,N′-carbonyldiimidazole (7.5 mg, 0.05 mmol) and triethylamine (12.0 mg, 0.11 mmol) were added, and the obtained system was stirred at room temperature for half an hour. Then, Compound 8-3 (18.1 mg, 0.10 mmol) was added to continuously react at room temperature for 1 hour.

After LCMS showed that the reaction was completed, the mixed solution was purified by preparative high-performance liquid chromatography to obtain a Compound 8-4 (16.2 mg, yield 58.5%).

MS (ESI) M: 641.3 [M−H]⁻.

¹H NMR (400 MHz, DMSO-d₆) δ 7.91 (d, J=9.0 Hz, 1H), 7.77 (d, J=11.0 Hz, 1H), 7.31-7.23 (m, 5H), 6.51 (s, 1H), 5.48-5.34 (m, 3H), 5.19 (dd, J=45.5, 18.9 Hz, 2H), 4.55-4.46 (m, 2H), 3.79 (dd, J=13.8, 6.3 Hz, 1H), 3.73-3.67 (m, 2H), 3.64 (s, 3H), 3.28-3.18 (m, 1H), 3.09 (dd, J=7.2, 4.9 Hz, 2H), 2.38 (s, 3H), 2.14 (d, J=4.7 Hz, 2H), 2.00 (dd, J=14.4, 7.0 Hz, 1H), 1.92-1.78 (m, 2H), 0.87 (t, J=7.3 Hz, 3H).

Step D: Compound 8-4 (15.0 mg, 0.02 mmol) was dissolved with dichloromethane (4 mL), trifluoromethanesulfonic acid (1.0 mL) was added, and the obtained system was stirred at room temperature to react overnight.

After LCMS showed that the reaction was completed, the mixed solution was concentrated, diluted with methanol (5 mL), and then purified by preparative high-performance liquid chromatography to obtain the title Compound 8 (8.0 mg, yield 62%).

MS (ESI) M: 553.2 [M+H]⁺.

¹H NMR (400 MHz, DMSO-d₆) δ 7.87 (d, J=9.0 Hz, 1H), 7.73 (t, J=8.9 Hz, 1H), 7.31 (s, 1H), 6.51 (s, 1H), 5.40 (d, J=10.9 Hz, 3H), 5.15 (dd, J=72.0, 19.0 Hz, 2H), 4.75 (s, 1H), 3.62 (m, 7H), 3.18 (dd, J=64.2, 11.3 Hz, 2H), 2.37 (s, 3H), 2.20 (d, J=5.6 Hz, 2H), 1.95-1.76 (m, 2H), 0.88 (t, J=7.2 Hz, 3H).

### Example 9

### Preparation of 1-cyano-3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-(2-hydroxyethyl)urea (Compound 9)

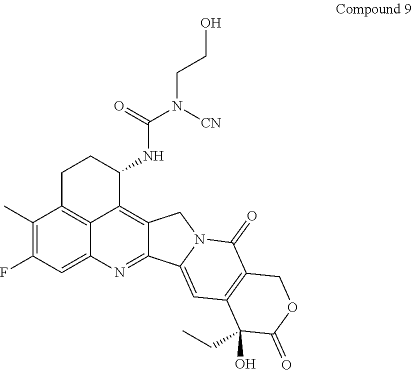 [SMILES: CC[C@@]1(O)C(=O)OCc2c1cc1n(c2=O)Cc2c-1nc1cc(F)c(C)c3c1c2[C@@H](NC(=O)N(C#N)CCO)CC3]

### Reaction Route:

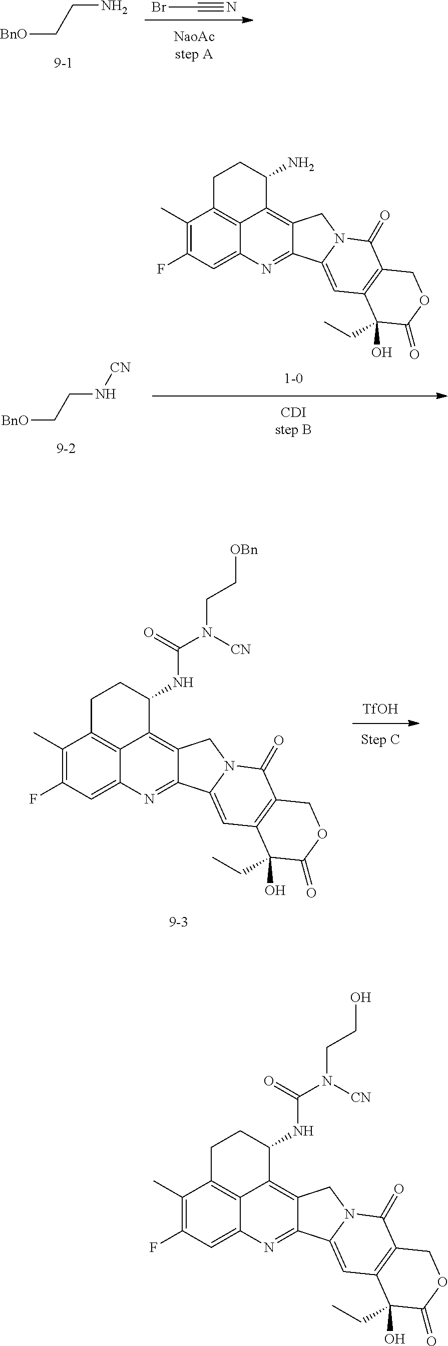

### Operation Steps:

Step A: Compound 9-1 (400.0 mg, 2.65 mmol) was added into a 25 ml single-necked flask, dry N,N-dimethylformamide (6.0 mL) was added, and then cyanogen bromide (561.4 mg, 5.30 mmol) and anhydrous sodium acetate (653.1 mg, 7.95 mmol) were added, and the reaction system reacted at room temperature for 6 hours under nitrogen protection.

After the reaction was completed, the reaction solution was filtered, and the filtrate was purified by preparative high-performance liquid chromatography to obtain Compound 9-2 (230.0 mg, yield 49.3%).

MS (ESI) M: 177.2 [M+H]⁺.

Step B: Compound 1-0 (150.0 mg, 0.34 mmol) was added into a 25 ml single-necked flask, a solvent: dry N,N-dimethylformamide (4.0 mL) was added, and six drops of N,N-diisopropylethylamine was dropwise added until the reaction solution was clear. The reaction solution was stirred at room temperature for 5 minutes, then N,N′-carbonyldiimidazole (110.3 mg, 0.68 mmol) and N,N-diisopropylethylamine (0.1 mL) were added, and the obtained system was stirred at room temperature to react for 1 hour. Then, Compound 9-2 (89.9 mg, 0.51 mmol) was added, and the reaction system reacted at room temperature for 15 hours under nitrogen protection.

After the reaction was completed, the reaction solution was filtered and purified by preparative high-performance liquid chromatography to obtain Compound 9-3 (68.0 mg, yield 37.8%).

MS (ESI) M: 638.4 [M+H]⁺.

¹H NMR (400 MHz, DMSO-d₆) δ 8.31 (d, J=8.0 Hz, 1H), 7.80 (d, J=10.9 Hz, 1H), 7.35-7.17 (m, 6H), 6.52 (s, 1H), 5.49-5.36 (m, 3H), 5.27 (d, J=18.8 Hz, 1H), 5.13 (d, J=19.1 Hz, 1H), 4.52 (s, 2H), 3.86-3.75 (m, 2H), 3.69-3.62 (m, 2H), 3.11 (t, J=6.3 Hz, 2H), 2.39 (s, 3H), 2.28-2.18 (m, 1H), 2.17-2.06 (m, 1H), 1.94-1.78 (m, 2H), 0.88 (t, J=7.3 Hz, 3H).

Step C: Compound 9-3 (30.0 mg, 0.34 mmol) was added into a 25 ml single-necked flask, a solvent: dichloromethane (4.0 mL) was added, and then trifluoromethanesulfonic acid (0.4 mL) was added, and the reaction system reacted at room temperature for 15 hours under nitrogen protection.

After the reaction was completed, a saturated NH₄HCO₃ solution was added, and then extracted and washed with ethyl acetate (20 mL×3), and organic phases were combined, then dried with anhydrous sodium sulfate, filtered, and finally concentrated under a reduced pressure. The crude product was purified by preparative high-performance liquid chromatography to obtain the title Compound 9 (5.1 mg, yield 19.9%).

MS (ESI) M: 548.3 [M+H]⁺.

¹H NMR (400 MHz, DMSO-d₆) δ 8.39 (s, 2H), 7.81 (d, J=10.8 Hz, 1H), 7.31 (s, 1H), 7.06 (s, 1H), 6.52 (s, 1H), 5.53 (s, 1H), 5.42 (s, 2H), 5.36 (d, J=8.1 Hz, 1H), 4.34-4.22 (m, 2H), 3.98-3.86 (m, 2H), 3.12-2.99 (m, 2H), 2.40 (s, 3H), 2.29-2.19 (m, 2H), 1.93-1.77 (m, 2H), 0.86 (t, J=7.3 Hz, 3H).

### Example 10

### Preparation of N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxyazetidine-1-carboxamide (Compound 10)

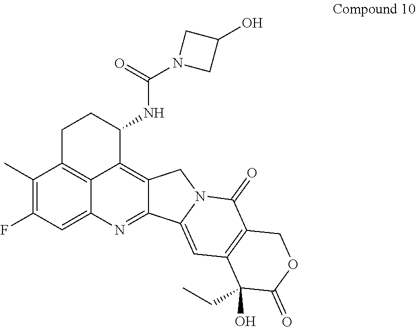 [SMILES: CC[C@@]1(O)C(=O)OCc2c1cc1n(c2=O)Cc2c-1nc1cc(F)c(C)c3c1c2[C@@H](NC(=O)N1CC(O)C1)CC3]

### Reaction Route:

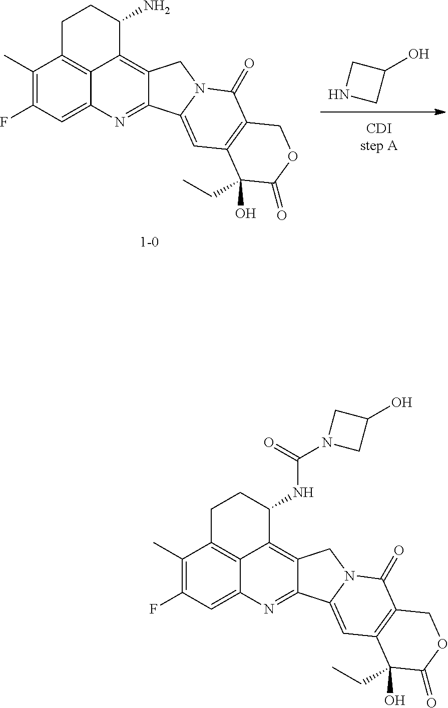 [SMILES: CC[C@@]1(O)C(=O)OCc2c1cc1n(c2=O)Cc2c-1nc1cc(F)c(C)c3c1c2[C@@H](N)CC3 ; OC1CNC1 ; CC[C@@]1(O)C(=O)OCc2c1cc1n(c2=O)Cc2c-1nc1cc(F)c(C)c3c1c2[C@@H](NC(=O)N1CC(O)C1)CC3]

### Operation Steps:

Step A: Compound 1-0 (20.0 mg, 0.04 mmol) was added into a 25 ml single-necked flask, a solvent: dry N,N-dimethylformamide (2.0 mL) was added, and two drops of N,N-diisopropylethylamine was dropwise added until the reaction solution was clear. The reaction solution was stirred at room temperature for 5 minutes, then N,N′-carbonyldiimidazole (12.2 mg, 0.08 mmol) and three drops of N,N-diisopropylethylamine were added, and the obtained system was stirred at room temperature to react for 1 hour. Then, azetidin-3-ol (8.2 mg, 0.08 mmol) was added, and the reaction system reacted at room temperature for 1 hour under nitrogen protection.

After the reaction was completed, the reaction solution was filtered and purified by preparative high-performance liquid chromatography to obtain the title Compound 10 (7.1 mg, yield 35.2%).

MS (ESI) M: 535.2 [M+H]⁺.

¹H NMR (400 MHz, DMSO-d₆) δ 7.73 (d, J=11.0 Hz, 1H), 7.29 (s, 1H), 6.91 (d, J=8.5 Hz, 1H), 6.54 (s, 1H), 5.63 (s, 1H), 5.42 (s, 2H), 5.32 (d, J=4.9 Hz, 1H), 5.23-5.05 (m, 2H), 4.42 (s, 1H), 4.12-3.95 (m, 2H), 3.64 (dd, J=13.8, 6.2 Hz, 2H), 3.13 (dd, J=43.7, 16.8 Hz, 2H), 2.36 (s, 3H), 2.21-2.03 (m, 2H), 1.93-1.77 (m, 2H), 0.87 (t, J=7.2 Hz, 3H).

### Example 11

### Preparation of 1-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-((1S,3R)-3-hydroxycyclobutyl)urea (Compound 11)

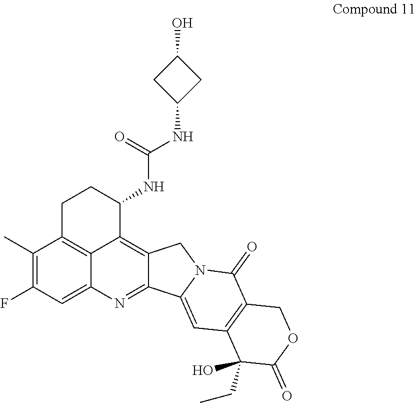 [SMILES: CC[C@@]1(O)C(=O)OCc2c1cc1n(c2=O)Cc2c-1nc1cc(F)c(C)c3c1c2[C@@H](NC(=O)N[C@H]1C[C@@H](O)C1)CC3]

### Reaction Route:

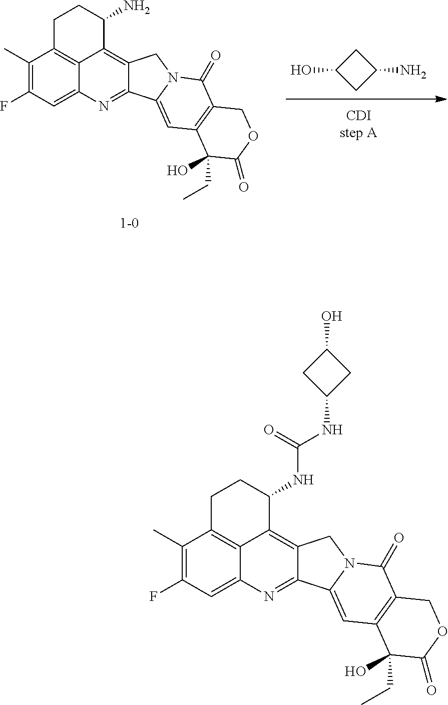 [SMILES: CC[C@@]1(O)C(=O)OCc2c1cc1n(c2=O)Cc2c-1nc1cc(F)c(C)c3c1c2[C@@H](N)CC3 ; N[C@H]1C[C@@H](O)C1 ; CC[C@@]1(O)C(=O)OCc2c1cc1n(c2=O)Cc2c-1nc1cc(F)c(C)c3c1c2[C@@H](NC(=O)N[C@H]1C[C@@H](O)C1)CC3]

### Operation Steps

Step A: Compound 1-0 (20.0 mg, 0.04 mmol) was added into a 25 ml vial, dissolved with N,N-dimethylformamide (1.0 mL), then N,N-diisopropylethylamine (10.0 mg, 0.08 mmol) was dropwise added, subsequently N,N′-carbonyldiimidazole (10.0 mg, 0.06 mmol) was added, and the obtained system was stirred at room temperature for 30 minutes under nitrogen atmosphere. Then, cis-3-aminocyclobutanol (10.0 mg, 0.08 mmol) was added to continuously react at room temperature for 1 hour.

After LCMS showed that the reaction was completed, the reaction solution was purified by preparative high-performance liquid chromatography to obtain the title Compound 11 (15 mg, yield 59.5%).

MS (ESI) M: 549.3 [M+H]⁺.

¹H NMR (400 MHz, DMSO-d₆) δ 7.78 (d, J=11.0 Hz, 1H), 7.29 (s, 1H), 6.52 (s, 2H), 6.13 (s, 1H), 5.42 (s, 2H), 5.25 (dd, J=37.5, 18.0 Hz, 3H), 5.00 (s, 1H), 3.74 (s, 1H), 3.60 (s, 1H), 3.14 (s, 2H), 2.53 (s, 1H), 2.39 (s, 3H), 2.34-2.30 (m, 1H), 2.13 (s, 2H), 1.86 (s, 2H), 1.64 (s, 2H), 1.23 (s, 1H), 0.87 (td, J=5.6, 0.8 Hz, 3H).

### Example 12

### Preparation of 1-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-((1r,3S)-3-hydroxycyclobutyl)urea (Compound 12)

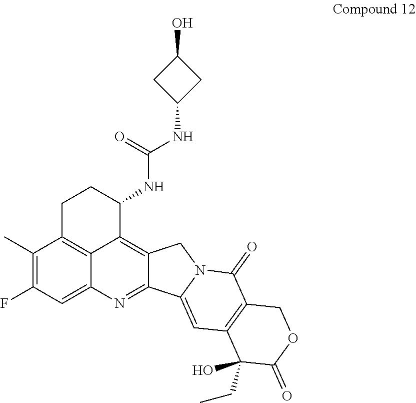 [SMILES: CC[C@@]1(O)C(=O)OCc2c1cc1n(c2=O)Cc2c-1nc1cc(F)c(C)c3c1c2[C@@H](NC(=O)N[C@H]1C[C@H](O)C1)CC3]

### Reaction Route:

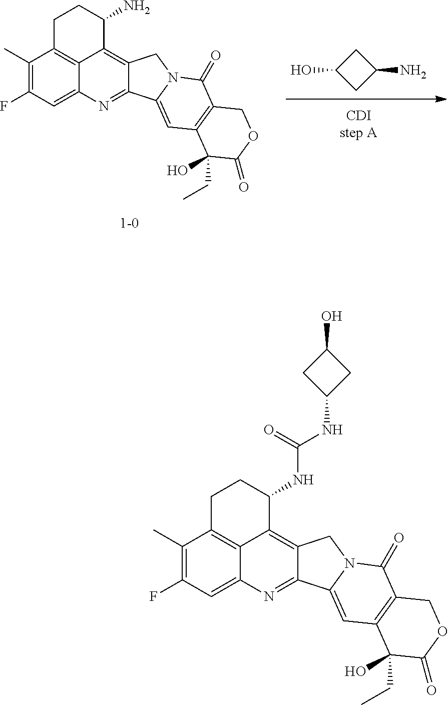 [SMILES: CC[C@@]1(O)C(=O)OCc2c1cc1n(c2=O)Cc2c-1nc1cc(F)c(C)c3c1c2[C@@H](N)CC3 ; N[C@H]1C[C@H](O)C1 ; CC[C@@]1(O)C(=O)OCc2c1cc1n(c2=O)Cc2c-1nc1cc(F)c(C)c3c1c2[C@@H](NC(=O)N[C@H]1C[C@H](O)C1)CC3]

### Operation Steps:

Step A: Compound 1-0 (20.0 mg, 0.04 mmol) was added into a 25 ml vial, dissolved with N,N-dimethylformamide (1.0 mL), then N,N-diisopropylethylamine (10.0 mg, 0.08 mmol) was dropwise added, subsequently N,N′-carbonyldiimidazole (10.0 mg, 0.06 mmol) was added, and stirred at room temperature for 30 minutes under nitrogen atmosphere. Then, trans-3-aminocyclobutanol (10.0 mg, 0.076 mmol) was added to continuously react at room temperature for 1 hour.

After LCMS showed that the reaction was completed, the reaction solution was purified by preparative high-performance liquid chromatography to obtain the title Compound 12 (21.0 mg, yield 83.4%).

MS (ESI) M: 549.3 [M+H]⁺.

¹H NMR (400 MHz, DMSO-d₆) δ 7.75 (d, J=11.0 Hz, 1H), 7.29 (s, 1H), 6.58 (d, J=8.8 Hz, 1H), 6.53 (s, 1H), 6.34 (d, J=6.7 Hz, 1H), 5.42 (s, 2H), 5.23 (dd, J=53.2, 19.4 Hz, 3H), 4.97 (s, 1H), 4.22 (s, 1H), 4.18-4.07 (m, 1H), 3.15 (s, 2H), 2.37 (s, 3H), 2.10 (d, J=5.9 Hz, 6H), 1.94-1.78 (m, 2H), 0.87 (t, J=7.2 Hz, 3H).

### Example 13

### Preparation of (S)—N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypyrrolidine-1-carboxamide (Compound 13)

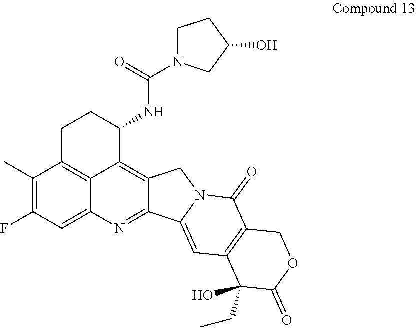 [SMILES: CC[C@@]1(O)C(=O)OCc2c1cc1n(c2=O)Cc2c-1nc1cc(F)c(C)c3c1c2[C@@H](NC(=O)N1CC[C@H](O)C1)CC3]

### Reaction Route:

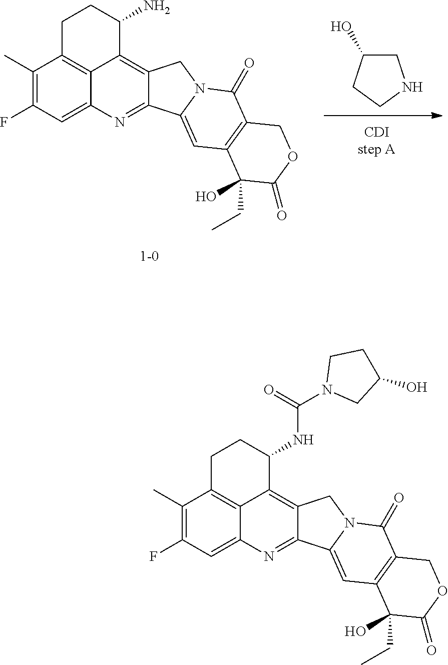 [SMILES: CC[C@@]1(O)C(=O)OCc2c1cc1n(c2=O)Cc2c-1nc1cc(F)c(C)c3c1c2[C@@H](N)CC3 ; O[C@H]1CCNC1 ; CC[C@@]1(O)C(=O)OCc2c1cc1n(c2=O)Cc2c-1nc1cc(F)c(C)c3c1c2[C@@H](NC(=O)N1CC[C@H](O)C1)CC3]

### Operation Steps

Step A: Compound 1-0 (20.0 mg, 0.04 mmol) was added into a 25 ml vial, dissolved with 1 mL of N,N-dimethylformamide, then N,N-diisopropylethylamine (10.0 mg, 0.08 mmol) was added dropwise, subsequently N,N′-carbonyldiimidazole (10.0 mg, 0.06 mmol) was added, and stirred at room temperature for 30 minutes under nitrogen atmosphere. Then, (R)-3-pyrrolidinol (10.0 mg, 0.08 mmol) was added to continuously react at room temperature for 1 hour.

After LCMS showed that the reaction was completed, the reaction solution was purified by preparative high-performance liquid chromatography to obtain the title Compound 13 (21 mg, yield 96.4%).

MS (ESI) M: 549.3 [M+H]⁺.

¹H NMR (400 MHz, DMSO-d₆) δ 7.77 (d, J=11.0 Hz, 1H), 7.31 (s, 1H), 6.66 (d, J=8.4 Hz, 1H), 6.52 (d, J=4.6 Hz, 1H), 5.45-5.35 (m, 3H), 5.20 (s, 2H), 4.91 (d, J=3.6 Hz, 1H), 4.27 (d, J=2.8 Hz, 1H), 3.45-3.35 (m, 3H), 3.27-3.16 (m, 2H), 3.14-3.05 (m, 1H), 2.39 (s, 3H), 2.17 (dd, J=12.4, 6.2 Hz, 2H), 1.88 (m, 7.1 Hz, 3H), 1.79 (s, 1H), 0.87 (t, J=7.3 Hz, 3H).

### Example 14

### Preparation of (R)—N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypyrrolidine-1-carboxamide (Compound 14)

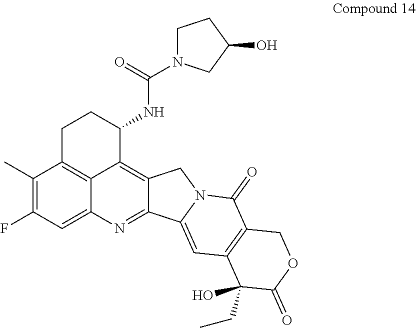 [SMILES: CC[C@@]1(O)C(=O)OCc2c1cc1n(c2=O)Cc2c-1nc1cc(F)c(C)c3c1c2[C@@H](NC(=O)N1CC[C@@H](O)C1)CC3]

### Reaction Route:

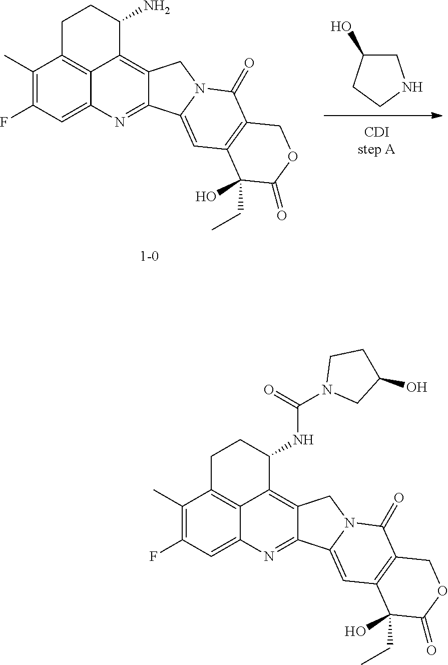 [SMILES: CC[C@@]1(O)C(=O)OCc2c1cc1n(c2=O)Cc2c-1nc1cc(F)c(C)c3c1c2[C@@H](N)CC3 ; O[C@@H]1CCNC1 ; CC[C@@]1(O)C(=O)OCc2c1cc1n(c2=O)Cc2c-1nc1cc(F)c(C)c3c1c2[C@@H](NC(=O)N1CC[C@@H](O)C1)CC3]

### Operation Steps:

Step A: Compound 1-0 (20.0 mg, 0.04 mmol) was added into a 25 ml vial, dissolved with N,N-dimethylformamide (1.0 mL), then N,N-diisopropylethylamine (10.0 mg, 0.076 mmol) was dropwise added, subsequently N,N′-carbonyldiimidazole (10.0 mg, 0.057 mmol) was added, the mixture was stirred at room temperature for 30 minutes under nitrogen atmosphere. Then, (S)-3-pyrrolidinol (10.0 mg, 0.076 mmol) was added to continuously react at room temperature for 1 hour.

After LCMS showed that the reaction was completed, the reaction solution was purified by preparative high-performance liquid chromatography to obtain the title Compound 14 (21.0 mg, yield 96.4%).

MS (ESI) M: 549.3 [M+H]⁺.

¹H NMR (400 MHz, DMSO-d₆) δ 7.77 (d, J=11.0 Hz, 1H), 7.31 (s, 1H), 6.66 (d, J=8.4 Hz, 1H), 6.52 (d, J=4.6 Hz, 1H), 5.45-5.35 (m, 3H), 5.20 (s, 2H), 4.91 (d, J=3.6 Hz, 1H), 4.27 (d, J=2.8 Hz, 1H), 3.45-3.35 (m, 3H), 3.27-3.16 (m, 2H), 3.14-3.05 (m, 1H), 2.39 (s, 3H), 2.17 (dd, J=12.4, 6.2 Hz, 2H), 1.88 (m, 7.1 Hz, 3H), 1.79 (s, 1H), 0.87 (t, J=7.3 Hz, 3H).

### Example 15

### Preparation of 3-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) azetidine-1-carboxamide (Compound 15)

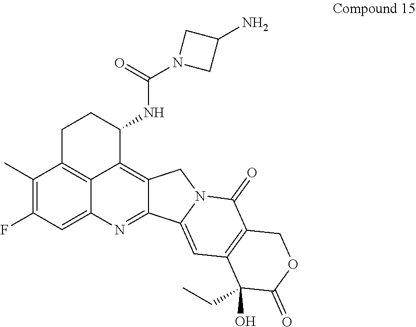 [SMILES: CC[C@@]1(O)C(=O)OCc2c1cc1n(c2=O)Cc2c-1nc1cc(F)c(C)c3c1c2[C@@H](NC(=O)N1CC(N)C1)CC3]

### Reaction Route:

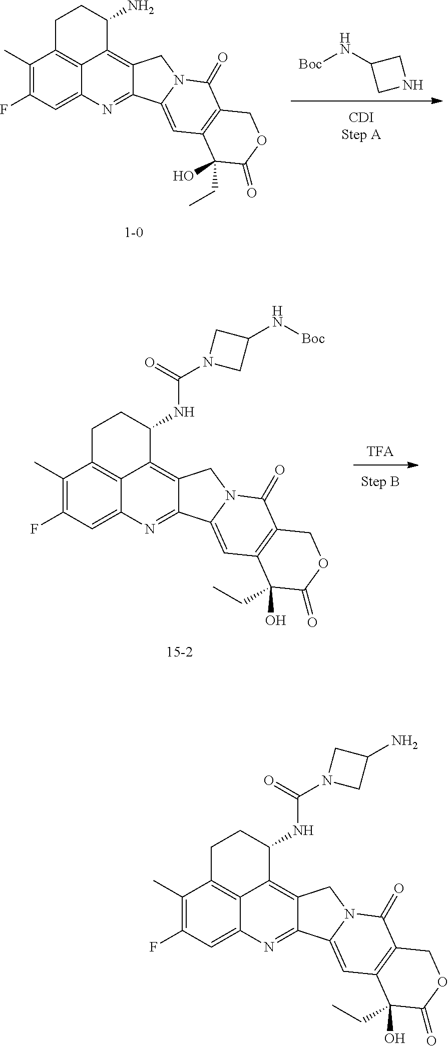 [SMILES: CC[C@@]1(O)C(=O)OCc2c1cc1n(c2=O)Cc2c-1nc1cc(F)c(C)c3c1c2[C@@H](N)CC3 ; CC(C)(C)OC(=O)NC1CNC1 ; CC[C@@]1(O)C(=O)OCc2c1cc1n(c2=O)Cc2c-1nc1cc(F)c(C)c3c1c2[C@@H](NC(=O)N1CC(NC(=O)OC(C)(C)C)C1)CC3 ; "TFA" ; CC[C@@]1(O)C(=O)OCc2c1cc1n(c2=O)Cc2c-1nc1cc(F)c(C)c3c1c2[C@@H](NC(=O)N1CC(N)C1)CC3]

### Operation Steps:

Step A: Compound 1-0 (35.0 mg, 0.08 mmol) was added into a 25 ml single-necked flask, dissolved with N,N-dimethylformamide (1.0 mL), N,N′-carbonyldiimidazole (19.0 mg, 0.12 mmol) and triethylamine (24.0 mg, 0.24 mmol) were added in sequence, and the mixture was stirred at room temperature for 30 minutes under nitrogen atmosphere. Then, 3-N-Boc-amino-azetidine (14.0 mg, 0.08 mmol) was added and stirred at room temperature for 1 hour under nitrogen atmosphere.

After LCMS showed that the reaction was completed, the reaction solution was directly purified by preparative high-performance liquid chromatography to obtain Compound 15-2 (30.0 mg, yield 59.1%).

MS (ESI) M: 634.2 [M+H]⁺.

Step B: Compound 15-2 (27.0 mg, 0.04 mmol) was added into a 25 ml single-necked flask, dissolved with dichloromethane (1.0 mL), trifluoroacetic acid (0.2 mL) was added, and the mixture was stirred at room temperature for 2 hours under nitrogen atmosphere.

After LCMS showed that the reaction was completed, the reaction solution was directly purified by preparative high-performance liquid chromatography to obtain the title Compound 15 (8.0 mg, yield 35.2%).

MS (ESI) M: 534.3 [M+H]⁺.

¹H NMR (400 MHz, DMSO-d₆) δ 7.78 (d, J=11.1 Hz, 1H), 7.32 (s, 1H), 6.93 (d, J=8.8 Hz, 1H), 6.52 (s, 1H), 5.43 (s, 2H), 5.36 (s, 1H), 5.23 (s, 2H), 4.12-3.98 (m, 2H), 3.72 (s, 2H), 3.61 (d, J=6.4 Hz, 2H), 3.14 (s, 2H), 2.67 (s, 1H), 2.36 (d, J=27.0 Hz, 3H), 2.14 (d, J=6.0 Hz, 2H), 1.95-1.80 (m, 2H), 0.87 (t, J=7.4 Hz, 3H).

### Example 16

### Preparation of 1-(1S,3R)-3-aminocyclobutyl)-3-(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)urea (Compound 16)

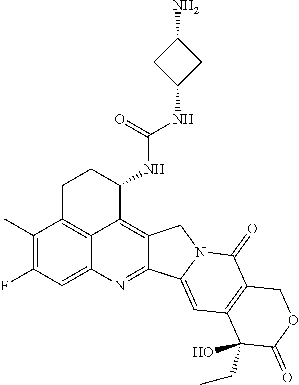 [SMILES: CC[C@@]1(O)C(=O)OCc2c1cc1n(c2=O)Cc2c-1nc1cc(F)c(C)c3c1c2[C@@H](NC(=O)N[C@H]1C[C@@H](N)C1)CC3]

### Compound 16 Reaction Route:

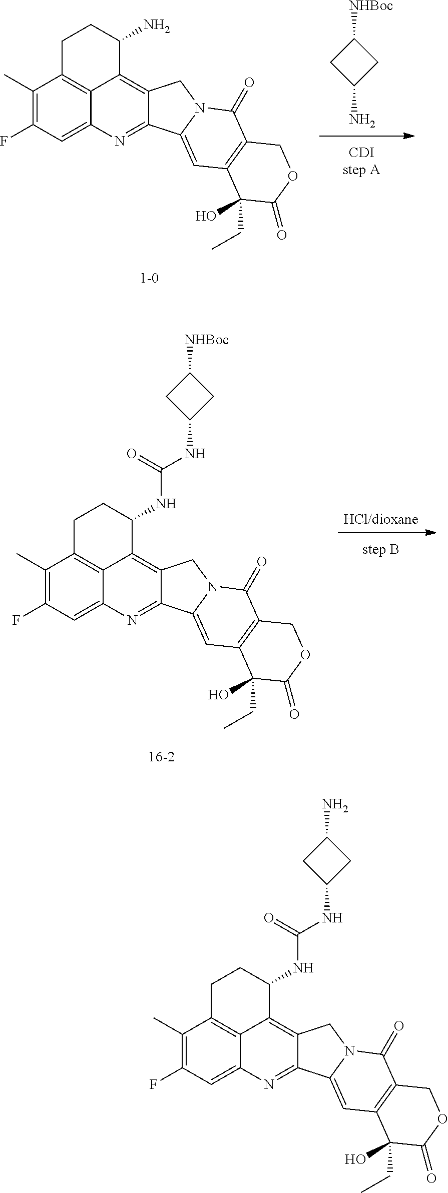 [SMILES: CC[C@@]1(O)C(=O)OCc2c1cc1n(c2=O)Cc2c-1nc1cc(F)c(C)c3c1c2[C@@H](N)CC3 ; CC(C)(C)OC(=O)N[C@H]1C[C@@H](N)C1 ; CC[C@@]1(O)C(=O)OCc2c1cc1n(c2=O)Cc2c-1nc1cc(F)c(C)c3c1c2[C@@H](NC(=O)N[C@H]1C[C@@H](NC(=O)OC(C)(C)C)C1)CC3 ; "HCl/dioxane" ; CC[C@@]1(O)C(=O)OCc2c1cc1n(c2=O)Cc2c-1nc1cc(F)c(C)c3c1c2[C@@H](NC(=O)N[C@H]1C[C@@H](N)C1)CC3]

### Operation Steps:

Step A: Compound 1-0 (20.0 mg, 0.04 mmol) was added into a vial, dissolved with N,N-dimethylformamide (1.0 mL), then 3 drops of N,N-diisopropylethylamine was added dropwise, and stirred for 5 minutes. Then, N,N′-carbonyldiimidazole (10.0 mg, 0.06 mmol) was added and continuously stirred at room temperature for 30 minutes. Then, cis-tert-butyl-3-amino-1-cyclobutyl carbamate (15.0 mg, 0.08 mmol) was added and the mixture was continuously stirred for 1 hour.

After LCMS showed that the reaction was completed, the reaction solution was purified by preparative high-performance liquid chromatography to obtain Compound 16-2 (18.0 mg, yield 60.0%).

MS (ESI) M: 648.6 [M+H]⁺.

Step B: Compound 16-2 (18.0 mg, 0.03 mmol) was added into a vial, dissolved with dichloromethane (1.0 mL), then 10 drops of hydrochloric acid in 1,4-dioxane solution were added, and the mixture was stirred at room temperature for 30 minutes.

After LCMS showed that the reaction was completed, the mixed solution was directly purified by preparative high-performance liquid chromatography to obtain the title Compound 16 (5.0 mg, yield 32.9%).

MS (ESI) M: 548.3 [M+H]⁺.

¹H NMR (400 MHz, DMSO-d₆) ¹H NMR (400 MHz, DMSO-d₆) δ 8.40 (s, 1H), 7.71 (d, J=10.5 Hz, 1H), 7.33 (s, 1H), 5.36 (dt, J=47.3, 18.1 Hz, 4H), 5.08 (d, J=19.5 Hz, 1H), 3.95 (s, 1H), 3.30 (s, 1H), 3.14 (d, J=18.8 Hz, 2H), 2.58 (d, J=15.0 Hz, 2H), 2.36 (s, 3H), 2.19 (s, 1H), 2.08 (s, 1H), 1.93 (d, J=23.2 Hz, 2H), 1.90-1.76 (m, 2H), 0.88 (t, J=7.2 Hz, 3H).

### Example 17

### Preparation of 1-(1R,3S)-3-aminocyclobutyl)-3-(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinoline-1-yl)urea (Compound 17)

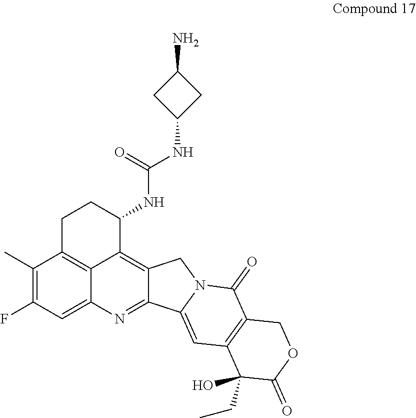 [SMILES: CC[C@@]1(O)C(=O)OCc2c1cc1n(c2=O)Cc2c-1nc1cc(F)c(C)c3c1c2[C@@H](NC(=O)N[C@H]1C[C@H](N)C1)CC3]

### Reaction Route:

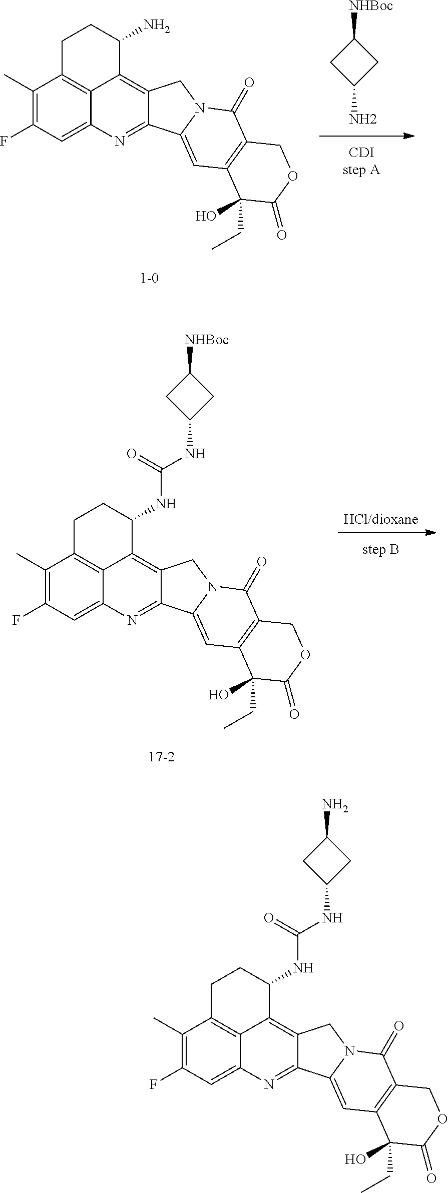 [SMILES: CC[C@@]1(O)C(=O)OCc2c1cc1n(c2=O)Cc2c-1nc1cc(F)c(C)c3c1c2[C@@H](N)CC3 ; CC(C)(C)OC(=O)N[C@H]1C[C@H](N)C1 ; CC[C@@]1(O)C(=O)OCc2c1cc1n(c2=O)Cc2c-1nc1cc(F)c(C)c3c1c2[C@@H](NC(=O)N[C@H]1C[C@H](NC(=O)OC(C)(C)C)C1)CC3 ; "HCl/dioxane" ; CC[C@@]1(O)C(=O)OCc2c1cc1n(c2=O)Cc2c-1nc1cc(F)c(C)c3c1c2[C@@H](NC(=O)N[C@H]1C[C@H](N)C1)CC3]

### Operation Steps:

Step A: Compound 1-0 (20.0 mg, 0.04 mmol) was added into a vial, dissolved with N,N-dimethylformamide (1.0 mL), then 3 drops of N,N-diisopropylethylamine was added dropwise, and the mixture was stirred for 5 minutes. Then, N,N′-carbonyldiimidazole (10.0 mg, 0.06 mmol) was added and continuously stirred at room temperature for 30 minutes. Then, trans-tert-butyl-3-amino-1-cyclobutyl carbamate (15.0 mg, 0.08 mmol) was added and the mixture was continuously stirred for 1 hour.

After LCMS showed that the reaction was completed, the reaction solution was purified by preparative high-performance liquid chromatography to obtain Compound 17-2 (22.0 mg, yield 73.9%).

MS (ESI) M: 648.6 [M+H]⁺.

Step B: Compound 17-2 (22.0 mg, 0.03 mmol) was added into a vial, dissolved with dichloromethane (1.0 mL), then 10 drops of hydrochloric acid in 1,4-dioxane solution were added, and the mixture was stirred at room temperature for 30 minutes.

After LCMS showed that the reaction was completed, the mixed solution was directly purified by preparative high-performance liquid chromatography to obtain the title Compound 17 (8.0 mg, yield 43.0%).

MS (ESI) M: 548.3 [M+H]⁺.

¹H NMR (400 MHz, DMSO-d₆) ¹H NMR (400 MHz, DMSO-d₆) δ 8.40 (s, 1H), 7.70 (d, J=10.7 Hz, 1H), 7.35 (s, 1H), 5.48-5.36 (m, 2H), 5.29 (d, J=19.7 Hz, 2H), 5.06 (d, J=19.6 Hz, 1H), 4.42 (s, 1H), 3.72 (s, 1H), 3.15 (d, J=23.6 Hz, 2H), 2.37 (d, J=14.1 Hz, 6H), 2.21 (s, 1H), 2.07 (s, 1H), 1.87 (dd, J=13.5, 7.0 Hz, 2H), 0.88 (t, J=7.2 Hz, 3H).

### Example 18

### Preparation of 1-(azetidin-3-yl)-3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)urea (Compound 18)

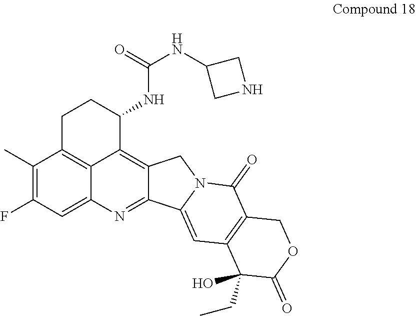 [SMILES: CC[C@@]1(O)C(=O)OCc2c1cc1n(c2=O)Cc2c-1nc1cc(F)c(C)c3c1c2[C@@H](NC(=O)NC1CNC1)CC3]

### Reaction Route:

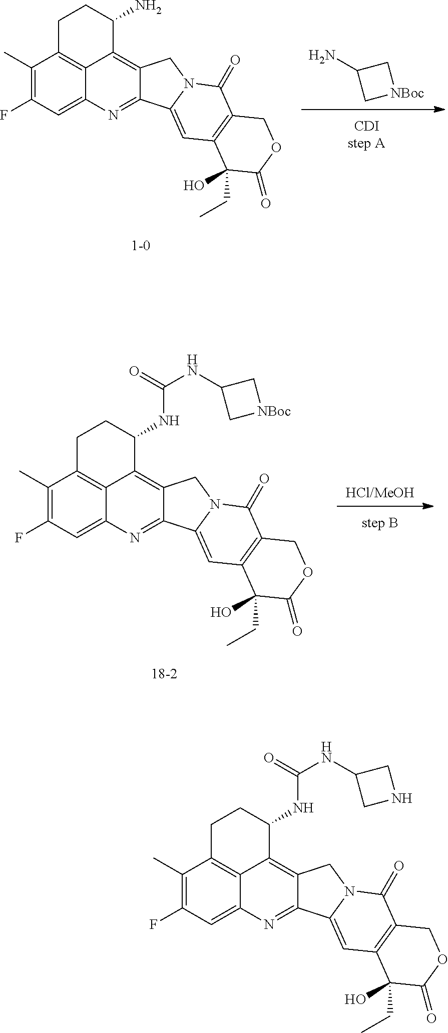 [SMILES: CC[C@@]1(O)C(=O)OCc2c1cc1n(c2=O)Cc2c-1nc1cc(F)c(C)c3c1c2[C@@H](N)CC3 ; CC(C)(C)OC(=O)N1CC(N)C1 ; CC[C@@]1(O)C(=O)OCc2c1cc1n(c2=O)Cc2c-1nc1cc(F)c(C)c3c1c2[C@@H](NC(=O)NC1CN(C(=O)OC(C)(C)C)C1)CC3 ; "HCl/MeOH" ; CC[C@@]1(O)C(=O)OCc2c1cc1n(c2=O)Cc2c-1nc1cc(F)c(C)c3c1c2[C@@H](NC(=O)NC1CNC1)CC3]

### Operation Steps:

Step A: Compound 1-0 (50.0 mg, 0.09 mmol) was added into a 25 ml single-necked flask, a solvent: dry N,N-dimethylformamide (3.0 mL) was added, and four drops of N,N-diisopropylethylamine were added until the reaction solution was clear. The reaction solution was stirred at room temperature for 5 minutes, then N,N′-carbonyldiimidazole (22.9 mg, 0.14 mmol) was added, and the mixture was stirred at room temperature to react for 1 hour. Then, 1-tert-butoxycarbonyl-3-aminocyclobutylamine (32.4 mg, 0.19 mmol) was added, and the reaction system reacted at room temperature for 1 hour under nitrogen protection.

After the reaction was completed, the reaction solution was filtered, and the filtrate was purified by preparative high-performance liquid chromatography to obtain Compound 18-2 (38.0 mg, yield 63.8%).

MS (ESI) M: 634.2 [M+H]⁺.

¹H NMR (400 MHz, DMSO-d₆) δ 7.76 (d, J=10.9 Hz, 1H), 7.30 (s, 1H), 6.77 (d, J=8.9 Hz, 1H), 6.68 (d, J=7.4 Hz, 1H), 6.51 (s, 1H), 5.42 (s, 2H), 5.31 (dd, J=18.6, 11.9 Hz, 2H), 5.17 (d, J=19.1 Hz, 1H), 4.40 (dd, J=13.0, 5.7 Hz, 1H), 4.06 (t, J=7.7 Hz, 2H), 3.71 (s, 2H), 3.17 (s, 2H), 2.38 (s, 3H), 2.14 (dd, J=14.3, 6.7 Hz, 2H), 1.86 (tt, J=14.1, 7.2 Hz, 2H), 1.43-1.31 (m, 9H), 0.87 (t, J=7.3 Hz, 3H).

Step B: Compound 18-2 (38.0 mg, 0.06 mmol) was added into a 25 ml single-necked flask, a solvent: methanol (2.0 mL) was added, and then hydrochloric acid in methanol solution (2.0 mL) was added, and the reaction system reacted at room temperature for 4 hours under nitrogen protection.

After the reaction was completed, the solvent was spin-dried under a reduced pressure, and the crude product was purified by preparative high-performance liquid chromatography to obtain the title Compound 18 (21.8 mg, yield 68.1%).

MS (ESI) M: 534.3 [M+H]⁺.

¹H NMR (400 MHz, DMSO-d₆) δ 8.35 (s, 1H), 7.75 (d, J=11.0 Hz, 1H), 7.43-7.26 (m, 3H), 5.41 (s, 2H), 5.31 (d, J=19.1 Hz, 2H), 5.15 (d, J=19.0 Hz, 1H), 4.58 (s, 1H), 3.96 (s, 2H), 3.83 (s, 2H), 3.28-3.04 (m, 2H), 2.37 (s, 3H), 2.14 (ddd, J=21.7, 9.7, 4.1 Hz, 2H), 1.86 (tt, J=14.1, 7.0 Hz, 2H), 0.87 (t, J=7.3 Hz, 3H).

### Example 19

### Preparation of (1S,3R)-3-aminocyclobutyl((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 19)

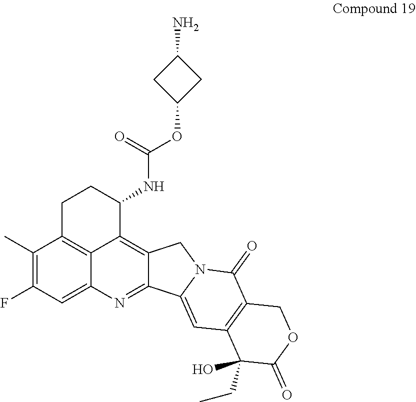 [SMILES: CC[C@@]1(O)C(=O)OCc2c1cc1n(c2=O)Cc2c-1nc1cc(F)c(C)c3c1c2[C@@H](NC(=O)O[C@H]1C[C@@H](N)C1)CC3]

### Reaction Route:

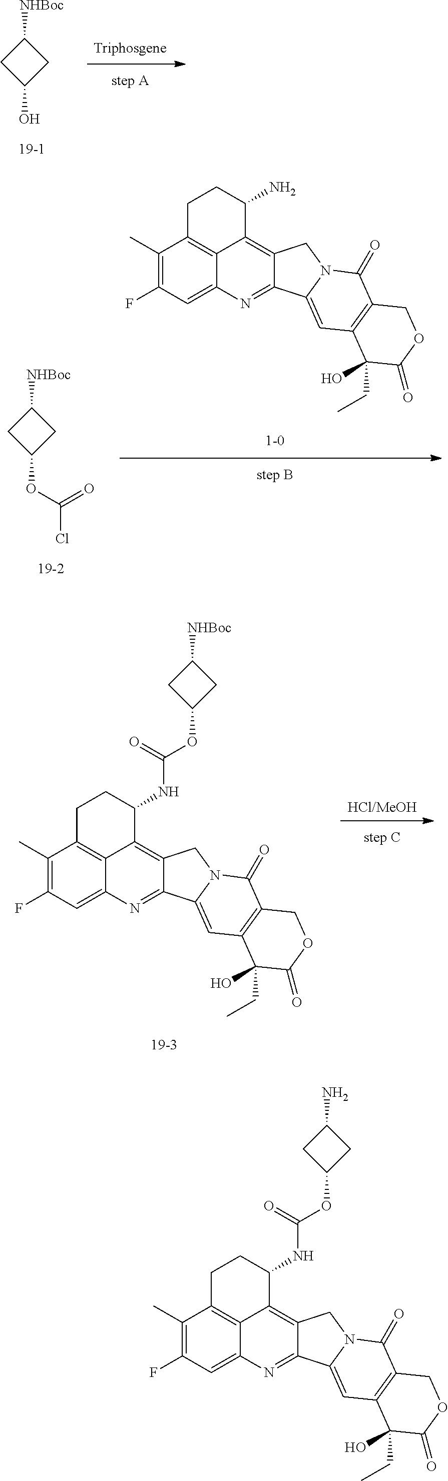

### Operation Steps:

Step A: triphosgene (180.0 mg, 0.61 mmol) was slowly added into a stirred solution of Compound 19-1 (70.0 mg, 0.41 mmol) and N,N-diisopropylethylamine (0.26 mL, 1.42 mmol) in tetrahydrofuran (2.0 mL) under ice bath at 0° C. The obtained mixture was stirred at room temperature for 2 hours. After the reaction was completed, the reaction mixture was filtered and washed with fresh tetrahydrofuran. The filtrate was concentrated under reduced pressure to obtain a crude product 19-2 (95.0 mg, yield 92.8%). The crude product was directly used in the next step without purification.

Step B: Compound 1-0 (30.0 mg, 0.06 mmol) was dispersed in dichloromethane (20.0 mL), and then the crude product of Compound 19-2 (95.0 mg, 0.38 mmol) and N,N-diisopropylethylamine (0.26 mL, 1.42 mmol) were added, and the system gradually became clear and reacted at room temperature for 0.5 hours.

After LCMS showed that the reaction was completed, the mixed solution was concentrated and then purified by preparative high-performance liquid chromatography to obtain Compound 19-3 (30.0 mg, yield 85%).

MS (ESI) M: 649.0 [M+H]⁺.

Step C: Compound 19-3 (20.0 mg, 0.03 mmol) was dissolved in a hydrochloric acid in methanol solution (10.0 mL) and stirred at room temperature for 5 hours. After LCMS showed that the reaction was completed, the mixed solution was concentrated and then purified by preparative high-performance liquid chromatography to obtain the title Compound 19 (10.0 mg, yield 59.5%).

MS (ESI) M: 549.7 [M+H]⁺.

¹H NMR (400 MHz, DMSO-d₆) δ 8.32 (s, 1H), 7.97 (d, J=8.8 Hz, 1H), 7.76 (d, J=10.9 Hz, 1H), 7.31 (s, 1H), 5.42 (s, 2H), 5.29-5.11 (m, 3H), 4.70-4.60 (m, 1H) 3.32-3.04 (m, 3H), 2.71-2.59 (m, 2H), 2.37 (s, 3H), 2.25-2.07 (m, 2H), 2.03-1.77 (m, 4H), 0.88 (t, J=7.3 Hz, 3H).

### Example 20

### Preparation of (1R,3S)-3-aminocyclobutyl((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 20)

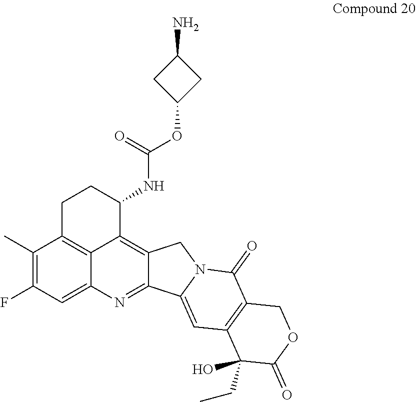 [SMILES: CC[C@@]1(O)C(=O)OCc2c1cc1n(c2=O)Cc2c-1nc1cc(F)c(C)c3c1c2[C@@H](NC(=O)O[C@H]1C[C@H](N)C1)CC3]

### Reaction Route:

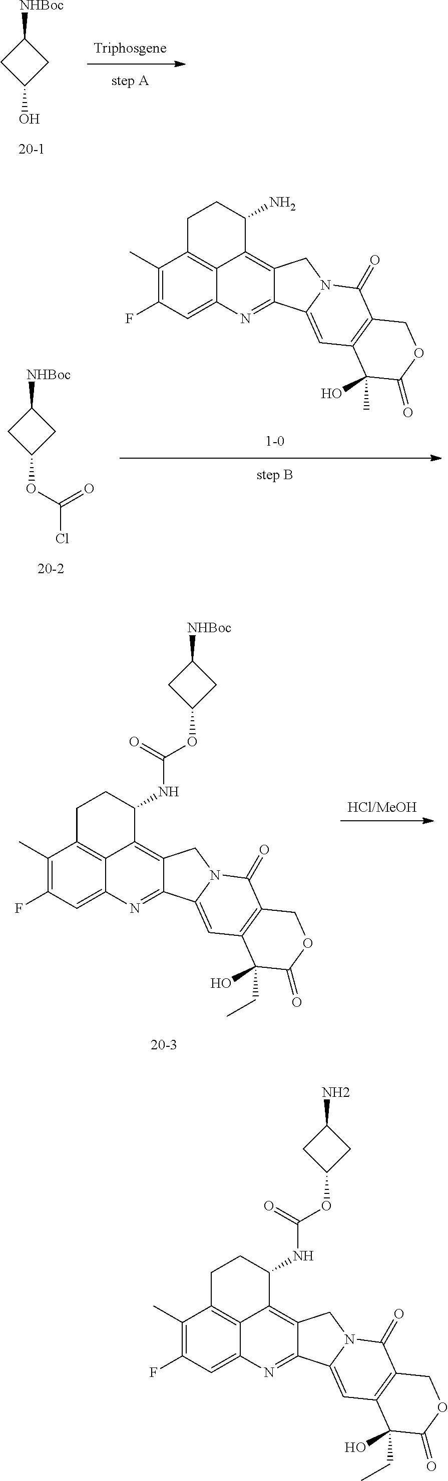

### Operation Steps:

Step A: triphosgene (180.0 mg, 0.61 mmol) was slowly added into a stirred solution of Compound 20-1 (70.0 mg, 0.41 mmol) and N,N-diisopropylethylamine (0.26 mL, 1.42 mmol) in tetrahydrofuran (2.0 mL) under ice bath at 0° C. The obtained mixture was stirred at room temperature for 2 hours. After the reaction was completed, the reaction mixture was filtered and washed with fresh tetrahydrofuran. The filtrate was concentrated under reduced pressure to obtain a crude product 20-2 (95.1 mg, yield 92.9%). The crude product was directly used in the next step without purification.

Step B: Compound 1-0 (30.0 mg, 0.06 mmol) was dispersed in dichloromethane (20.0 mL), and then Compound 30-2 (95.1 mg, 0.38 mmol) and N,N-diisopropylethylamine (0.26 mL, 1.42 mmol) were added, and the system gradually became clear and reacted at room temperature for 0.5 hours.

After LCMS showed that the reaction was completed, the mixed solution was concentrated and then purified by preparative high-performance liquid chromatography to obtain Compound 20-3 (30.0 mg, yield 81.9%).

MS (ESI) M: 649.0 [M+H]⁺.

Step C: Compound 20-3 (20.0 mg, 0.03 mmol) was dissolved in a hydrochloric acid in methanol solution (10.0 mL) and stirred at room temperature for 5 hours. After LCMS showed that the reaction was completed, the mixed solution was concentrated and then purified by preparative high-performance liquid chromatography to obtain the title Compound 20 (10.0 mg, yield 59.5%).

MS (ESI) M: 549.7 [M+H]⁺.

¹H NMR (400 MHz, DMSO-d₆) δ 8.34 (s, 1H), 8.02 (d, J=9 Hz, 1H), 7.76 (d, J=9.6 Hz, 1H), 7.30 (s, 1H), 5.42 (s, 2H), 5.34-5.05 (m, 4H), 3.68 (s, 1H), 3.23 (s, 1H), 3.12 (s, 1H), 2.45 (s, 1H), 2.37 (s, 3H), 2.34-2.18 (m, 4H), 2.11 (dd, J=13.4, 9.5 Hz, 1H), 1.87 (tt, J=14.0, 7.1 Hz, 1H), 0.88 (t, J=7.3 Hz, 3H).

### Example 21

### Preparation of azetidine-3-yl((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 21)

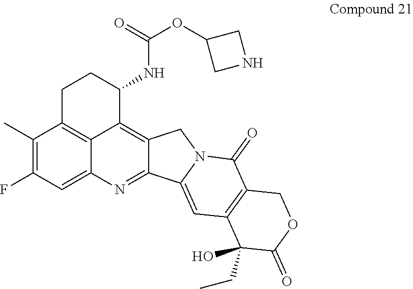 [SMILES: CC[C@@]1(O)C(=O)OCc2c1cc1n(c2=O)Cc2c-1nc1cc(F)c(C)c3c1c2[C@@H](NC(=O)OC1CNC1)CC3]

### Reaction Route:

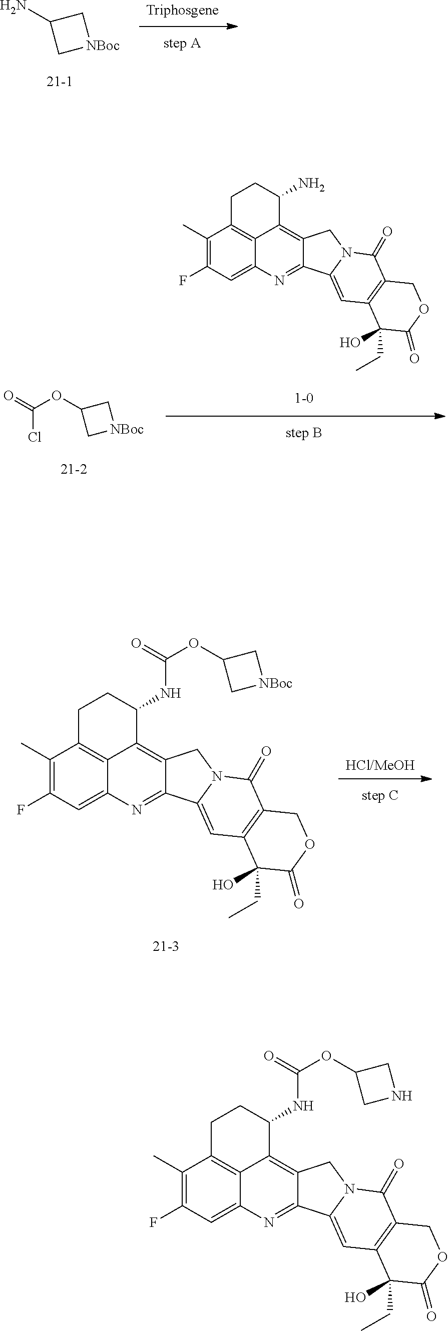

### Operation Steps:

Step A: triphosgene (180.0 mg, 0.61 mmol) was slowly added into a stirred solution of compound 21-1 (70.0 mg, 0.41 mmol) and N,N-diisopropylethylamine (0.26 mL, 1.42 mmol) in tetrahydrofuran (2.0 mL) under ice bath at 0° C. The obtained mixture was stirred at room temperature for 2 hours. After the reaction was completed, the reaction mixture was filtered and washed with fresh tetrahydrofuran. The filtrate was concentrated under reduced pressure to obtain a crude product 21-2 (95.1 mg, yield 92.9%). The crude product was directly used in the next step without purification.

Step B: Compound 1-0 (30.0 mg, 0.06 mmol) was dispersed in dichloromethane (20.0 mL), and then Compound 21-2 (95.1 mg, 0.38 mmol) and N,N-diisopropylethylamine (0.26 mL, 1.42 mmol) were added, and the system gradually became clear and reacted at room temperature for 0.5 hours.

After LCMS showed that the reaction was completed, the mixed solution was concentrated and then purified by preparative high-performance liquid chromatography to obtain Compound 21-3 (30.0 mg, yield 85%).

MS (ESI) M: 635.1 [M+H]⁺.

Step C: Compound 21-3 (20.0 mg, 0.03 mmol) was dissolved in a hydrochloric acid in methanol solution (10.0 mL) and stirred at room temperature for 5 hours. After LCMS showed that the reaction was completed, the mixed solution was concentrated and then purified by preparative high-performance liquid chromatography to obtain the title Compound 21 (10.0 mg, yield 59.5%).

MS (ESI) M: 533.1 [M−H]⁻.

¹H NMR (400 MHz, DMSO-d₆) δ 8.31 (s, 1H), 8.17 (d, J=8.7 Hz, 1H), 7.75 (d, J=10.8 Hz, 1H), 7.30 (s, 1H), 5.42 (s, 2H), 5.35-5.10 (m, 4H), 3.91 (s, 2H), 3.73 (d, J=33.7 Hz, 2H), 3.17 (dd, J=58.1, 18.1 Hz, 2H), 2.36 (s, 3H), 2.27-2.07 (m, 2H), 1.96-1.78 (m, 2H), 1.16 (s, 1H), 0.88 (t, J=7.3 Hz, 3H).

### Example 22

### Preparation of 3-((1S,9S)-9-ethyl-5-fluoro-9-hydroxy-4-methyl-10,13-dioxo-2,3,9,10,13,15-hexahydro-1H,12H-benzo[d]pyrano[3′,4′:6,7]indolizino[1,2-b]quinolin-1-yl)-1-(2-hydroxyethoxy)-1-isopropylurea (Compound 22)

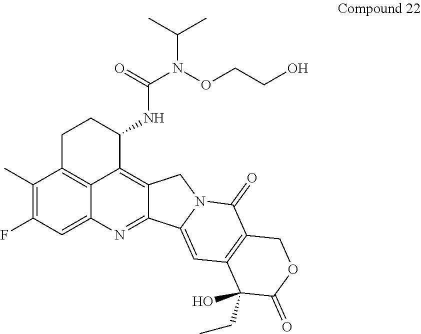 [SMILES: CC[C@@]1(O)C(=O)OCc2c1cc1n(c2=O)Cc2c-1nc1cc(F)c(C)c3c1c2[C@@H](NC(=O)N(OCCO)C(C)C)CC3]

### Reaction Route:

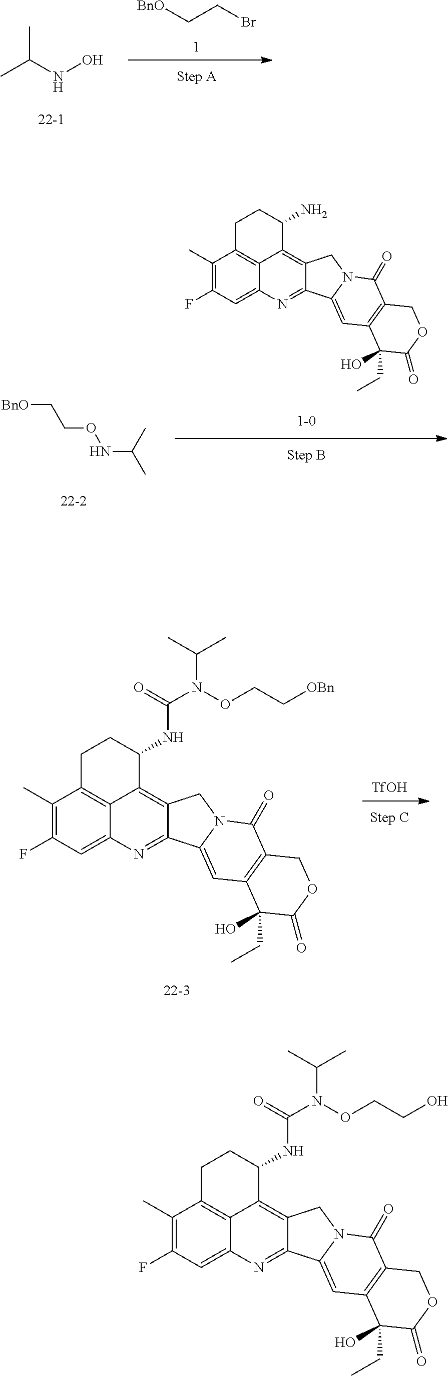

### Step A:

Compound 22-1 (500 mg, 6.66 mmol) was added into a 100 mL single-necked flask, a solvent: THF (30 mL) was added, and then NaH (479 mg, 12.0 mmol) was added, and the reaction system reacted at room temperature for 1 hour under nitrogen protection. Then, a reactant 1 (1.72 g, 7.99 mmol) was added and reacted for 14 hours under argon protection. After the reaction was completed, water was slowly added to the reaction solution, the mixed solution was extracted with ethyl acetate (30 mL×3), and organic phases were combined, then dried with anhydrous sodium sulfate, filtered, and finally concentrated under reduced pressure. The obtained residue was purified by Flash (ACN/0.1% TFA/H₂O) to obtain Compound 22-2 (110 mg, yield 7.9%).

MS (ESI) M: 210.2 [M+H]⁺.

¹H NMR (400 MHz, DMSO-d₆) δ7.39-7.23 (m, 5H), 6.26 (s, 1H), 4.48 (s, 2H), 3.75-3.66 (m, 2H), 3.60-3.51 (m, 2H), 3.00 (dt, J=12.4, 6.2 Hz, 1H), 0.94 (d, J=6.3 Hz, 6H).

### Step B:

Compound 22-2 (23.6 mg, 0.11 mmol) was added into a 25 mL single-necked flask, a solvent: THF (2 mL) was added, and triphosgene (840 mg, 0.28 mmol) and 8 drops of DIEA were added to react at room temperature for 16 hours, then the reaction solution was filtered, and the filtrate was allowed to stand for later use. Compound 1-0 (50 mg, 0.094 mmol) was added into a single-necked flask, a solvent: THF (2 mL) was added, then DIEA (0.1 mL) was added, and the mixture was stirred for 5 minutes. Then, the above filtrate was dropwise added into the mixed solution to react at 40° C. for 24 hours. After the reaction was completed, the reaction solution was concentrated, and the crude product was purified by Prep-HPLC (ACN/0.1% TFA/H₂O) to obtain Compound 22-3 (20 mg, yield 32%).

MS (ESI) M: 671.7 [M+H]⁺.

¹H NMR (400 MHz, DMSO-d6) δ 7.75 (d, J=10.9 Hz, 1H), 7.48 (d, J=9.0 Hz, 1H), 7.29 (s, 1H), 6.89 (dd, J=14.4, 7.3 Hz, 5H), 5.43 (s, 2H), 5.41-5.29 (m, 1H), 5.16 (q, J=19.0 Hz, 2H), 4.32 (dt, J=13.3, 6.5 Hz, 1H), 4.04 (s, 4H), 3.55 (s, 2H), 2.14-2.10 (m, 2H), 2.33 (s, 3H), 2.11 (s, 1H), 1.94-1.80 (m, 3H), 1.22 (d, J=6.8 Hz, 6H), 0.90 (t, J=7.3 Hz, 3H).

### Step C:

Compound 22-3 (50 mg, 0.075 mmol) was added into a 25 ml single-necked flask, and solvents: DCM (3 mL) and trifluoromethanesulfonic acid (0.6 mL) were added, and the reaction system reacted at room temperature for 3 hours under argon protection. After the reaction was completed, water was slowly added to the reaction solution, the mixed solution was extracted with DCM (30 mL×3), and organic phases were combined, then dried with anhydrous sodium sulfate, filtered, and finally concentrated under reduced pressure. The obtained residue was purified by Prep-HPLC (ACN/0.1% TFA/H₂O) to obtain Compound 22 (15.24 mg, yield 35%).

MS (ESI) M: 581.5 [M+H]⁺.

¹H NMR (400 MHz, DMSO-d₆ and D₂O) δ 7.97 (d, J=9.0 Hz, 1H), 7.71 (d, J=10.9 Hz, 1H), 7.33 (s, 1H), 5.41 (q, J=16.1 Hz, 3H), 5.27-5.06 (m, 2H), 4.38-4.26 (m, 1H), 3.96-3.82 (m, 2H), 3.54-3.40 (m, 2H), 3.28-3.18 (m, 1H), 3.18-3.04 (m, 1H), 2.36 (s, 3H), 2.29-2.19 (m, 1H), 2.19-2.07 (m, 1H), 1.86 (dq, J=14.0, 7.1 Hz, 2H), 1.22 (dd, J=18.0, 6.6 Hz, 6H), 0.88 (t, J=7.3 Hz, 3H).

### Example 23

### Preparation of 3-((1S,9S)-9-ethyl-5-fluoro-9-hydroxy-4-methyl-10,13-dioxo-2,3,9,10,13,15-hexahydro-1H,12H-benzo[d]pyrano[3′,4′:6,7]indolizino[1,2-b]quinolin-1-yl)-1-(2-hydroxyethyl)-1-(thietan-3-yl)urea (Compound 23)

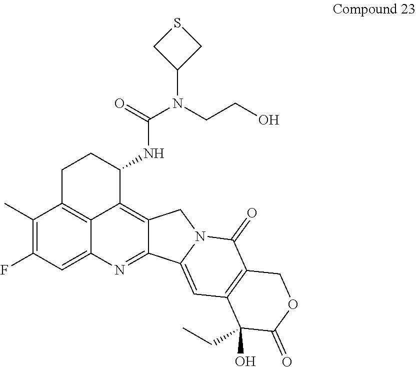 [SMILES: CC[C@@]1(O)C(=O)OCc2c1cc1n(c2=O)Cc2c-1nc1cc(F)c(C)c3c1c2[C@@H](NC(=O)N(CCO)C1CSC1)CC3]

### Reaction Route:

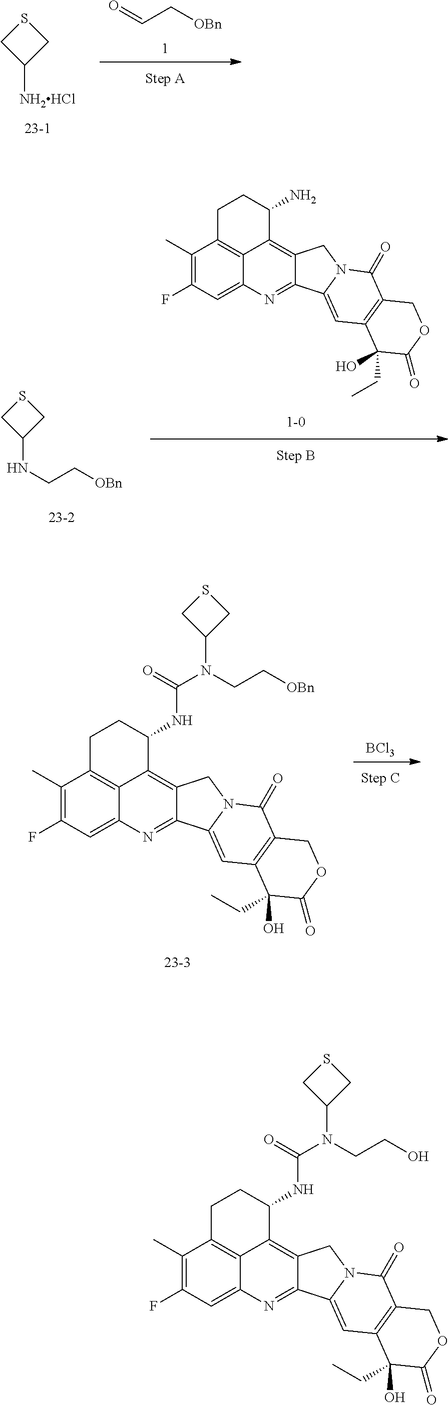

### Operation Steps:

### Step A:

Compound 23-1 (200 mg, 1.59 mmol) was added into a 25 ml single-necked flask, a solvent: methanol (8 mL) was added, and then reactant 1 (358.1 mg, 2.39 mmol), NaBH₃CN (149.8 mg, 2.39 mmol) and three drops of anhydrous acetic acid were added, and the reaction system reacted at room temperature for 2 hours under nitrogen protection. After the reaction was completed, the mixed solution was diluted with DCM (30 mL), and washed with water (10 mL×3) and a saturated sodium chloride solution (10 mL×1) sequentially, and organic phases were combined, then dried with anhydrous sodium sulfate, filtered, and finally concentrated under reduced pressure. 150 mg of the obtained residue was purified by Flash (ACN/0.1% TFA/H₂O) to obtain Compound 23-2 (110 mg, yield 31%).

MS (ESI) M: 224.1 [M+H]⁺.

### Step B:

Compound 1-0 (50 mg, 0.094 mmol) was added into a 25 ml single-necked flask, a solvent: dry DMF (3 mL) was added, and DIEA was dropwise added until the reaction solution was clear. The reaction solution was stirred at room temperature for 5 minutes, then CDI (22.8 mg, 0.14 mmol) was added, and stirred at room temperature to react for 1 hour. Then, Compound 23-2 (31.4 mg, 0.14 mmol) was added, and the reaction system reacted at room temperature for 1 hour under nitrogen protection. After the reaction was completed, the reaction solution was filtered, and the crude product was prepared by Prep-HPLC (ACN/0.1% FA/H₂O) to obtain Compound 23-3 (35 mg, yield 54%).

MS (ESI) M: 685.3 [M+H]⁺.

¹H NMR (400 MHz, DMSO-d₆) δ 7.75 (d, J=11.0 Hz, 1H), 7.28 (s, 1H), 7.14 (s, 5H), 6.98 (d, J=8.4 Hz, 1H), 6.50 (s, 1H), 5.46-5.33 (m, 3H), 5.29-5.16 (m, 1H), 5.11 (s, 2H), 4.47-4.33 (m, 2H), 3.79 (t, J=9.1 Hz, 2H), 3.62-3.45 (m, 4H), 3.12 (dd, J=21.5, 12.7 Hz, 4H), 2.36 (s, 3H), 2.16-2.00 (m, 2H), 1.87 (dt, J=13.7, 6.8 Hz, 2H), 0.88 (t, J=7.3 Hz, 3H).

### Step C:

Compound 23-3 (20 mg, 0.029 mmol) was added into a 25 ml single-necked flask, a solvent: DCM (3 mL) was added, and BCl₃ (0.5 mL) was added at 0° C., and the reaction system reacted at room temperature for 2 hours under nitrogen protection. After the reaction was completed, the mixed solution was diluted with DCM (15 mL), and washed with water (10 mL×3) and a saturated sodium chloride solution (10 mL×1) sequentially, and organic phases were combined, then dried with anhydrous sodium sulfate, filtered, and finally concentrated under reduced pressure. 18 mg of the obtained residue was purified by Prep-HPLC (ACN/0.1% FA/H₂O) to obtain Compound 23 (5.19 mg, yield 30%).

MS (ESI) M: 595.3 [M+H]⁺.

¹H NMR (400 MHz, DMSO-d₆) δ 7.77 (d, J=10.9 Hz, 1H), 7.31 (s, 1H), 7.22 (d, J=8.0 Hz, 1H), 6.52 (s, 1H), 5.48-5.36 (m, 3H), 5.36-5.25 (m, 1H), 5.25-5.16 (m, 2H), 5.09 (dd, J=9.1, 5.9 Hz, 1H), 3.74 (td, J=9.4, 3.3 Hz, 2H), 3.54-3.41 (m, 5H), 3.16 (t, J=8.3 Hz, 3H), 2.39 (s, 3H), 2.23-2.09 (m, 2H), 1.93-1.78 (m, 2H), 0.87 (t, J=7.3 Hz, 3H).

### Example 24

### Preparation of 1-(1-cyanocyclopropyl)-3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-(2-hydroxyethyl)urea (Compound 24)

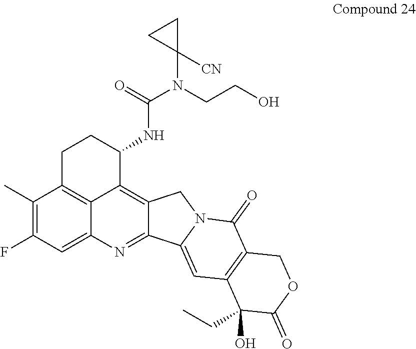 [SMILES: CC[C@@]1(O)C(=O)OCc2c1cc1n(c2=O)Cc2c-1nc1cc(F)c(C)c3c1c2[C@@H](NC(=O)N(CCO)C1(C#N)CC1)CC3]

### Reaction Route:

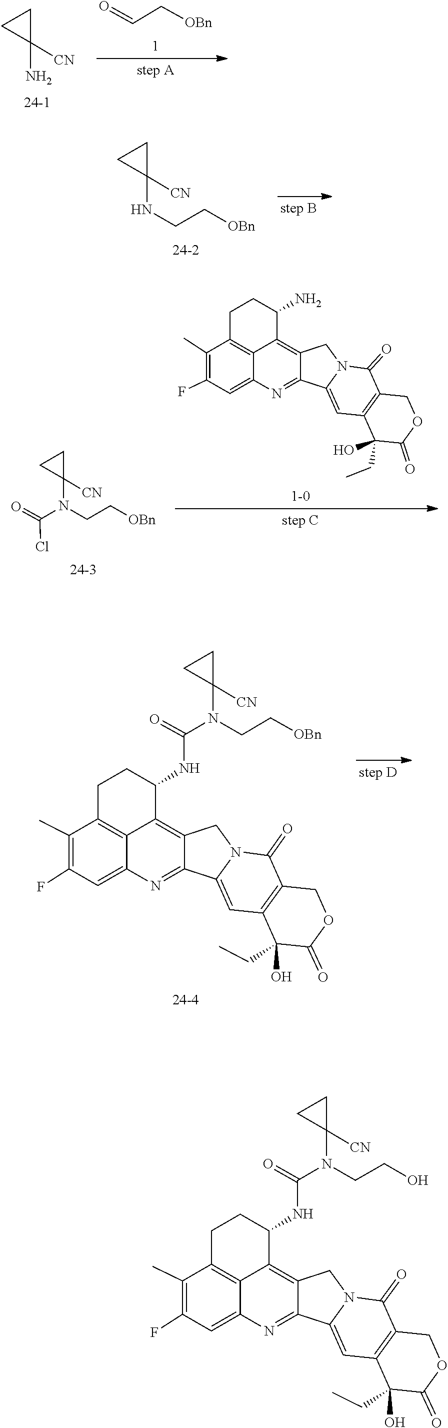

### Operation Steps:

### Step A:

Compound 24-1 (336 mg, 4 mmol) and Compound 1 (300 mg, 2 mmol) were stirred in an MeOH (2 mL) solution at room temperature for 5 minutes, and NaBH₃CN (124 mg, 2 mmol) was added. The obtained mixture was stirred at room temperature for 2 hours. After the reaction was completed, the reaction mixture was filtered and then purified by FLASH (ACN/0.1% FA/H₂O) to obtain Compound 24-2 (220 mg, yield 85%).

MS (ESI) M: 217.2 [M+H]⁺.

### Step B:

Triphosgene (300 mg, 1.02 mmol) was slowly added into a stirred solution of Compound 24-2 (220 mg, 1.2 mmol) and DIEA (0.26 mL, 1.416 mmol) in DCM (2 mL) at room temperature. The obtained mixture was stirred at room temperature for 2 hours. After the reaction was completed, the reaction mixture was filtered and washed with fresh DCM to remove an inorganic salt. The filtrate was concentrated under a reduced pressure to obtain a crude product 24-3 (200 mg). The crude product was directly used in the next step without purification.

### Step C:

Compound 1-0 (30 mg, 0.0565 mmol) was dispersed in DMF (2 mL), and then Compound 24-3 (95 mg, crude) and DIEA (0.26 mL, 1.416 mmol) were added, and the system gradually became clear and reacted at room temperature for 0.5 hours. After LCMS showed that the reaction was completed, the mixed reaction was filtered and then purified by FLASH (ACN/0.1% FA/H₂O) to obtain Compound 24-4 (20 mg, yield 85%).

MS (ESI) M: 678.1 [M+H]⁺.

¹H NMR (400 MHz, DMSO-d6) δ 7.76 (d, J=11.0 Hz, 1H), 7.29 (s, 1H), 7.26 (d, J=8.6 Hz, 1H), 7.21 (dd, J=6.6, 2.9 Hz, 2H), 7.15 (dd, J=5.0, 1.9 Hz, 2H), 5.48-5.31 (m, 3H), 5.10 (s, 2H), 4.47 (s, 2H), 3.66 (d, J=7.0 Hz, 2H), 3.52 (dd, J=9.4, 4.9 Hz, 2H), 3.20-3.03 (m, 2H), 2.38 (s, 3H), 2.20-2.07 (m, 2H), 2.00 (dd, J=14.7, 7.2 Hz, 2H), 1.93-1.80 (m, 2H), 1.59 (dd, J=20.3, 10.4 Hz, 2H), 1.52-1.39 (m, 2H), 0.88 (t, J=7.3 Hz, 3H).

### Step D:

Compound 24-4 (20 mg, 0.029 mmol) was added into a 25 ml single-necked flask, a solvent: DCM (3 mL) was added, and TfOH (0.5 mL) was added at room temperature, and the reaction system reacted at room temperature for 2 hours under nitrogen protection. After the reaction was completed, the reaction solution was concentrated under a reduced pressure. 18 mg of the obtained residue was purified by Prep-HPLC (ACN/0.1% FA/H₂O) to obtain Compound 24 (5.04 mg, yield 30%).

MS (ESI) M: 588.2 [M+H]⁺.

¹H NMR (400 MHz, DMSO-d6) δ 7.76 (d, J=10.9 Hz, 1H), 7.46 (d, J=8.6 Hz, 1H), 7.31 (s, 1H), 6.52 (s, 1H), 5.50-5.38 (m, 3H), 5.25-5.05 (m, 3H), 3.61 (d, J=5.2 Hz, 2H), 3.45 (ddd, J=53.1, 15.1, 6.0 Hz, 2H), 3.28-3.07 (m, 2H), 2.39 (s, 3H), 2.21 (d, J=5.9 Hz, 2H), 1.94-1.77 (m, 2H), 1.67-1.37 (m, 4H), 0.87 (t, J=7.3 Hz, 3H).

### Example 25

### Preparation of N—((S)-7-benzyl-17-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinoline-1-yl)carbamoyl)-2,5,8,11-tetraoxo-14-oxa-3,6,9,12,17-pentaazaicos-19-yn-1-yl)-6-(2,5-dioxo-2,5-dihydro-1H-pyrrol-1-yl)hexanamide (Compound 25)

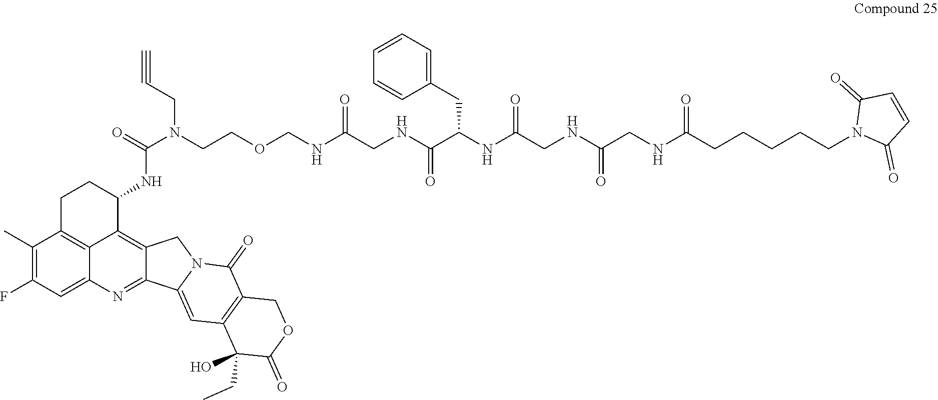 [SMILES: C#CCN(CCOCNC(=O)CNC(=O)[C@H](Cc1ccccc1)NC(=O)CNC(=O)CNC(=O)CCCCCN1C(=O)C=CC1=O)C(=O)N[C@H]1CCc2c(C)c(F)cc3nc4c(c1c23)Cn1c-4cc2c(c1=O)COC(=O)[C@]2(O)CC]

### Reaction Route:

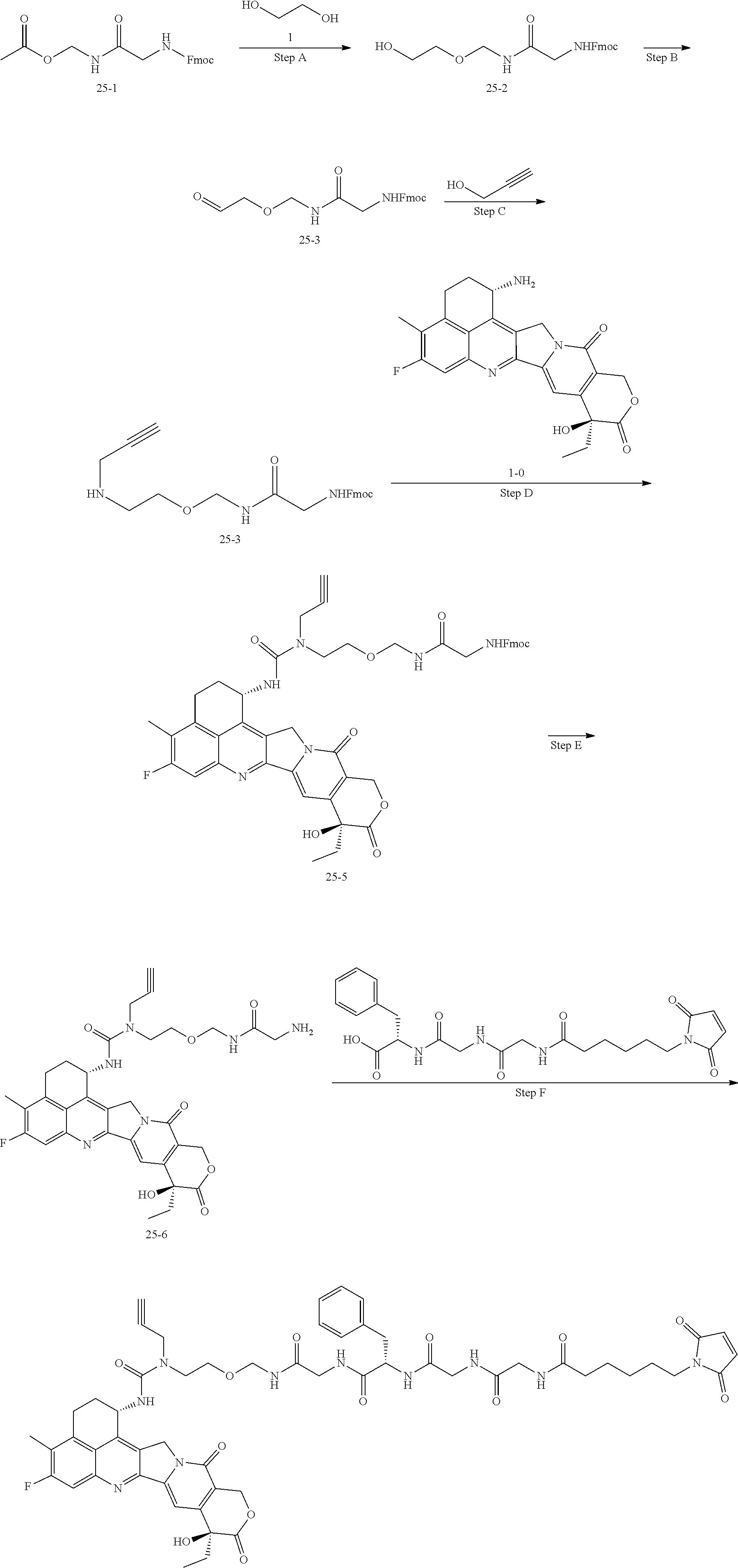

### Operation Steps:

### Step A:

Compound 25-1 (4 g, 10.8 mmol), Compound 1 (13.5 g, 217 mmol) and PPTS (545 mg, 2.17 mmol) were added into a 500 mL single-necked flask, and a solvent: DCM (200 mL) was added, and the reaction system reacted at room temperature for 15 hours under argon protection. After the reaction was completed, the solvent was spin-dried, and the crude product was purified by Flash to obtain Compound 25-2 (3.2 g, yield 79%).

MS (ESI) M: 393.1 [M+H]⁺.

### Step B:

Compound 25-2 (3.2 g, 8.64 mmol) and IBX (4.84 g, 17.28 mmol) were added into a 250 mL single-necked flask, and a solvent: acetonitrile (130 mL) was added, and the reaction system reacted at 80° C. for 2 hours under argon protection. After the reaction was completed, the reaction solution was cooled to room temperature and filtered, and the filtrate was concentrated to obtain Compound 25-3 (3.7 g, crude product), which was directly used in the next step without further purification.

### Step C:

the crude product of Compound 25-3 (1.5 g, 4.07 mmol) and propargylamine (0.9 g, 16.3 mmol) were added into a 100 mL single-necked flask, and a solvent: methanol (50 mL) was added, and the reaction system reacted at room temperature for 0.5 hours under argon protection. Then, NaBH₃CN (0.51 g, 8.14 mmol) was added, and the reaction system reacted at room temperature for 1 hour under argon protection. After the reaction was completed, the reaction solution was quenched by adding water, and an organic phase and a water phase were respectively concentrated and then combined. The crude product was purified by Flash to obtain Compound 25-4 (0.85 g, yield 50%).

MS (ESI) M: 408.2 [M+H]⁺.

### Step D:

Compound 1-0 (500 mg, 0.94 mmol) was added into a 25 mL single-necked flask, a solvent: dry DMF (8 mL) was added, and DIEA was dropwise added until the reaction solution was clear. The reaction solution was stirred at room temperature for 5 minutes, then CDI (304.8 mg, 1.88 mmol) was added, and the mixture was stirred at room temperature to react for 1 hour. Then, Compound 25-4 (31.5 mg, 0.14 mmol) was added, and the reaction system reacted at room temperature for 1 hour under nitrogen protection. After the reaction was completed, the reaction solution was filtered, and the crude product of filtrate was purified by Flash to obtain Compound 25-5 (0.42 g, yield 51%).

MS (ESI) M: 869.3 [M+H]⁺.

### Step E:

Compound 25-5 (420 mg, 0.48 mmol) was added into a 100 mL single-necked flask, a solvent: DCM (40 mL) was added, and then diethylamine (8 mL) was added, and the reaction system reacted at room temperature for 3 hours under nitrogen protection. After the reaction was completed, the solvent was spin-dried, and the crude product was dissolved with DMF and purified by Flash to obtain Compound 25-6 (260 mg, yield 83%).

MS (ESI) M: 647.3 [M+H]⁺.

¹H NMR (400 MHz, DMSO-d6) δ 8.76 (s, 1H), 8.26 (s, 1H), 7.78 (d, J=11.0 Hz, 1H), 7.32 (s, 1H), 7.00 (d, J=8.2 Hz, 1H), 5.52-5.35 (m, 3H), 5.22 (s, 2H), 4.65-4.51 (m, 2H), 4.17 (qd, J=18.1, 2.3 Hz, 3H), 3.56 (d, J=5.3 Hz, 2H), 3.49 (d, J=5.3 Hz, 2H), 3.26-3.11 (m, 5H), 2.40 (s, 3H), 2.18 (dd, J=18.2, 10.5 Hz, 2H), 1.94-1.79 (m, 2H), 0.88 (t, J=7.3 Hz, 3H).

### Step F:

Compound 25-6 (30 mg, 0.046 mmol), Mc-Gly-Gly-Phe (26.08 mg, 0.055 mmol, CAS: 1599440-15-9) and HATU (34.98 mg, 0.092 mmol) were added into a 25 mL single-necked flask, a solvent: dry DMF (3 mL) was added, and then DIEA (23.78 mg, 0.18 mmol) was added, and the reaction system reacted at room temperature for 2 hours under nitrogen protection. After the reaction was completed, the reaction solution was directly purified by Prep-HPLC to obtain Compound 25 (12.5 mg, yield 24%).

MS (ESI) M: 1102.0 [M+H]⁺.

¹H NMR (400 MHz, DMSO-d6) o 8.50 (t, J=6.6 Hz, 1H), 8.28 (t, J=5.6 Hz, 1H), 8.11 (d, J=8.1 Hz, 1H), 8.06 (t, J=5.7 Hz, 1H), 8.00 (t, J=5.6 Hz, 1H), 7.77 (d, J=10.9 Hz, 1H), 7.31 (s, 1H), 7.29-7.12 (m, 5H), 6.98 (d, J=7.9 Hz, 2H), 6.51 (s, 1H), 5.50-5.31 (m, 3H), 5.22 (s, 2H), 4.49 (ddd, J=12.8, 10.7, 5.3 Hz, 3H), 4.24-4.08 (m, 2H), 3.70 (ddd, J=18.5, 15.1, 5.9 Hz, 5H), 3.60 (d, J=5.5 Hz, 1H), 3.58-3.52 (m, 2H), 3.48 (d, J=5.0 Hz, 2H), 3.36 (d, J=7.1 Hz, 2H), 3.25-2.96 (m, 5H), 2.78 (dd, J=13.9, 9.8 Hz, 1H), 2.36 (d, J=24.6 Hz, 3H), 2.25-2.02 (m, 4H), 1.94-1.78 (m, 2H), 1.54-1.37 (m, 4H), 1.18 (dt, J=15.0, 7.3 Hz, 2H), 0.87 (t, J=7.3 Hz, 3H).

### Example 26

### Preparation of (S)—N-(2-(((2-(3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-(prop-2-yn-1-yl) ureido) ethoxy)methyl)amino)-2-oxoethyl)-2-(2-(2-(3-(2-(methylsulfonyl)pyrimidine)-5-sulfonamido)propanamido)acetamido)acetamido)-3-phenylpropanamide (Compound 26)

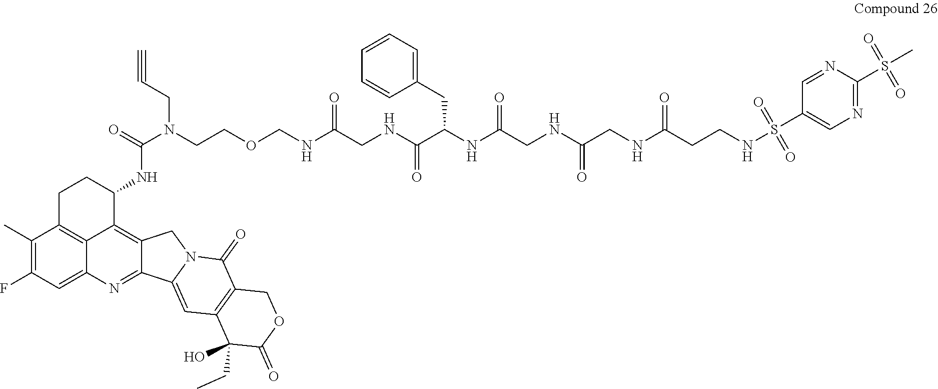 [SMILES: C#CCN(CCOCNC(=O)CNC(=O)[C@H](Cc1ccccc1)NC(=O)CNC(=O)CNC(=O)CCNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

### Reaction Route:

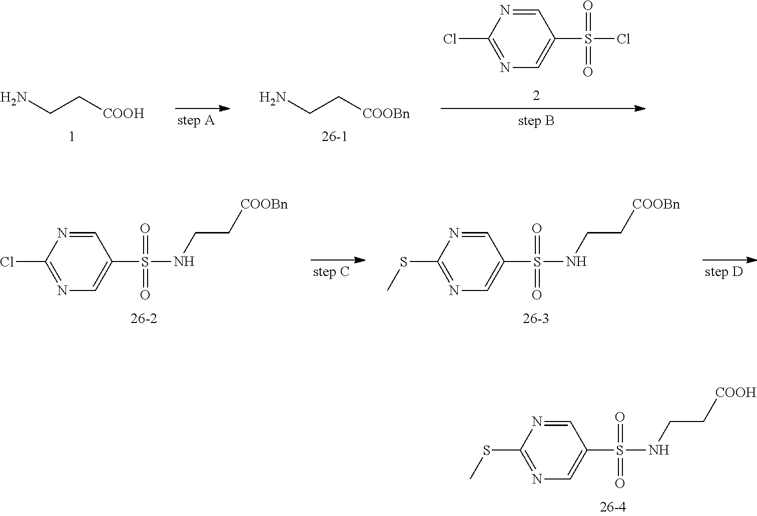

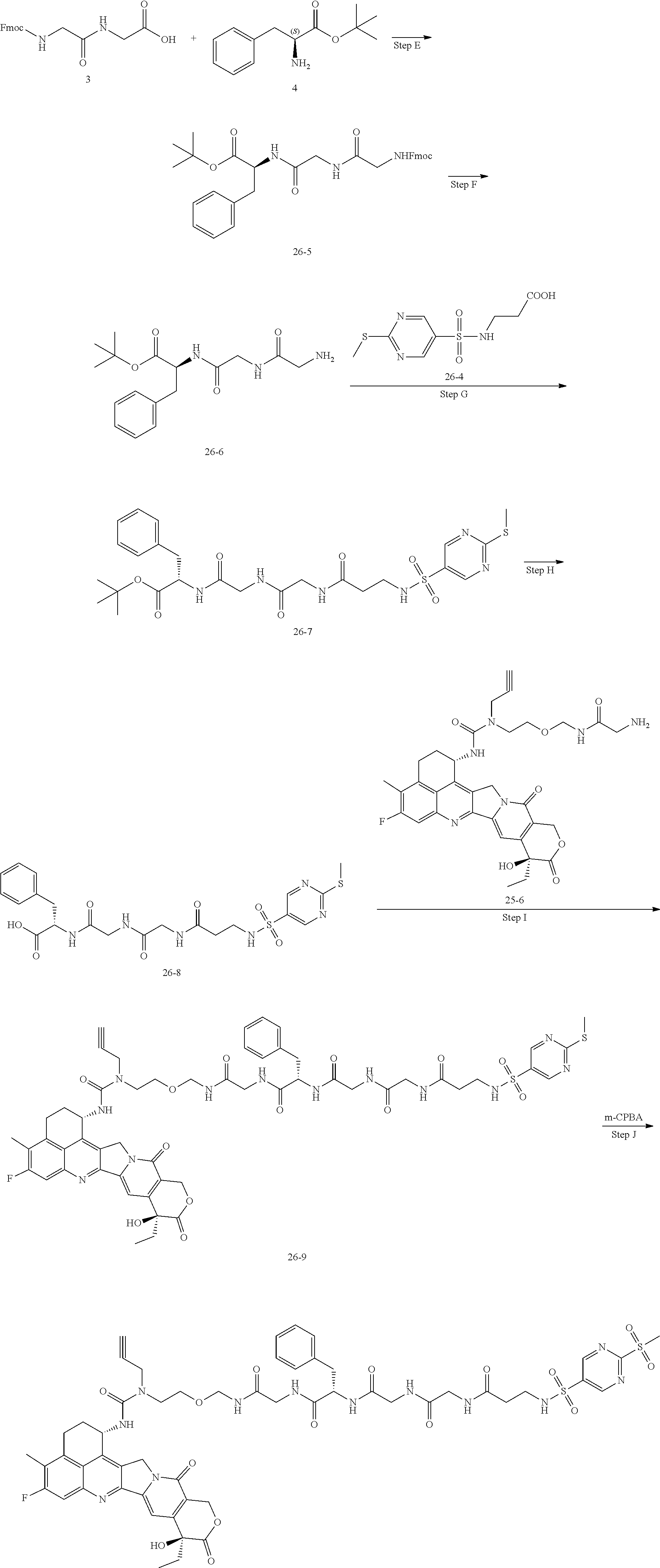

### Operation Steps:

### Step A:

Compound 1 (4 g, 44.90 mmol) was dissolved with toluene (50 mL), and BnOH (31.35 g, 289.90 mmol) and TsOH (10.02 g, 58.19 mmol) were added, and the reaction system was refluxed at 130° C. overnight (a water separator was needed) under nitrogen protection.

After the reaction was completed, the reaction solution was cooled to room temperature to precipitate a large amount of crude product, and then the crude product was added into ether and stirred to precipitate a white flocculent solid, and then filtered. The filter cake was respectively washed with ether and ethyl acetate sequentially (two timex each), and dried to obtain a crude p-toluenesulfonate product (15 g, 50% purity) of a product 26-1.

### Step B:

the Compound 26-1 (0.6 g, 1.67 mmol) was dissolved with THF (20 mL), TEA (1.19 mL) was added, the mixture was cooled to −78° C., and then Compound 2 (1.0 g, 4.69 mmol) was added, and the reaction system was stirred at this temperature for 0.5 hours. After the reaction was completed, the reaction solution was diluted with THF and then purified by Prep-HPLC to obtain a Compound 26-2 (300 mg, yield 50.4%).

MS (ESI) M: 356.2 [M+H]⁺.

### Step C:

Compound 26-2 (150 mg, 0.42 mmol) was dissolved with DMF (10 mL) and NaSCH₃ (59 mg, 0.84 mmol) was added, and the reaction system was stirred at 80° C. for 3 hours. After the reaction was completed, the reaction solution was purified by Prep-HPLC to obtain Compound 26-3 (60 mg, yield 38.7%).

MS (ESI) M: 366.1 [M+H]⁺.

### Step D:

Compound 26-3 (50 mg, 0.14 mmol) was dissolved with THF (10 mL) and Pd/C (15 mg) was added, and the reaction system was subjected to H₂ purging for three times and then stirred at 50° C. to react for 5 hours. After the reaction was completed, the reaction solution was filtered and purified by Prep-HPLC to obtain Compound 26-4 (20 mg, yield 53.3%).

MS (ESI) M: 276.0 [M−H]⁻.

### Step E:

aw material 3 (42 mmol, 15 g), raw material 4 (46 mmol, 12 g) and HATU (46 mmol, 17.7 g) were added into a round-bottom flask, 250 mL of DMF and 21 mL of DIEA were added, and the mixture was stirred at room temperature for 15 hours. 200 mL of ethyl acetate was added to the reaction solution, the system was washed with 300 mL of water once, and washed with 200 mL of saturated salt water twice, and an organic phase was collected, dried with anhydrous sodium sulfate, filtered, and concentrated under reduced pressure. The concentrated product was fully dissolved with dichloromethane, then concentrated under reduced pressure until a large number of solids were precipitated, 300 mL of petroleum ether was added, and filtered. The filter cake was washed with a mixed solution of dichloromethane and petroleum ether, and the filter cake was Compound 26-5 (28 g, yield 78.1%).

MS (ESI) M: 580.5 [M+Na]⁺.

### Step F:

Compound 26-5 (8.97 mmol, 5 g) was added into a round-bottom flask, dissolved with 45 mL of dichloromethane, 9 mL of diethylamine was added, and the mixture was stirred at room temperature for 2 hours. The reaction solution was subjected to solvent removal under reduced pressure, dissolved with DMF, and then purified by Prep-HPLC to obtain Compound 26-6 (2.04 g, yield 67.83%).

MS (ESI) M: 336.2 [M+H]⁺.

### Step G:

Compound 26-4 (90 mg, 0.32 mmol), Compound 26-6 (128.8 mg, 0.38 mmol) and HATU (243.3 mg, 0.64 mmol) were added into a 25 mL single-necked flask, a solvent: dry DMF (4 mL) was added, and then DIEA (0.21 mL, 1.28 mmol) was added, and the reaction system reacted at room temperature for 2 hours under argon protection. After the reaction was completed, the reaction solution was filtered, and the filtrate was purified by Flash to obtain a Compound 26-7 (150 mg, yield 77%).

MS (ESI) M: 595.2 [M+H]⁺.

### Step H:

Compound 26-7 (140 mg, 0.24 mmol) was added into a 100 mL single-necked flask, a solvent: DCM (20 mL) was added, and then trifluoroacetic acid (5 mL) was added, and the reaction system reacted at room temperature for 2 hours under argon protection. After the reaction was completed, the solvent was spin-dried, and the crude product was purified by Flash to obtain Compound 26-8 (90 mg, yield 71%).

¹H NMR (400 MHz, DMSO-d6) δ 12.72 (s, 1H), 8.92 (d, J=8.5 Hz, 2H), 8.21 (t, J=5.7 Hz, 1H), 8.13 (d, J=8.0 Hz, 1H), 8.04 (t, J=5.8 Hz, 1H), 7.96 (t, J=5.7 Hz, 1H), 7.30-7.24 (m, 2H), 7.24-7.16 (m, 3H), 4.41 (td, J=8.7, 5.1 Hz, 1H), 3.77-3.59 (m, 4H), 3.08-3.00 (m, 3H), 2.90-2.83 (m, 1H), 2.58 (s, 3H), 2.34 (t, J=7.1 Hz, 2H). LCMS (ESI): m/z [M+H]⁺ calcd for C₂₁H₂₆N₆O₇S₂; 538.1, mass found: 539.1.

### Step I:

Compound 25-6 (50 mg, 0.077 mmol), Compound 26-8 (45.6 mg, 0.085 mmol) and HATU (58.6 mg, 0.15 mmol) were added into a 25 mL single-necked flask, a solvent: dry DMF (3 mL) was added, and then DIEA (0.051 mL, 0.31 mmol) was added, and the reaction system reacted at room temperature for 2 hours under argon protection. After the reaction was completed, the reaction solution was filtered, and the filtrate was purified by Flash to obtain Compound 26-9 (35 mg, yield 38%).

MS (ESI) M: 1167.4 [M+H]⁺.

### Step J:

Compound 26-9 (30 mg, 0.026 mmol) and m-CPBA (26.9 mg, 0.16 mmol) were added into a 25 mL single-necked flask, and a solvent: DCM (12 mL) was added, and the reaction system reacted at room temperature for 8 hours under argon protection. After the reaction was completed, the solvent: DCM was drained at room temperature, the crude product was dissolved with DMSO (2 mL) and then purified by Flash (ACN/0.1% FA and water) to obtain a crude product, and the crude product was further purified by Prep-HPLC to obtain Compound 26 (13.2 mg, yield 43%).

MS (ESI) M: 1200.0 [M+H]⁺.

¹H NMR (400 MHz, DMSO-d6) δ 8.52 (s, 2H), 8.30 (s, 1H), 8.22 (t, J=5.8 Hz, 1H), 8.16-8.07 (m, 1H), 8.03 (t, J=5.5 Hz, 1H), 7.77 (d, J=11.0 Hz, 1H), 7.64 (t, J=5.9 Hz, 1H), 7.31 (s, 1H), 7.22 (d, J=6.5 Hz, 4H), 7.16 (d, J=6.6 Hz, 1H), 6.98 (d, J=8.4 Hz, 1H), 6.51 (s, 1H), 5.42 (s, 3H), 5.23 (s, 2H), 4.53 (dd, J=13.8, 8.0 Hz, 3H), 4.16 (q, J=19.8 Hz, 2H), 3.79-3.62 (m, 6H), 3.57 (dd, J=23.1, 5.3 Hz, 3H), 3.52-3.43 (m, 3H), 3.15 (d, J=17.1 Hz, 4H), 3.06-3.00 (m, 2H), 2.78 (d, J=9.7 Hz, 1H), 2.40 (d, J=9.5 Hz, 4H), 2.37-2.30 (m, 3H), 2.18 (d, J=13.1 Hz, 2H), 1.86 (dt, J=14.0, 6.9 Hz, 2H), 0.87 (t, J=7.2 Hz, 3H).

### Example 27

### Preparation of (S)—N-(2-(((2-(3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-(prop-2-yn-1-yl) ureido) ethoxy)methyl)amino)-2-oxoethyl)-2-(2-(2-(2-((2-((methylsulfonyl)pyrimidine)-5-sulfonamido)acetamino)acetamido)-3-phenylpropanamide (Compound 27)

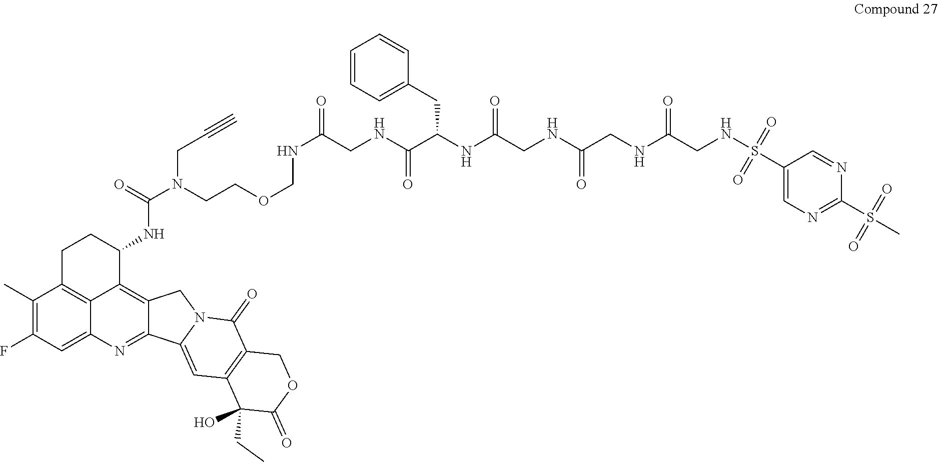 [SMILES: C#CCN(CCOCNC(=O)CNC(=O)[C@H](Cc1ccccc1)NC(=O)CNC(=O)CNC(=O)CNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

### Reaction Route:

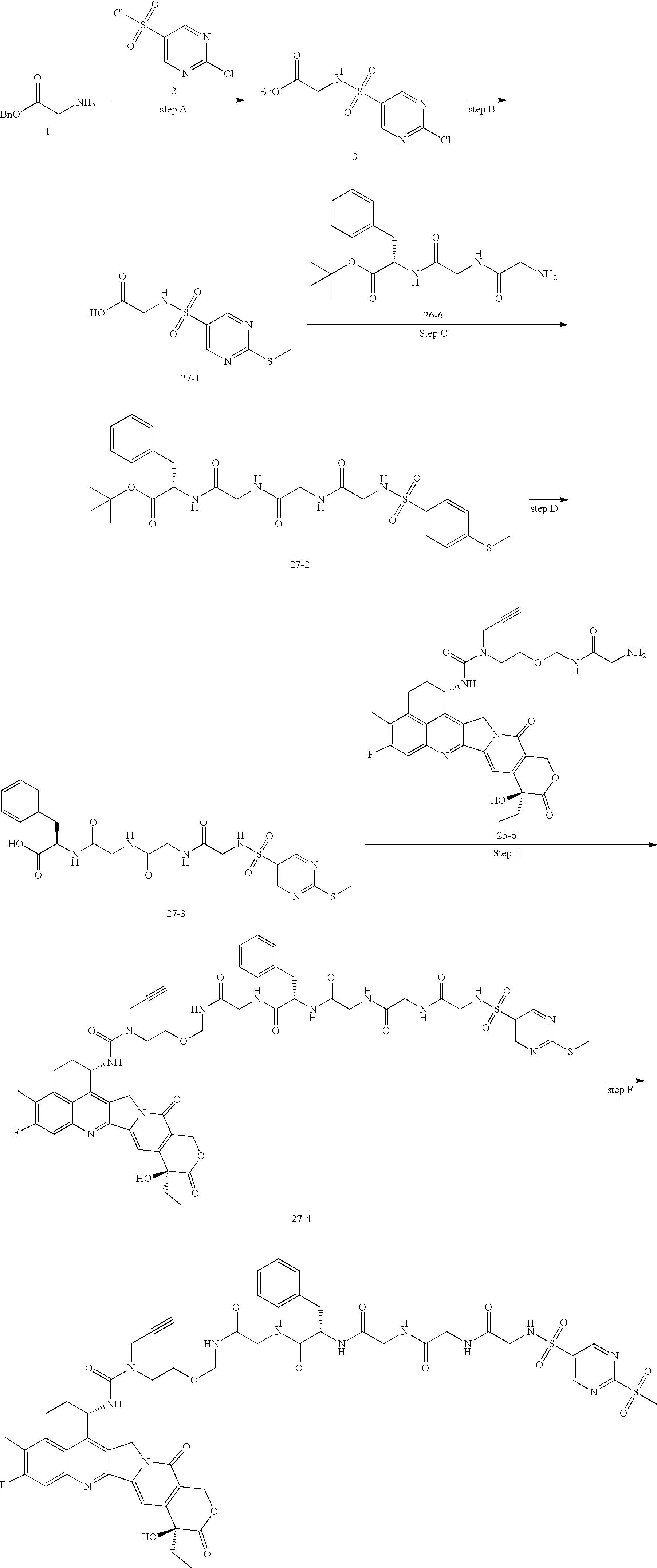

### Operation Steps:

### Step A:

Compound 2 (4.24 g, 19.9 mmol) was added into a 250 mL three-necked flask at −78° C. and 50 mL of DCM was added to the mixture to form a suspension, and a DCM solution (1 mL/50 mL) containing Compound 1 (2.00 g, 9.9 mmol) and triethylamine was prepared in another vial. The solution of Compound 1 was dropwise added into the suspension containing Compound 2 drop by drop to react for 1 hour.

The reaction solution was subjected to solvent removal under reduced pressure, and the solid was dissolved with DMF and purified by reversed-phase Flash. Most of the solvent was removed from the prepared solution under reduced pressure, and the residue was freeze-dried to obtain Compound 3 (1.1 g, yield 30.3%).

MS (ESI) M: 342.0 [M+H]⁺.

¹H NMR (400 MHz, CDCl₃) δ 8.99 (s, 2H), 7.41-7.34 (m, 3H), 7.28 (dd, J=6.6, 2.6 Hz, 2H), 5.53 (t, J=5.3 Hz, 1H), 5.10 (s, 2H), 3.97 (d, J=5.7 Hz, 2H).

### Step B:

Compound 3 (1.10 g, 3.22 mmol) was added into a reaction flask, dissolved with 10 mL of DMF, and then sodium thiomethoxide (0.90 g, 12.84 mmol) was added to react at 80° C. for 1 hour, and then 10 ml of water was added to the system and the mixture was continuously stirred for 2 hours. After the reaction was completed, a pH value was adjusted to 5-6 with a hydrochloric acid aqueous solution, and then the reaction solution was directly purified with reverse Flash. The prepared solution was extracted with ethyl acetate for three times, and organic phases were combined, dried with saturated salt water and anhydrous sodium sulfate, then filtered, and spin-dried to obtain Compound 27-1 (0.68 g, yield 79.89%).

MS (ESI) M: 264.2 [M+H]⁺.

### Step C:

Compound 27-1 (0.50 g, 1.90 mmol), Compound 26-6 (0.53 g, 1.58 mmol) and HATU (0.90 g, 2.37 mmol) were added into a reaction flask, dissolved with DMF (10 mL), then subjected to nitrogen purging for three times, DIEA (0.41 g, 3.16 mmol) was dropwise added, and stirred at room temperature for 1 hour. After the reaction was completed, the system was directly purified by reverse Flash. The prepared solution was extracted with ethyl acetate for three times, and organic phases were combined, dried with saturated salt water and anhydrous sodium sulfate, then filtered, and spin-dried to obtain Compound 27-2 (0.72 g, yield 65%).

MS (ESI) M: 581.2 [M+H]⁺.

### Step D:

Compound 27-2 (722 mg, 1.24 mmol) was added into a reaction flask, DCM (6 mL) and TFA (1.2 mL) were added, and the mixture was stirred at room temperature overnight. The system was spin-dried, dissolved with DMF, and then purified by reverse Flash. The prepared solution was extracted with ethyl acetate for three times, and organic phases were combined, dried with saturated salt water and anhydrous sodium sulfate, then filtered, and spin-dried to obtain Compound 27-3 (635 mg, yield 97%).

MS (ESI) M: 525.3 [M+H]⁺.

### Step E:

Compound 25-6 (50 mg, 0.077 mmol), Compound 27-3 (44.4 mg, 0.085 mmol) and HATU (58.5 mg, 0.15 mmol) were added into a 25 mL single-necked flask, a solvent: dry DMF (3 mL) was added, and then DIEA (0.051 mL, 0.31 mmol) was added, and the reaction system reacted at room temperature for 2 hours under argon protection. After the reaction was completed, the reaction solution was filtered, and the filtrate was purified by Prep-HPLC to obtain Compound 27-4 (13 mg, yield 14%).

MS (ESI) M: 1153.4 [M+H]⁺.

### Step F:

Compound 27-4 (10 mg, 0.0087 mmol) and m-CPBA (12.01 mg, 0.070 mmol) were added into a 25 mL single-bottlenecked flask, and a solvent: DCM (12 mL) was added, and the reaction system reacted at room temperature for 8 hours under argon protection. After the reaction was completed, the solvent: DCM was drained at room temperature, the crude product was dissolved with DMF (2 mL) and then purified by Flash (ACN/0.1% FA and water) to obtain 11 mg of crude product, and the crude product was purified by Prep-HPLC to obtain Compound 27 (4.6 mg, yield 45%).

MS (ESI) M: 1186.0 [M+H]⁺.

¹H NMR (400 MHz, DMSO-d₆) δ 8.59 (s, 1H), 8.40-8.19 (m, 4H), 8.04 (s, 1H), 7.77 (d, J=11.0 Hz, 1H), 7.31 (s, 1H), 7.18 (d, J=23.5 Hz, 5H), 7.06 (d, J=7.8 Hz, 1H), 6.66 (s, 1H), 6.52 (s, 1H), 5.42 (s, 3H), 5.23 (s, 2H), 4.50 (d, J=19.9 Hz, 2H), 4.17 (d, J=6.5 Hz, 2H), 3.78-3.62 (m, 5H), 3.59-0.44 (m, 6H), 3.22-3.00 (m, 5H), 2.81 (d, J=10.0 Hz, 1H), 2.39 (s, 3H), 2.31 (s, 1H), 2.20 (s, 2H), 1.99 (d, J=14.3 Hz, 2H), 1.92-1.80 (m, 2H), 1.75 (s, 2H), 0.87 (t, J=6.9 Hz, 3H).

### Example 28

### Preparation of N—((S)-7-benzyl-17-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)-2,5,8,11-tetraoxo-14-oxa-3,6,9,12,17-pentazaicos-19-en-1-yl)-6-(2,5-dioxo-2,5-dihydro-1H-pyrrol-1-yl)hexanamide (Compound 28)

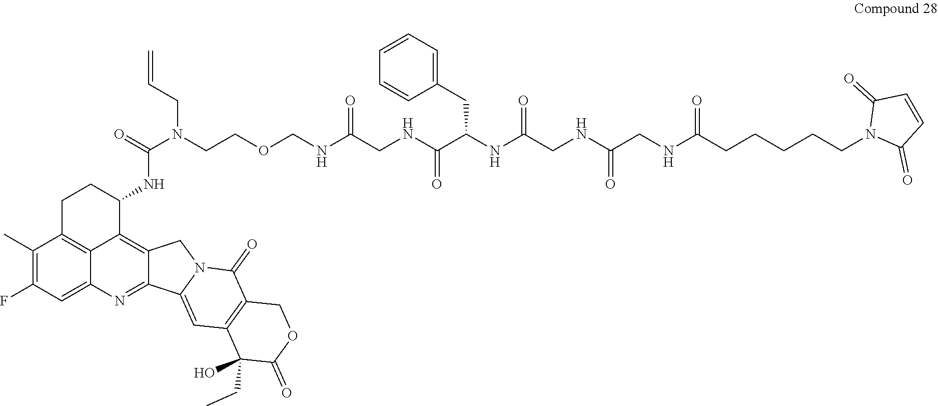 [SMILES: C=CCN(CCOCNC(=O)CNC(=O)[C@H](Cc1ccccc1)NC(=O)CNC(=O)CNC(=O)CCCCCN1C(=O)C=CC1=O)C(=O)N[C@H]1CCc2c(C)c(F)cc3nc4c(c1c23)Cn1c-4cc2c(c1=O)COC(=O)[C@]2(O)CC]

### Reaction Route:

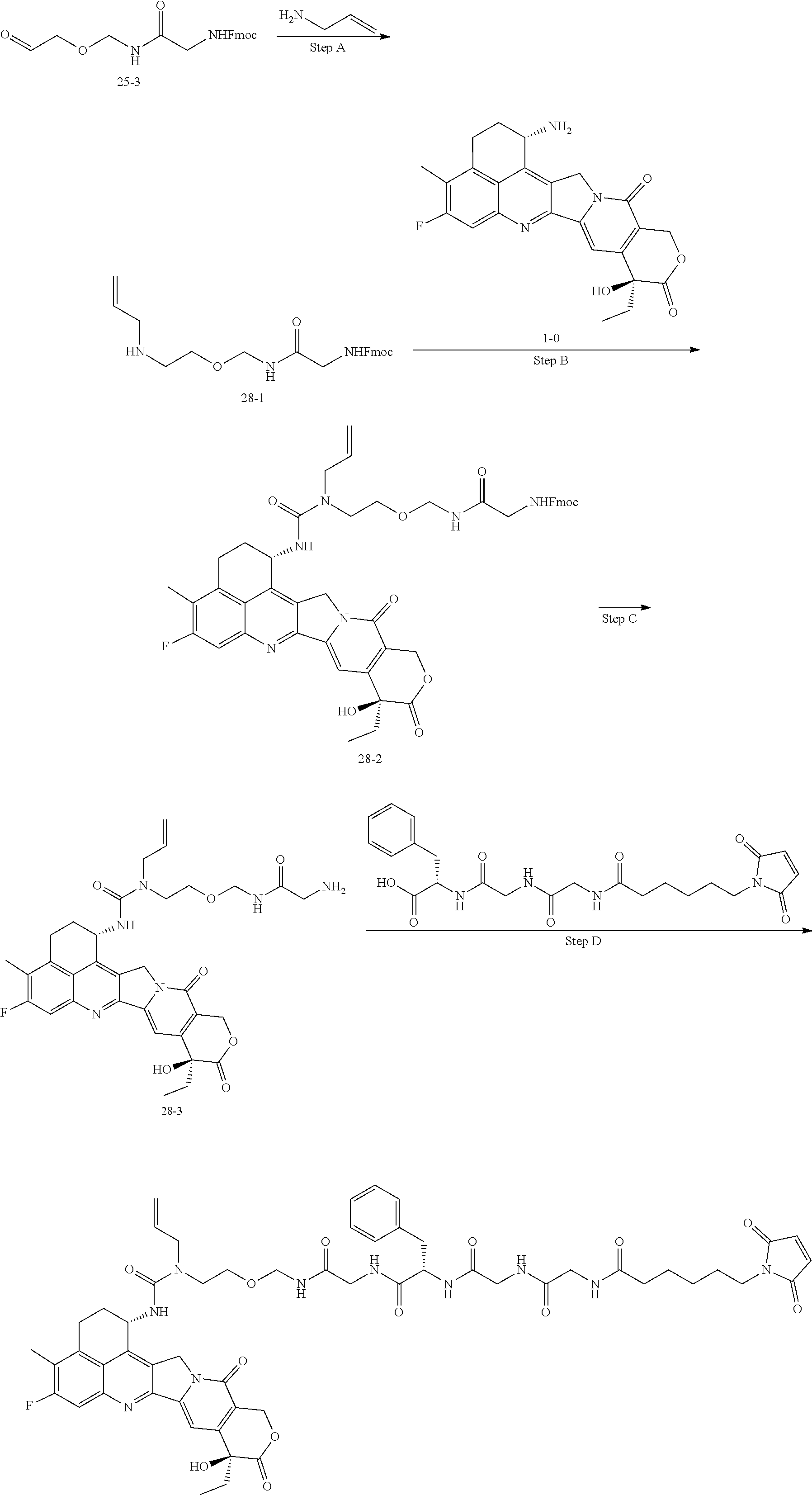

### Operation Steps:

### Step A:

a crude product of Compound 25-3 (1.5 g, 4.07 mmol) and allylamine (0.9 g, 16.3 mmol) were added into a 100 mL single-necked flask, and a solvent: methanol (50 mL) was added, and the reaction system reacted at room temperature for 0.5 hours under argon protection. Then, NaBH₃CN (0.51 g, 8.14 mmol) was added, and the reaction system reacted at room temperature for 1 hour under argon protection. After the reaction was completed, the reaction solution was quenched by adding water, and an organic phase and a water phase were respectively concentrated and then combined. The crude product was purified by Flash chromatography to obtain Compound 28-1 (0.85 g, yield 50%).

MS (ESI) M: 410.2 [M+H]⁺.

### Step B:

Compound 1-0 (500 mg, 0.94 mmol) was added into a 25 mL single-bottlenecked flask, an ultra-dry solvent: DMF (8 mL) was added, and DIEA (2 mL) was dropwise added until the reaction solution was clear. The reaction solution was stirred at room temperature for 5 minutes, then CDI (304.8 mg, 1.88 mmol) was added, and the mixture was stirred at room temperature to react for 1 hour. Then, Compound 28-1 (315 mg, 0.77 mmol) was added, and the reaction system reacted at room temperature for 1 hour under nitrogen protection. After the reaction was completed, the reaction solution was filtered, and the crude product of filtrate was purified by Flash to obtain Compound 28-2 (0.42 g, yield 51%).

MS (ESI) M: 871.4 [M+H]⁺.

### Step C:

Compound 28-2 (420 mg, 0.48 mmol) was added into a 100 mL single-necked flask, a solvent: DCM (40 mL) was added, and then diethylamine (8 mL) was added, and the reaction system reacted at room temperature for 3 hours under nitrogen protection. After the reaction was completed, the DCM solvent was removed by rotary evaporation, and the crude product was dissolved with DMF and purified by Flash to obtain Compound 28-3 (260 mg, yield 83%).

MS (ESI) M: 649.3 [M+H]⁺.

### Step D:

Compound 28-3 (30 mg, 0.046 mmol), Mc-Gly-Gly-Phe (26.08 mg, 0.055 mmol) and HATU (34.98 mg, 0.092 mmol) were added into a 25 mL single-necked flask, a solvent: dry DMF (3 mL) was added, and then DIEA (23.78 mg, 0.18 mmol) was added, and the reaction system reacted at room temperature for 2 hours under nitrogen protection. After the reaction was completed, the reaction solution was directly purified by Prep-HPLC to obtain Compound 28 (12.5 mg, yield 24%).

MS (ESI) M: 1103.4 [M+H]⁺.

¹H NMR (400 MHz, DMSO-d₆) δ 8.50 (t, J=6.6 Hz, 1H), 8.28 (t, J=5.6 Hz, 1H), 8.05 (ddd, J=26.5, 14.2, 7.0 Hz, 3H), 7.77 (d, J=10.9 Hz, 1H), 7.31 (s, 1H), 7.26-7.13 (m, 5H), 6.99 (s, 1H), 6.81 (d, J=8.2 Hz, 1H), 6.51 (d, J=5.8 Hz, 1H), 5.81 (ddd, J=15.5, 10.2, 5.0 Hz, 1H), 5.41 (d, J=8.0 Hz, 2H), 5.23-5.10 (m, 3H), 4.66-4.36 (m, 3H), 3.95 (ddd, J=25.6, 17.2, 8.0 Hz, 2H), 3.77-3.63 (m, 5H), 3.62-3.44 (m, 4H), 3.42-3.34 (m, 5H), 3.30-3.01 (m, 4H), 2.78 (dd, J=13.7, 9.7 Hz, 1H), 2.39 (s, 3H), 2.16-1.98 (m, 4H), 1.94-1.78 (m, 2H), 1.50-1.41 (m, 4H), 1.22-1.13 (m, 2H), 0.87 (t, J=7.1 Hz, 3H).

### Example 29

### Preparation of N—((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)-2,5,8,11-tetraoxo-14-oxa-3,6,9,12,17-pentaazaicos-19-yn-1-yl)-6-(2-(methylsulfonyl)pyrimidin-5-yl)hex-5-ynamide (Compound 29)

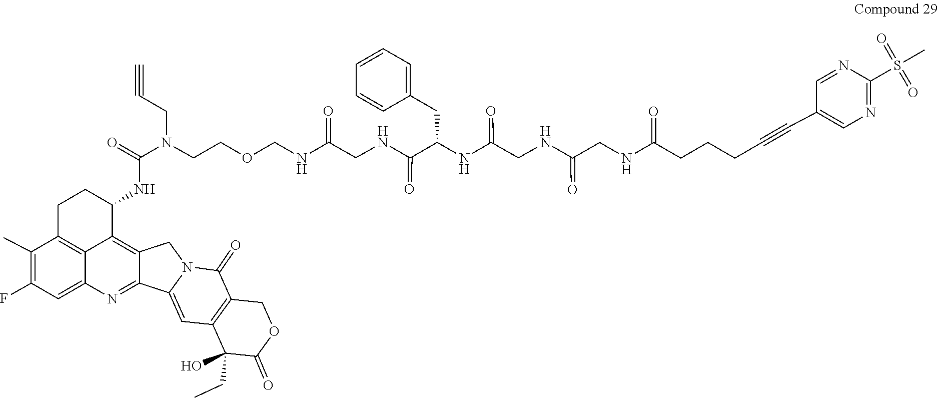

### Reaction Route:

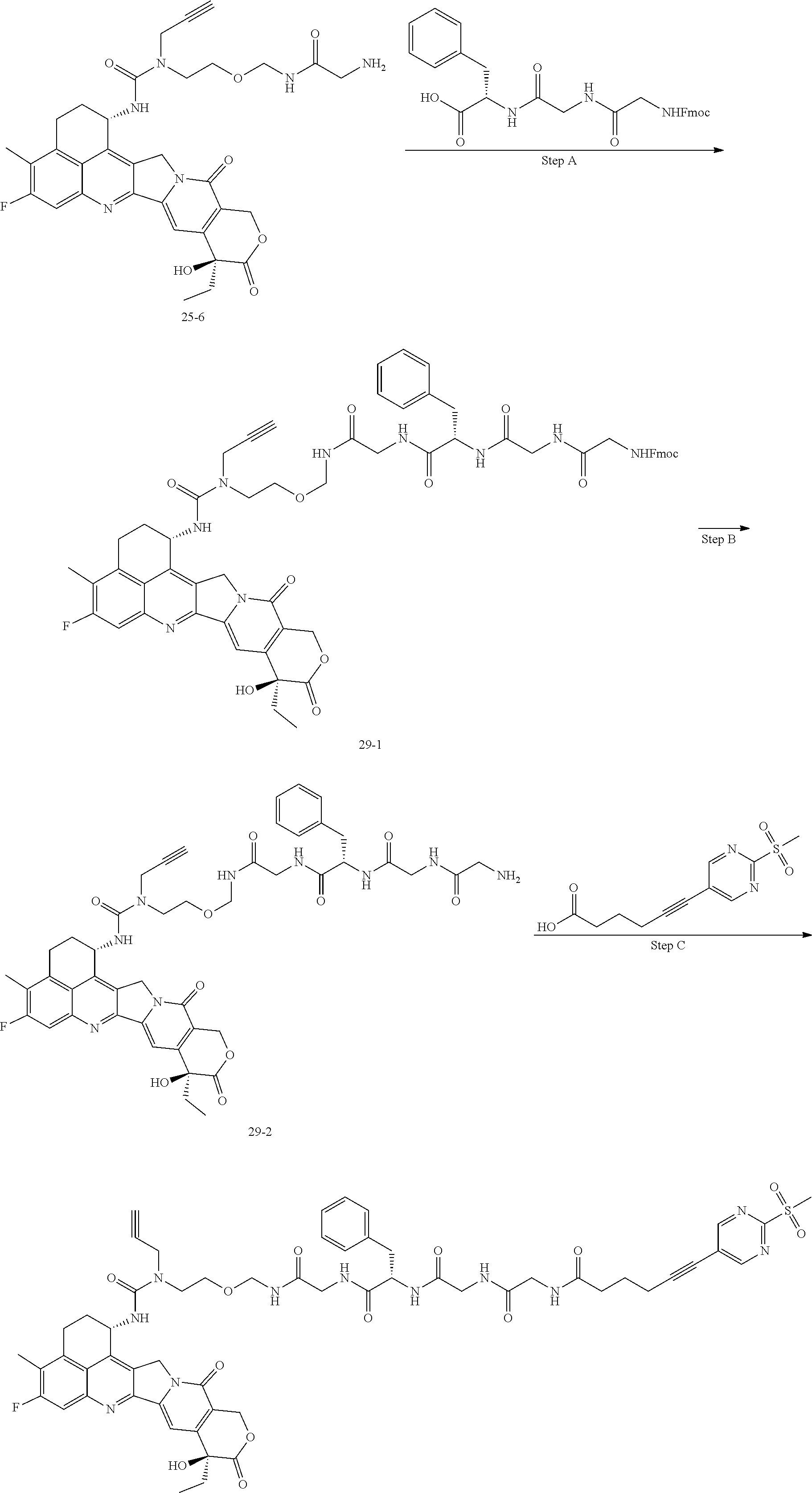

### Operation Steps:

### Step A:

Compound 25-6 (75 mg, 0.12 mmol), Fmoc-Gly-Gly-Phe-OH (78.2 mg, 0.16 mmol) and HATU (68.1 mg, 0.18 mmol) were added into a 25 mL single-necked flask, a solvent: dry DMF (4 mL) was added, and then DIEA (0.08 mL, 0.48 mmol) was added, and the reaction system reacted at room temperature for 2 hours under argon protection. After the reaction was completed, the reaction solution was filtered, and the filtrate was purified by Flash to obtain Compound 29-1 (45 mg, yield 34%).

MS (ESI) M: 1130.3 [M+H]⁺.

### Step B:

Compound 29-1 (35 mg, 0.026 mmol) was added into a 25 mL single-necked flask, a solvent: DCM (15 mL) was added, and then diethylamine (3 mL) was added, and the reaction system reacted at room temperature for 3 hours under argon protection. After the reaction was completed, the solvent: DCM was spin-dried, and the crude product was dissolved with DMF (2 mL) and then purified by Flash to obtain Compound 29-2 (35 mg, yield 97%).

MS (ESI) M: 908.3 [M+H]⁺.

### Step C:

Compound 29-2 (35 mg, 0.039 mmol), 6-(2-(methylsulfonyl)pyrimidine-5-yl)hexan-5-ynoic acid (12.6 mg, 0.047 mmol) and PyBOP (30.4 mg, 0.058 mmol) were added into a 25 mL single-necked flask, a solvent: dry DMF (3 mL) was added, and then DIEA (0.026 mL, 0.16 mmol) was added, and the reaction system reacted at room temperature for 2 hours under argon protection. After the reaction was completed, the reaction solution was filtered, and the filtrate was purified by Prep-HPLC to obtain Compound 29 (8.58 mg, yield 19%).

MS (ESI) M: 1158.9 [M+H]⁺.

¹H NMR (400 MHz, DMSO-d6) δ 9.09 (s, 2H), 8.52 (t, J=6.6 Hz, 1H), 8.30 (d, J=5.8 Hz, 1H), 8.19 (t, J=5.7 Hz, 1H), 8.13 (d, J=8.1 Hz, 1H), 8.05 (d, J=5.6 Hz, 1H), 7.77 (d, J=11.0 Hz, 1H), 7.31 (s, 1H), 7.22 (d, J=5.8 Hz, 4H), 7.16 (d, J=6.4 Hz, 1H), 6.98 (d, J=8.1 Hz, 1H), 6.52 (s, 1H), 5.48-5.35 (m, 3H), 5.22 (s, 2H), 4.52 (dd, J=15.8, 9.7 Hz, 3H), 4.16 (q, J=18.4 Hz, 2H), 3.79-3.64 (m, 5H), 3.61 (d, J=5.3 Hz, 1H), 3.54 (d, J=5.0 Hz, 2H), 3.48 (s, 2H), 3.40 (s, 3H), 3.15 (d, J=16.8 Hz, 3H), 3.06-2.98 (m, 1H), 2.85-2.74 (m, 1H), 2.60-2.53 (m, 2H), 2.39 (s, 3H), 2.32 (t, J=7.3 Hz, 2H), 2.16 (s, 2H), 1.90-1.73 (m, 4H), 0.87 (t, J=7.2 Hz, 3H).

### Example 30

### Preparation of (S)—N-(3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)-9-oxo-2,6-dioxa-3,8-diazadecan-10-yl)-2-(2-(2-(2-((2-(methylsulfonyl)pyrimidine)-5-sulfonamido)acetamido)acetamide)acetamido)-3-phenylpropanamide (Compound 30)

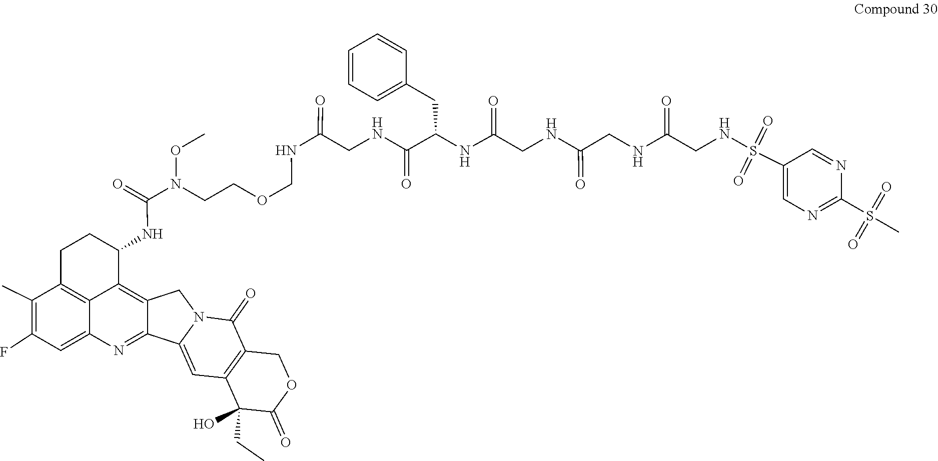

### Reaction Route:

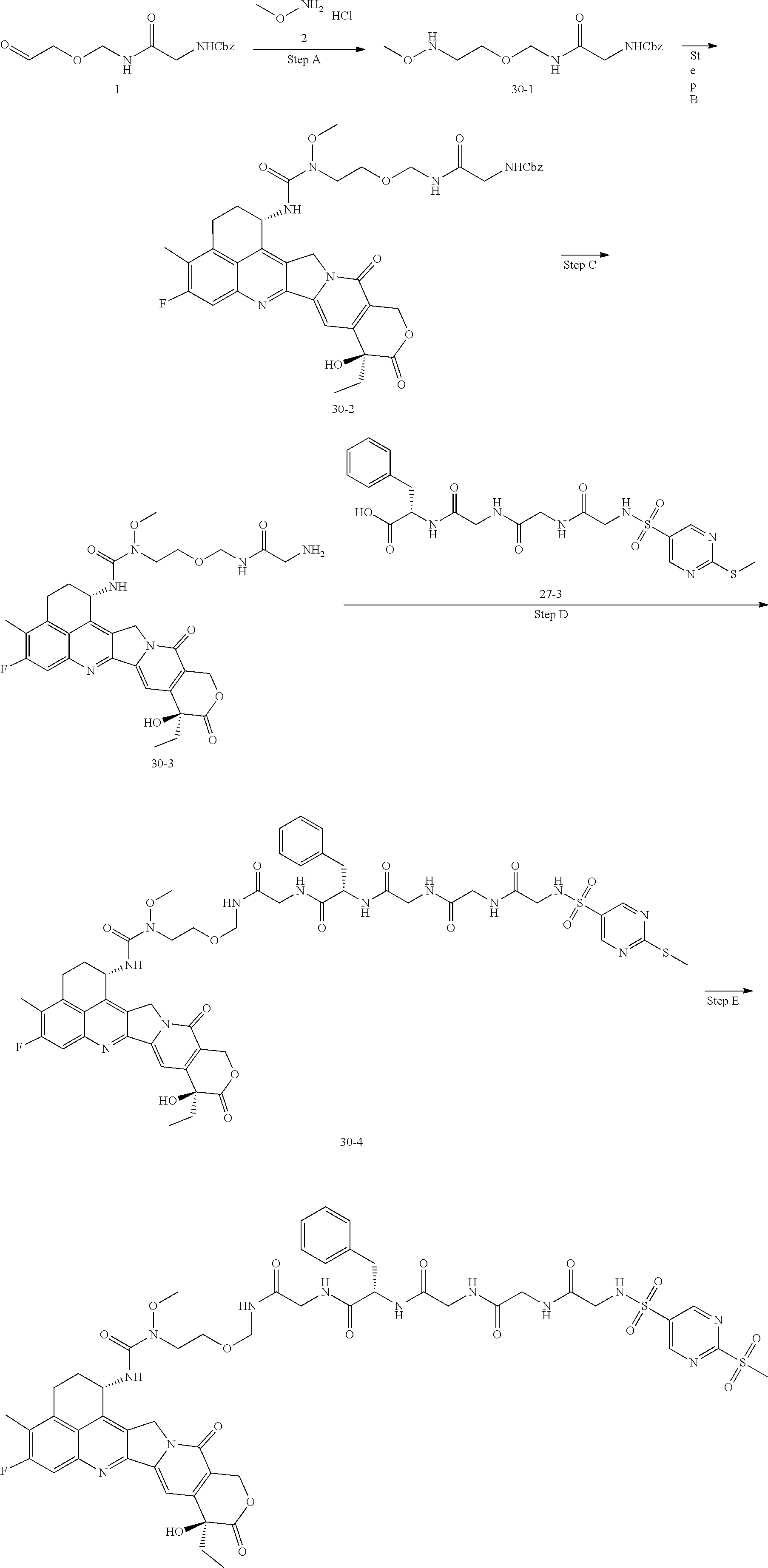

### Operation Steps:

### Step A:

a crude product of Compound 1 (300 mg, 1.07 mmol) and Compound 2 (357.5 mg, 4.28 mmol) were added into a 100 mL single-necked flask, and a solvent: methanol (20 mL) was added, and the reaction system reacted at room temperature for 0.5 hours under argon protection. Then, NaBH₃CN (134.5 mg, 4.14 mmol) was added, and the reaction system reacted at room temperature for 1 hour under argon protection. After the reaction was completed, the reaction solution was quenched by adding water, and an organic phase and a water phase were respectively concentrated and then combined. The crude product was purified by Flash to obtain Compound 30-1 (100 mg, yield 30%).

MS (ESI) M: 312.1 [M+H]⁺.

¹H NMR (400 MHz, DMSO-d6) δ 8.74 (t, J=6.5 Hz, 1H), 7.51 (t, J=6.0 Hz, 1H), 7.45-7.25 (m, 6H), 5.04 (s, 2H), 4.58 (d, J=6.6 Hz, 2H), 3.65 (t, J=2.9 Hz, 5H), 3.57 (dd, J=12.1, 6.7 Hz, 2H), 3.20 (dd, J=19.2, 13.9 Hz, 2H).

### Step B:

Exatecan mesylate (110 mg, 0.21 mmol) was added into a 25 mL single-necked flask, a solvent: dry DMF (8 mL) was added, and DIEA was added dropwise until the reaction solution was clear. The reaction solution was stirred at room temperature for 5 minutes, then CDI (44.3 mg, 0.27 mmol) was added, and stirred at room temperature to react for 0.5 hours. Then, Compound 30-1 (71.9 mg, 0.23 mmol) was added, and the reaction system reacted at 40° C. for 14 hours under nitrogen protection. After the reaction was completed, the reaction solution was filtered, and the crude product of filtrate was purified by Flash to obtain Compound 30-2 (60 mg, yield 37%).

MS (ESI) M: 773.4 [M+H]⁺.

¹H NMR (400 MHz, DMSO-d6 and D₂O) δ 7.58 (d, J=10.8 Hz, 1H), 7.37-7.15 (m, 5H), 5.35 (dd, J=35.1, 16.2 Hz, 3H), 5.14 (d, J=18.9 Hz, 1H), 5.03-4.79 (m, 3H), 4.65-4.53 (m, 2H), 3.63 (d, J=14.6 Hz, 8H), 3.19 (d, J=17.3 Hz, 1H), 2.99 (s, 1H), 2.27 (s, 3H), 2.19 (s, 1H), 2.09 (d, J=9.7 Hz, 1H), 1.80 (d, J=11.8 Hz, 2H), 0.82 (t, J=7.3 Hz, 3H).

### Step C:

Compound 30-2 (55 mg, 0.071 mmol) was added into a 100 mL single-necked flask, a solvent: Methanol (25 mL) was added, and then palladium on carbon (30 mg) was added, and the reaction system reacted at room temperature for 3 hours under hydrogen protection. After the reaction was completed, the reaction solution was filtered, the filtrate was concentrated, and then the crude product was dissolved with DMF and purified by Flash to obtain Compound 30-3 (30 mg, yield 66%).

MS (ESI) M: 639.3 [M+H]⁺.

### Step D:

Compound 30-3 (30 mg, 0.047 mmol), compound 27-3 (29.6 mg, 0.056 mmol) and HATU (26.8 mg, 0.071 mmol) were added into a 25 mL single-necked flask, a solvent: dry DMF (3 mL) was added, and then DIEA (24.3 mg, 0.19 mmol) was added, and the reaction system reacted at room temperature for 2 hours under nitrogen protection. After the reaction was completed, the reaction solution was directly purified by Prep-HPLC to obtain Compound 30-4 (18 mg, yield 33%).

MS (ESI) M: 1146.0 [M+H]⁺.

### Step E:

Compound 30-4 (16 mg, 0.014 mmol) and m-CPBA (14.5 mg, 0.084 mmol) were added into a 25 mL single-necked flask, and a solvent of DCM (10 mL) was added, and the reaction system reacted at room temperature for 6 hours under argon protection. After the reaction was completed, the solvent: DCM was drained, the crude product was dissolved with DMSO (2 mL) and then purified by Flash (ACN/0.1% FA and water) to obtain 11 mg of crude product, and the crude product was dissolved with DMSO and then purified by Prep-HPLC to obtain Compound 30 (4.5 mg, yield 27%).

MS (ESI) M: 1178.0 [M+H]⁺.

¹H NMR (400 MHz, DMSO-d6) δ 8.60 (s, 1H), 8.41 (d, J=21.5 Hz, 3H), 8.23 (dd, J=21.4, 12.4 Hz, 2H), 8.07 (s, 1H), 7.95 (d, J=8.5 Hz, 1H), 7.77 (d, J=11.1 Hz, 1H), 7.32 (s, 1H), 7.20 (t, J=15.2 Hz, 5H), 6.53 (s, 1H), 5.40 (d, J=15.9 Hz, 3H), 5.26 (d, J=18.9 Hz, 1H), 5.13 (d, J=18.9 Hz, 1H), 4.62 (s, 2H), 4.50 (s, 1H), 4.16 (s, 1H), 3.74 (dd, J=16.2, 5.0 Hz, 3H), 3.67-3.56 (m, 11H), 3.06 (d, J=9.9 Hz, 4H), 2.82 (d, J=11.2 Hz, 2H), 2.38 (s, 3H), 2.19 (s, 2H), 2.01 (s, 2H), 1.94-1.79 (m, 2H), 0.87 (t, J=7.2 Hz, 3H).

### Example 31

### Preparation of N—((S)-13-benzyl-3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)-9,12,15,18-tetraoxo-2,6-dioxa-3,8,11,14,17-pentaazanonadecan-19-yl)-6-(2,5-dioxo-2,5-dihydro-1H-pyrrol-1-yl)hexanamide (Compound 31)

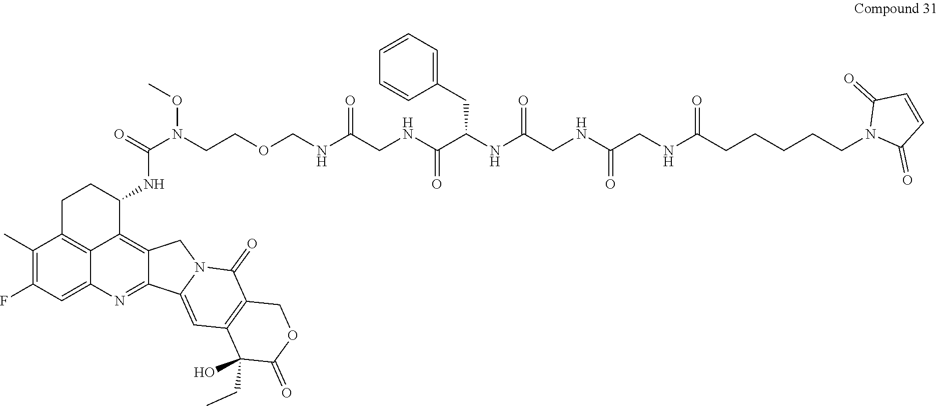 [SMILES: CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCCCCN1C(=O)C=CC1=O)OC)CC3]

### Reaction Route:

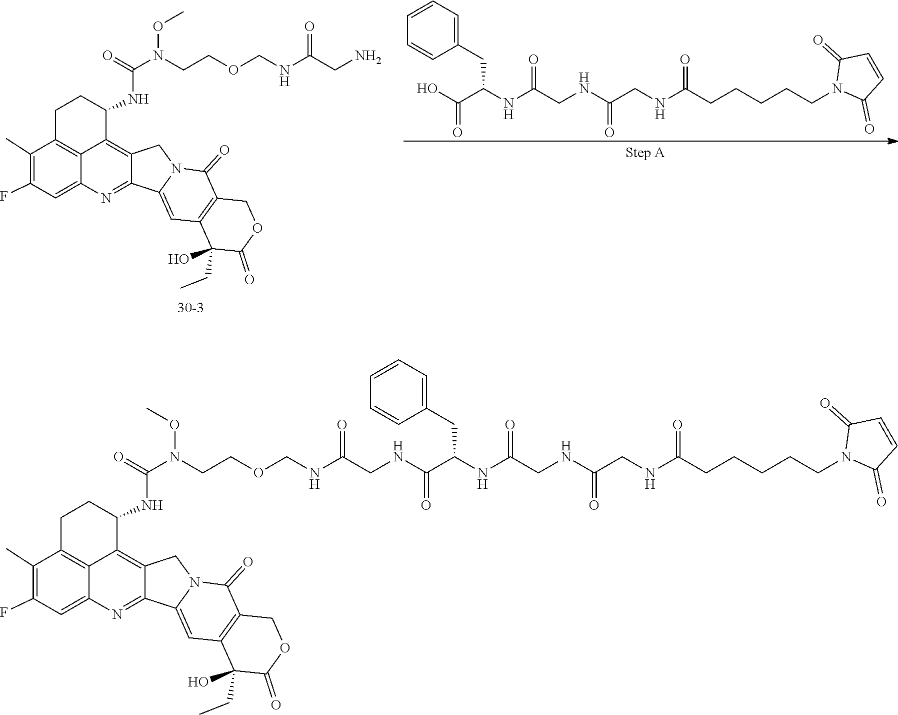 [SMILES: CC[C@@]1(O)C(=O)OCc2c1cc1n(c2=O)Cc2c-1nc1cc(F)c(C)c3c1c2[C@@H](NC(=O)N(CCOCNC(=O)CN)OC)CC3 ; O=C(CCCCCN1C(=O)C=CC1=O)NCC(=O)NCC(=O)N[C@@H](Cc1ccccc1)C(=O)O ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCCCCN1C(=O)C=CC1=O)OC)CC3]

### Operation Steps:

### Step A:

Compound 30-3 (15 mg, 0.023 mmol), Mc-Gly-Gly-Phe (13.0 mg, 0.028 mmol) and HATU (13.1 mg, 0.035 mmol) were added into a 25 mL single-necked flask, a solvent: dry DMF (3 mL) was added, and then DIEA (0.015 mL, 0.092 mmol) was added, and the reaction system reacted at room temperature for 2 hours under argon protection. After the reaction was completed, the reaction solution was filtered, and the filtrate was purified by Prep-HPLC to obtain Compound 31 (6.5 mg, yield 25%).

MS (ESI) M: 1093.9 [M+H]⁺.

¹H NMR (400 MHz, DMSO-d6) δ 8.56 (d, J=6.3 Hz, 1H), 8.46 (s, 1H), 8.34 (s, 1H), 8.16 (d, J=7.8 Hz, 1H), 8.05 (d, J=22.1 Hz, 2H), 7.92 (d, J=9.0 Hz, 1H), 7.77 (d, J=10.9 Hz, 1H), 7.32 (s, 1H), 7.19 (d, J=26.3 Hz, 5H), 6.98 (s, 1H), 6.53 (s, 1H), 5.42 (s, 2H), 5.19 (dd, J=49.2, 18.9 Hz, 2H), 4.61 (s, 2H), 4.50 (s, 1H), 3.83-3.50 (m, 12H), 3.24 (s, 3H), 3.06 (d, J=9.2 Hz, 3H), 2.87-2.74 (m, 1H), 2.38 (s, 3H), 2.12 (dd, J=25.3, 18.2 Hz, 4H), 1.93-1.76 (m, 2H), 1.46 (dd, J=14.2, 7.0 Hz, 3H), 1.30-0.98 (m, 4H), 0.87 (t, J=7.1 Hz, 3H).

### Example 32

### Preparation of (S)—N-(3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)-9-oxo-2,6-dioxa-3,8-diazadecan-10-yl)-2-(2-(2-(3-((2-(methylsulfonyl)pyrimidine)-5-sulfonamido)propanamido)acetamido)acetamino)-3-phenylpronanamide

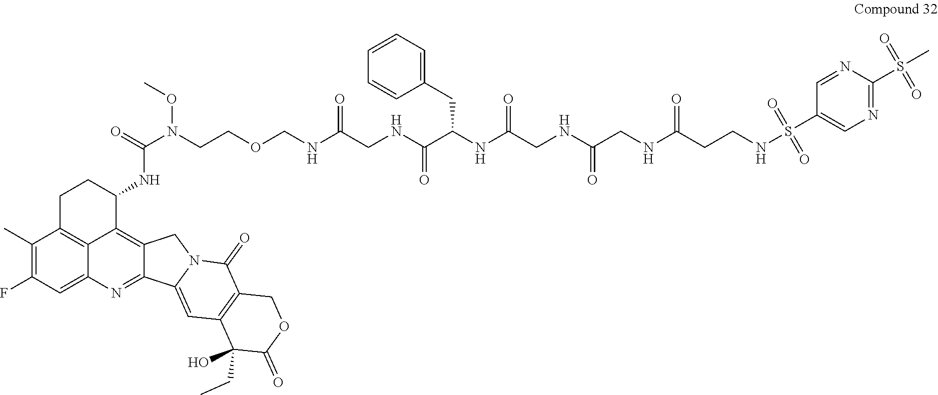 [SMILES: CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCNS(=O)(=O)c1cnc(S(C)(=O)=O)nc1)OC)CC3]

### Reaction Route:

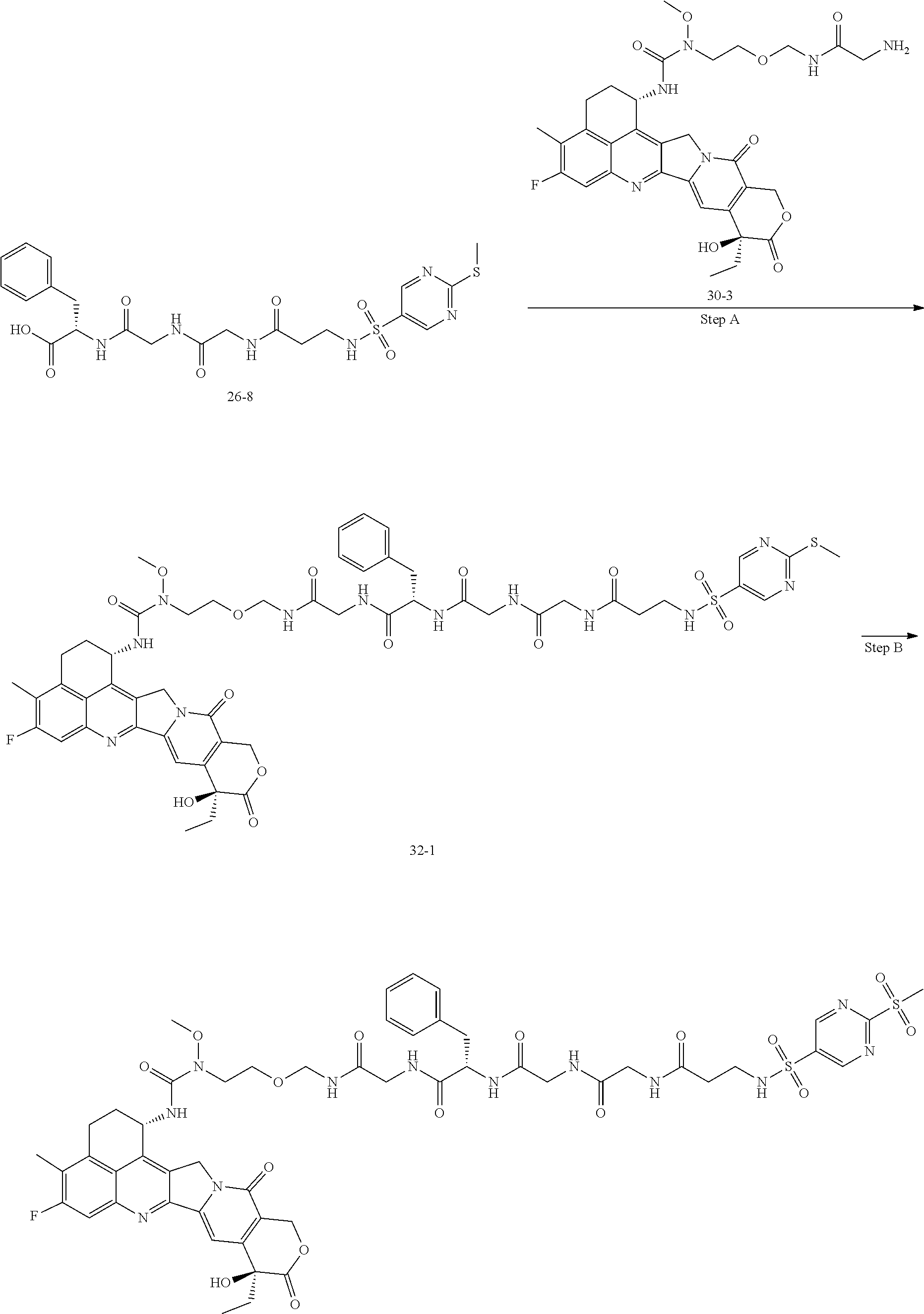 [SMILES: CSc1ncc(S(=O)(=O)NCCC(=O)NCC(=O)NCC(=O)N[C@@H](Cc2ccccc2)C(=O)O)cn1 ; CC[C@@]1(O)C(=O)OCc2c1cc1n(c2=O)Cc2c-1nc1cc(F)c(C)c3c1c2[C@@H](NC(=O)N(CCOCNC(=O)CN)OC)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCNS(=O)(=O)c1cnc(SC)nc1)OC)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCNS(=O)(=O)c1cnc(S(C)(=O)=O)nc1)OC)CC3]

### Operation Steps:

### Step A:

Compound 30-3 (30 mg, 0.047 mmol), Compound 26-8 (30.4 mg, 0.056 mmol) and HATU (26.8 mg, 0.071 mmol) were added into a 25 mL single-necked flask, a solvent: dry DMF (3 mL) was added, and then DIEA (0.031 mL, 0.19 mmol) was added, and the reaction system reacted at room temperature for 2 hours under argon protection. After the reaction was completed, the reaction solution was filtered, and the filtrate was purified by Flash to obtain compound 32-1 (20.5 mg, yield 38%).

MS (ESI) M: 1159.5 [M+H]⁺.

### Step B:

Compound 32-1 (20 mg, 0.017 mmol) and m-CPBA (17.6, 0.10 mmol) were added into a 25 mL single-necked flask, and a solvent: DCM (12 mL) was added, and the reaction system reacted at room temperature for 8 hours under argon protection. After the reaction was completed, the solvent: DCM was drained, the crude product was dissolved with DMSO (2 mL) and then purified by Flash (ACN/0.1% FA and water) to obtain 14 mg of crude product, and the crude product (dissolved with DMSO) was further purified by Prep-HPLC to obtain Compound 32 (6.40 mg, yield 31%).

MS (ESI) M: 1191.9 [M+H]⁺.

¹H NMR (400 MHz, DMSO-d6) δ8.63 (d, J=6.5 Hz, 1H), 8.43 (d, J=7.9 Hz, 2H), 8.28 (d, J=20.8 Hz, 2H), 8.17 (t, J=8.6 Hz, 1H), 8.01 (d, J=7.7 Hz, 1H), 7.93 (d, J=8.8 Hz, 1H), 7.76 (d, J=10.7 Hz, 1H), 7.32 (s, 1H), 7.22 (d, J=2.9 Hz, 4H), 7.16 (s, 1H), 6.51 (s, 1H), 5.40 (d, J=16.2 Hz, 3H), 5.25 (d, J=18.9 Hz, 1H), 5.13 (d, J=19.0 Hz, 1H), 4.61 (d, J=3.3 Hz, 2H), 4.51 (s, 1H), 3.78-3.61 (m, 12H), 3.23 (s, 3H), 3.14-2.96 (m, 4H), 2.73 (dt, J=29.8, 12.3 Hz, 2H), 2.43-2.27 (m, 5H), 2.19 (s, 2H), 2.01 (s, 2H), 1.92-1.77 (m, 2H), 0.87 (t, J=7.2 Hz, 3H).

### Example 33

### Preparation of (S)—N-(2-(((2-(1-allyl-3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) ureido) ethoxy)methyl)amino)-2-oxoethyl)-2-(2-(2-(3-((2-(methylsulfonyl)pyrimidine)-5-sulfonamido)propanamido)acetamido)acetamido)-3-phenylpropanamide (Compound 33)

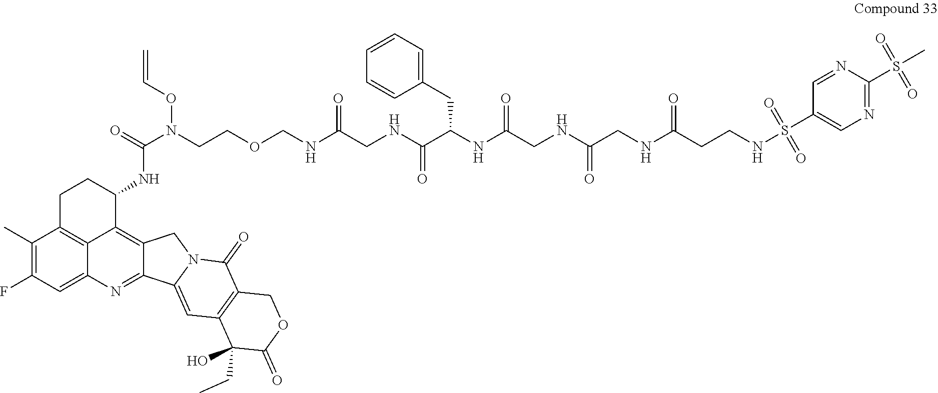

### Reaction Route:

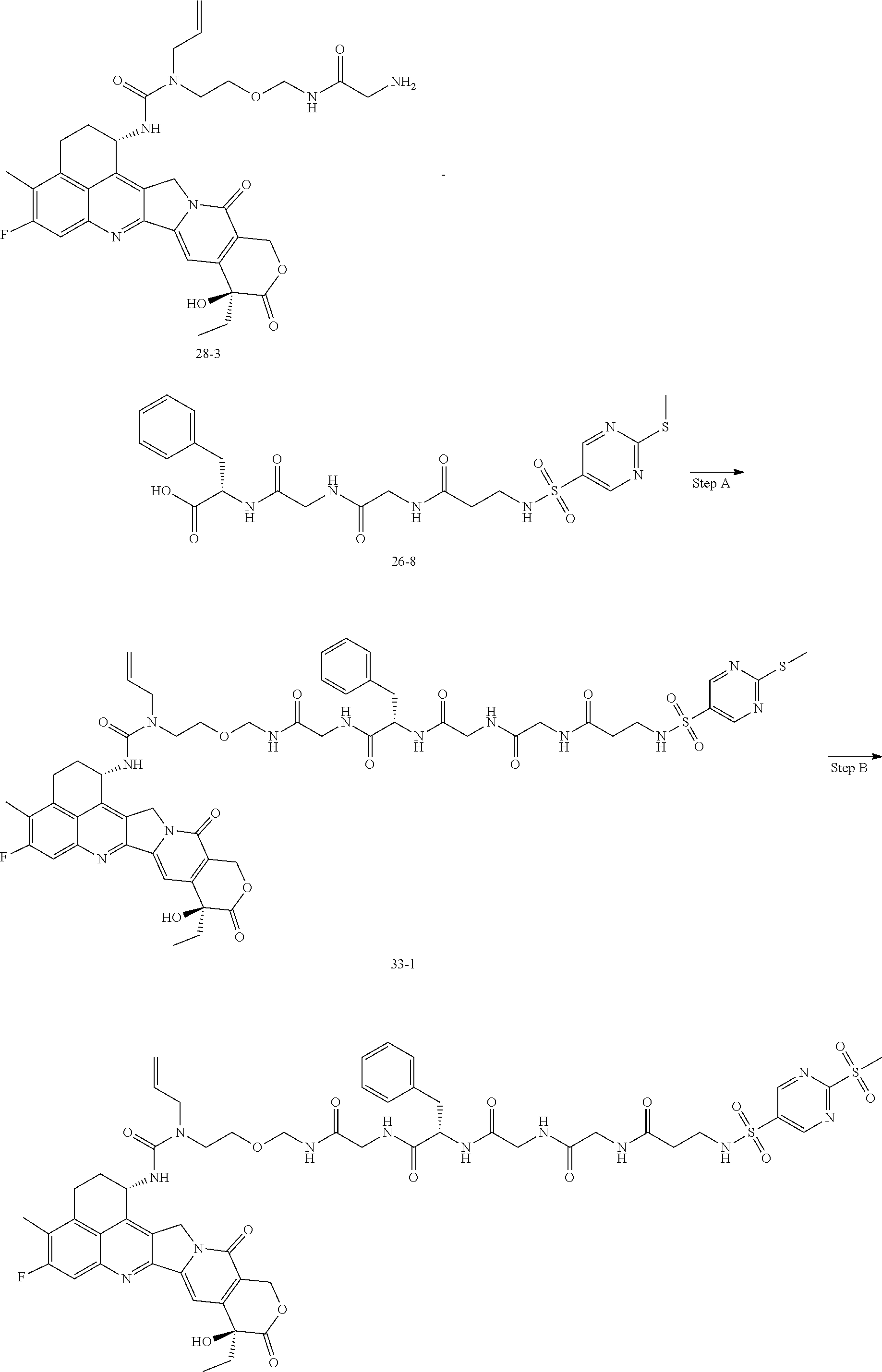 [SMILES: C=CCN(CCOCNC(=O)CN)C(=O)N[C@H]1CCc2c(C)c(F)cc3nc4c(c1c23)Cn1c-4cc2c(c1=O)COC(=O)[C@]2(O)CC ; CSc1ncc(S(=O)(=O)NCCC(=O)NCC(=O)NCC(=O)N[C@@H](Cc2ccccc2)C(=O)O)cn1 ; C=CCN(CCOCNC(=O)CNC(=O)[C@H](Cc1ccccc1)NC(=O)CNC(=O)CNC(=O)CCNS(=O)(=O)c1cnc(SC)nc1)C(=O)N[C@H]1CCc2c(C)c(F)cc3nc4c(c1c23)Cn1c-4cc2c(c1=O)COC(=O)[C@]2(O)CC ; C=CCN(CCOCNC(=O)CNC(=O)[C@H](Cc1ccccc1)NC(=O)CNC(=O)CNC(=O)CCNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

### Operation Steps:

### Step A:

Compound 28-3 (50 mg, 0.077 mmol), Compound 26-8 (45.6 mg, 0.085 mmol) and HATU (58.6 mg, 0.15 mmol) were added into a 25 mL single-necked flask, a solvent: dry DMF (3 mL) was added, and then DIEA (0.051 mL, 0.31 mmol) was added, and the reaction system reacted at room temperature for 2 hours under argon protection. After the reaction was completed, the reaction solution was filtered, and the filtrate was purified by Flash (ACN/0.1% FA and water) to obtain Compound 33-1 (35 mg, yield 38%).

MS (ESI) M: 1169.4 [M+H]⁺.

### Step B:

Compound 33-1 (30 mg, 0.026 mmol) and m-CPBA (26.9 mg, 0.16 mmol) were added into a 25 mL single-necked flask, and a solvent: DCM (12 mL) was added, and the reaction system reacted at room temperature for 8 hours under argon protection. After the reaction was completed, the solvent: DCM was drained, the crude product was dissolved with DMSO (2 mL) and then purified by Flash (ACN/0.1% FA and water) to obtain 21 mg of crude product, and the crude product was purified by Prep-HPLC (ACN/0.1% FA and water) to obtain Compound 33 (13.2 mg, yield 43%).

MS (ESI) M: 1201.5 [M+H]⁺.

¹H NMR (400 MHz, DMSO-d6) δ 8.89 (s, 2H), 8.51 (t, J=6.6 Hz, 1H), 8.29 (t, J=5.3 Hz, 1H), 8.21 (t, J=5.5 Hz, 1H), 8.09 (d, J=8.2 Hz, 1H), 8.03 (t, J=5.4 Hz, 1H), 7.95 (t, J=5.4 Hz, 1H), 7.76 (d, J=11.0 Hz, 1H), 7.31 (s, 1H), 7.27-7.11 (m, 5H), 6.81 (d, J=8.3 Hz, 1H), 5.90-5.72 (m, 1H), 5.48-5.31 (m, 3H), 5.28-5.02 (m, 4H), 4.62-4.41 (m, 3H), 4.04-3.90 (m, 2H), 3.78-3.67 (m, 6H), 3.56-3.48 (m, 3H), 3.48-3.31 (m, 3H), 3.24-3.15 (m, 1H), 3.14-2.96 (m, 4H), 2.82-2.71 (m, 1H), 2.57 (s, 3H), 2.38 (s, 3H), 2.20-2.10 (m, 2H), 2.05-1.94 (m, 1H), 1.91-1.79 (m, 2H), 0.87 (t, J=7.0 Hz, 3H).

### Example 34

### Preparation of (S)—N-(2-((2-(1-allyl-3-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) ureido) ethoxy)methyl)amino)-2-oxoethyl)-2-(2-(2-(2-((2-(methylsulfonyl)pyrimidine)-5-sulfonamido)acetamido)acetamido)acetamido)-3-phenylpropanamide (Compound 34)

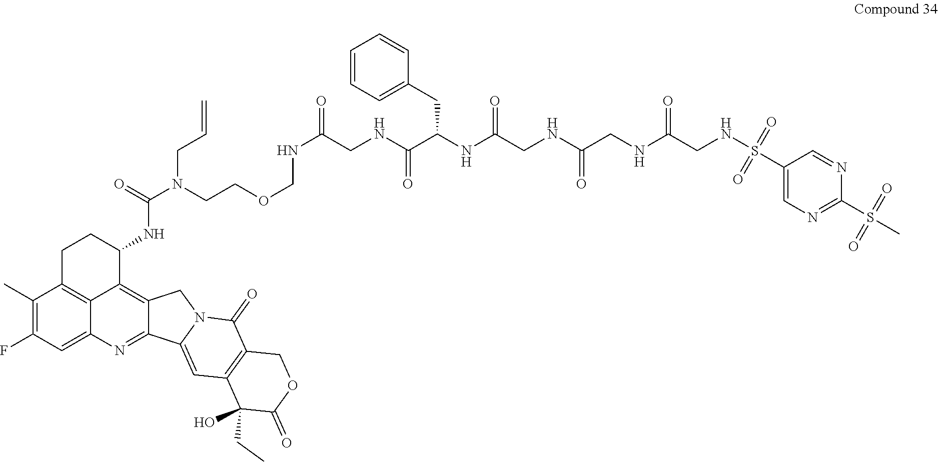 [SMILES: C=CCN(CCOCNC(=O)CNC(=O)[C@H](Cc1ccccc1)NC(=O)CNC(=O)CNC(=O)CNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

### Reaction Route:

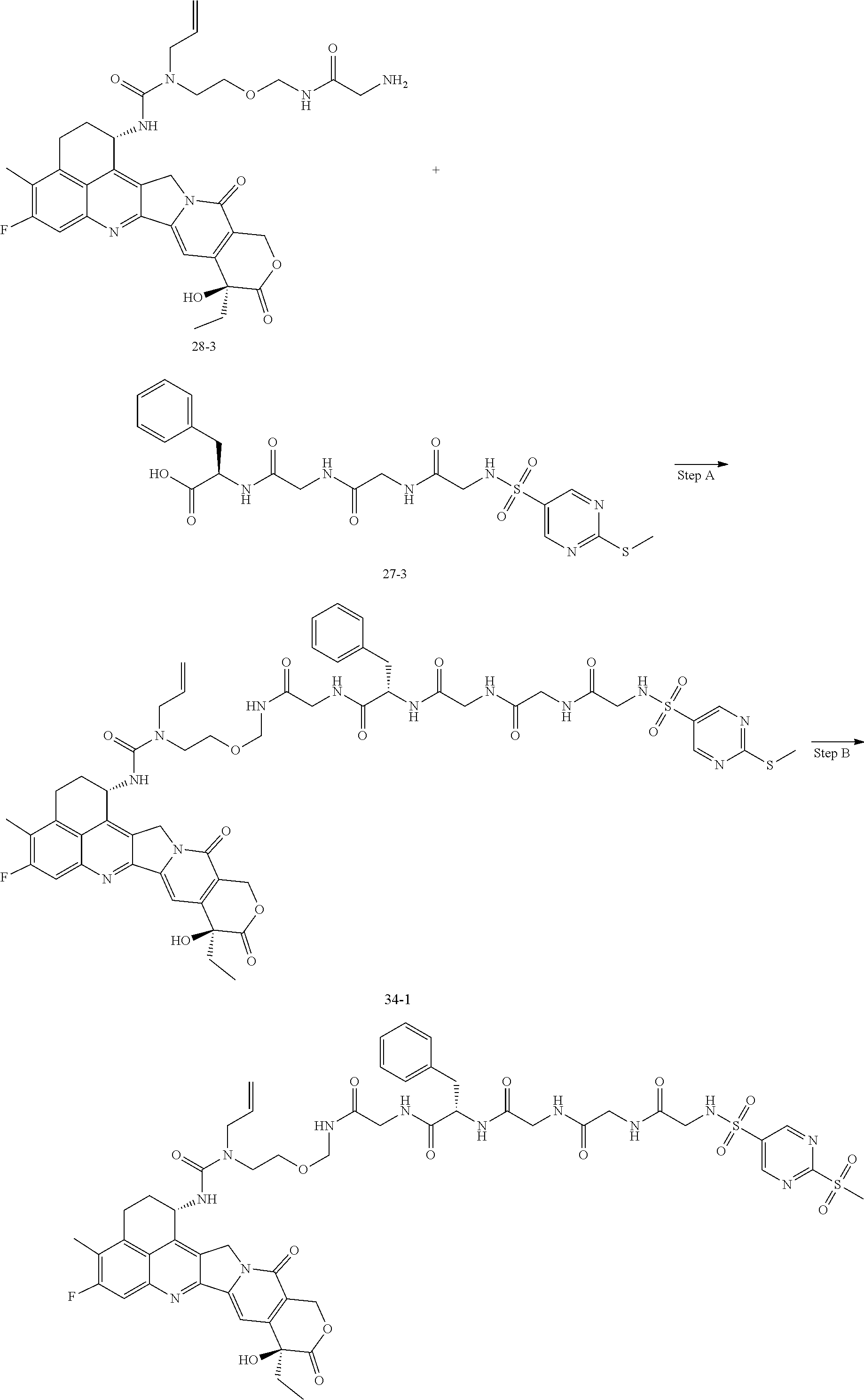 [SMILES: C=CCN(CCOCNC(=O)CN)C(=O)N[C@H]1CCc2c(C)c(F)cc3nc4c(c1c23)Cn1c-4cc2c(c1=O)COC(=O)[C@]2(O)CC ; CSc1ncc(S(=O)(=O)NCC(=O)NCC(=O)NCC(=O)N[C@H](Cc2ccccc2)C(=O)O)cn1 ; C=CCN(CCOCNC(=O)CNC(=O)[C@H](Cc1ccccc1)NC(=O)CNC(=O)CNC(=O)CNS(=O)(=O)c1cnc(SC)nc1)C(=O)N[C@H]1CCc2c(C)c(F)cc3nc4c(c1c23)Cn1c-4cc2c(c1=O)COC(=O)[C@]2(O)CC ; C=CCN(CCOCNC(=O)CNC(=O)[C@H](Cc1ccccc1)NC(=O)CNC(=O)CNC(=O)CNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

### Operation Steps:

### Step A:

Compound 28-3 (50 mg, 0.077 mmol), Compound 27-3 (44.4 mg, 0.085 mmol) and HATU (58.5 mg, 0.15 mmol) were added into a 25 mL single-necked flask, a solvent: dry DMF (3 mL) was added, and then DIEA (0.051 mL, 0.31 mmol) was added, and the reaction system reacted at room temperature for 2 hours under argon protection. After the reaction was completed, the reaction solution was filtered, and the filtrate was purified by Prep-HPLC to obtain Compound 34-1 (13 mg, yield 14%).

MS (ESI) M: 1155.4 [M+H]⁺.

### Step B:

Compound 34-1 (10 mg, 0.0087 mmol) and m-CPBA (12.0 mg, 0.070 mmol) were added into a 25 mL single-necked flask, and a solvent: DCM (12 mL) was added, and the reaction system reacted at room temperature for 3 hours under argon protection. After the reaction was completed, the solvent: DCM was drained (heating was forbidden), the crude product was dissolved with DMF (2 mL) and then purified by Flash (ACN/0.1% FA and water) to obtain 11 mg of crude product, and the crude product was purified by Prep-HPLC to obtain Compound 34 (4.6 mg, yield 5%).

MS (ESI) M: 1185.3 [M+H]⁺.

¹H NMR (400 MHz, DMSO-d6) δ 8.87 (s, 2H), 8.54 (t, J=6.3 Hz, 1H), 8.32 (t, J=5.4 Hz, 1H), 8.24 (t, J=5.1 Hz, 1H), 8.12 (d, J=8.0 Hz, 1H), 8.05 (t, J=5.3 Hz, 1H), 7.73 (d, J=10.9 Hz, 1H), 7.30 (s, 1H), 7.27-7.10 (m, 5H), 6.80 (d, J=8.0 Hz, 1H), 6.51 (s, 1H), 5.90-5.74 (m, 1H), 5.50-5.34 (m, 3H), 5.26-4.99 (m, 4H), 4.63-4.43 (m, 3H), 4.07-3.84 (m, 2H), 3.78-3.65 (m, 5H), 3.61 (dd, J=12.7, 5.5 Hz, 3H), 3.53 (dd, J=17.8, 5.1 Hz, 3H), 3.27-2.98 (m, 4H), 2.81-2.69 (m, 1H), 2.56 (s, 3H), 2.37 (s, 3H), 2.23-2.11 (m, 2H), 2.09-1.93 (m, 1H), 1.87 (dd, J=13.6, 6.8 Hz, 2H), 0.87 (t, J=7.1 Hz, 3H).

### Example 35

### Preparation of N—((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)-2,5,8,11-tetraoxo-14-oxa-3,6,9,12,17-pentaazaicos-19-en-1-yl)-6-(2-(methylsulfonyl)pyrimidin-5-yl)hex-5-ynamide (Compound 35)

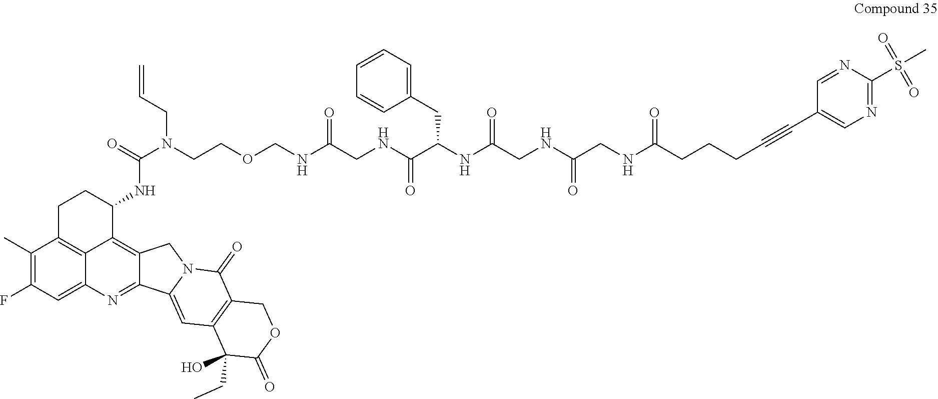 [SMILES: C=CCN(CCOCNC(=O)CNC(=O)[C@H](Cc1ccccc1)NC(=O)CNC(=O)CNC(=O)CCCC#Cc1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

### Reaction Route:

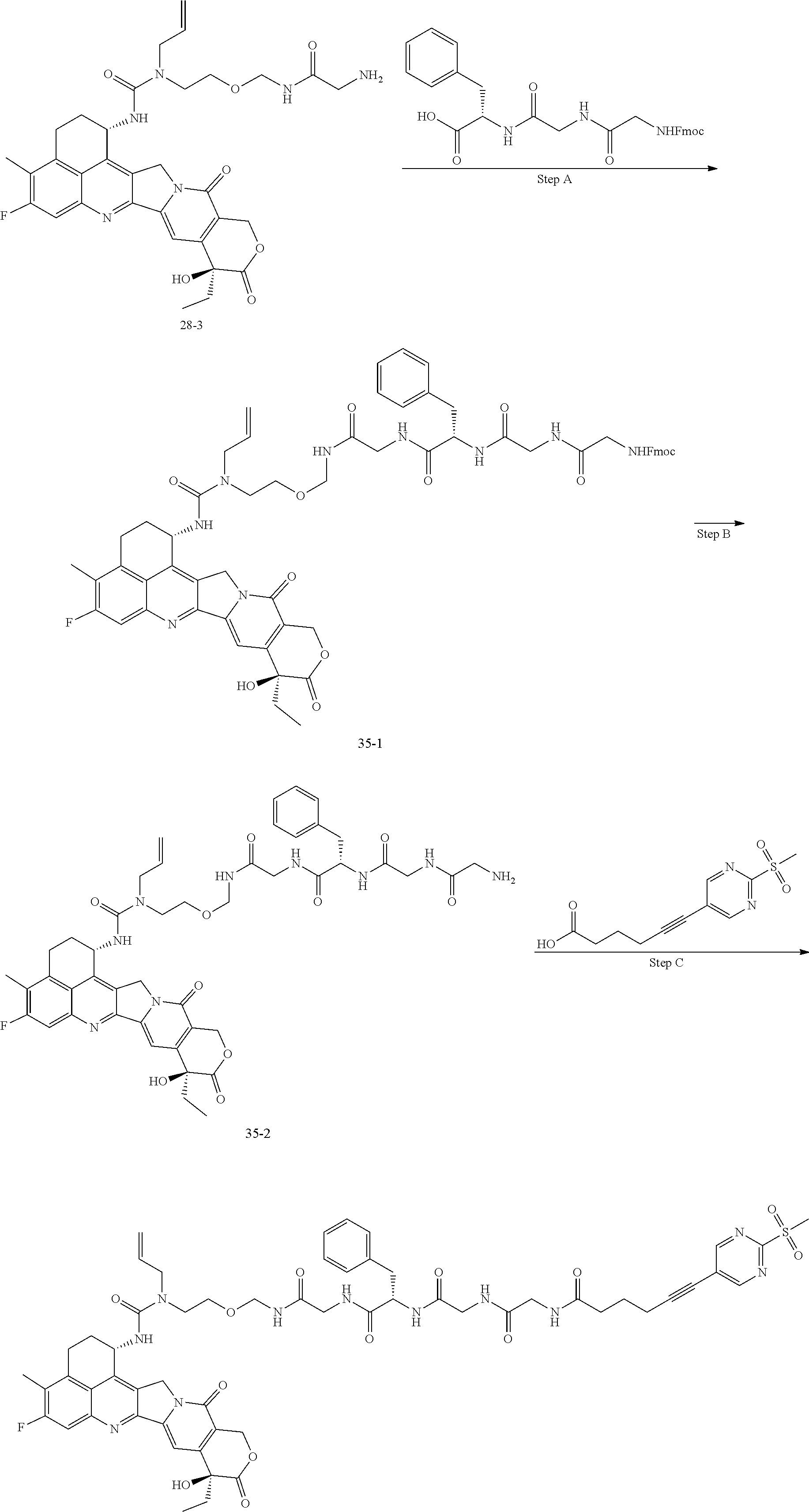

### Operation Steps:

### Step A:

Compound 28-3 (75 mg, 0.12 mmol), Fmoc-Gly-Gly-Phe-OH (78.2 mg, 0.16 mmol) and HATU (68.1 mg, 0.18 mmol) were added into a 25 mL single-necked flask, anhydrous DMF (4 mL) was added, and then DIEA (0.08 mL, 0.48 mmol) was added, and the reaction system reacted at room temperature for 2 hours under argon protection. After the reaction was completed, the reaction solution was filtered, and the filtrate was prepared and purified by Flash under a medium pressure to obtain Compound 35-1 (45 mg, yield 34%).

MS (ESI) M: 1132.5 [M+H]⁺.

### Step B:

Compound 35-1 (35 mg, 0.026 mmol) was added into a 25 mL single-necked flask, DCM (15 mL) was added, and then diethylamine (3 mL) was added, and the reaction system reacted at room temperature for 3 hours under argon protection. After the reaction was completed, the solvent: DCM was spin-dried, and the crude product was dissolved with DMF (2 mL) and then prepared and purified by Flash (ACN/0.1% FA and water) under a medium pressure to obtain Compound 35-2 (35 mg, yield 97%).

MS (ESI) M: 910.4 [M+H]⁺.

### Step C:

Compound 35-2 (35 mg, 0.039 mmol), 6-(2-(methylsulfonyl)pyrimidine-5-yl)hexan-5-ynoic acid (12.6 mg, 0.047 mmol) and PyBOP (30.4 mg, 0.058 mmol) were added into a 25 mL single-necked flask, anhydrous DMF (3 mL) was added, and then DIEA (0.026 mL, 0.16 mmol) was added, and the reaction system reacted at room temperature for 2 hours under argon protection. After the reaction was completed, the reaction solution was filtered, and the filtrate was purified by Prep-HPLC to obtain Compound 35 (2.02 mg, yield 19%).

MS (ESI) M: 1160.4 [M+H]⁺.

¹H NMR (400 MHz, DMSO-d6) δ 9.09 (s, 2H), 8.51 (t, J=6.7 Hz, 1H), 8.29 (t, J=5.7 Hz, 1H), 8.18 (t, J=5.6 Hz, 1H), 8.11 (d, J=8.0 Hz, 1H), 8.04 (t, J=5.6 Hz, 1H), 7.76 (d, J=11.0 Hz, 1H), 7.31 (s, 1H), 7.26-7.11 (m, 5H), 6.81 (d, J=8.2 Hz, 1H), 6.51 (s, 1H), 5.81 (ddd, J=15.6, 10.4, 5.2 Hz, 1H), 5.50-5.35 (m, 3H), 5.27-5.09 (m, 4H), 4.59-4.43 (m, 3H), 4.08-3.84 (m, 2H), 3.79-3.66 (m, 5H), 3.62 (dd, J=9.8, 3.8 Hz, 1H), 3.58-3.46 (m, 3H), 3.40 (s, 3H), 3.27-3.16 (m, 1H), 3.16-2.88 (m, 3H), 2.85-2.73 (m, 1H), 2.54 (s, 2H), 2.38 (s, 3H), 2.32 (t, J=7.3 Hz, 2H), 2.21-2.10 (m, 2H), 1.92-1.74 (m, 4H), 0.87 (t, J=7.2 Hz, 3H).

### Example 36

### Preparation of N—((S)-13-benzyl-3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)-9,12,15,18-tetraoxo-2,6-dioxa-3,8,11,14,17-pentaazanonadecan-19-yl)-6-(2-(methylsulfonyl)pyrimidin-5-yl)hex-5-ynamide (Compound 36)

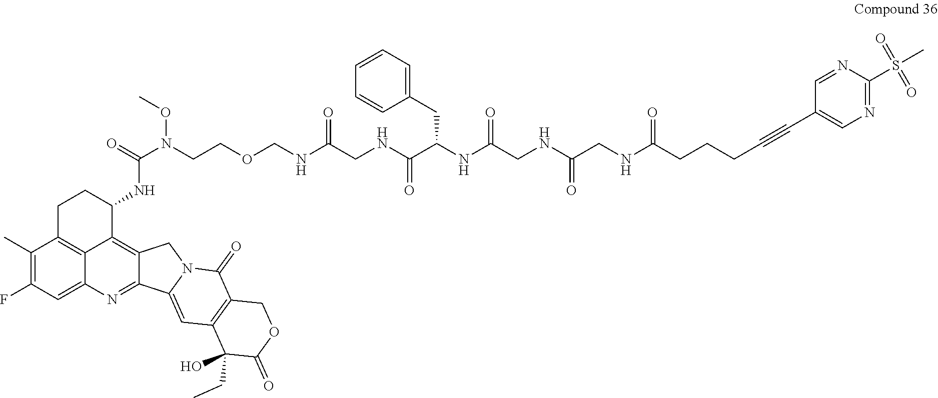 [SMILES: CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCCC#Cc1cnc(S(C)(=O)=O)nc1)OC)CC3]

### Reaction Route:

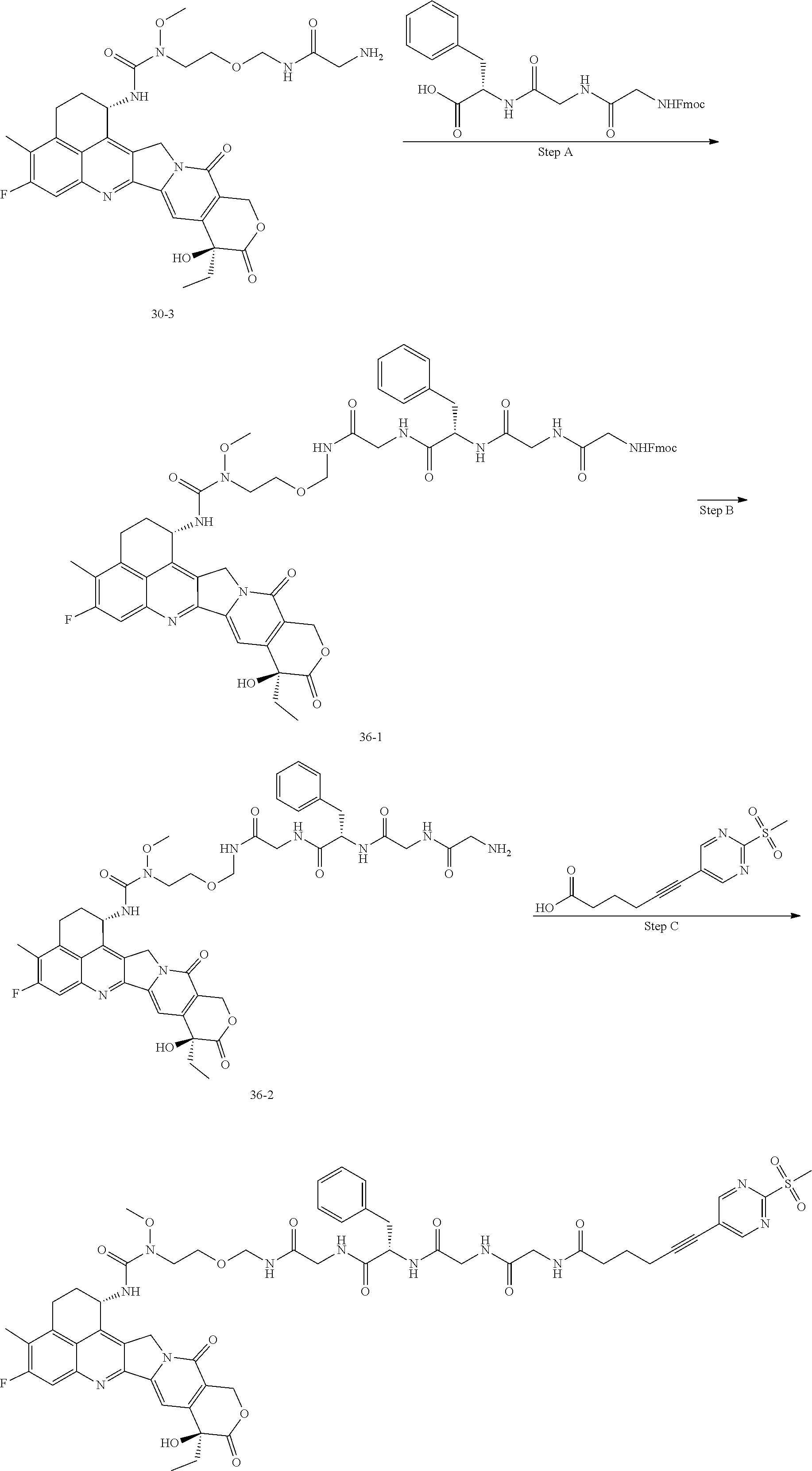

### Operation Steps:

Step A: Compound 30-3 (270.0 mg, 0.42 mmol), compound Fmoc-Gly-Gly-Phe-OH (252.8 mg, 0.50 mmol) and 2-(7-benzotriazole oxide)-N,N,N′,N′-tetramethylurea hexafluorophosphate (239.5 mg, 0.64 mmol) were added into a 25 mL single-necked flask, a solvent: dry N,N-dimethylformamide (7 mL) was added, and then N,N-diisopropylethylamine (0.28 mL, 1.68 mmol) was added, and the reaction system reacted at room temperature for 2 hours under argon protection.

After the reaction was completed, the reaction solution was filtered, and the filtrate was purified by reverse column chromatography (eluent: acetonitrile (containing 0.1% formic acid) and water) to obtain Compound 36-1 (250.0 mg, yield 52.70%).

MS (ESI) M: 1122.3 [M+H]⁺.

Step B: Compound 36-1 (250.0 mg, 0.22 mmol) was added into a 25 ml single-necked flask, a solvent: dichloromethane (40 mL) was added, and then diethylamine (8 mL) was added, and the reaction system reacted at room temperature for 3 hours under argon protection.

After the reaction was completed, the solvent: DCM was spin-dried, and the crude product was dissolved with DMF (5 mL) and then purified by reverse column chromatography (eluent: acetonitrile (containing 0.1% formic acid) and water) to obtain Compound 36-2 (130 mg, yield 64.84%).

MS (ESI) M: 900.2 [M+H]⁺.

¹H NMR (400 MHz, DMSO-d6) δ 8.59 (t, J=6.7 Hz, 1H), 8.40 (t, J=5.7 Hz, 1H), 8.29 (d, J=10.4 Hz, 2H), 8.19 (s, 1H), 7.90 (t, J=14.5 Hz, 1H), 7.75 (t, J=14.3 Hz, 1H), 7.32 (s, 1H), 7.29-7.12 (m, 5H), 5.49-5.34 (m, 3H), 5.25 (d, J=18.8 Hz, 1H), 5.12 (d, J=19.0 Hz, 1H), 4.69-4.57 (m, 2H), 4.53 (td, J=9.2, 4.5 Hz, 1H), 3.82-3.69 (m, 10H), 3.25 (d, J=22.7 Hz, 4H), 3.07 (dd, J=13.6, 4.2 Hz, 3H), 2.83 (ddd, J=23.6, 14.1, 8.5 Hz, 2H), 2.38 (s, 3H), 2.20 (d, J=5.2 Hz, 2H), 1.95-1.78 (m, 2H), 0.88 (t, J=7.3 Hz, 3H).

Step C: Compound 36-2 (25 mg, 0.028 mmol), 6-(2-(methylsulfonyl)pyrimidine-5-yl)hexan-5-ynoic acid (9.0 mg, 0.034 mmol) and 1H-benzotriazole-1-yloxytripyrrolidinyl hexafluorophosphate (21.9 mg, 0.042 mmol) c were added into a 25 mL single-necked flask, a solvent: dry N,N-dimethylformamide (1.5 mL) was added, and then N,N-diisopropylethylamine (0.019 mL, 0.11 mmol) was added, and the reaction system reacted at room temperature for 2 hours under argon protection.

After the reaction was completed, the reaction solution was filtered, and the filtrate was prepared and purified by high-performance liquid chromatography (eluent: acetonitrile (containing 0.1% formic acid) and water) to obtain the title Compound 36 (6.45 mg, yield 20.19%).

MS (ESI) M: 1150.9 [M+H]⁺.

¹H NMR (400 MHz, DMSO-d6) δ 9.09 (s, 2H), 8.56 (t, J=6.6 Hz, 1H), 8.33 (t, J=5.8 Hz, 1H), 8.19 (t, J=5.8 Hz, 1H), 8.15 (d, J=8.1 Hz, 1H), 8.07 (t, J=5.6 Hz, 1H), 7.92 (d, J=9.0 Hz, 1H), 7.77 (d, J=10.9 Hz, 1H), 7.32 (s, 1H), 7.23 (d, J=4.4 Hz, 4H), 7.16 (dd, J=8.7, 4.5 Hz, 1H), 6.52 (s, 1H), 5.48-5.34 (m, 3H), 5.25 (d, J=18.9 Hz, 1H), 5.12 (d, J=18.9 Hz, 1H), 4.61 (d, J=10.5 Hz, 2H), 4.50 (dd, J=12.7, 9.3 Hz, 1H), 3.83-3.48 (m, 13H), 3.40 (s, 3H), 3.23 (s, 1H), 3.14-3.02 (m, 2H), 2.80 (dd, J=13.7, 9.9 Hz, 1H), 2.56 (t, J=7.1 Hz, 2H), 2.38 (s, 3H), 2.32 (t, J=7.3 Hz, 2H), 2.19 (d, J=5.1 Hz, 2H), 1.92-1.77 (m, 4H), 0.87 (t, J=7.3 Hz, 3H).

An antibody sequence of Pertuzumab was as follows:

light chain:
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
heavy chain:
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG

### Example 37: ADC-1 (Control Compound)

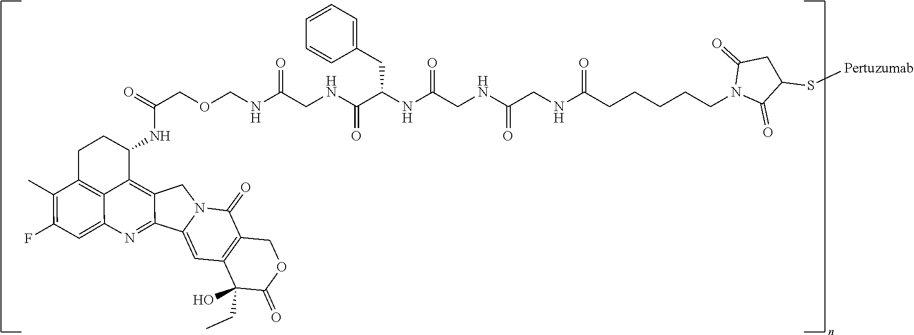

### Operation Steps:

0.33 mL of Pertuzumab antibody (anti-HER2, 30.2 mg/mL) was diluted with 0.63 mL of PBS+EDTA buffer solution (pH 7.0), a pH value was adjusted to 7.0 with a Na₂HPO₄ solution, and then 10 mM TCEP (i.e. tris(2-carboxyethyl)phosphorus, 0.04 mL, 0.40 μmol) solution was added, mixed evenly, and allowed to stand at 37° C. for 3 hours. Dimethyl sulfoxide (0.11 mL) solution of a commercially available compound deruxtecan (0.77 mg, 11 times the molar amount of the antibody substance) was added to the above solution system and the obtained system was mixed evenly to react at 15° C. for 2 hours, then 13 μL of 100 mM cysteine was added to terminate the reaction, and finally, the buffer solution was replaced with 10 mM histidine buffer solution at pH 5.5 by a G-25 gel column to obtain a coupling product ADC-1 of the compound deruxtecan and the Pertuzumab antibody. It was determined that a DAR value n was 7.83.

A structural formula of deruxtecan was as follows:

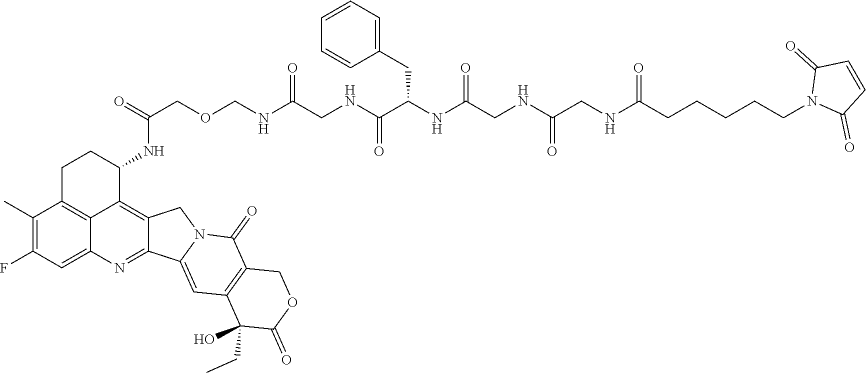 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

### Example 38: ADC-2

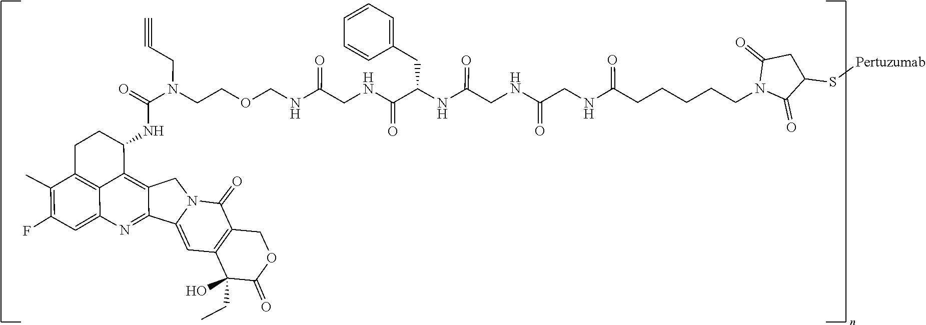

### Operation Steps:

0.33 mL of Pertuzumab antibody (anti-HER2, 30.2 mg/mL) was diluted with 0.60 mL of PBS+EDTA buffer solution (pH 7.0), a pH value was adjusted to 7.0 with a Na₂HPO₄ solution, and 10 mM TCEP (i.e. tris(2-carboxyethyl)phosphorus, 0.067 mL, 0.67 μmol) solution was added, mixed evenly, and allowed to stand at 37° C. for 3 hours. A dimethyl sulfoxide (0.25 mL) solution of Compound 25 (1.49 mg, 20 times the molar amount of the antibody substance) was added to the above solution system and the system was mixed evenly to react at 15° C. for 2 hours, then the reaction solution was added with 13 μL of 100 mM cysteine to terminate the reaction, and finally, the buffer solution was replaced with 20 mM histidine buffer solution at pH 5.5 by a G-25 gel column to obtain a coupling product ADC-2 of Compound 25 and the Pertuzumab antibody. It was determined that a DAR value n was 7.82.

### Example 39: ADC-3

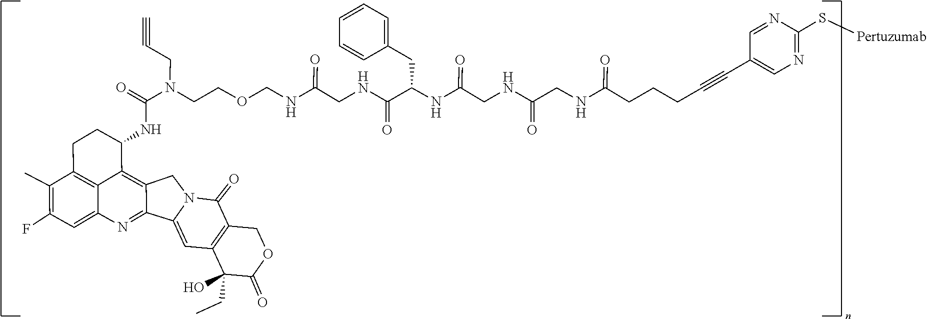

### Operation Steps:

0.33 mL of Pertuzumab antibody (anti-HER2, 30.2 mg/mL) was diluted with 0.60 mL of PBS+EDTA buffer solution (pH 7.0), a pH value was adjusted to 7.0 with a Na₂HPO₄ solution, and 10 mM TCEP (i.e. tris(2-carboxyethyl)phosphorus, 0.067 mL, 0.67 μmol) solution was added, the system was mixed evenly, and allowed to stand at 37° C. for 3 hours. A dimethyl sulfoxide (0.25 mL) solution of Compound 29 (1.57 mg, 20 times the molar amount of the antibody substance) was added to the above solution system and mixed evenly to react at 25° C. for 2 hours, then the reaction solution was added with 13 μL of 100 mM cysteine to terminate the reaction, and finally, the buffer solution was replaced with 20 mM histidine buffer solution at pH 5.5 by a G-25 gel column to obtain a coupling product ADC-3 of Compound 29 and the Pertuzumab antibody. It was determined that a DAR value n was 7.75.

### Example 40: ADC-4

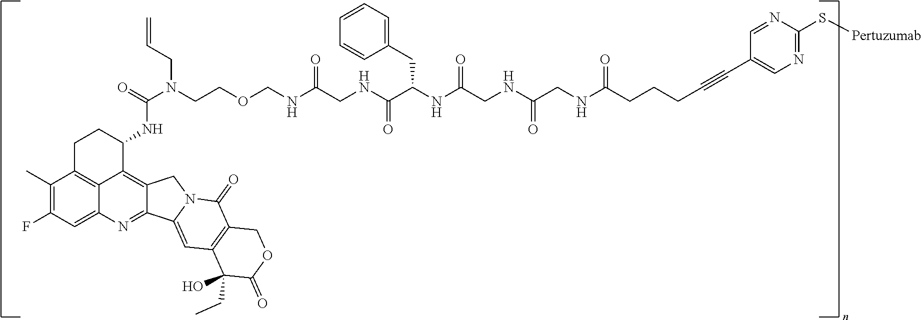

### Operation Steps:

0.33 mL of Pertuzumab antibody (anti-HER2, 30.2 mg/mL) was diluted with 0.60 mL of PBS+EDTA buffer solution (pH 7.0), a pH value was adjusted to 7.0 with a Na₂HPO₄ solution, and 10 mM TCEP (i.e. tris(2-carboxyethyl)phosphorus, 0.067 mL, 0.67 μmol) solution was added, the system was mixed evenly, and allowed to stand at 37° C. for 3 hours. A dimethyl sulfoxide (0.25 mL) solution of Compound 35 (1.57 mg, 20 times the molar amount of the antibody substance) was added to the above solution system and the system was mixed evenly to react at 15° C. for 2 hours, then the reaction solution was added with 13 μL of 100 mM cysteine to terminate the reaction, and finally, the buffer solution was replaced with 20 mM histidine buffer solution at pH 5.5 by a G-25 gel column to obtain a coupling product ADC-4 of Compound 35 and the Pertuzumab antibody. It was determined that a DAR value n was 7.72.

### Example 41: ADC-5

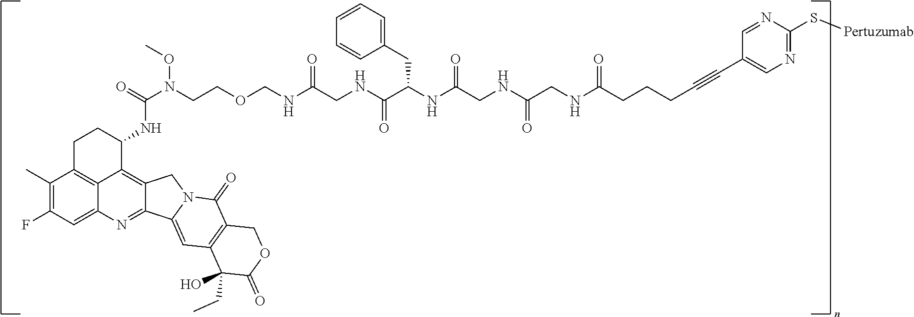

### Operation Steps:

0.33 mL of Pertuzumab antibody (anti-HER2, 30.2 mg/mL) was diluted with 0.60 mL of PBS+EDTA buffer solution (pH 7.0), a pH value was adjusted to 7.0 with a Na₂HPO₄ solution, and 10 mM TCEP (i.e. tris(2-carboxyethyl)phosphorus, 0.067 mL, 0.67 μmol) solution was added, the system was mixed evenly, and allowed to stand at 37° C. for 3 hours. A dimethyl sulfoxide (0.25 mL) solution of Compound 36 (1.55 mg, 20 times the molar amount of the antibody substance) was added to the above solution system and the system was mixed evenly to react at 15° C. for 2 hours, then the reaction solution was added with 13 μL of 100 mM cysteine to terminate the reaction, and finally, the buffer solution was replaced with 20 mM histidine buffer solution at pH 5.5 by a G-25 gel column to obtain a coupling product ADC-5 of Compound 36 and the Pertuzumab antibody. It was determined that a DAR value n was 7.37.

### Biological Test Examples

### Test Example 1: Cell Inhibitory Activity Experiment of Cytotoxic Drug

Human pancreatic adenocarcinoma cells BxPC-3 (ATCC, article number: CRL-1687) and human pharyngeal squamous cell carcinoma cells FaDu (ATCC, article number: HTB-43) were washed with PBS, then the cells were digested with 0.25% Trypsin-EDTA trypsin for about 3 minutes, and the digestion was terminated with a complete cell culture medium. The cells were centrifuged at 1000 rpm for 5 minutes, the supernatant was discarded, and the cells were resuspended with a complete cell culture medium, counted by a cell counter, and adjusted to a required density. Each well was added with 50 μL of the cell suspension according to 3000 cells per well, and the cell plate was placed into a 5% CO₂ cell incubator at 37° C. to incubate the cells for 12-16 hours.

The cell plate was taken out of the incubator to observe a cell adhesion state, and after cell adhesion, each well was added with 50 μL of the sample (starting from 10 μM, diluted 5 times, 9 concentrations), shaken gently, and then placed into an incubator to incubate the cells for 72 hours. Each well was added with a Cell Titer-GLO™ (Promega, article number: G7572) working solution according to 100 μL/well and shaken gently to lyse the cells, and the plate was read on a microplate reader.

$$A calculation formula of a cell - proliferation inhibition rate was : \\ cell - proliferation inhibition rate = ( 1 - sample well / control well ) \times 100 \% .$$

Data analysis: a graph was made by GraphPad Prism 7.0 software with a Log value of a sample concentration as an abscissa and Cytotoxicity % as an ordinate, and data were subjected to Nonlinear regression (curve fit) analysis to obtain an IC₅₀ value of each compound. IC₅₀ values of inhibitory activities of the compounds in examples on the BxPC-3 cells and the FaDu cells are shown in Table 1.

TABLE 1
Test results of inhibitory effects on BxPC-3 cells and FaDu cells

| Serial number of example | BxPC-3/IC₅₀ (nM) | Fadu/IC₅₀ (nM) |
|---|---|---|
| Example 1 | 8.27 | 6.68 |
| Example 2 | 0.96 | 1.49 |
| Example 3 | 0.76 | 1.09 |
| Example 4 | 1.84 | 2.95 |
| Example 5 | <0.1 | 0.75 |
| Example 7 | 0.76 | <0.1 |
| Example 8 | 1.45 | 0.13 |
| Example 9 | 2.15 | 2.34 |
| Example 10 | 5.1 | 6.04 |
| Example 12 | 5.77 | 42.02 |
| Example 13 | 2.49 | 36.04 |
| Example 14 | 6.27 | 57.82 |
| Example 15 | 20.54 | 11.45 |
| Example 16 | 7.93 | 4.33 |
| Example 17 | 4.04 | 2.26 |
| Example 18 | 40.76 | 16.91 |
| Example 19 | 3.69 | 3.97 |
| Example 20 | 3.05 | 2.00 |
| Example 21 | 29.23 | 19.78 |

Conclusion: the above compounds have a good cell-proliferation inhibitory effect.

### Test Example 2: Inhibitory Activity Experiment of Cytotoxic Drug on NCI-N87, KPL-4 or MDA-MB-468

The experiment was intended to detect in-vitro inhibitory activities of the compounds of the present disclosure on NCI-N87 (human gastric cancer cells), KPL-4 (human breast cancer cells) or MDA-MB-468 (human breast cancer cells).

### Experimental Instruments:

96-well transparent flat-bottom white plate (Corning, #3610)
RPMI1640 culture medium (Gibco, #A10491-01)
Fetal Bovine Serum (Sigma-Aldrich, #F8687-500ML)
CellTiter-Glo® Luminescent Cell Viability Assay (Promega, #G7572/G7571)

### Experimental Method:

Human gastric cancer cells NCI-N87 (ATCC, article number: CRL-5822), human breast cancer cells MDA-MB-468 (Cell Bank of the Committee on Type Culture Collection, Chinese Academy of Sciences, article number: TCHu136) and human breast cancer cells KPL-4 (Nanjing Cobioer Biosciences Co., Ltd., article number: CBP60379) were washed with PBS, then the cells were digested with 0.25% Trypsin-EDTA trypsin for about 3-10 minutes, and the digestion was terminated with a complete cell culture medium. The cells were centrifuged at 1000 rpm for 5 minutes, the supernatant was discarded, and the cells were resuspended with a complete cell culture medium, counted by a cell counter, and adjusted to a required density. Each well was added with 90.0 μL of the cell suspension according to 3000 cells per well, and the cell plate was placed into a 5% CO₂ cell incubator at 37° C. to incubate the cells overnight.

In the experiment, a DMSO control group, a positive control group and a test sample group were provided respectively, and the compound Dxd was used as a positive control compound. The cell plate was taken out of the incubator to observe a cell adhesion state, and after cell adhesion, each well was added with 10.00 μL of the sample (starting from a final concentration 10 μM, diluted 5 times, 9 concentrations), shaken gently, and then placed into an incubator to incubate the cells.

After incubation for 3 days, each well was added with a CellTiter-Glo™ (Promega, article number: G7572/G7571) working solution according to 100.0 μL/well and oscillated by a constant-temperature oscillator to lyse the cells, and 10 minutes later, the plate was read on a microplate reader.

A calculation formula of a cell-proliferation inhibition rate was: cell-proliferation inhibition rate=(1−RLU_{test sample well}/RLU_{DMSO control well})×100%.

Data analysis: a graph was made by GraphPad Prism 8.0 software with a Log value of a sample concentration as an abscissa and Inhibition % as an ordinate, and data were subjected to Nonlinear regression (curve fit) analysis to obtain an IC₅₀ value of each test sample. Activity data are shown in Table 2.

A structural formula of Dxd of the control group was as follows:

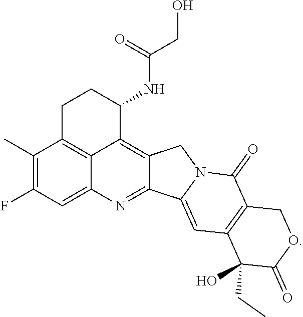 [SMILES: CC[C@@]1(O)C(=O)OCc2c1cc1n(c2=O)Cc2c-1nc1cc(F)c(C)c3c1c2[C@@H](NC(=O)CO)CC3]

TABLE 2
Cell-proliferation inhibitory activities
of compounds of the present disclosure

| Compound | KPL- | NCI- | MDA-MB- |
|---|---|---|---|
| of example | 4/IC₅₀ (nM) | N87/IC₅₀ (nM) | 468/IC₅₀ (nM) |
| Dxd | 32.29 | 22.59 | 2.71 |
| Example 2 | 29.65 | 10.98 | 2.41 |
| Example 3 | 8.82 | 6.39 | 0.99 |

Conclusion: it can be seen from Table 2 that, compared with Dxd, the compounds of the present disclosure have a better cell-proliferation inhibitory activity.

### Test Example 3: Metabolism Stability Test in Liver Microsomes

The experiment was intended to detect metabolic stabilities of the compounds in the present disclosure in human liver microsomes.

### Experimental Materials:

Human Liver Microsomes (Corning, Cat No. 452117)

Experimental method: each incubation system contained a phosphate buffer solution (PBS, pH 7.4), a liver microsomal protein, a to-be-tested sample (acetonitrile solution) and NADPH, incubated in water bath at 37° C., and the same volume of cold acetonitrile (comprising an internal standard) was added 0, 5, 15, 30, 45 and 60 minutes after reaction respectively to terminate the reaction. The sample solution was centrifuged, 80 μL of the supernatant was taken and 240 μL of ultrapure water was added, and the remaining amount of the prototype substrate was detected by LC-MS/MS method. The negative control was incubated with a human liver microsome NCF group (without NADPH). Parameters of liver microsome metabolism calculated by first-order kinetic equation are shown in the following table.

TABLE 3
Stabilities of compounds in the present
disclosure in human liver microsomes

Human liver microsomes

| Compound of | T_{1/2} | CL_{int(mic)} | CLᵢₙₜ₍ₗᵢᵥₑᵣ₎ |
|---|---|---|---|
| example | (min) | (μL/min/mg) | (mL/min/kg) |
| Example 2 | 62.4 | 22.2 | 20.0 |
| Example 3 | 52.6 | 26.4 | 23.7 |
| Dxd | 101.6 | 13.6 | 12.3 |

Conclusion: compared with the Dxd of the control group, the compounds in Examples 2 and 3 of the present disclosure have a faster metabolic speed and a shorter metabolic half-life period in stability in the human liver microsomes, which can reduce the risk of toxic and side effects caused by payload shedding of corresponding ADCs, so that the compounds of the present disclosure have better metabolic properties as ADC payloads.

### Test Example 4: hERG Experiment

Inhibitory effects of the compound of Example 2, the compound of Example 3 and Dxd on an hERG potassium channel were studied by a manual patch clamp technique (a gold standard of hERG safety evaluation), and the risk of ventricular repolarization toxicity caused was evaluated.

### 1. Experimental Program

### 1.1 Test Samples and Preparation Method:

The control compound Dxd and some compounds of the present disclosure.

The weighed test sample was prepared into a 10 mM stock solution with DMSO, and then the stock solution of the test object was diluted into 3.33 mM intermediate dilution with DMSO. Then, 30 μL of the dilution was added into 10 mL of extracellular fluid to prepare 10 μM working solution, it was ensured that a DMSO content was 0.3%, then 50 μL of F68 was added to the extracellular fluid to enhance a solubility of the test object, and an F68 concentration was 0.5%. Solutions at all concentrations were subjected to ultrasound treatment for 20 minutes. The solubility of the sample was visually observed, and there was no visible precipitation in the 10 μM to-be-tested solution of the test object.

### 1.2 Experimental Cells:

An HEK-293 cell line stably expressing the hERG potassium channel was adopted, and hERG potassium channel cells were purchased from Creacell Company (article number: A-0320). Cell culture method:

The HEK-293 cell line stably expressing the hERG potassium channel was cultured in a DMEM culture medium containing 10% fetal bovine serum and 0.8 mg/mL G418 at a culture temperature of 37° C. under a carbon dioxide concentration of 5%.

Cell passage: the old culture medium was removed, and the cells were washed once with PBS, then 1 mL of TrypLE™ Express solution was added, and the mixture was incubated at 37° C. for about 0.5 minutes. When the cells were separated from a dish bottom, about 5 mL of complete culture medium preheated at 37° C. was added. A cell suspension was gently blown with a straw to separate the aggregated cells. The cell suspension was transferred to a sterile centrifuge tube and centrifuged at 1000 rpm for 5 minutes to collect the cells. In amplification or maintenance of culture, the cells were inoculated into a 6 cm cell culture dish, and a number of cells inoculated into each cell culture dish was 2.5×10⁵ cells (final volume: 5 mL). In order to maintain an electrophysiological activity of cells, a cell density should not be greater than 80%.

Before patch clamp detection, the cells were separated by TrypLE™ Express, and 4×10³ cells were spread on a cover slip and cultured in a 24-well plate (final volume: 500 μL). Experimental detection was carried out 18 hours later.

### 1.3 Experimental Results:

An inhibitory effect of the test object on the hERG channel was detected by the manual patch clamp technique. The experimental results are summarized as follows.

TABLE 4
Inhibition rate results of compounds in the
present disclosure on hERG channel current

| Compound of | Concentration | hERG current inhibition rate % |
|---|---|---|
| example | (μM) | (Mean ± SE, n = 2) |
| Dxd | 10 | 10.32% ± 1.97% |
| Example 2 | 10 | 2.89% ± 1.86% |
| Example 3 | 10 | 15.77% ± 0.43% |

Conclusion: at the same concentration, compared with the Dxd of the control group, the inhibition rates of the compounds in the present disclosure on the hERG channel current are significantly lower than that of the Dxd of the control group, which suggests that the risk of cardiac QT prolongation is lower and the compounds in the present disclosure are safer.

### Test Example 5: In-Vivo Pharmacokinetics Experiment of Compounds of the Present Disclosure in SD Rats

SD rats were taken as test animals to study in-vivo pharmacokinetics behaviors after injection administration of the compounds of the present disclosure to male and female SD rats at dosages of 1 mg/kg and 3 mg/kg.

### 1. Experimental Scheme

### 1.1 Test Samples:

The control compound Dxd and some compounds of the present disclosure.

### 1.2 Experimental Animals

### Basic Information of Animals

Species: male and female SD rats (2 female rats and 2 male rats/group)
Grade: SPF grade
Weight: about 200 g
Source: Hunan SJA Laboratory Animal Co., Ltd.

### 1.3 Administration

Male and female SD rats were injected with the test sample solution, and Compound 2 of the example and the Dxd were given in a dosage of 1 mg/kg and a dosage volume of 4 mL/kg. Compound 3 of the example and the Dxd were given in a dosage of 3 mg/kg and a dosage volume of 5 mL/kg.

Preparation of test sample solution: the test sample was dissolved with a solvent (10% DMSO-60% PEG400-30% (20% HP-β-CD-water)), and DMSO, PEG 400 and 20% HP-β-CD-water were added sequentially during preparation.

Control group: the structural formula of Dxd was the same as above.

Experimental group: some compounds of the present disclosure.

### 1.5 Sample Collection:

0 before administration, and 5 minutes, 15 minutes, 0.5 hours, 1 hour, 2 hours, 3 hours, 5 hours and 8 hours after administration.

0.2 mL of blood was collected and placed into an EDTA-2K anticoagulant tube. The anticoagulant tube was turned upside down gently to fully mix the anticoagulant (EDTA-2K) with the blood, and then immediately placed into wet ice, and plasma was centrifuged for separation within 1 hour after blood collection at 4° C. and 6800 g for 6 minutes. After centrifugation, the plasma was separated and stored in an ultra-low-temperature refrigerator as soon as possible until sample analysis.

### 2. Experiment Results and Analysis

Pharmacokinetics parameters are shown in Table 5 below. In-vivo pharmacokinetics results of the present disclosure in the rats shows that, compared with the Dxd of the control group, the compounds of the present disclosure had a faster in-vivo clearance speed, a shorter half-life period and a lower in-vivo exposure, wherein, Compound 2 of the example was obviously superior to the Dxd in pharmacokinetics properties as an ADC payload.

TABLE 5
Pharmacokinetics parameters after intravenous
administration to SD rats

| Compound of | Dose | CL | Vz | | AUC_{0-8 h} |
|---|---|---|---|---|---|
| example | (mg/kg) | (mL/min/kg) | (mL/kg) | T_{1/2} (hr) | (hr*ng/mL) |
| Dxd | 1 | 4150 | 7960 | 1.30 | 240 |
| Example 2 | 1 | 12700 | 29000 | 1.59 | 77.9 |
| Dxd | 3 | 3940 | 10900 | 1.96 | 760 |
| Example 3 | 3 | 7460 | 18700 | 1.73 | 405 |

### Test Example 6: Toxicity Comparison Experiment after Repeated Administration to SD Rats for 7 Days

SD rats were taken as test animals to study toxic reactions after intravenous injection of the compounds of the present disclosure to male and female SD rats at a dosage of 0.1 mg/kg for 7 consecutive days.

### 1. Experimental Program

### 1.1 Test Samples:

The control compound Dxd and some compounds of the present disclosure.

### 1.2 Experimental Animals

### Basic Information of Animals

Species: male and female SD rats (5 female rats and 5 male rats/group)
Grade: SPF grade
Weight: ♂ 252.0-287.4 g and ♀ 185.7-224.3 g
Age: 8-12 weeks
Source: Zhejiang Vital River Laboratory Animal Technology Co., Ltd.

### 1.3 Administration

Male and female SD rats were injected with the test sample solution, and Compound 2 of the example and the Dxd were given in a dosage of 0.1 mg/kg and a dosage volume of 10 mL/kg.

Preparation of test sample solution: the test sample was dissolved with a solvent (5% DMSO+95% Solutol HS-15 in water).

Control group: the structural formula of Dxd was the same as above.

Experimental group: some compounds of the present disclosure.

### 1.4 Experimental Indexes:

Observational index

| | |
|---|---|
| Clinical pathology | Hematology: blood collection on 3^{rd}/8^{th} day |
| | Blood biochemistry: blood collection on 8^{th} day |
| Organ coefficient | Brain, thymus, heart, liver, spleen, kidney and adrenal gland |
| Histopathological | Main organs of animals |
| examination | |

### 1.5 Sample Collection and Analysis:

About 0.2 mL of blood was collected from the jugular sinus of the animals into an anticoagulant tube containing an anticoagulant (EDTA-K2) on D3 after administration, and all to-be-dissected animals were fasted for about 12 hours on the eve of D8 before dissection. Dying animals were euthanized, about 2 mL of blood was collected from abdominal aorta into an anticoagulant tube containing an anticoagulant (EDTA-K2) at the time of dissection, and hematological indexes were detected by a Sysmex XN-1000 hemocytometer. Specific detection indexes were as follows.

| | |
|---|---|
| White blood cell count (WBC) | Differential blood count and |
| Platelet count (PLT) | percentage (N, L, M, E, B) |
| Mean platelet | Red blood cell distribution |
| volume (MPV) | width (RDW) |
| Hemoglobin (HGB) | Red blood cell count (RBC) |
| Mean corpuscular volume (MCV) | Hematocrit (HCT) |
| Mean corpusular | Mean corpusular hemoglobin (MCH) |
| hemoglobin | Platelet volume |
| concentration (MCHC) | distribution width (PDW) |

### 2. Experimental Results

Hematological parameters of rats at the end of the experiment were as shown in Table 6 below. The experimental results showed that a change degree of blood toxicity in the rats administered with the compound of Example 2 in the present disclosure at a dosage of 0.1 mg/kg for 7 consecutive days was lower than that in the rats administered with the Dxd. These examination indexes with a low change degree comprised: a white blood cell count (WBC), proportions of various white blood cells comprising neutrophils (Neut), monocytes (Mono), eosinophils (Eos) and basophils (Baso), a red blood cell count (RBC), a hemoglobin concentration (HGB), a hematocrit value (HCT), a mean corpuscular count (MCV), a mean corpuscular hemoglobin concentration (MCHC), a reticulocyte proportion (Retic), and the like.

TABLE 6
Hematological parameters at the end
of toxicological study in SD rats

| | Dxd group | | Compound 2 | |
|---|---|---|---|---|
| Index | ♂ | ♀ | ♂ | ♀ |
| WBC | / | −47.6%* | / | / |
| RBC | −15.3%** | −13.4%** | / | −10.9%** |
| HGB | −12.8%** | −12.7%** | / | −10.7%** |
| HCT | −15.7%** | −15.8%** | / | −12.0%** |
| MCHC | 3.2%** | 3.6%** | / | / |
| #NEUT | −66.9%** | −66.1%* | −72.2%** | / |
| #MONO | −77.2%** | −71.3%** | −68.3%** | −54.1%** |
| #EOS | −70.2%* | −84.44%** | −71.4%** | / |
| #BASO | −82.1%* | / | / | / |
| #LUC | / | −66.9%** | / | / |
| #RETIC | −95.5%* | −96.9%** | −87.3%** | −86.4%** |

Note:
a positive value indicates an increase compared with the control group, a negative value indicates a decrease compared with the control group,
“*” indicates P < 0.05, and
“**” indicates P < 0.01.

Conclusion: compared with the Dxd group, according to an overall trend, there are fewer abnormal items in blood indexes of male and female groups in the rat toxicology experiment of Compound 2, and the safety is higher.

### Test Example 7: In-Vitro Plasma Stabilities of ADCs

The experiment was intended to detect a stability of an HER2-ADC drug of the present disclosure in plasma across various species in vitro.

Experimental method: an ADC sample, healthy human plasma, cynomolgus monkey plasma (Nanjing SenBeiJia Biological Technology Co., Ltd.) and 1% BSA (made in the laboratory with BSA powder, which was purchased from Shanghai Sangon Biotech Co., Ltd.) were respectively filtered by a 0.22 μm filter under a sterile environment. A drug stock solution was diluted to 100 μg/mL with plasma of different species and 1% BSA, and incubated in a cell incubator at 37° C., the day of incubation was marked as a day 0, and then the sample was taken out on the 21^{st} day for free toxin detection and DAR value detection.

Detection method of free toxin: 300 μL of acetonitrile solution containing an internal standard (internal standard: 0.1 ng/mL Aprepitant) was added to the prepared biological sample or the biological sample (50 μL) with unknown concentration, the mixture was vortexed evenly for 5 minutes, and centrifuged at 4000 rpm and a low temperature (4° C.) for 10 minutes, and 100 μL of supernatant was added into 100 μL of aqueous solution containing 0.2% formic acid, the mixture was vortexed for 3 minutes, and then transferred to a 96-well sample plate for LC-MS/MS analysis (Shimadzu Exion LC-AB Triple Quad®5500+). Free toxin release rates of Her2-ADC compounds of some examples of the present disclosure in plasma are shown in Table 7-1.

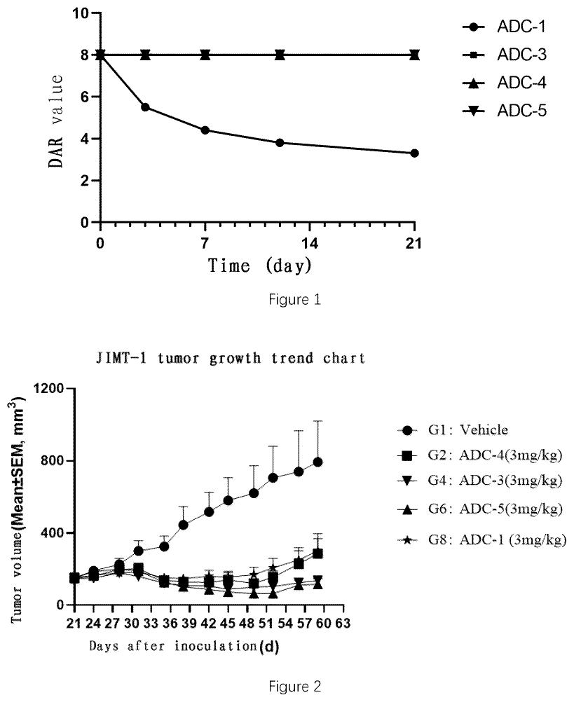

Detection method of DAR value: 30 μL of 1% BSA sample was captured by protein A beads, the cynomolgus monkey plasma sample was captured by mouse anti human IgG1 beads and the human plasma sample was captured by human her2 protein beads, PBST was added to the samples respectively, then the mixture was incubated under shaking at 1000 rpm for 1 hour, washed with 200 μL of PBST, PBS, ultrapure water and 10% acetonitrile sequentially, and then eluted with 100 μL of 20% acetonitrile (containing 0.5% formic acid) for 5 minutes, and all supernatants were taken, 20 μL of 100 mM TCEP was added, the mixture was shaken at room temperature at 1000 rpm to react for 1 hour, and then subjected to sample analysis (QTOF-MS). A change of DAR is shown in FIG. 1.

TABLE 7
Experimental results of in-vitro plasma stabilities of ADCs

| | Free toxin release rate (%) of ADC sample on the 21^{st}day | | |
|---|---|---|---|
| Name of | Human | Monkey | PBS + 1% |
| sample | plasma | plasma | BSA |
| ADC-1 | 1.38% | 1.35% | 0.29% |
| ADC-4 | 0.19% | 0.17% | 0.17% |
| ADC-5 | 0.21% | 0.32% | 0.30% |
| ADC-3 | 0.12% | 0.26% | 0.17% |

Conclusion: it can be seen from Table 7 that free toxin shedding rates of the ADCs in the examples of the present disclosure incubated in the human plasma and the monkey plasma for 21 days respectively are all lower than 2%, and free toxin release rates of the ADCs are much lower than those of the control ADC-1; and in-vitro plasma stabilities of the ADC-3, the ADC-4 and the ADC-5 of the present disclosure are better than that of the positive control ADC-1.

It can be seen from FIG. 1 that, compared with the ADC-1, the ADCs in the examples of the present disclosure are very stable after being incubated in the human plasma for 21 days, and it is detected that DAR values of the ADC-3, the ADC-4 and the ADC-5 are not decreased after the ADCs are incubated in the human plasma for a total of 21 days. However, a DAR value of the ADC-1 is decreased by nearly 59% after being incubated in the human plasma for 21 days (FIG. 1), which indicates that some linkers-payloads will fall off from an antibody during incubation. The stability of the linkers indicates that more payloads are delivered to a targeted tumor, while the release of off-target free payloads is reduced, thus improving a therapeutic index.

### Test Example 8: In-Vivo Efficacy Evaluation of Test Object in JIMT-1 Human Breast Cancer Model

BALB/c nude mice were taken as experimental animals to evaluate in-vivo efficacies of the ADCs in the examples of the present disclosure on a JIMT-1 transplanted tumor.

Female BALB/c nude mice, purchased from Beijing Vital River Laboratory Animal Technology Co., Ltd., were adaptively fed in an SPF animal house for 7 days for follow-up research. JIMT-1 cells were inoculated subcutaneously in a right axilla part of the nude mice according to 5×10⁶ cells/0.1 mL. When a mean tumor volume reached about 150 mm³, 54 mice with a moderate tumor volume were selected and divided into 9 groups, with 6 mice in each group: G1: Vehicle, G2: ADC-4 (3 mg/kg), G3: ADC-4 (10 mg/kg), G4: ADC-3 (3 mg/kg), G5: ADC-3 (10 mg/kg), G6: ADC-5 (3 mg/kg), G7: ADC-5 (10 mg/kg), G8: ADC-1 (3 mg/kg) and G9: ADC-1 (10 mg/kg). The mice started to be intravenously administered on the day of grouping, once a week, twice in total, wherein the Vehicle was given 0.9% sodium chloride injection. Tumor size and weight were measured twice a week. Tumor long diameter and short diameter were measured by a vernier caliper, tumor volume (mm³)=0.5×long diameter×short diameter.

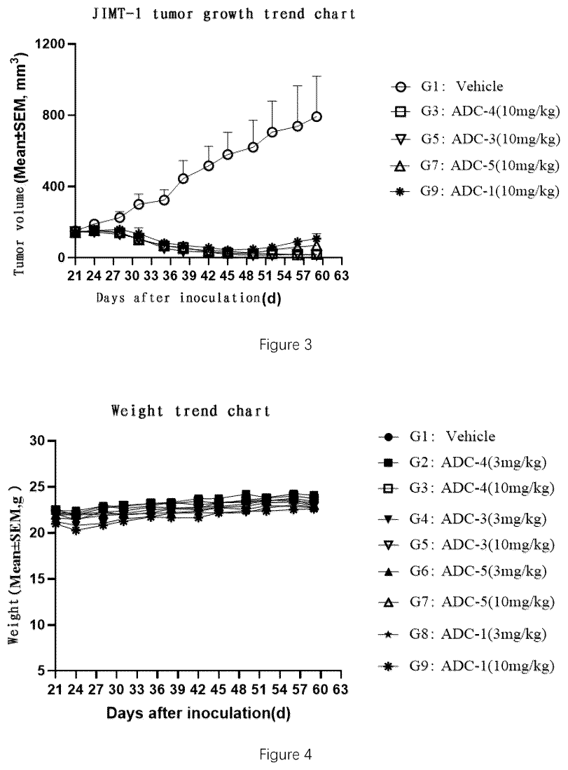

Experimental results are shown in Table 8 and FIG. 2-FIG. 4. Compared with the Vehicle (solvent control), various test compounds could all significantly inhibit JIMT-1 tumor growth. In the low-dose group, compared with the ADC-1 (3 mg/kg), the ADC-3 and the ADC-5 had a slightly better efficacy and the ADC-4 had basically the same efficacy at the same dosage. In the high-dose group, compared with the ADC-1 (10 mg/kg), the 3 test compounds had a better efficacy than that of the ADC-1 at the same dosage. Tumors in two of 6 tumor-bearing mice in the ADC-5 (10 mg/kg) group completely disappeared on a 21^{st} day and a 31^{st} day after drug withdrawal, while tumors in the tumor-bearing mice in the ADC-1 (10 mg/kg) group began to rebound obviously on the 21^{st} day after drug withdrawal. Weights of the mice in various administration groups showed an increasing trend, indicating well tolerance.

To sum up, the drug of the Exatecan derivative of the present disclosure has good efficacy and safety, and has great research and application values.

TABLE 8
Evaluation index of anti-tumor efficacy of test object

| Group | Test object | D59^{a} (mm³) | TGI^{b} (%) |
|---|---|---|---|
| G1 | Vehicle | 791.5 ± 227.1 | / |
| G2 | ADC-4 (3 mg/kg) | 284.9 ± 82.5 | 79.3 |
| G3 | ADC-4 (10 mg/kg) | 19.3 ± 3.2 | 119.6 |
| G4 | ADC-3 (3 mg/kg) | 136.0 ± 21.5 | 101.6 |
| G5 | ADC-3 (10 mg/kg) | 15.3 ± 6.0 | 120.5 |
| G6 | ADC-5 (3 mg/kg) | 115.0 ± 32.4 | 105.1 |
| G7 | ADC-5 (10 mg/kg) | 68.6 ± 23.2 | 111.4 |
| G8 | ADC-1 (3 mg/kg) | 300.2 ± 94.1 | 75.3 |
| G9 | ADC-1 (10 mg/kg) | 107.7 ± 27.6 | 105.3 |

Notes:
^{a}mean value ± standard error; D 59^{a} refers to a tumor volume on the 59^{th} day (D 59) after tumor inoculation; and
^{b}tumor growth inhibition rate TGI (%) = [1 − (T₄₆-T₂₆)/(V₄₆-V₂₆)] × 100%, the greater the TGI data, the better the efficacy.

### Test Example 9: In-Vivo Pharmacokinetics Experiment of ADCs in Examples of the Present Disclosure in SD Rats

SD rats were taken as test animals to study in-vivo pharmacokinetics behaviors after injection administration of the ADCs of the present disclosure to male and female SD rats.

### 1. Experimental Scheme

### 1.1 Test Samples:

The control compound ADC-1 and some compounds of the present disclosure.

### 1.2 Experimental Animals

### Basic Information of Animals

Species: male and female SD rats (2 female rats and 2 male rats/group)
Grade: SPF grade
Weight: about 200 g

### 1.3 Administration:

Male and female SD rats were intravenously injected with the test sample solution, and the ADCs of the present disclosure and the control ADC-1 were given in a dosage of 30 mg/kg and a dosage volume of 10 mL/kg.

Preparation of test sample solution: the test sample was diluted to 3 mg/mL with a normal saline.

Control group: ADC-1.

Experimental groups: some ADCs of the present disclosure.

### 1.5 Sample Collection:

0 before administration, and 0.083 hours, 2 hours, 6 hours, 24 hours, 72 hours, 120 hours, 168 hours, 336 hours, 504 hours and 672 hours after administration, 0.2 mL of blood was collected and placed into a marker-containing EDTA-2K anticoagulant tube respectively. The anticoagulant tube was turned upside down gently to fully mix an anticoagulant (EDTA-2K) with the blood, and then immediately placed into wet ice, and plasma was centrifuged for separation within 1 hour after blood collection at 4° C. and 6800 g for 6 minutes. The plasma separated after centrifugation was divided into two tubes: 50 μL of the plasma was transferred to an EP tube containing 2 μL of 13% formic acid-water (payload detection tube, the sample contained 0.5% formic acid); and the remaining plasma was transferred into another marked EP tube (macromolecular detection tube) and stored in an ultra-low-temperature refrigerator as soon as possible until sample analysis.

In-vivo pharmacokinetics results of the rats on the 7th day are shown in Table 9.

TABLE 9
Pharmacokinetics parameters on the 7^{th} day after intravenous administration to SD rats

| | | | | AUC_{last(ADC)}/ |
|---|---|---|---|---|
| Serial number of sample | Detection item | Cₘₐₓ (μg/mL) | AUCₗₐₛₜ (h*μg/mL) | AUC_{last(total antibody)} |
| ADC-3 | Total antibody | 515.175 | 23579.968 | 0.995 |
| | ADC | 504.575 | 23460.054 | |
| | Toxin | 0.000665 | 0.00355 | |
| ADC-4 | Total antibody | 501.45 | 22177.393 | 1.017 |
| | ADC | 523.075 | 22556.205 | |
| | Toxin | 0.000979 | 0.022092 | |
| ADC-1 | Total antibody | 603.775 | 25974.614 | 0.897 |
| | ADC | 609.5 | 23294.748 | |
| | Toxin | 0.00115 | 0.020051837 | |

Note:
Cₘₐₓ represents a peak concentration; AUCₗₐₛₜ represents an exposure quantity from an administration time to blood sampling analysis, and AUC_{last(ADC)}/AUC_{last(total antibody)} represents an exposure ratio of a complete ADC to the total antibody.

Conclusion: test data on the 7^{th} day show that an exposure quantity of free toxin of the ADC-3 is one order of magnitude lower than that of the ADC-1, which indicates that the ADC-3 is more stable, with less toxin shedding. AUC_{last(ADC)}/AUC_{last(total antibody)} reflects a stability of linker-payload, and the closer the value is to 1, the more stable the linker is. AUC_{last(ADC)}/AUC_{last(total antibody)} ratios of the ADC-3 and the ADC-4 are higher than that of the ADC-1, which further indicates that in-vivo stabilities of the ADC-3 and the ADC-4 are significantly better than that of the ADC-1.

1. A cytotoxic drug as shown by formula (D′), a stereoisomer thereof or a pharmaceutically acceptable salt thereof, 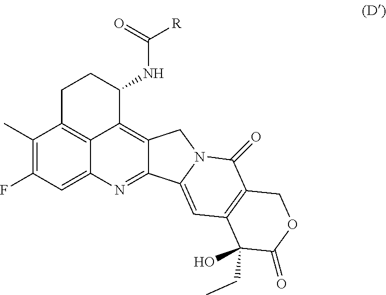
wherein, R is —NR¹R² or —OR³;
when R¹ is —CH₂CH₂OH, R² is selected from OH, CN, C₁₋₄ alkyl, C₁₋₄ alkoxy, C₂₋₄ alkenyl, C₂₋₄ alkynyl, —O—C₃₋₆ cycloalkyl, 3-6 membered heterocyclyl or C₃₋₆ cycloalkyl, and the C₁₋₄ alkyl, the C₃₋₆ cycloalkyl and the 3-6 membered heterocyclyl are optionally further substituted by 1, 2 or 3 CN or ═O; and the 3-6 membered heterocyclyl contains 1, 2 or 3 heteroatoms selected from N, O or S;
or, when R¹ is selected from H, C₁₋₄ alkyl or C₃₋₆ cycloalkyl, R² is selected from 3-6 membered heterocyclyl or —O—CH₂CH₂OH;
or, when R¹ is C₁₋₄ alkoxy, R² is selected from C₃₋₆ cycloalkyl or 3-6 membered heterocyclyl, and the C₃₋₆ cycloalkyl is optionally further substituted by one or more substituents selected from OH or NH₂; and
R³ is selected from C₃₋₆ cycloalkyl or 3-6 membered heterocyclyl, and the C₃₋₆ cycloalkyl is optionally further substituted by one or more substituents selected from OH or NH₂.

2. The cytotoxic drug, the stereoisomer thereof or the pharmaceutically acceptable salt thereof according to claim 1, wherein:
when R¹ is —CH₂CH₂OH, R² is selected from OH, CN, isopropyl, —CH₂CN, —CH₂C≡CH, —CH₂CH═CH₂, methoxy, 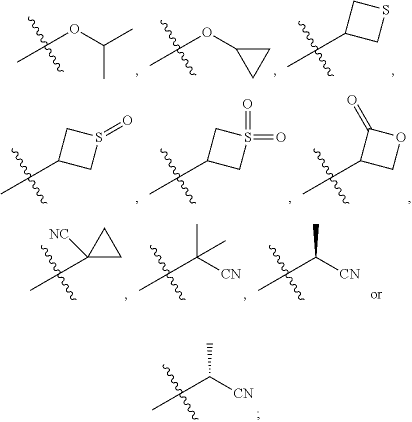
or when R¹ is H, R² is selected from —O—CH₂CH₂OH or 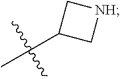 [SMILES: *C1CNC1]
or, when R¹ is selected from methyl, isopropyl or cyclopropyl, R² is —O—CH₂CH₂OH;
or, when R¹ is methoxy, R² is selected from cyclobutyl or 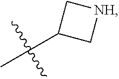 [SMILES: *C1CNC1]
and the cyclobutyl is optionally further substituted by NH₂.

3. (canceled)

4. The cytotoxic drug, the stereoisomer thereof or the pharmaceutically acceptable salt thereof according to claim 1, wherein:
R³ is selected from cyclobutyl or azacyclobutyl, and the cyclobutyl is optionally further substituted by one or more substituents selected from OH or NH₂.

5. The cytotoxic drug, the stereoisomer thereof or the pharmaceutically acceptable salt thereof according to claim 1, which is selected from:

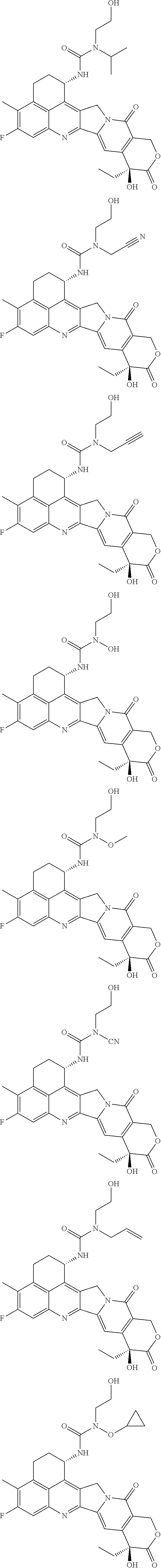

[chemical structure]

[chemical structure]

6. An antibody drug conjugate, a stereoisomer thereof or a pharmaceutically acceptable salt thereof, which is selected from a structure as shown by formula 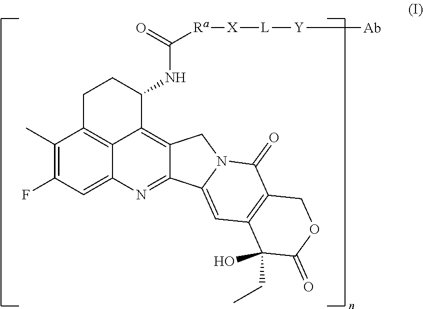
wherein, Ab is an antibody or an antigen-binding fragment;
Y is a linker part linked to the Ab;
L is a peptide residue consisting of 2 to 4 amino groups selected from phenylalanine, glycine, valine, citrulline, alanine, glutamic acid and aspartic acid;
X is a structural fragment connecting R^{a} to L;
R^{a} is a structure of R in the absence of H; and the R is as defined in claim 1; and
n is selected from numerical values of 4-8,
wherein X is selected from —CH₂—NH or 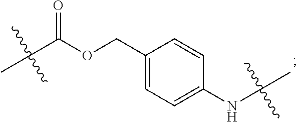 [SMILES: *Nc1ccc(COC(*)=O)cc1]
optionally, wherein L is selected from 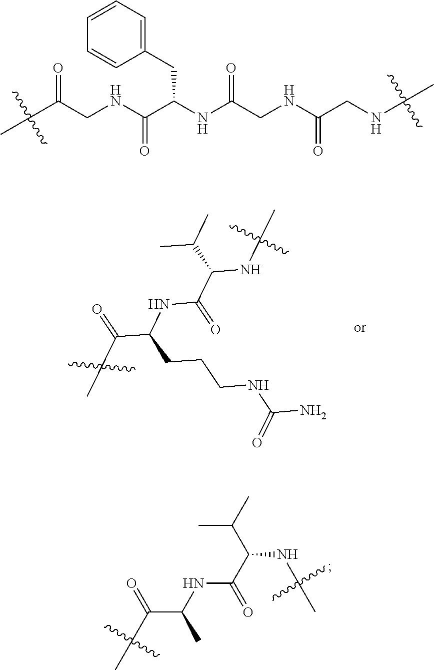
wherein Y is selected from —CO—Y¹—Y²—S—;
Y² is selected from 5-6 membered heteroaryl or 5-6 membered heterocyclyl;
Y¹ is selected from —(CH₂)ₘ—, (CH₂)ₘ—NH—SO₂—, —CH₂—N⁺(CH₃)₂—(CH₂)ₘ—NH—SO₂—, —C₂₋₄ alkynylene-NH—SO₂—, —C₂₋₅ alkynylene-, —(CH₂)ₘ—O—C₂₋₄ alkynylene-, —C₃₋₅ cycloalkyl-C₂₋₄ alkynylene-, —(CH₂)ₘ-5-6 membered heterocyclyl-SO₂—, —C₆₋₁₀ aryl-SO₂—NH— and —C₆₋₁₀ aryl-NH—SO₂—; and
m is selected from 0, 1, 2, 3, 4 or 5;
optionally, wherein Y is selected from —CO—Y¹—Y²—S—;
Y¹ is selected from —(CH₂)₅—, —CH₂—NH—SO₂—, —(CH₂)₂—NH—SO₂—, —(CH₂)₃—NH—SO₂—CH₂—N⁺(CH₃)₂—(CH₂)₂—NH—SO₂—, —C≡C—CH₂—NH—SO₂—, —(CH₂)₃—C≡C—, —CH₂—O—CH₂—C≡C—, 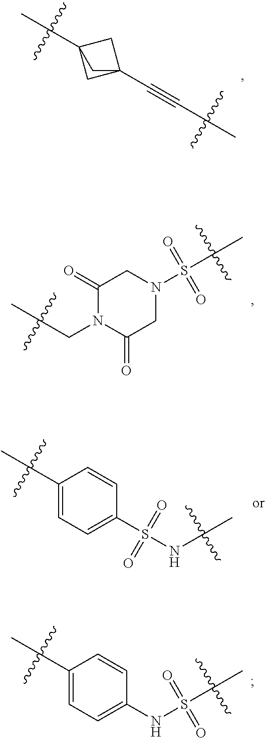 [SMILES: *C#CC12CC(*)(C1)C2 ; *CN1C(=O)CN(S(*)(=O)=O)CC1=O ; *NS(=O)(=O)c1ccc(*)cc1 ; *c1ccc(NS(*)(=O)=O)cc1]
and
Y² is selected from 5-6 membered N-containing heteroaryl or 5-6 membered N-containing heterocyclyl; and optionally, Y² is selected from 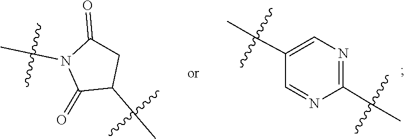
or
optionally, wherein Y is selected from

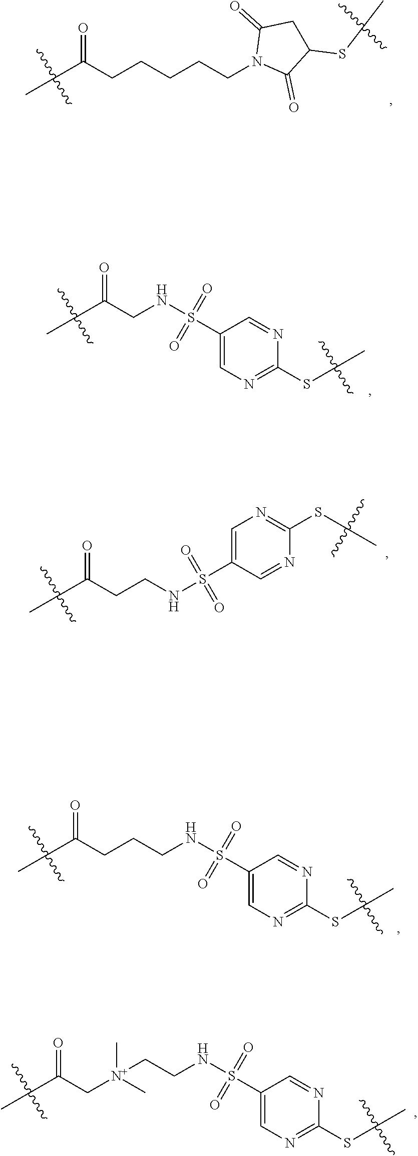 [SMILES: *SC1CC(=O)N(CCCCCC(*)=O)C1=O ; *Sc1ncc(S(=O)(=O)NCC(*)=O)cn1 ; *Sc1ncc(S(=O)(=O)NCCC(*)=O)cn1 ; *Sc1ncc(S(=O)(=O)NCCCC(*)=O)cn1 ; *Sc1ncc(S(=O)(=O)NCC[N+](C)(C)CC(*)=O)cn1]

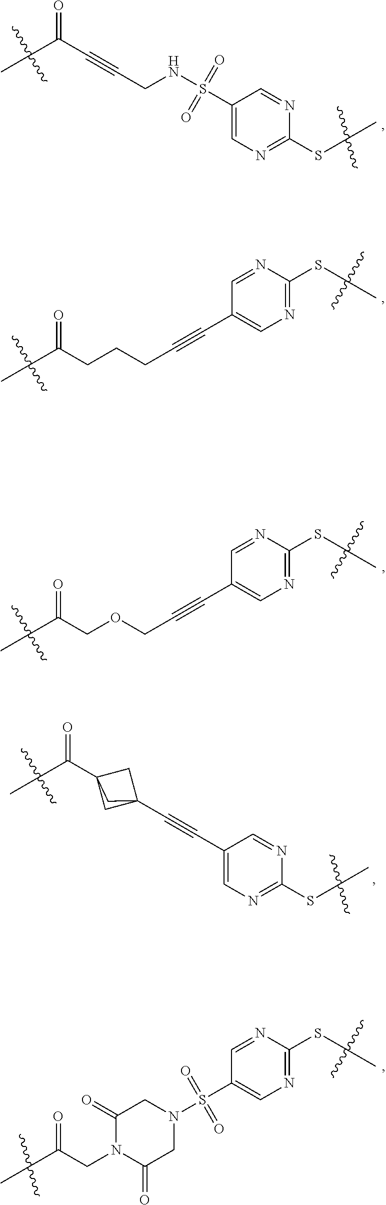 [SMILES: *Sc1ncc(S(=O)(=O)NCC#CC(*)=O)cn1 ; *Sc1ncc(C#CCCCC(*)=O)cn1 ; *Sc1ncc(C#CCOCC(*)=O)cn1 ; *Sc1ncc(C#CC23CC(C(*)=O)(C2)C3)cn1 ; *Sc1ncc(S(=O)(=O)N2CC(=O)N(CC(*)=O)C(=O)C2)cn1]

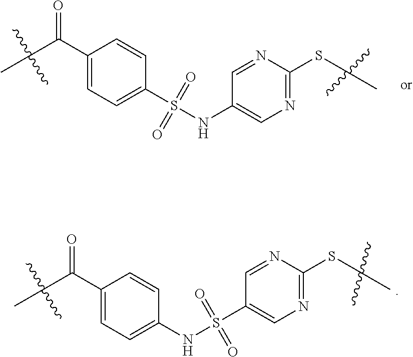 [SMILES: *Sc1ncc(NS(=O)(=O)c2ccc(C(*)=O)cc2)cn1 ; *Sc1ncc(S(=O)(=O)Nc2ccc(C(*)=O)cc2)cn1]

7.-11. (canceled)

12. The antibody drug conjugate, the stereoisomer thereof or the pharmaceutically acceptable salt thereof according to claim 6, wherein constitutional unit —X-L-Y— is selected from:

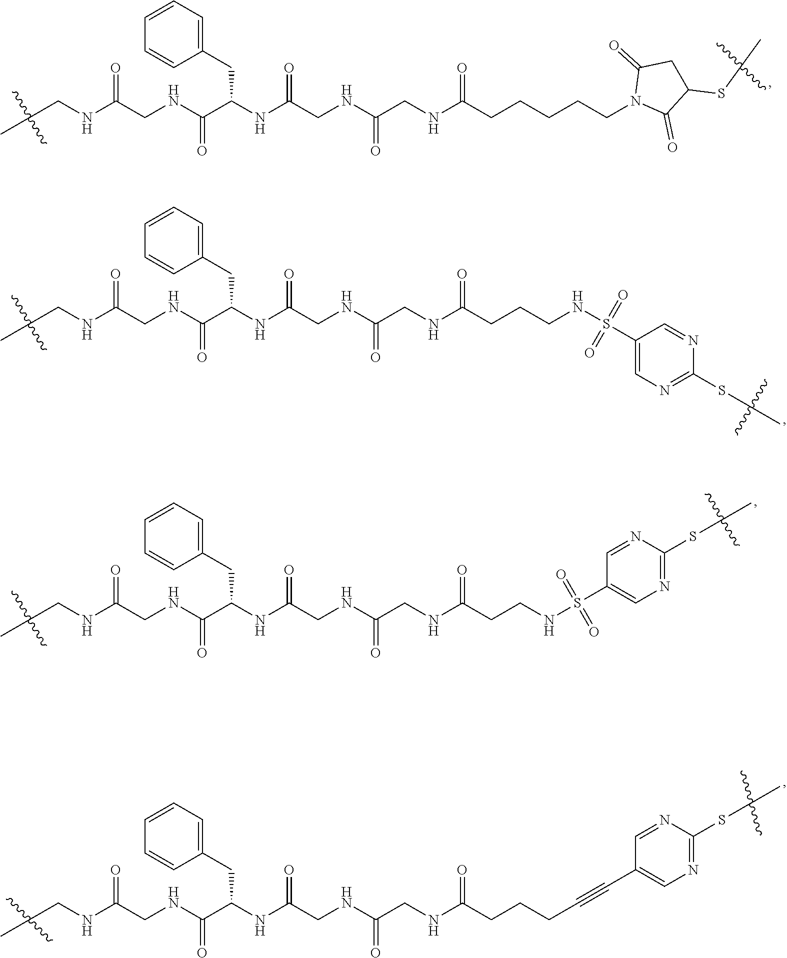 [SMILES: *CNC(=O)CNC(=O)[C@H](Cc1ccccc1)NC(=O)CNC(=O)CNC(=O)CCCCCN1C(=O)CC(S*)C1=O ; *CNC(=O)CNC(=O)[C@H](Cc1ccccc1)NC(=O)CNC(=O)CNC(=O)CCCNS(=O)(=O)c1cnc(S*)nc1 ; *CNC(=O)CNC(=O)[C@H](Cc1ccccc1)NC(=O)CNC(=O)CNC(=O)CCNS(=O)(=O)c1cnc(S*)nc1 ; *CNC(=O)CNC(=O)[C@H](Cc1ccccc1)NC(=O)CNC(=O)CNC(=O)CCCC#Cc1cnc(S*)nc1]

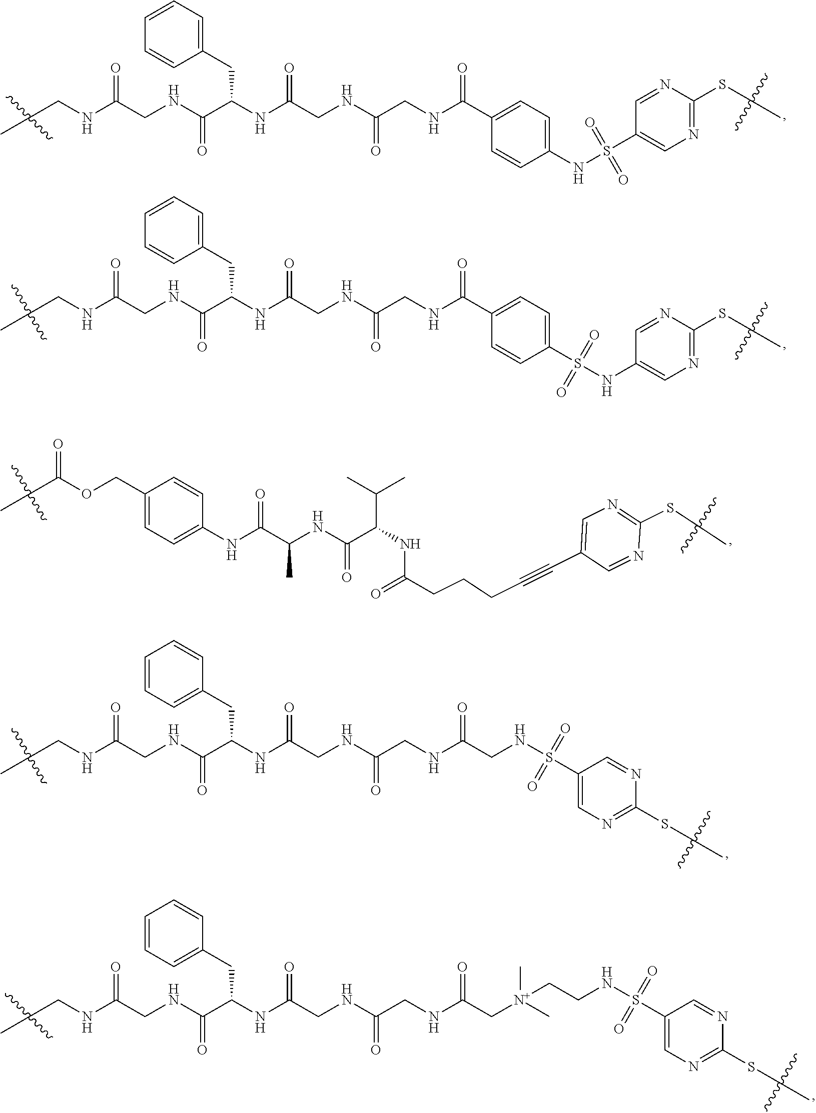 [SMILES: *CNC(=O)CNC(=O)[C@H](Cc1ccccc1)NC(=O)CNC(=O)CNC(=O)c1ccc(NS(=O)(=O)c2cnc(S*)nc2)cc1 ; *CNC(=O)CNC(=O)[C@H](Cc1ccccc1)NC(=O)CNC(=O)CNC(=O)c1ccc(S(=O)(=O)Nc2cnc(S*)nc2)cc1 ; *Sc1ncc(C#CCCCC(=O)N[C@H](C(=O)N[C@@H](C)C(=O)Nc2ccc(COC(*)=O)cc2)C(C)C)cn1 ; *CNC(=O)CNC(=O)[C@H](Cc1ccccc1)NC(=O)CNC(=O)CNC(=O)CNS(=O)(=O)c1cnc(S*)nc1 ; *CNC(=O)CNC(=O)[C@H](Cc1ccccc1)NC(=O)CNC(=O)CNC(=O)C[N+](C)(C)CCNS(=O)(=O)c1cnc(S*)nc1]

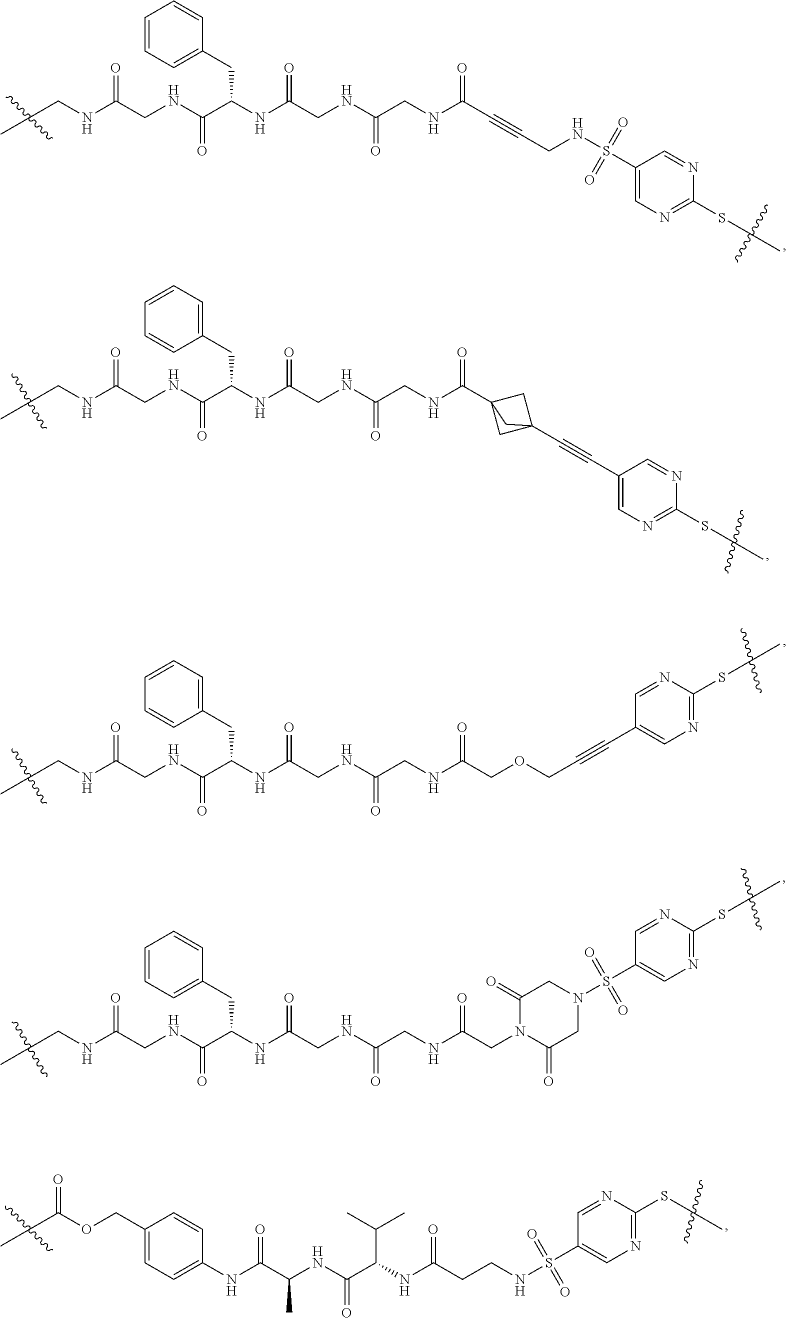 [SMILES: *CNC(=O)CNC(=O)[C@H](Cc1ccccc1)NC(=O)CNC(=O)CNC(=O)C#CCNS(=O)(=O)c1cnc(S*)nc1 ; *CNC(=O)CNC(=O)[C@H](Cc1ccccc1)NC(=O)CNC(=O)CNC(=O)C12CC(C#Cc3cnc(S*)nc3)(C1)C2 ; *CNC(=O)CNC(=O)[C@H](Cc1ccccc1)NC(=O)CNC(=O)CNC(=O)COCC#Cc1cnc(S*)nc1 ; *CNC(=O)CNC(=O)[C@H](Cc1ccccc1)NC(=O)CNC(=O)CNC(=O)CN1C(=O)CN(S(=O)(=O)c2cnc(S*)nc2)CC1=O ; *Sc1ncc(S(=O)(=O)NCCC(=O)N[C@H](C(=O)N[C@@H](C)C(=O)Nc2ccc(COC(*)=O)cc2)C(C)C)cn1]

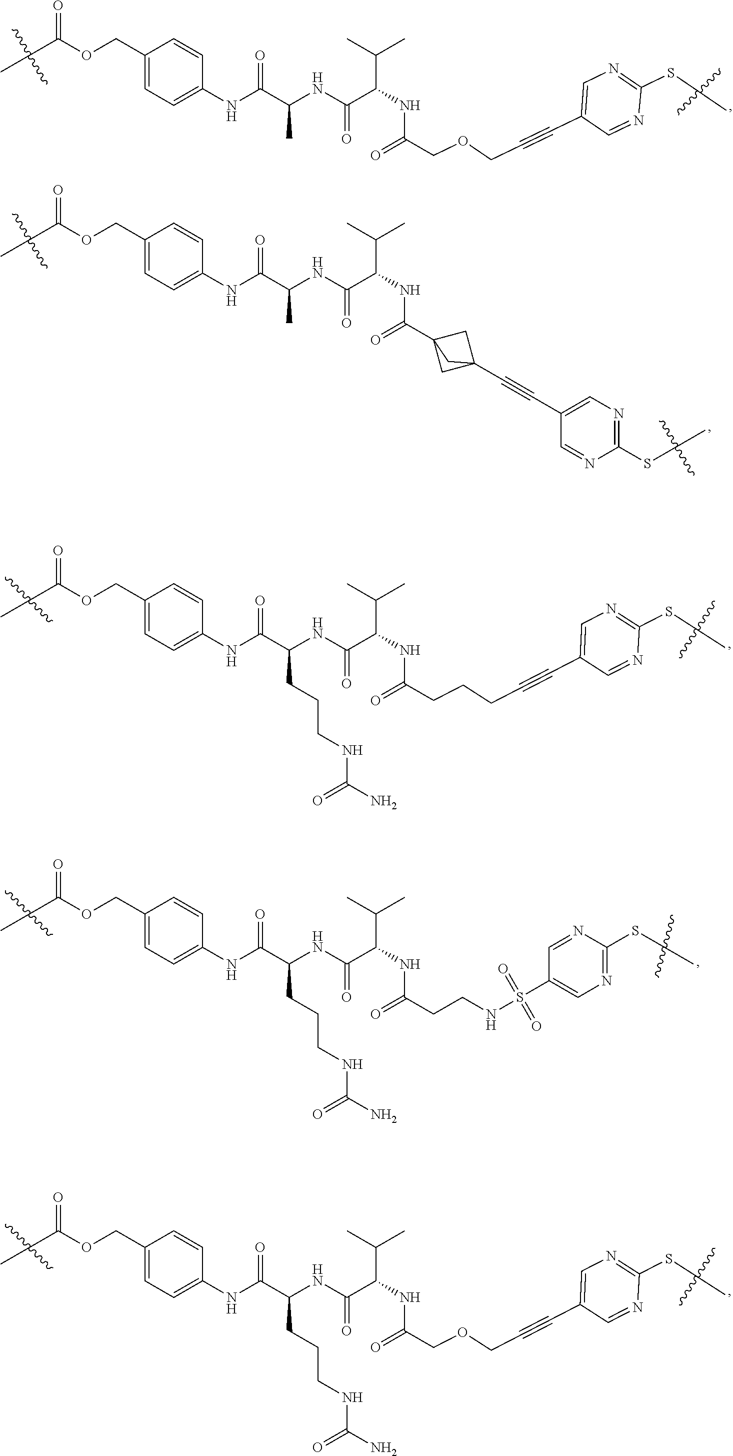 [SMILES: *Sc1ncc(C#CCOCC(=O)N[C@H](C(=O)N[C@@H](C)C(=O)Nc2ccc(COC(*)=O)cc2)C(C)C)cn1 ; *Sc1ncc(C#CC23CC(C(=O)N[C@H](C(=O)N[C@@H](C)C(=O)Nc4ccc(COC(*)=O)cc4)C(C)C)(C2)C3)cn1 ; *Sc1ncc(C#CCCCC(=O)N[C@H](C(=O)N[C@@H](CCCNC(N)=O)C(=O)Nc2ccc(COC(*)=O)cc2)C(C)C)cn1 ; *Sc1ncc(S(=O)(=O)NCCC(=O)N[C@H](C(=O)N[C@@H](CCCNC(N)=O)C(=O)Nc2ccc(COC(*)=O)cc2)C(C)C)cn1 ; "," ; *Sc1ncc(C#CCOCC(=O)N[C@H](C(=O)N[C@@H](CCCNC(N)=O)C(=O)Nc2ccc(COC(*)=O)cc2)C(C)C)cn1]

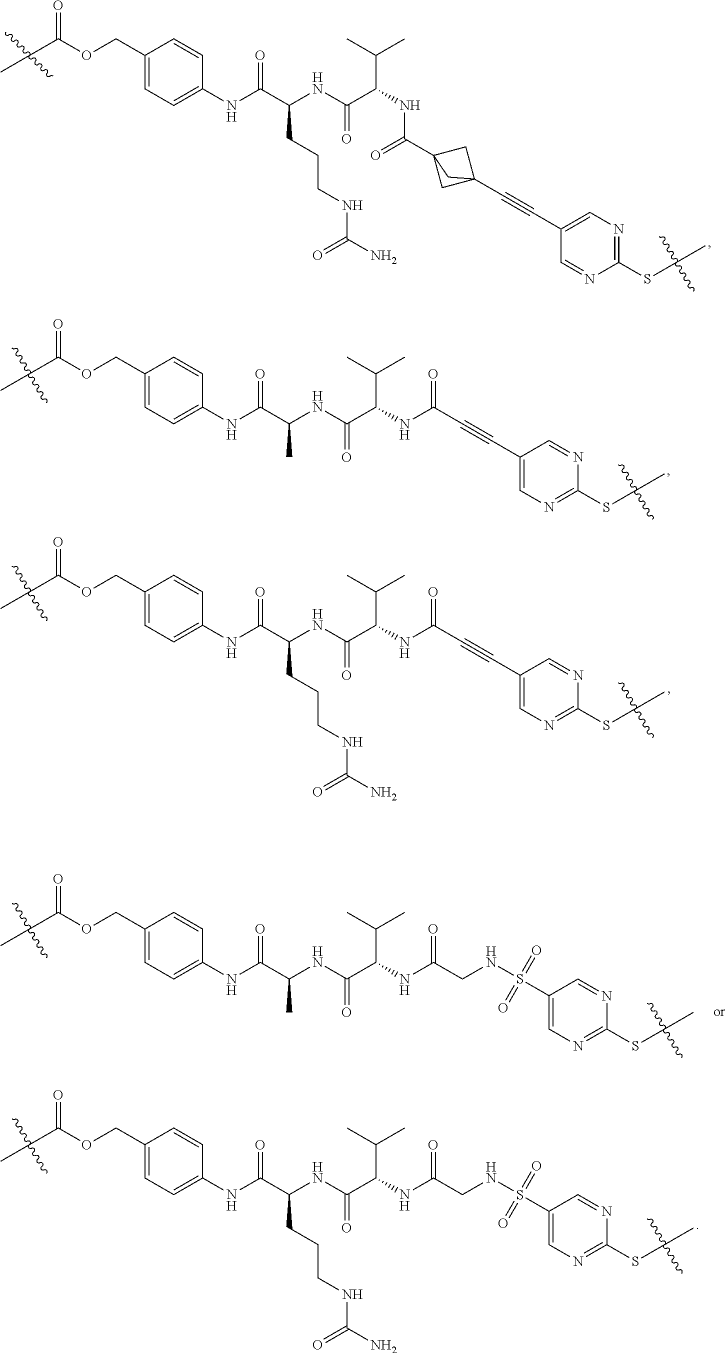 [SMILES: *Sc1ncc(C#CC23CC(C(=O)N[C@H](C(=O)N[C@@H](CCCNC(N)=O)C(=O)Nc4ccc(COC(*)=O)cc4)C(C)C)(C2)C3)cn1 ; *Sc1ncc(C#CC(=O)N[C@H](C(=O)N[C@@H](C)C(=O)Nc2ccc(COC(*)=O)cc2)C(C)C)cn1 ; *Sc1ncc(C#CC(=O)N[C@H](C(=O)N[C@@H](CCCNC(N)=O)C(=O)Nc2ccc(COC(*)=O)cc2)C(C)C)cn1 ; *Sc1ncc(S(=O)(=O)NCC(=O)N[C@H](C(=O)N[C@@H](C)C(=O)Nc2ccc(COC(*)=O)cc2)C(C)C)cn1 ; *Sc1ncc(S(=O)(=O)NCC(=O)N[C@H](C(=O)N[C@@H](CCCNC(N)=O)C(=O)Nc2ccc(COC(*)=O)cc2)C(C)C)cn1]

13. The antibody drug conjugate, the stereoisomer thereof or the pharmaceutically acceptable salt thereof according to claim 6, wherein R^{a} is —NR^{a1}R^{a2} and —OR^{a3};
when R^{a1} is connected to X, R^{a1} is —CH₂CH₂O—, R^{a2} is selected from OH, CN, C₁₋₄ alkyl, C₁₋₄ alkoxy, C₂₋₄ alkenyl, C₂₋₄ alkynyl, —O—C₃₋₆ cycloalkyl, 3-6 membered heterocyclyl or C₃₋₆ cycloalkyl, and the C₁₋₄ alkyl and the C₃₋₆ cycloalkyl are optionally further substituted by CN; and the 3-6 membered heterocyclyl contains 1, 2 or 3 heteroatoms selected from N, O or S;
when R^{a2} is connected to X, R^{a1} is selected from H, C₁₋₄ alkyl or C₃₋₆ cycloalkyl, and R^{a2} is selected from 3-6 membered N-containing heterocyclyl or —O—CH₂CH₂O—;
or, when R^{a2} is connected to X, R^{a1} is C₁₋₄ alkoxy, R^{a2} is selected from C₃₋₆ cycloalkyl or 3-6 membered N-containing heterocyclyl, and the C₃₋₆ cycloalkyl is optionally further substituted by one or more substituents selected from OH or NH₂; and
R^{a3} is selected from C₃₋₆ cycloalkyl or 3-6 membered N-containing heterocyclyl, and the C₃₋₆ cycloalkyl is optionally further substituted by one or more substituents selected from OH or NH₂; or
wherein R^{a} is —NR^{a1}R^{a2}, and when R^{a1} is connected to X, R^{a1} is —CH₂CH₂O—, and R^{a2} is selected from OH, CN, isopropyl, —CH₂CN, —CH₂C≡CH, —CH₂CH═CH₂, methoxy, 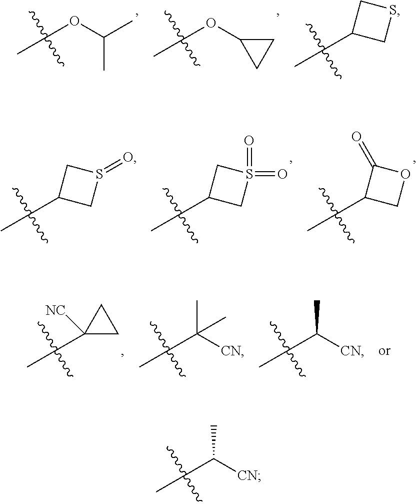
or
wherein R^{a} is —NR^{a1}R^{a2}, and when R^{a2} is connected to X;
R^{a1} is H, and R^{a2} is selected from —O—CH₂CH₂O— or 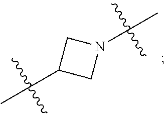 [SMILES: *C1CN(*)C1]
or
when R^{a1} is selected from methyl, isopropyl or cyclopropyl, R^{a2} is —O—CH₂CH₂O—, or
when R^{a1} is methoxy, R^{a2} is selected from C₄₋₅ cycloalkyl or 4-5 membered N-containing heterocycyl, and optionally, R^{a2} is selected from 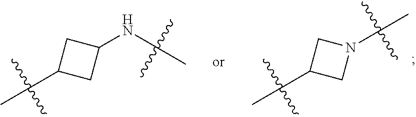 [SMILES: *NC1CC(*)C1 ; *C1CN(*)C1]
or
wherein R^{a} is —OR^{a3}, R^{a3} is selected from cyclobutyl or azacyclobutyl and the cyclobutyl is optionally further substituted by one or more substituents selected from OH or NH₂.

14.-16. (canceled)

17. The antibody drug conjugate, the stereoisomer thereof or the pharmaceutically acceptable salt thereof according to claim 6, wherein R^{a} is selected from

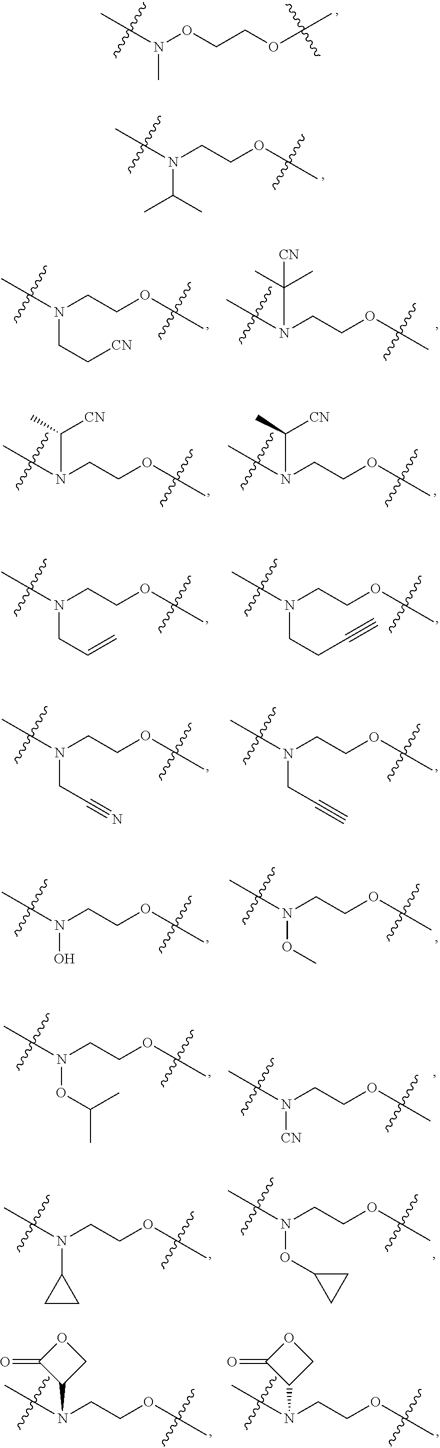

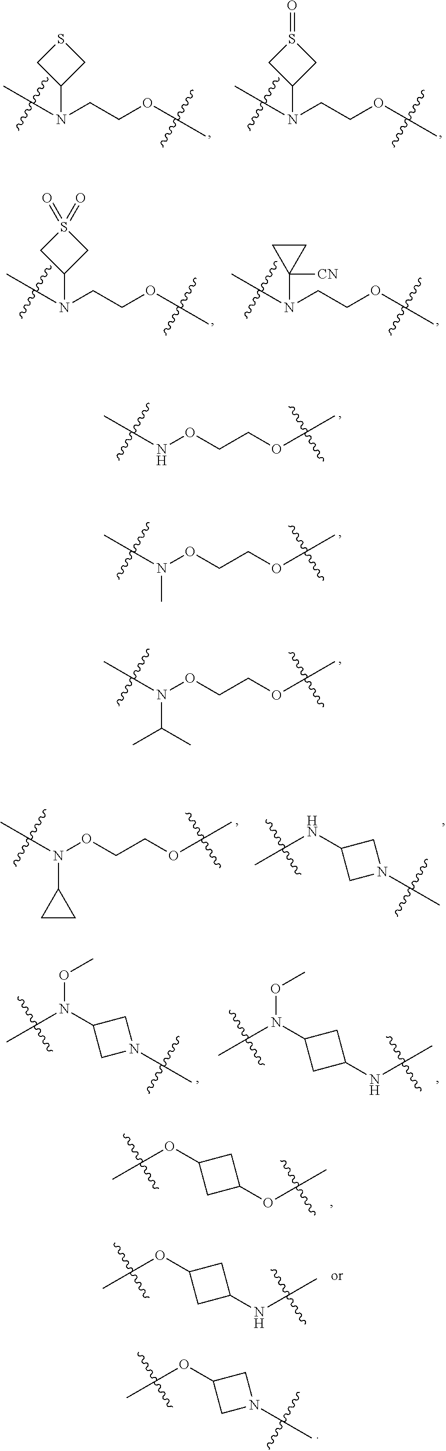

18. The antibody drug conjugate, the stereoisomer thereof or the pharmaceutically acceptable salt thereof according to claim 6, which is selected from: 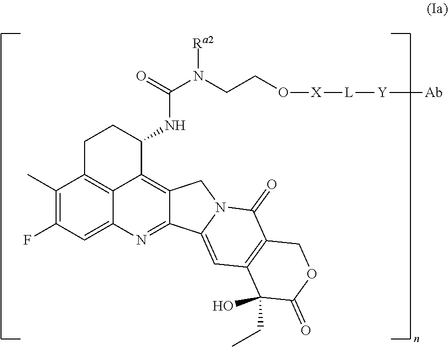
wherein, Ab, Y, L, X, R^{a2} and n are as defined in claim 6.

19. A cytotoxic drug-linker, a stereoisomer thereof or a pharmaceutically acceptable salt thereof, wherein the cytotoxic drug-linker has a structure as shown by formula D-X-L-Y′, wherein Y′ is a structure before Y is linked to the Ab in claim 6, D is a structure of a compound as shown by formula (D′) in the absence of H, X and Y are as defined in claim 6, and the compound as shown by formula (D′) is selected from: 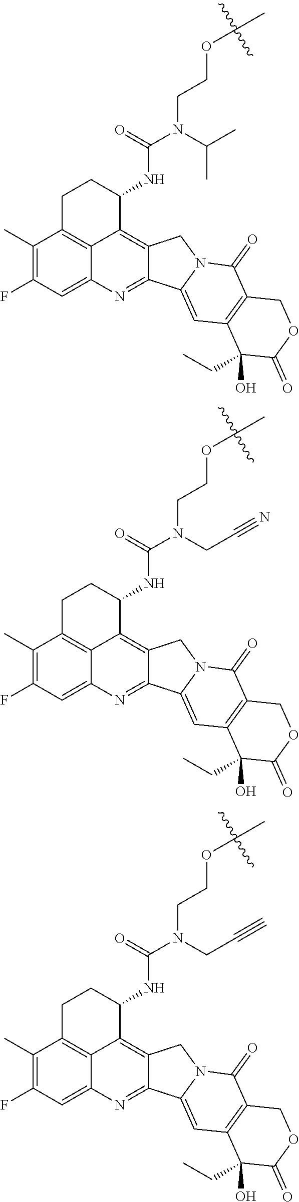 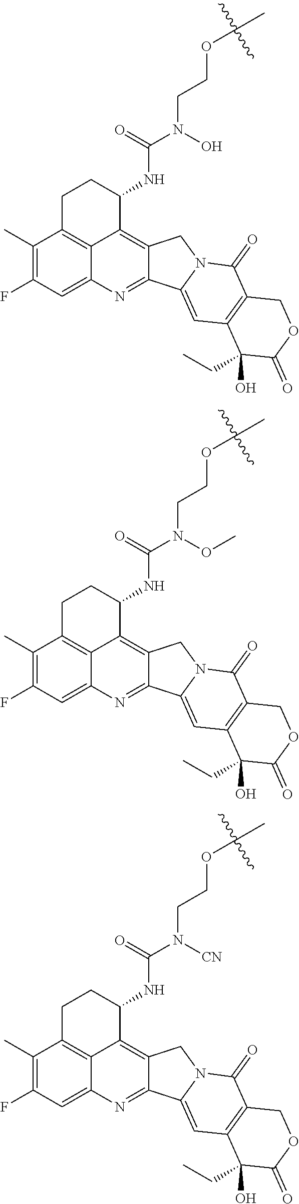 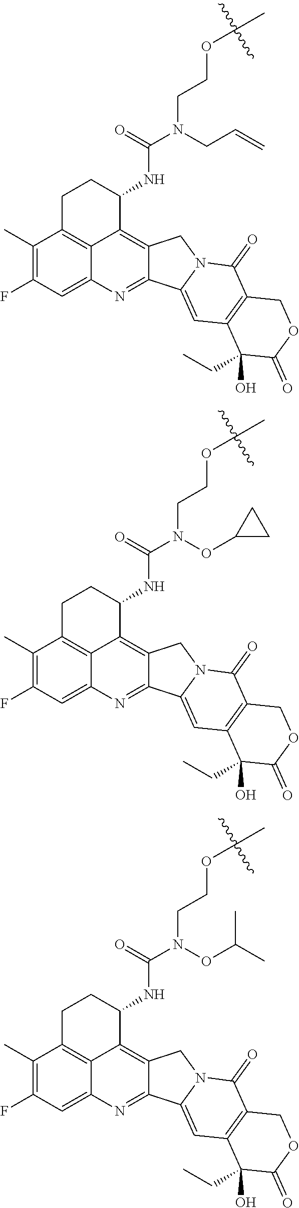 [SMILES: *OCCN(CC=C)C(=O)N[C@H]1CCc2c(C)c(F)cc3nc4c(c1c23)Cn1c-4cc2c(c1=O)COC(=O)[C@]2(O)CC ; *OCCN(OC1CC1)C(=O)N[C@H]1CCc2c(C)c(F)cc3nc4c(c1c23)Cn1c-4cc2c(c1=O)COC(=O)[C@]2(O)CC ; *OCCN(OC(C)C)C(=O)N[C@H]1CCc2c(C)c(F)cc3nc4c(c1c23)Cn1c-4cc2c(c1=O)COC(=O)[C@]2(O)CC] 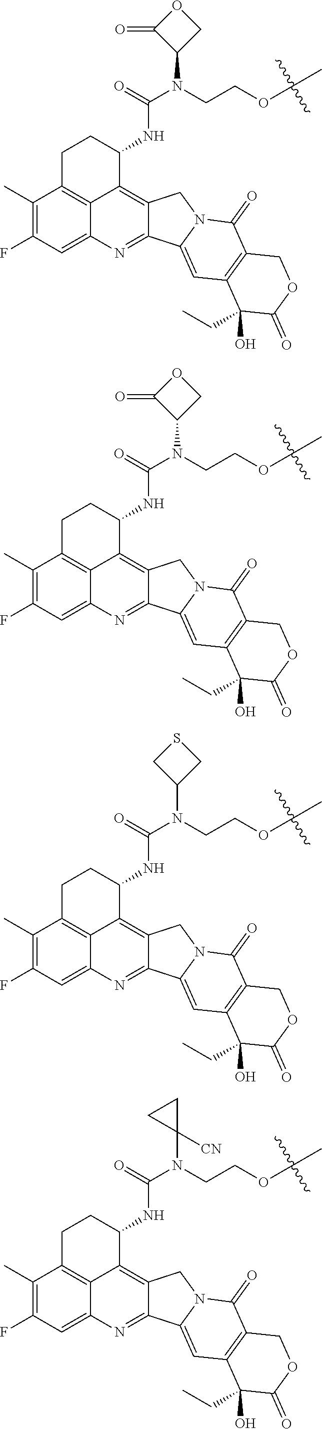 [SMILES: *OCCN(C(=O)N[C@H]1CCc2c(C)c(F)cc3nc4c(c1c23)Cn1c-4cc2c(c1=O)COC(=O)[C@]2(O)CC)[C@@H]1COC1=O ; *OCCN(C(=O)N[C@H]1CCc2c(C)c(F)cc3nc4c(c1c23)Cn1c-4cc2c(c1=O)COC(=O)[C@]2(O)CC)[C@H]1COC1=O ; *OCCN(C(=O)N[C@H]1CCc2c(C)c(F)cc3nc4c(c1c23)Cn1c-4cc2c(c1=O)COC(=O)[C@]2(O)CC)C1CSC1 ; *OCCN(C(=O)N[C@H]1CCc2c(C)c(F)cc3nc4c(c1c23)Cn1c-4cc2c(c1=O)COC(=O)[C@]2(O)CC)C1(C#N)CC1] 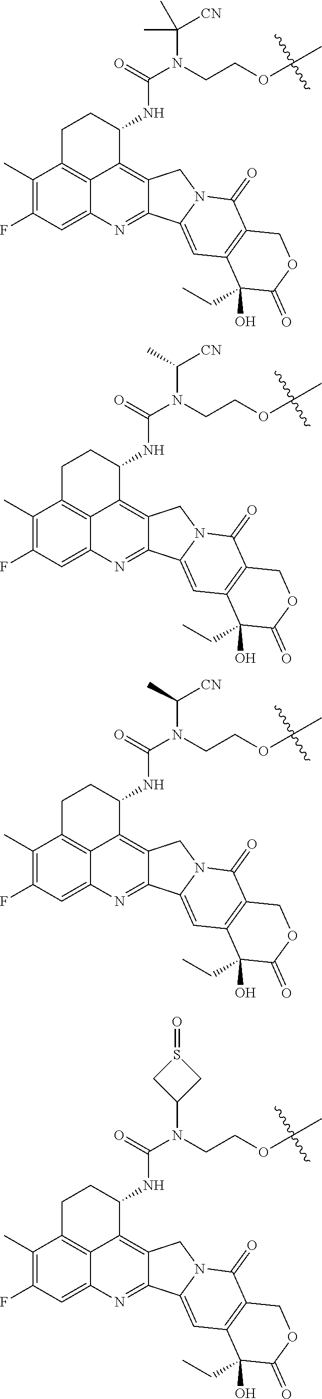 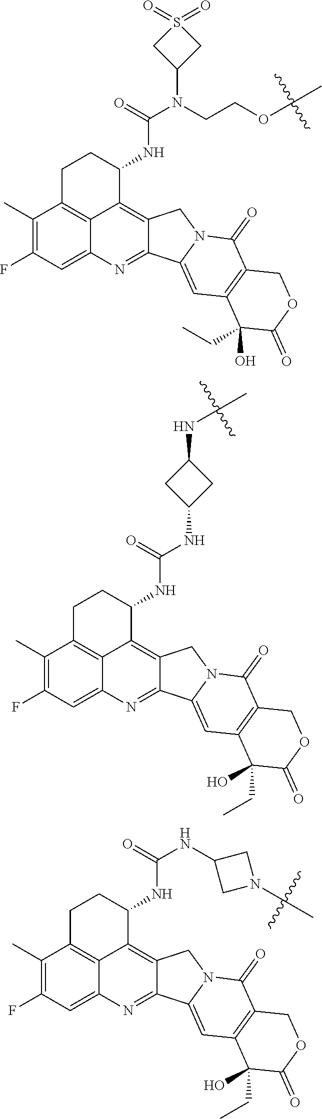 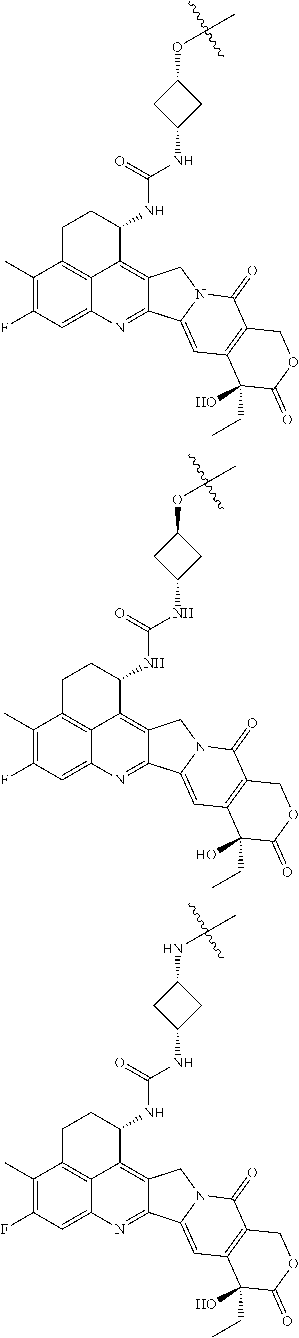 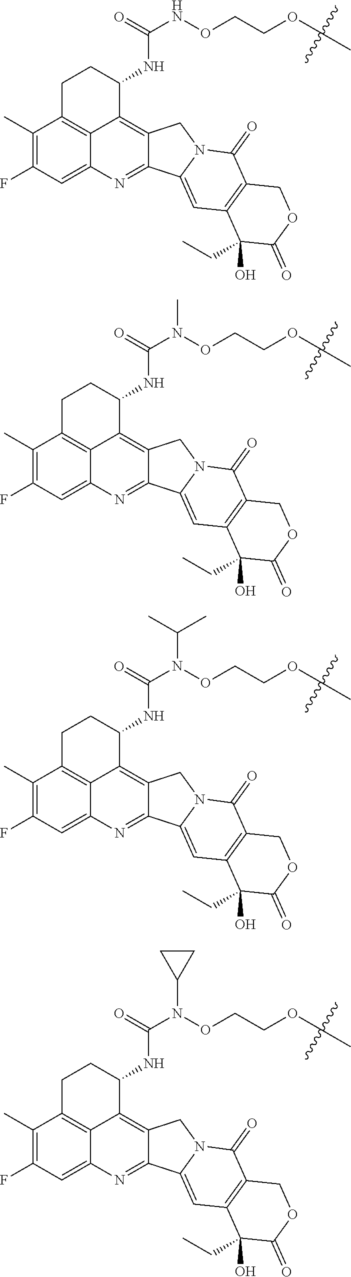 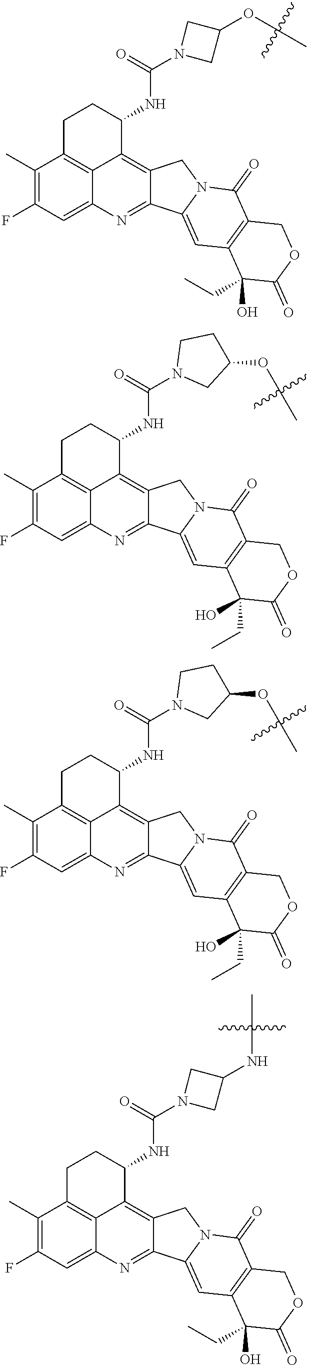 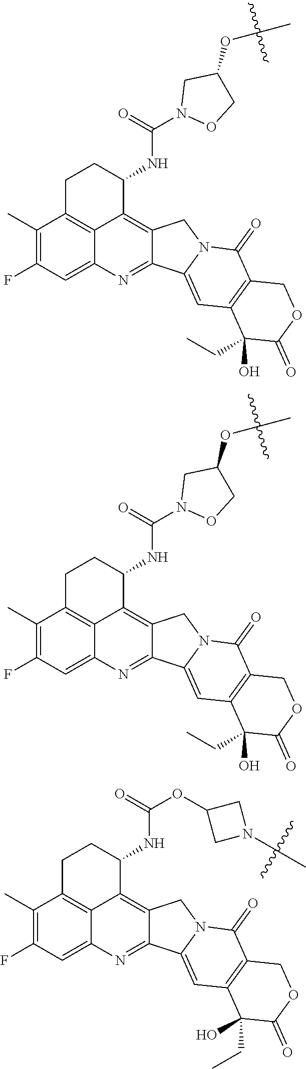 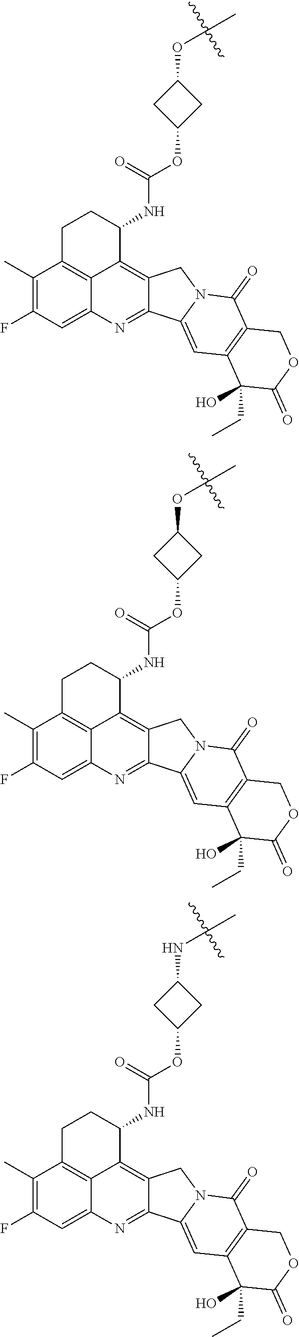 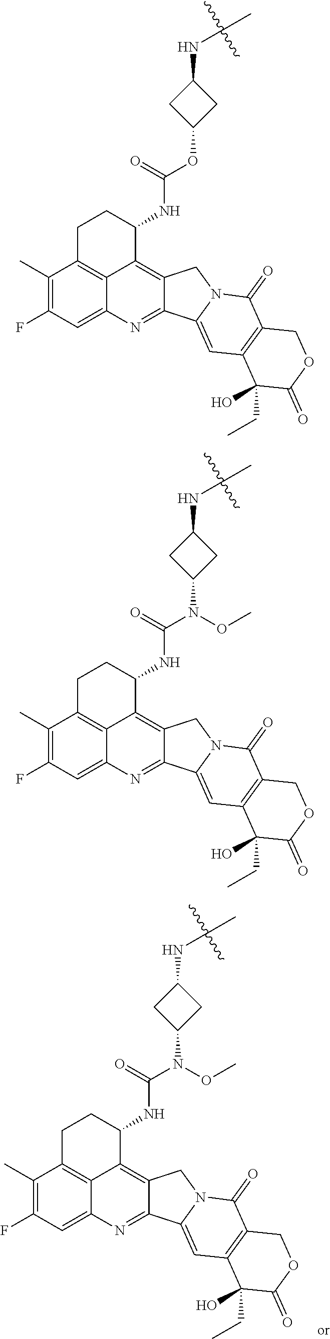 [SMILES: *N[C@H]1C[C@H](OC(=O)N[C@H]2CCc3c(C)c(F)cc4nc5c(c2c34)Cn2c-5cc3c(c2=O)COC(=O)[C@]3(O)CC)C1 ; *N[C@H]1C[C@H](N(OC)C(=O)N[C@H]2CCc3c(C)c(F)cc4nc5c(c2c34)Cn2c-5cc3c(c2=O)COC(=O)[C@]3(O)CC)C1 ; *N[C@H]1C[C@@H](N(OC)C(=O)N[C@H]2CCc3c(C)c(F)cc4nc5c(c2c34)Cn2c-5cc3c(c2=O)COC(=O)[C@]3(O)CC)C1] 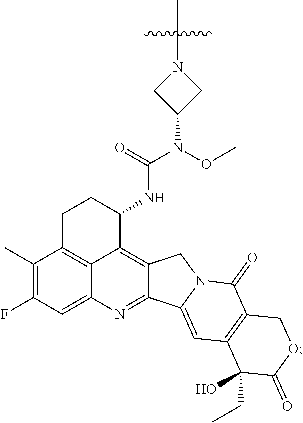
optionally, Y′ is selected from: 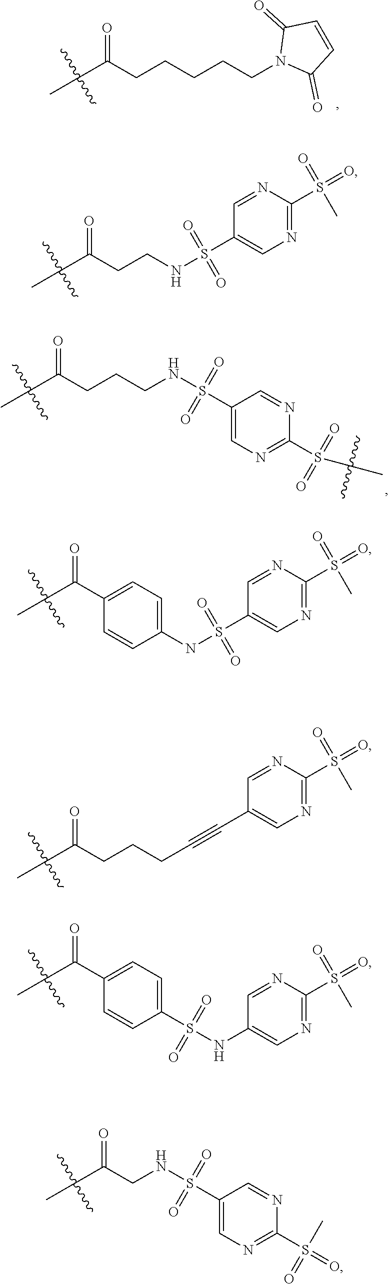 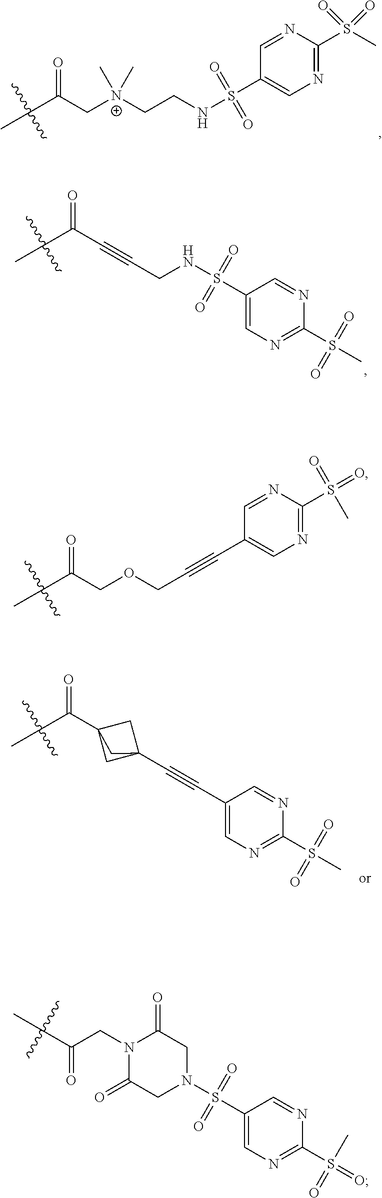
wherein L is selected from 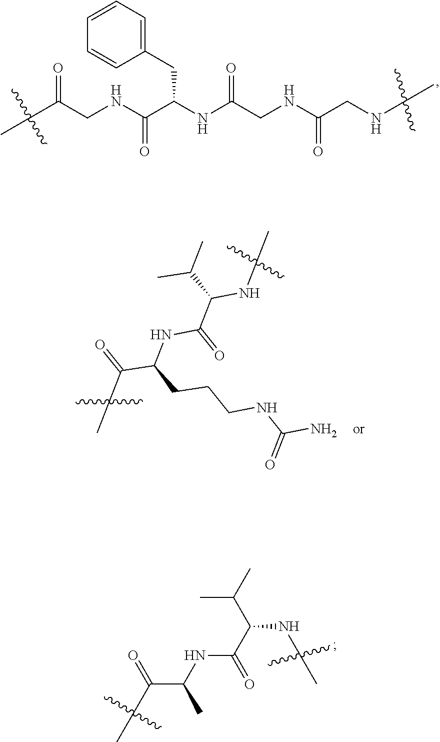 [SMILES: *NCC(=O)NCC(=O)N[C@@H](Cc1ccccc1)C(=O)NCC(*)=O ; *N[C@H](C(=O)N[C@@H](CCCNC(N)=O)C(*)=O)C(C)C ; *N[C@H](C(=O)N[C@@H](C)C(*)=O)C(C)C]
or
wherein X is selected from —CH₂—NH or 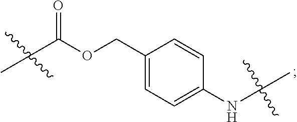 [SMILES: *Nc1ccc(COC(*)=O)cc1]
or
wherein constitutional unit —X-L-Y′ is selected from:

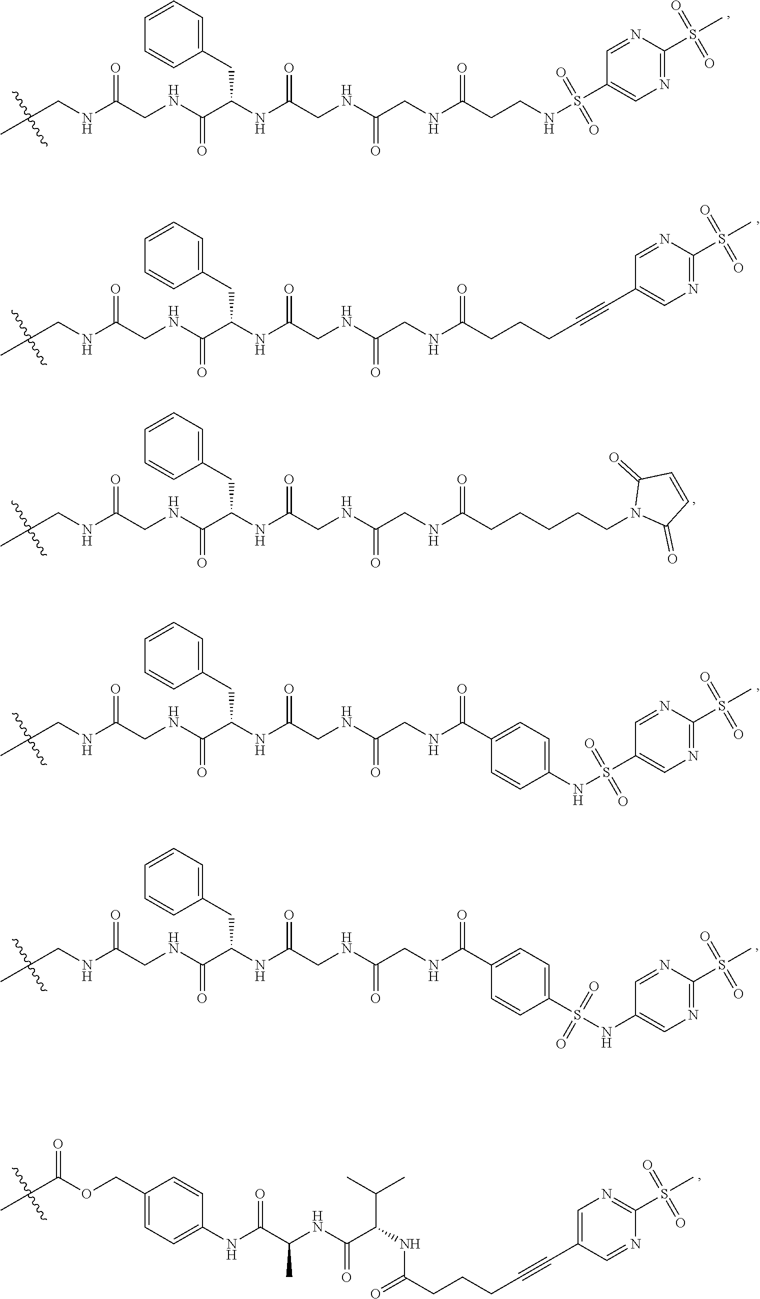

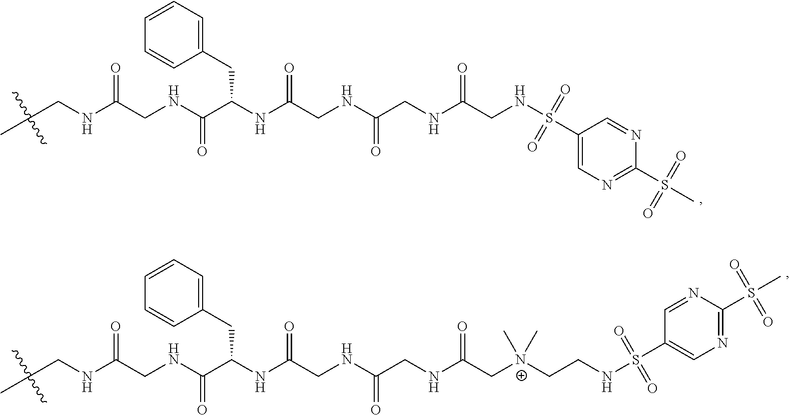 [SMILES: *CNC(=O)CNC(=O)[C@H](Cc1ccccc1)NC(=O)CNC(=O)CNC(=O)CNS(=O)(=O)c1cnc(S(C)(=O)=O)nc1 ; *CNC(=O)CNC(=O)[C@H](Cc1ccccc1)NC(=O)CNC(=O)CNC(=O)C[N+](C)(C)CCNS(=O)(=O)c1cnc(S(C)(=O)=O)nc1]

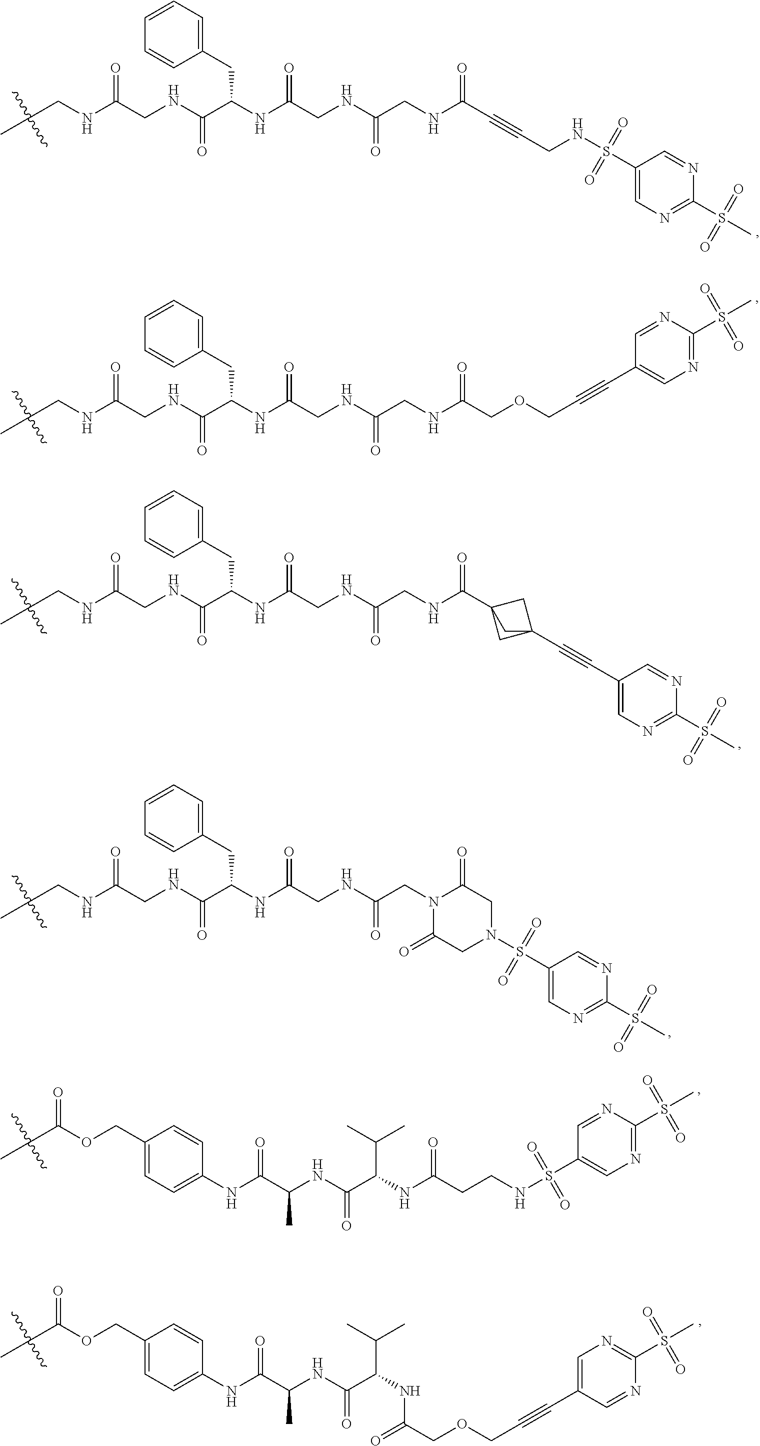

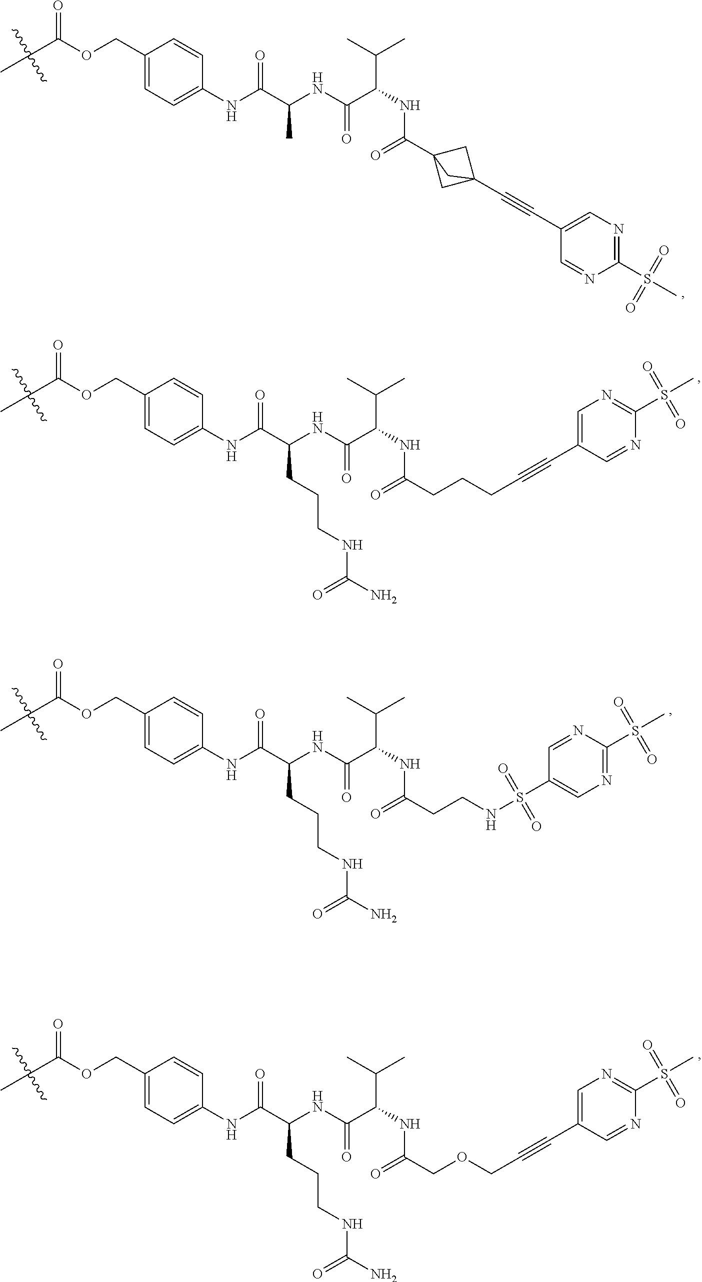 [SMILES: *C(=O)OCc1ccc(NC(=O)[C@H](C)NC(=O)[C@@H](NC(=O)C23CC(C#Cc4cnc(S(C)(=O)=O)nc4)(C2)C3)C(C)C)cc1 ; *C(=O)OCc1ccc(NC(=O)[C@H](CCCNC(N)=O)NC(=O)[C@@H](NC(=O)CCCC#Cc2cnc(S(C)(=O)=O)nc2)C(C)C)cc1 ; *C(=O)OCc1ccc(NC(=O)[C@H](CCCNC(N)=O)NC(=O)[C@@H](NC(=O)CCNS(=O)(=O)c2cnc(S(C)(=O)=O)nc2)C(C)C)cc1 ; *C(=O)OCc1ccc(NC(=O)[C@H](CCCNC(N)=O)NC(=O)[C@@H](NC(=O)COCC#Cc2cnc(S(C)(=O)=O)nc2)C(C)C)cc1]

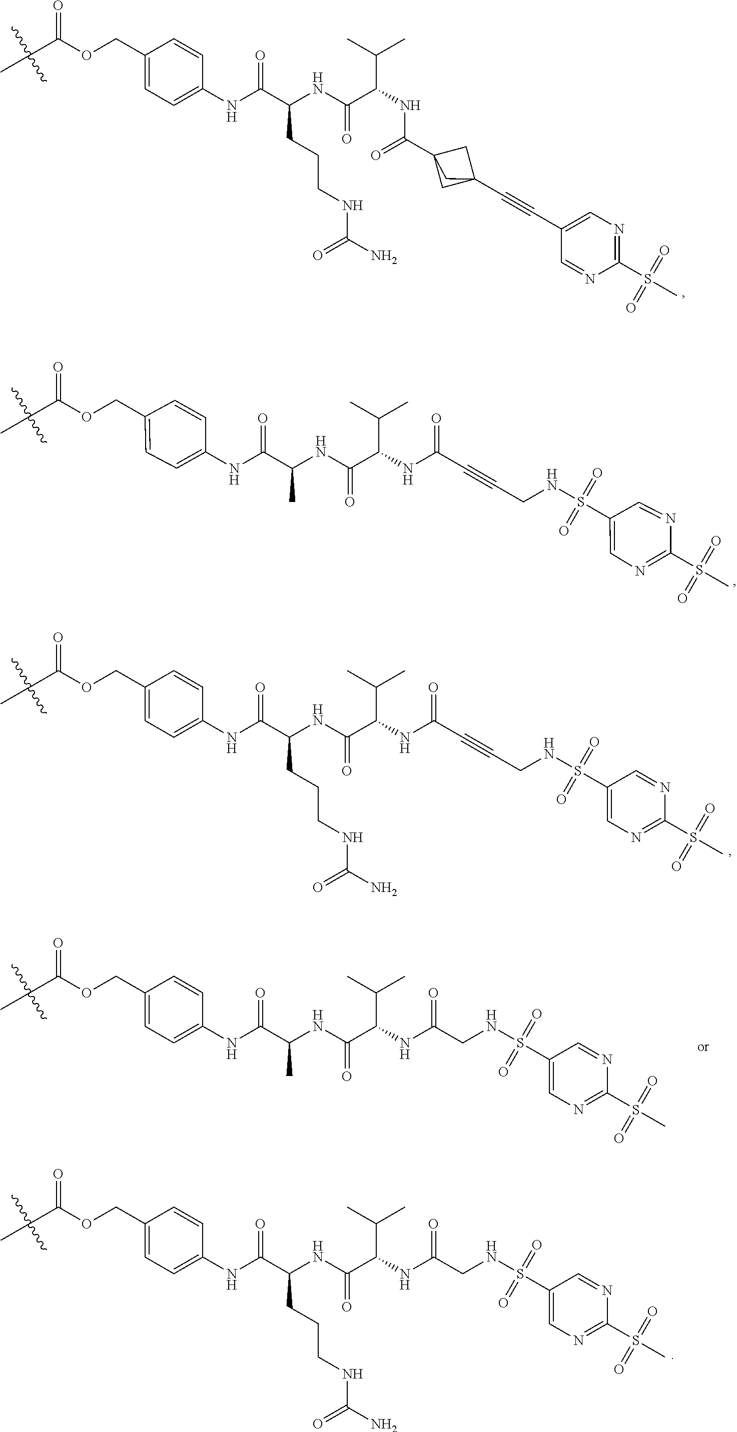 [SMILES: *C(=O)OCc1ccc(NC(=O)[C@H](CCCNC(N)=O)NC(=O)[C@@H](NC(=O)C23CC(C#Cc4cnc(S(C)(=O)=O)nc4)(C2)C3)C(C)C)cc1 ; *C(=O)OCc1ccc(NC(=O)[C@H](C)NC(=O)[C@@H](NC(=O)C#CCNS(=O)(=O)c2cnc(S(C)(=O)=O)nc2)C(C)C)cc1 ; *C(=O)OCc1ccc(NC(=O)[C@H](CCCNC(N)=O)NC(=O)[C@@H](NC(=O)C#CCNS(=O)(=O)c2cnc(S(C)(=O)=O)nc2)C(C)C)cc1 ; *C(=O)OCc1ccc(NC(=O)[C@H](C)NC(=O)[C@@H](NC(=O)CNS(=O)(=O)c2cnc(S(C)(=O)=O)nc2)C(C)C)cc1 ; *C(=O)OCc1ccc(NC(=O)[C@H](CCCNC(N)=O)NC(=O)[C@@H](NC(=O)CNS(=O)(=O)c2cnc(S(C)(=O)=O)nc2)C(C)C)cc1]

20. The cytotoxic drug-linker, the stereoisomer thereof or the pharmaceutically acceptable salt thereof according to claim 19, wherein the cytotoxic drug-linker is selected from:

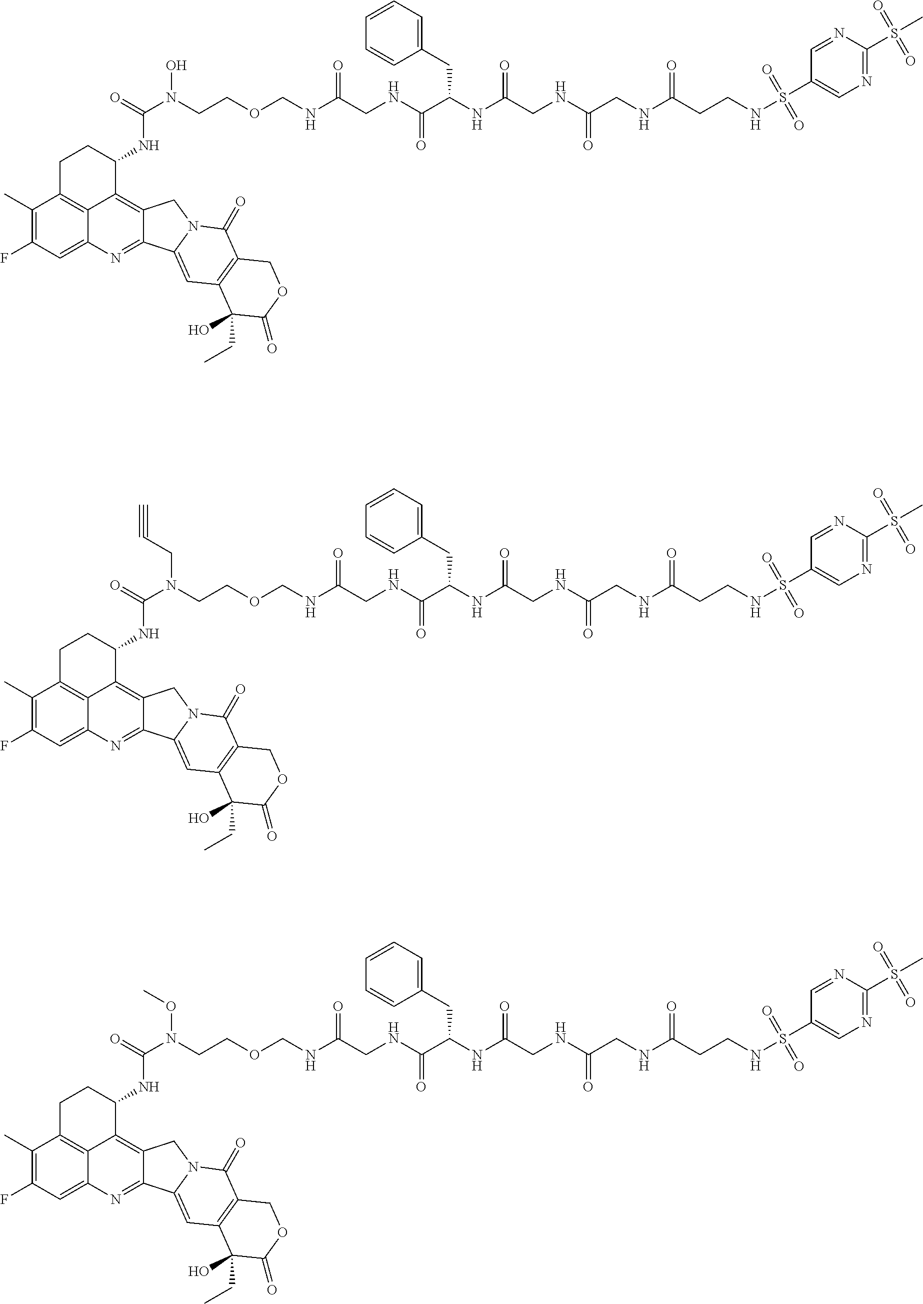 [SMILES: CC[C@@]1(O)C(=O)OCc2c1cc1n(c2=O)Cc2c-1nc1cc(F)c(C)c3c1c2[C@@H](NC(=O)N(O)CCOCNC(=O)CNC(=O)[C@H](Cc1ccccc1)NC(=O)CNC(=O)CNC(=O)CCNS(=O)(=O)c1cnc(S(C)(=O)=O)nc1)CC3 ; C#CCN(CCOCNC(=O)CNC(=O)[C@H](Cc1ccccc1)NC(=O)CNC(=O)CNC(=O)CCNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCNS(=O)(=O)c1cnc(S(C)(=O)=O)nc1)OC)CC3]

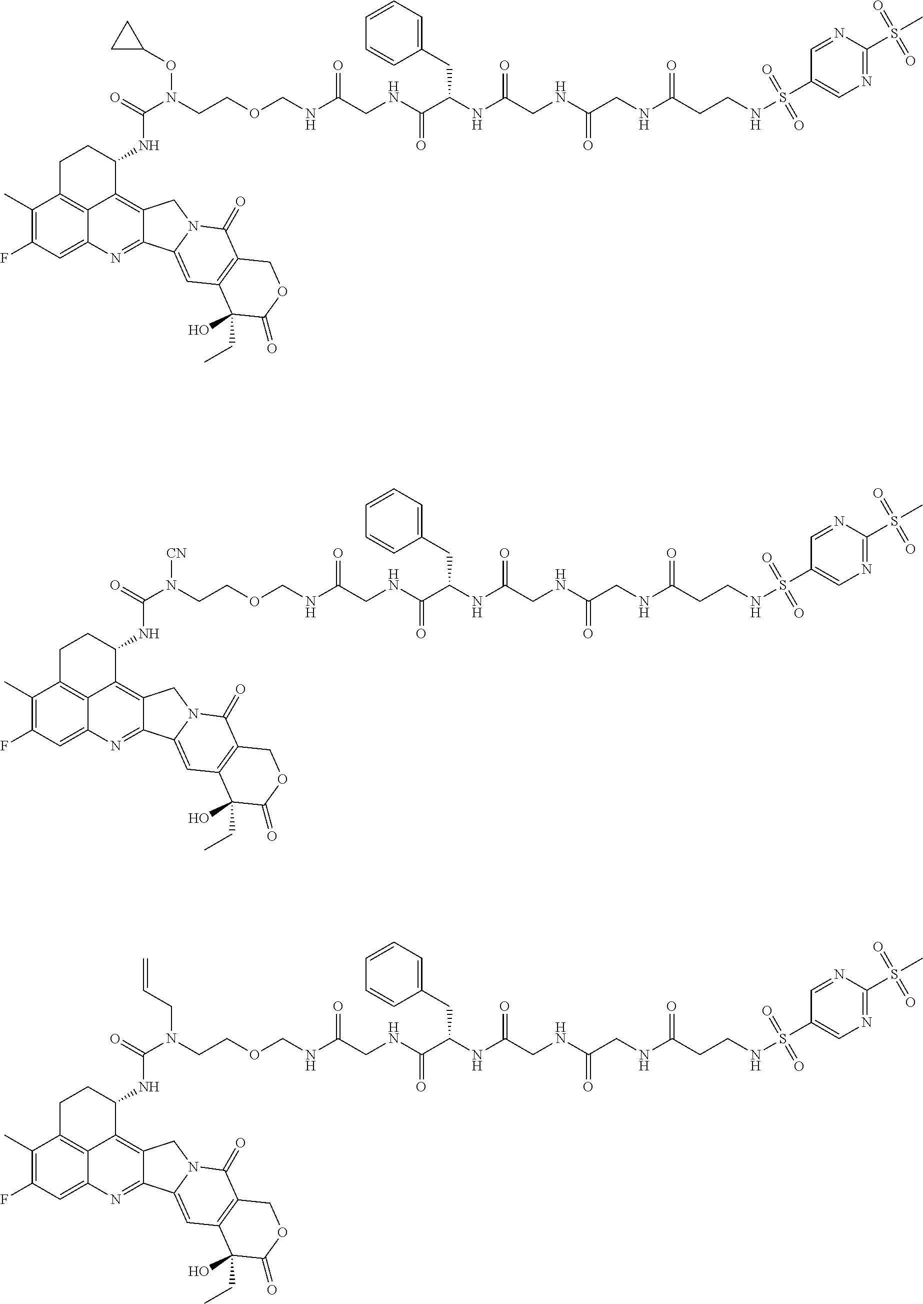 [SMILES: CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCNS(=O)(=O)c1cnc(S(C)(=O)=O)nc1)OC1CC1)CC3 ; CC[C@@]1(O)C(=O)OCc2c1cc1n(c2=O)Cc2c-1nc1cc(F)c(C)c3c1c2[C@@H](NC(=O)N(C#N)CCOCNC(=O)CNC(=O)[C@H](Cc1ccccc1)NC(=O)CNC(=O)CNC(=O)CCNS(=O)(=O)c1cnc(S(C)(=O)=O)nc1)CC3 ; C=CCN(CCOCNC(=O)CNC(=O)[C@H](Cc1ccccc1)NC(=O)CNC(=O)CNC(=O)CCNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

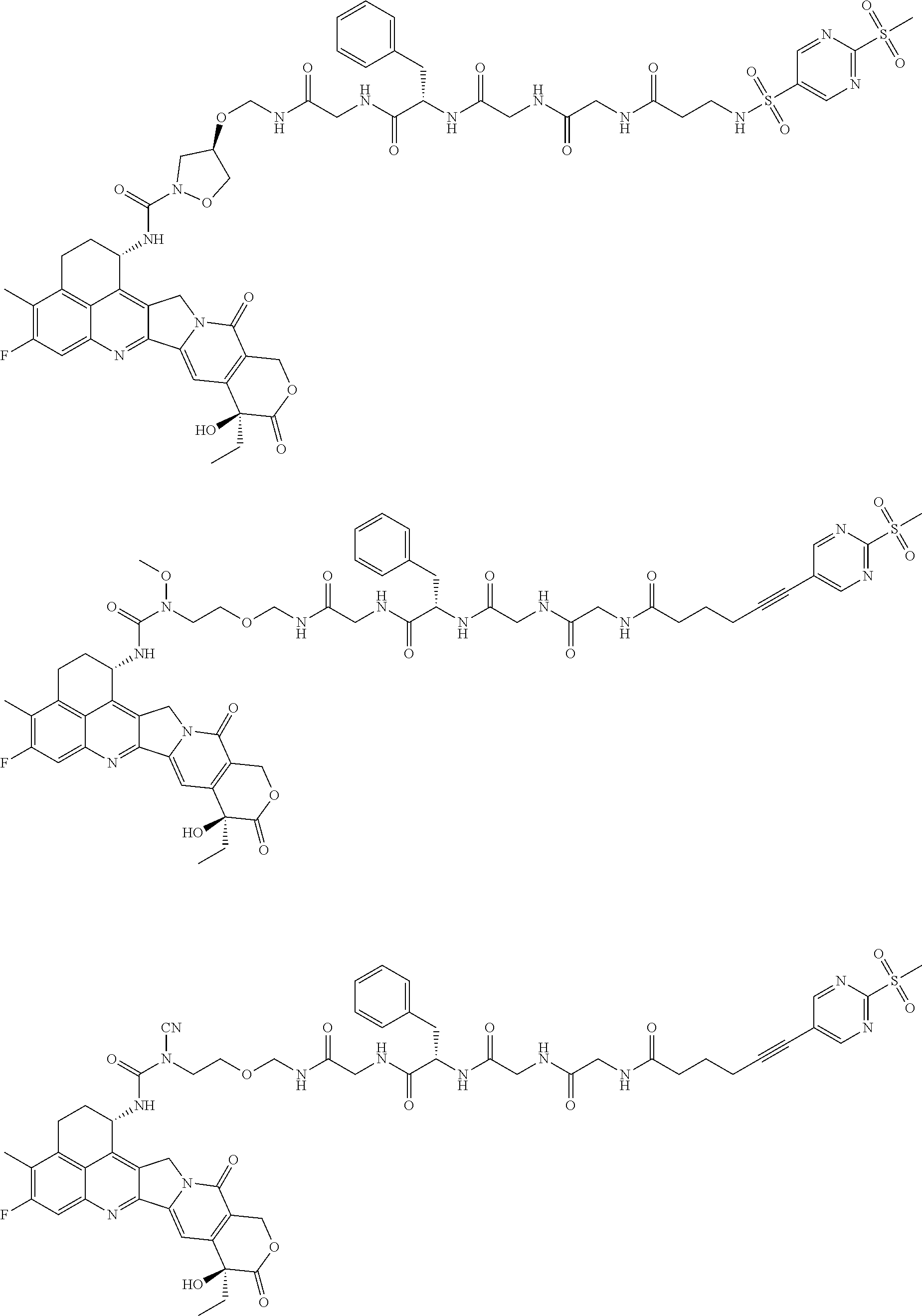 [SMILES: CC[C@@]1(O)C(=O)OCc2c1cc1n(c2=O)Cc2c-1nc1cc(F)c(C)c3c1c2[C@@H](NC(=O)N1C[C@@H](OCNC(=O)CNC(=O)[C@H](Cc2ccccc2)NC(=O)CNC(=O)CNC(=O)CCNS(=O)(=O)c2cnc(S(C)(=O)=O)nc2)CO1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCCC#Cc1cnc(S(C)(=O)=O)nc1)OC)CC3 ; CC[C@@]1(O)C(=O)OCc2c1cc1n(c2=O)Cc2c-1nc1cc(F)c(C)c3c1c2[C@@H](NC(=O)N(C#N)CCOCNC(=O)CNC(=O)[C@H](Cc1ccccc1)NC(=O)CNC(=O)CNC(=O)CCCC#Cc1cnc(S(C)(=O)=O)nc1)CC3]

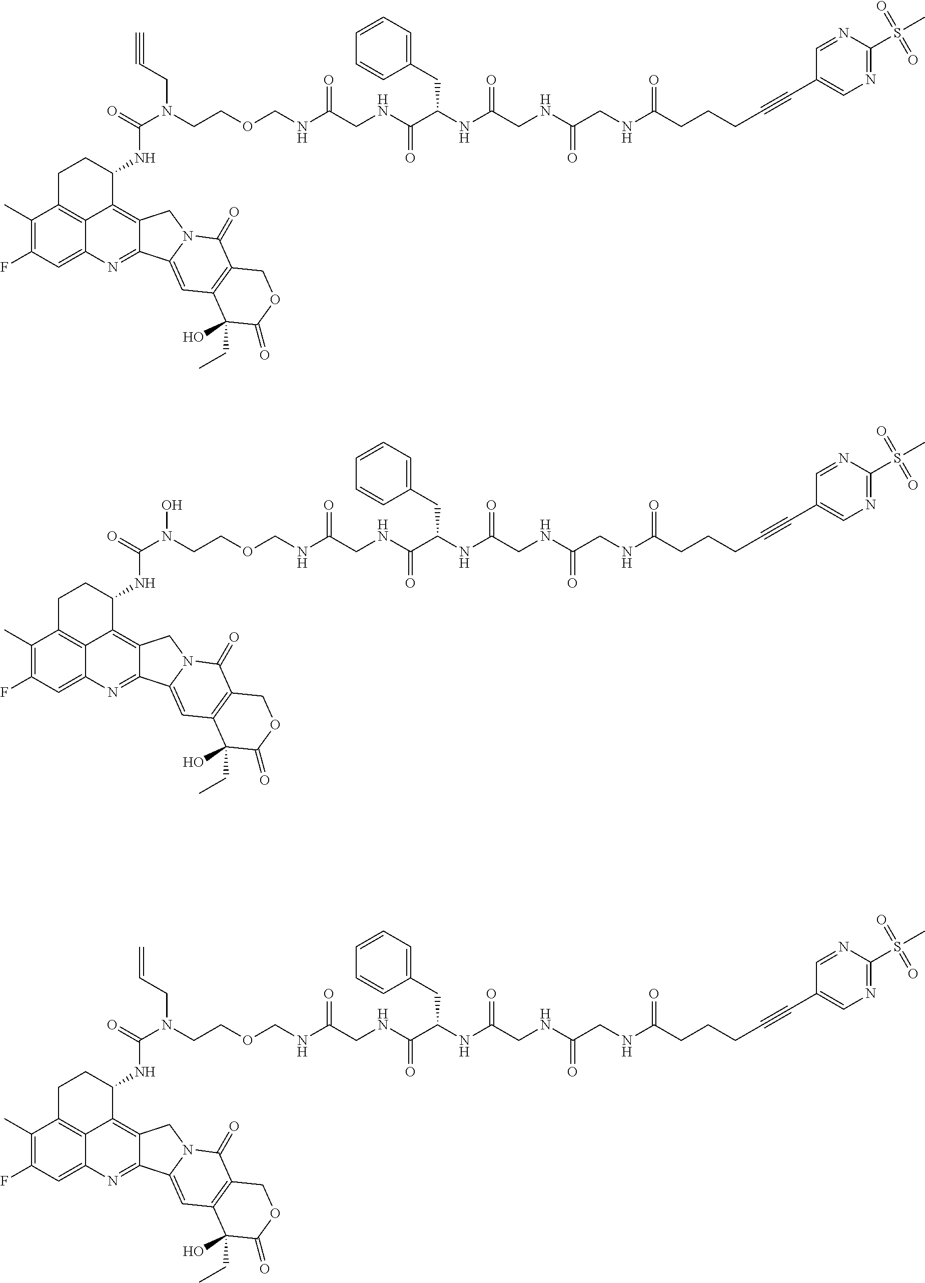 [SMILES: C#CCN(CCOCNC(=O)CNC(=O)[C@H](Cc1ccccc1)NC(=O)CNC(=O)CNC(=O)CCCC#Cc1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(O)CCOCNC(=O)CNC(=O)[C@H](Cc1ccccc1)NC(=O)CNC(=O)CNC(=O)CCCC#Cc1cnc(S(C)(=O)=O)nc1)CC3 ; C=CCN(CCOCNC(=O)CNC(=O)[C@H](Cc1ccccc1)NC(=O)CNC(=O)CNC(=O)CCCC#Cc1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC]

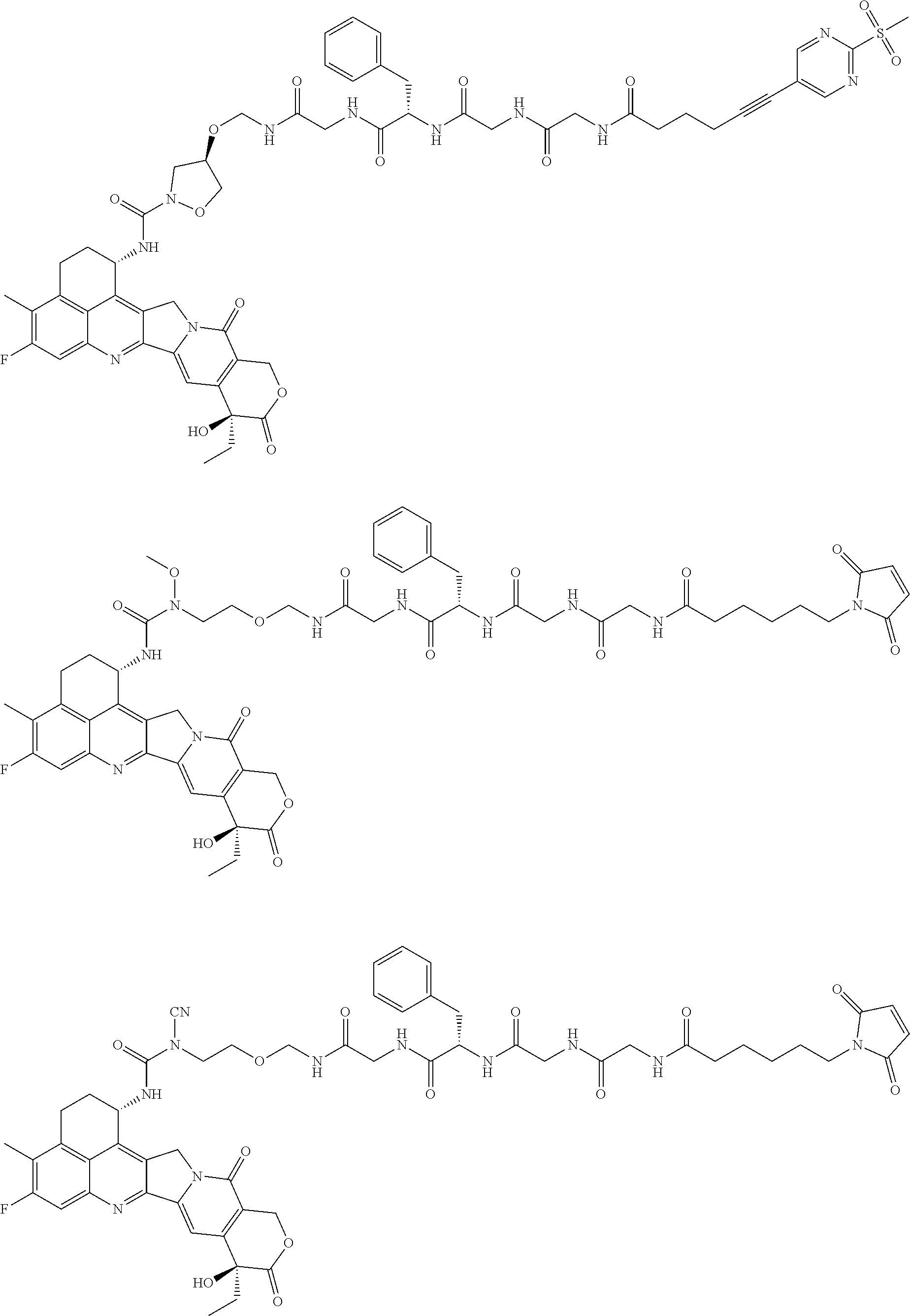 [SMILES: CC[C@@]1(O)C(=O)OCc2c1cc1n(c2=O)Cc2c-1nc1cc(F)c(C)c3c1c2[C@@H](NC(=O)N1C[C@@H](OCNC(=O)CNC(=O)[C@H](Cc2ccccc2)NC(=O)CNC(=O)CNC(=O)CCCC#Cc2cnc(S(C)(=O)=O)nc2)CO1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCCCCN1C(=O)C=CC1=O)OC)CC3 ; CC[C@@]1(O)C(=O)OCc2c1cc1n(c2=O)Cc2c-1nc1cc(F)c(C)c3c1c2[C@@H](NC(=O)N(C#N)CCOCNC(=O)CNC(=O)[C@H](Cc1ccccc1)NC(=O)CNC(=O)CNC(=O)CCCCCN1C(=O)C=CC1=O)CC3]

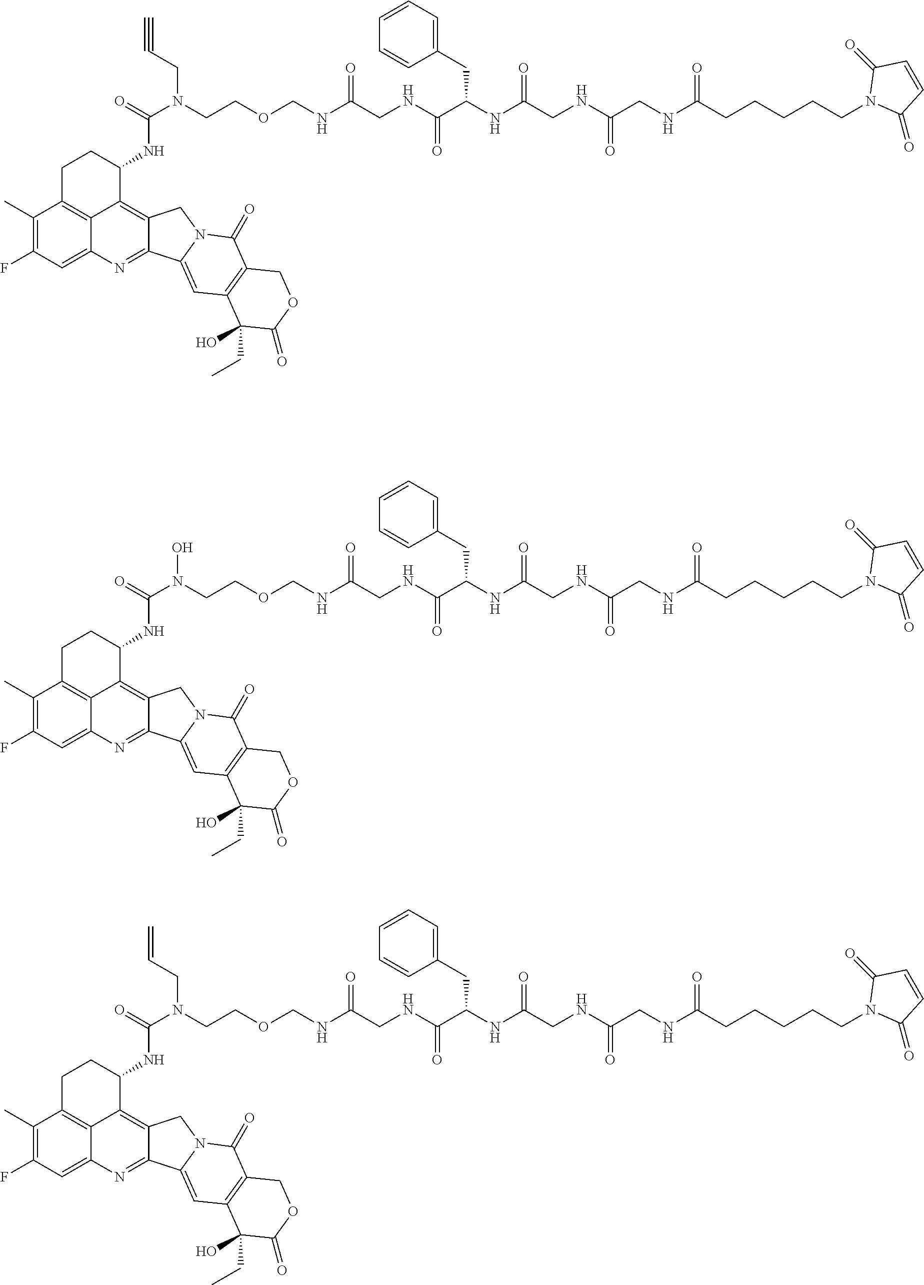 [SMILES: C#CCN(CCOCNC(=O)CNC(=O)[C@H](Cc1ccccc1)NC(=O)CNC(=O)CNC(=O)CCCCCN1C(=O)C=CC1=O)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(O)CCOCNC(=O)CNC(=O)[C@H](Cc1ccccc1)NC(=O)CNC(=O)CNC(=O)CCCCCN1C(=O)C=CC1=O)CC3 ; C=CCN(CCOCNC(=O)CNC(=O)[C@H](Cc1ccccc1)NC(=O)CNC(=O)CNC(=O)CCCCCN1C(=O)C=CC1=O)C(=O)N[C@H]1CCc2c(C)c(F)cc3nc4c(c1c23)Cn1c-4cc2c(c1=O)COC(=O)[C@]2(O)CC]

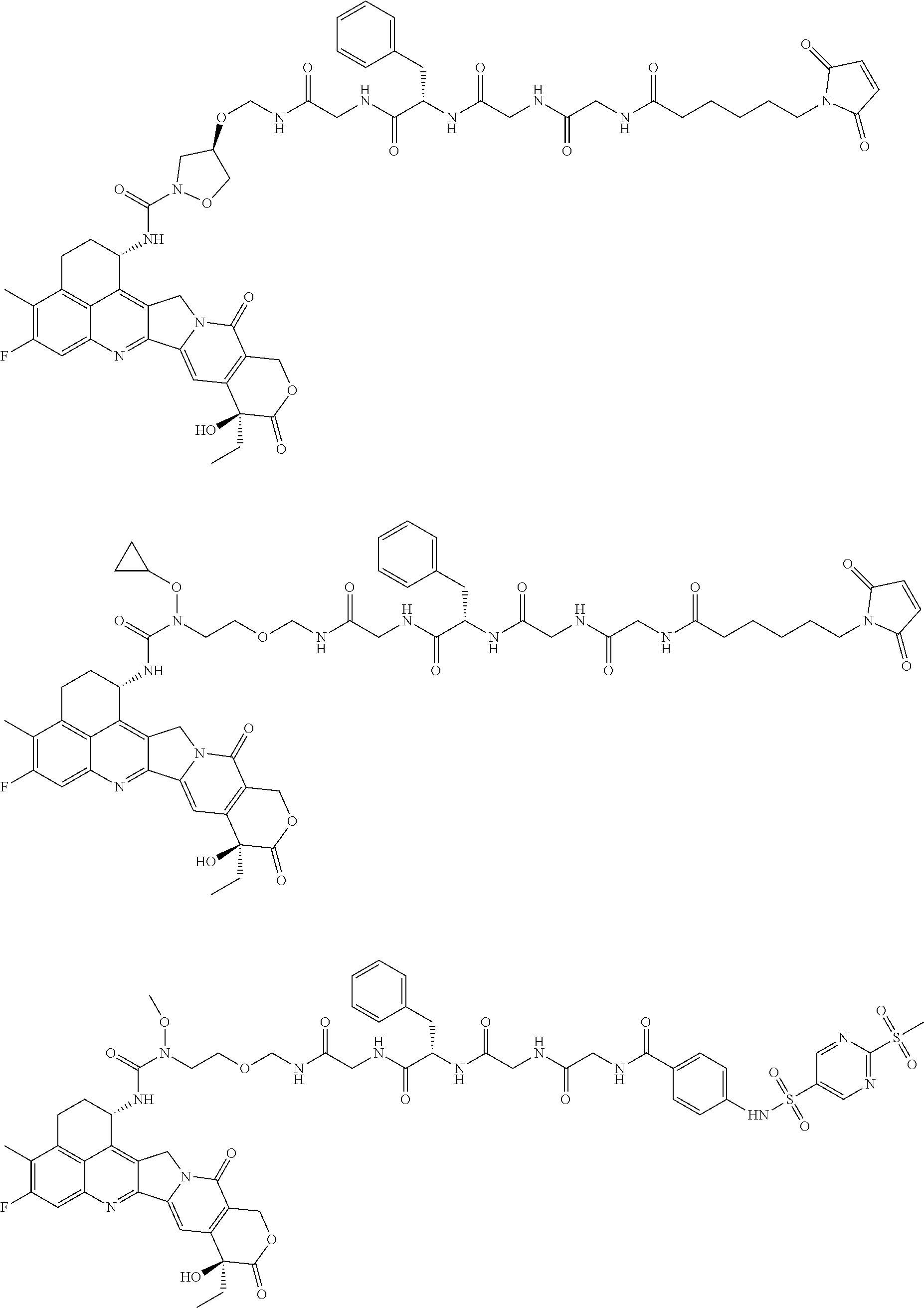 [SMILES: CC[C@@]1(O)C(=O)OCc2c1cc1n(c2=O)Cc2c-1nc1cc(F)c(C)c3c1c2[C@@H](NC(=O)N1C[C@@H](OCNC(=O)CNC(=O)[C@H](Cc2ccccc2)NC(=O)CNC(=O)CNC(=O)CCCCCN2C(=O)C=CC2=O)CO1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)CCCCCN1C(=O)C=CC1=O)OC1CC1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)c1ccc(NS(=O)(=O)c2cnc(S(C)(=O)=O)nc2)cc1)OC)CC3]

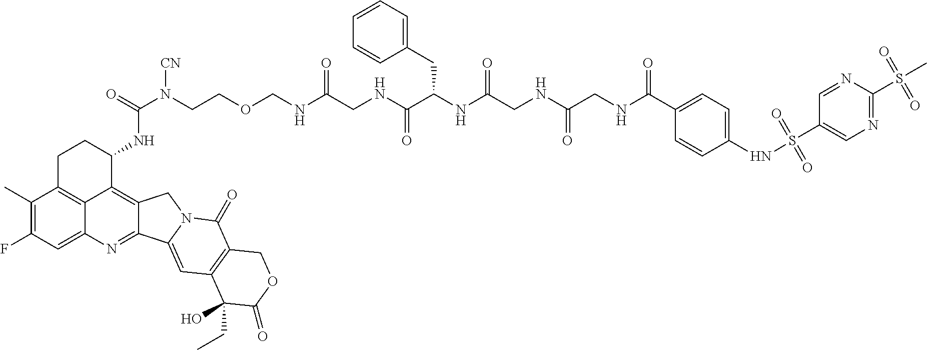 [SMILES: CC[C@@]1(O)C(=O)OCc2c1cc1n(c2=O)Cc2c-1nc1cc(F)c(C)c3c1c2[C@@H](NC(=O)N(C#N)CCOCNC(=O)CNC(=O)[C@H](Cc1ccccc1)NC(=O)CNC(=O)CNC(=O)c1ccc(NS(=O)(=O)c2cnc(S(C)(=O)=O)nc2)cc1)CC3]

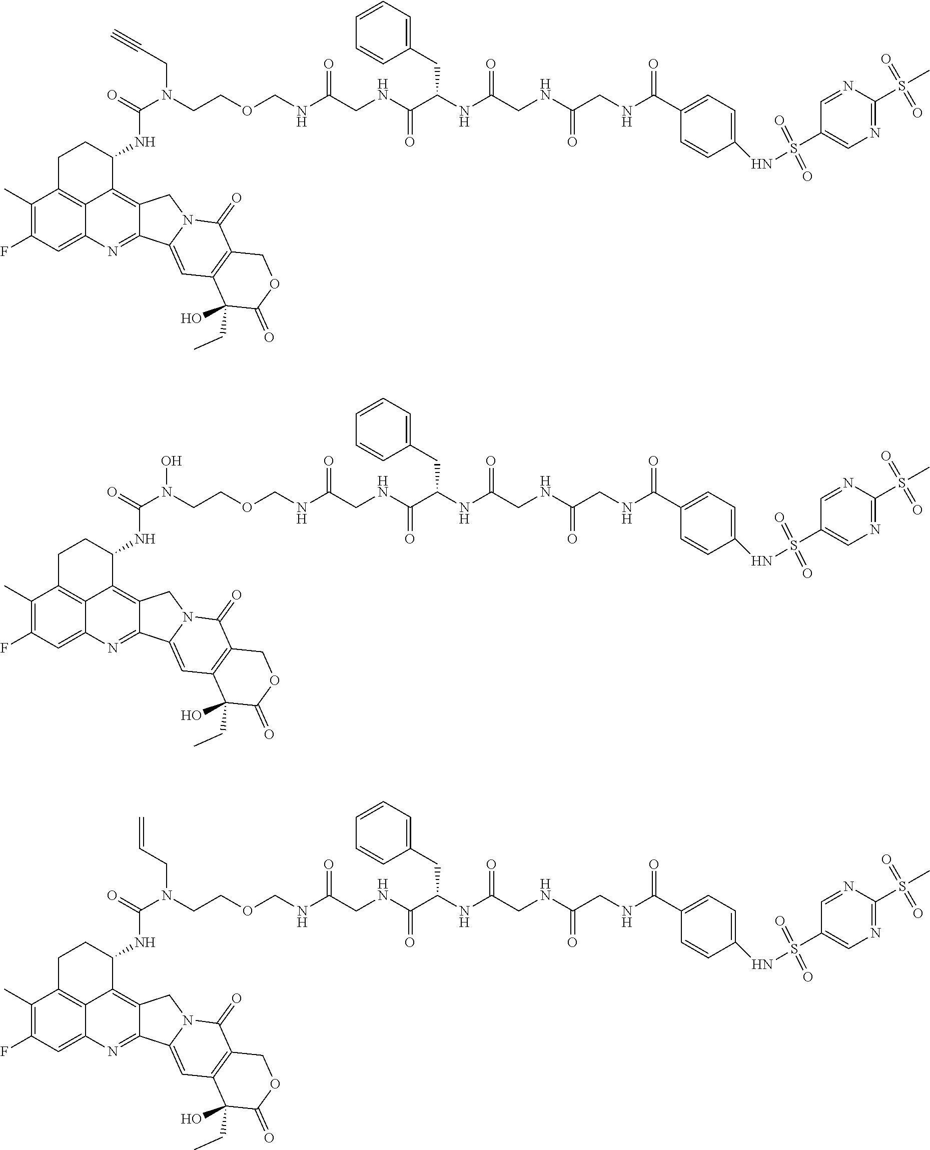

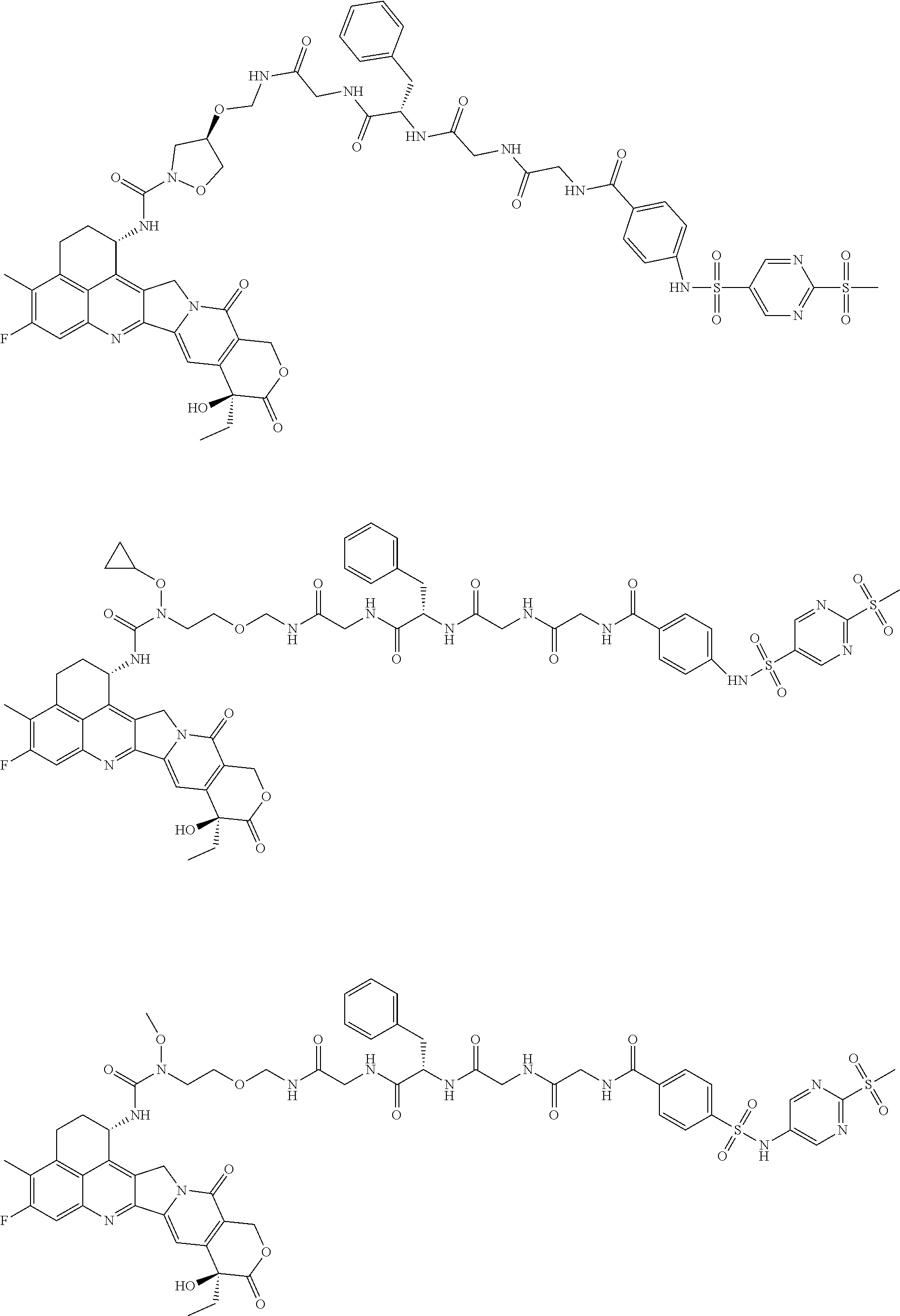

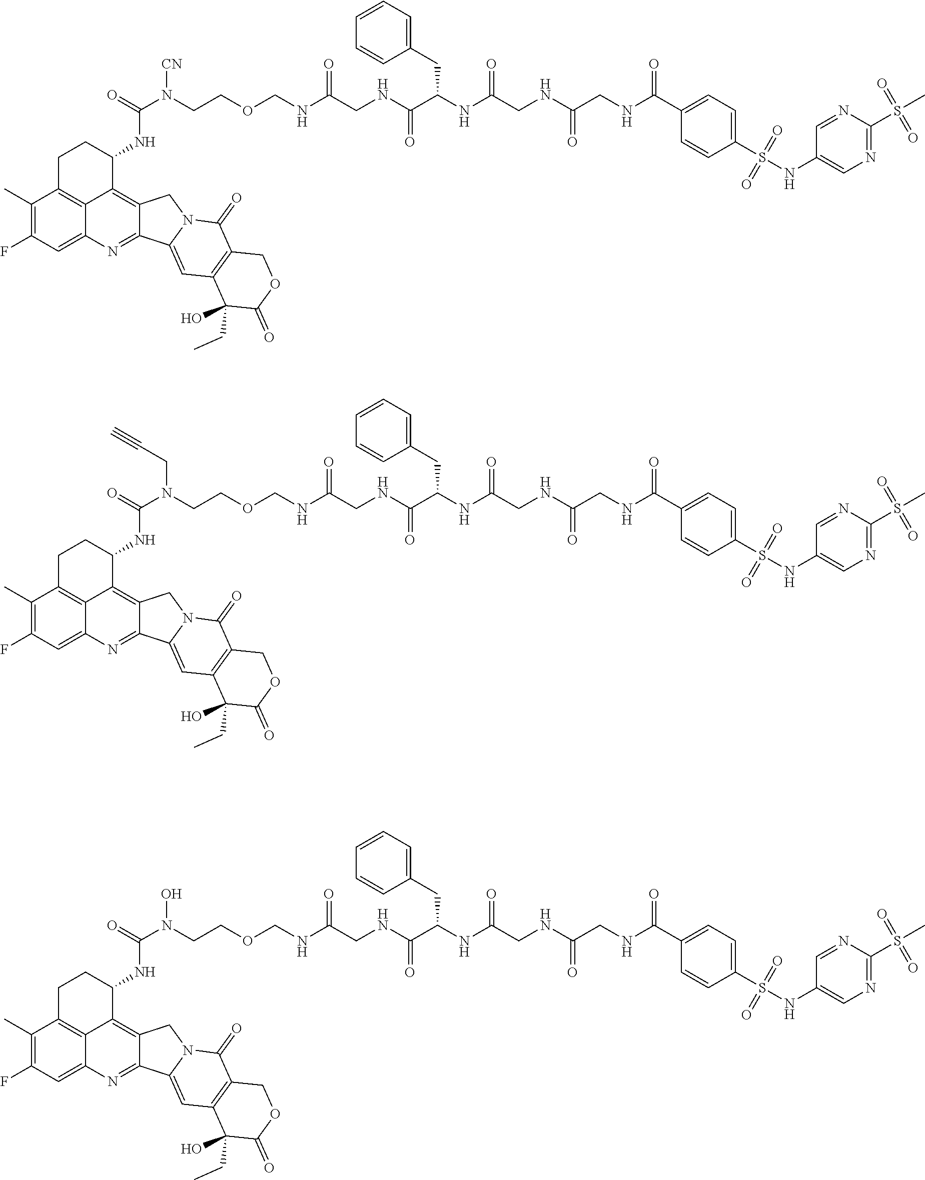

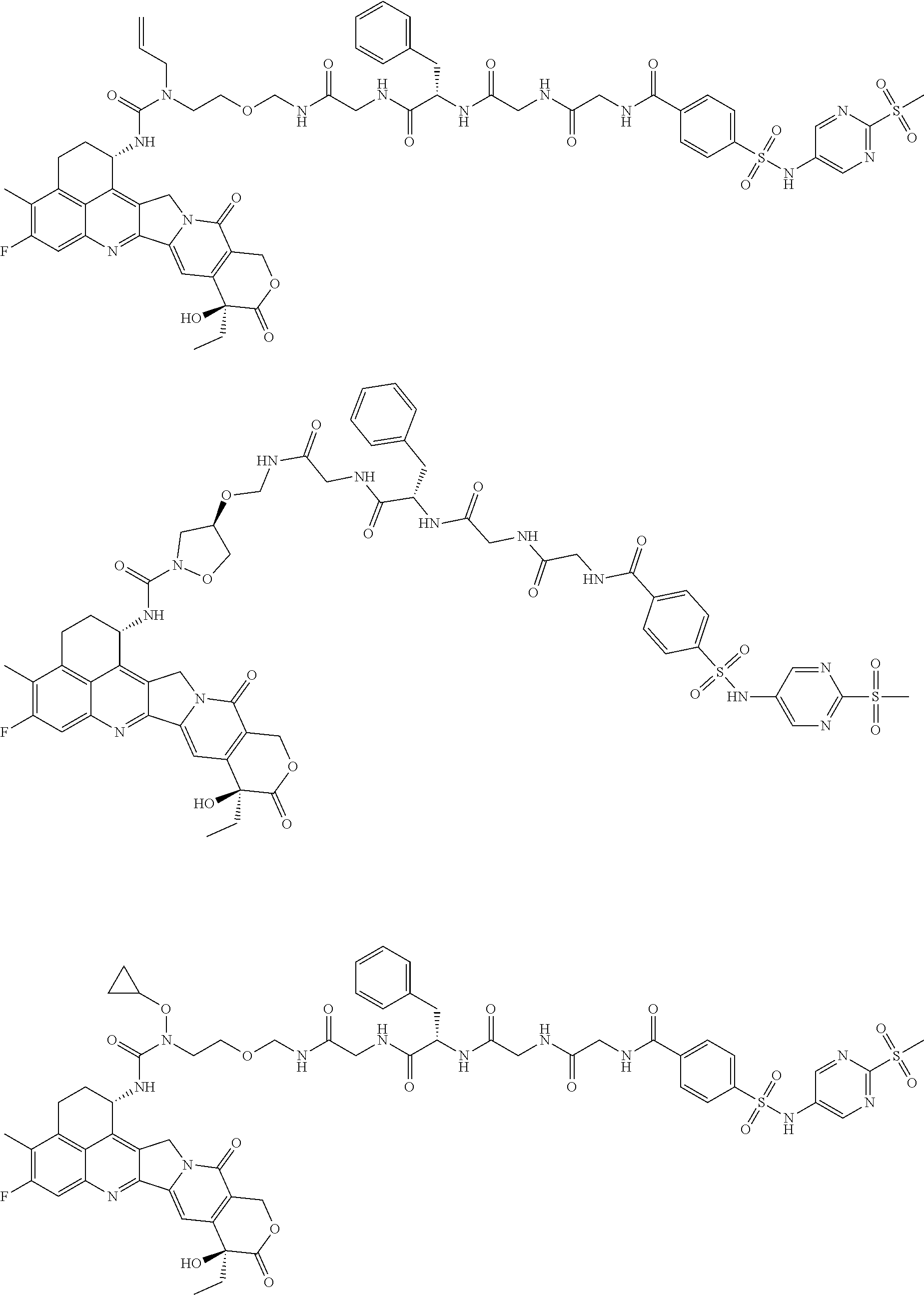

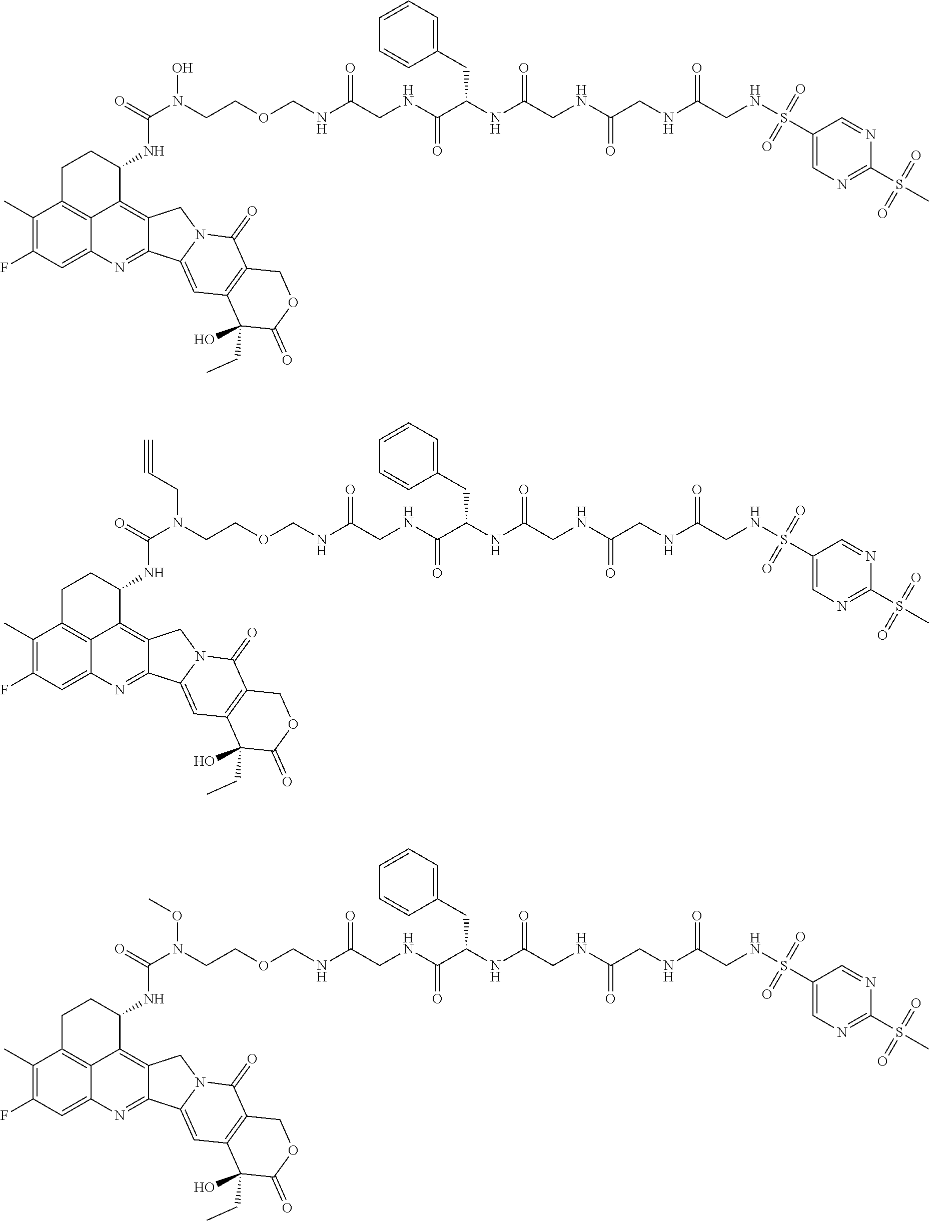

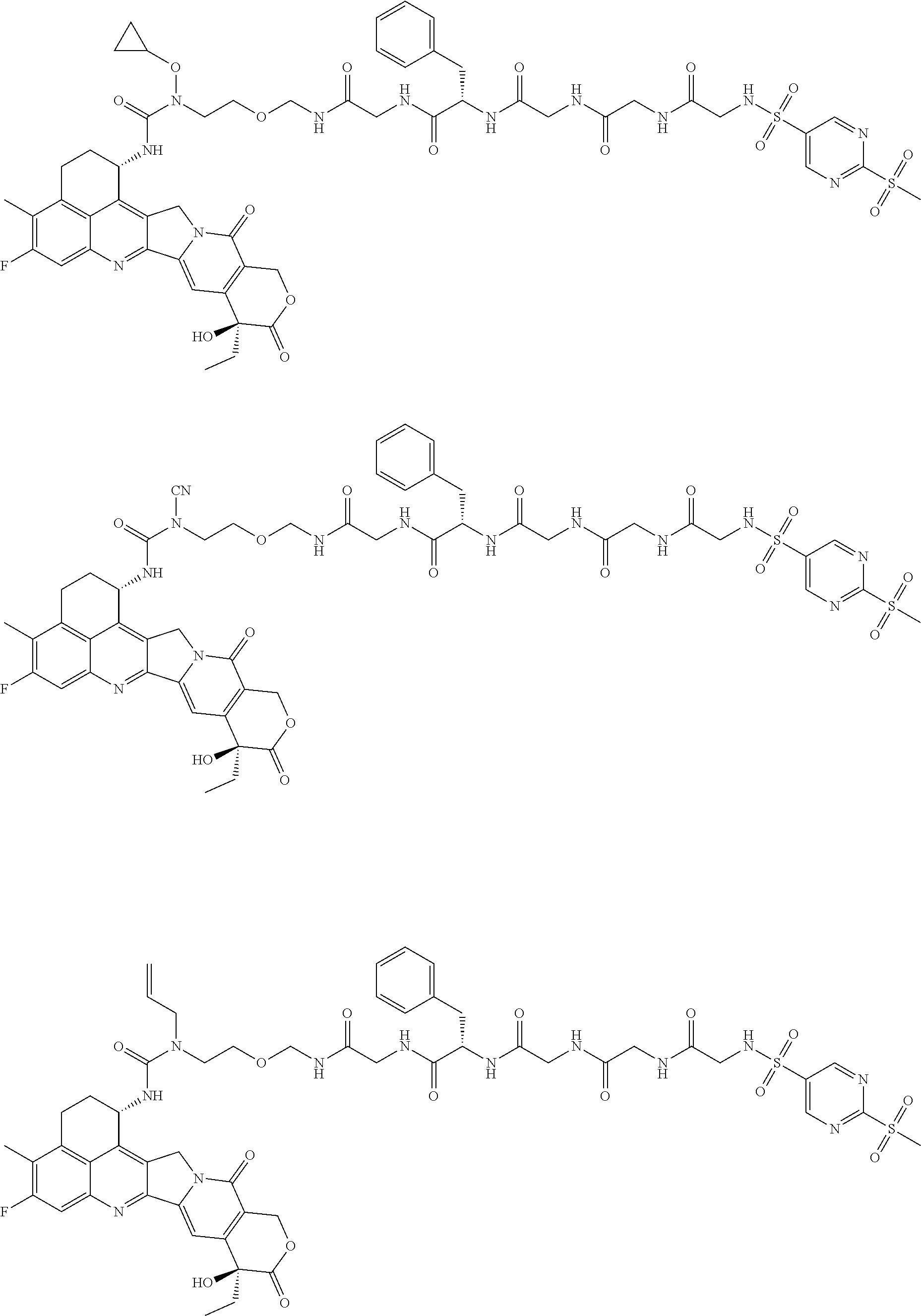

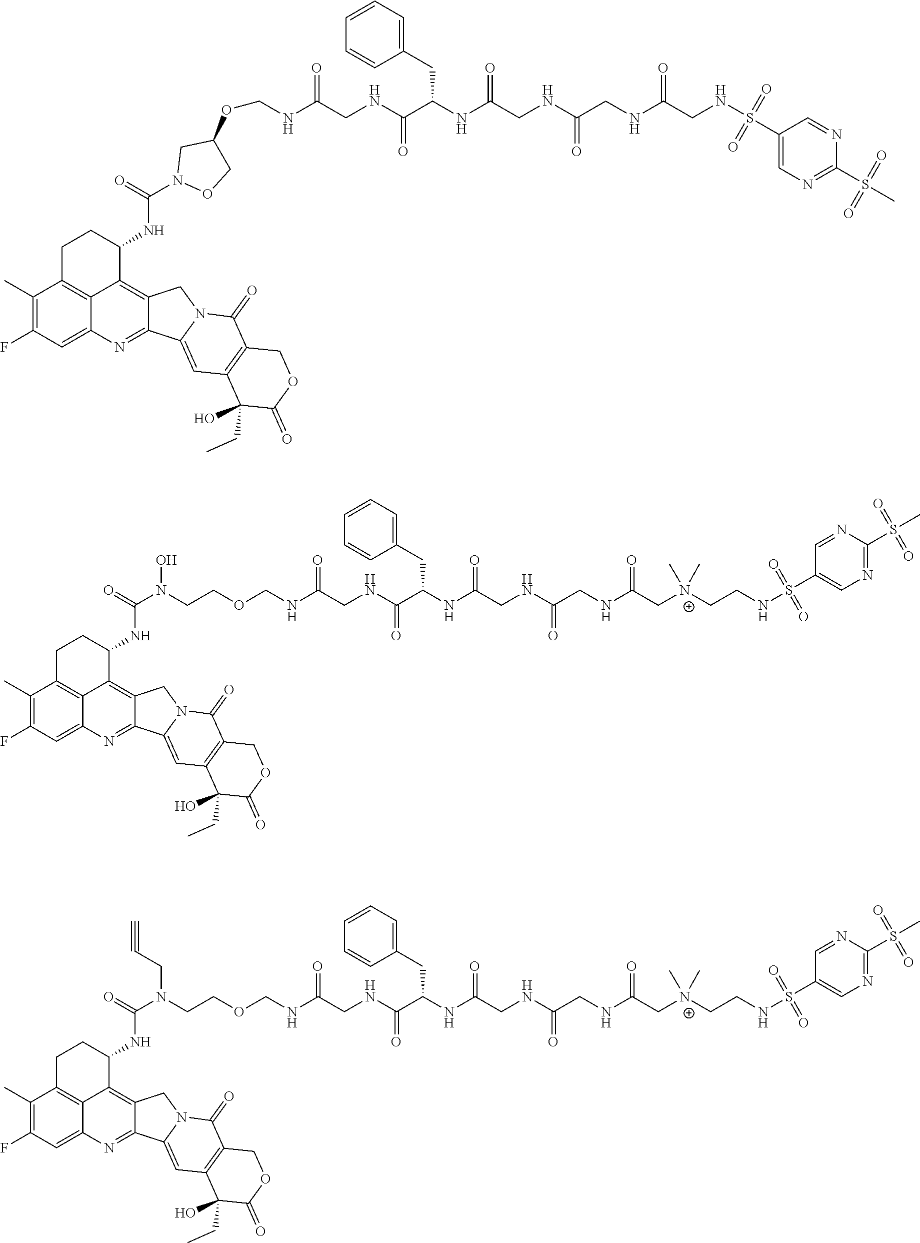

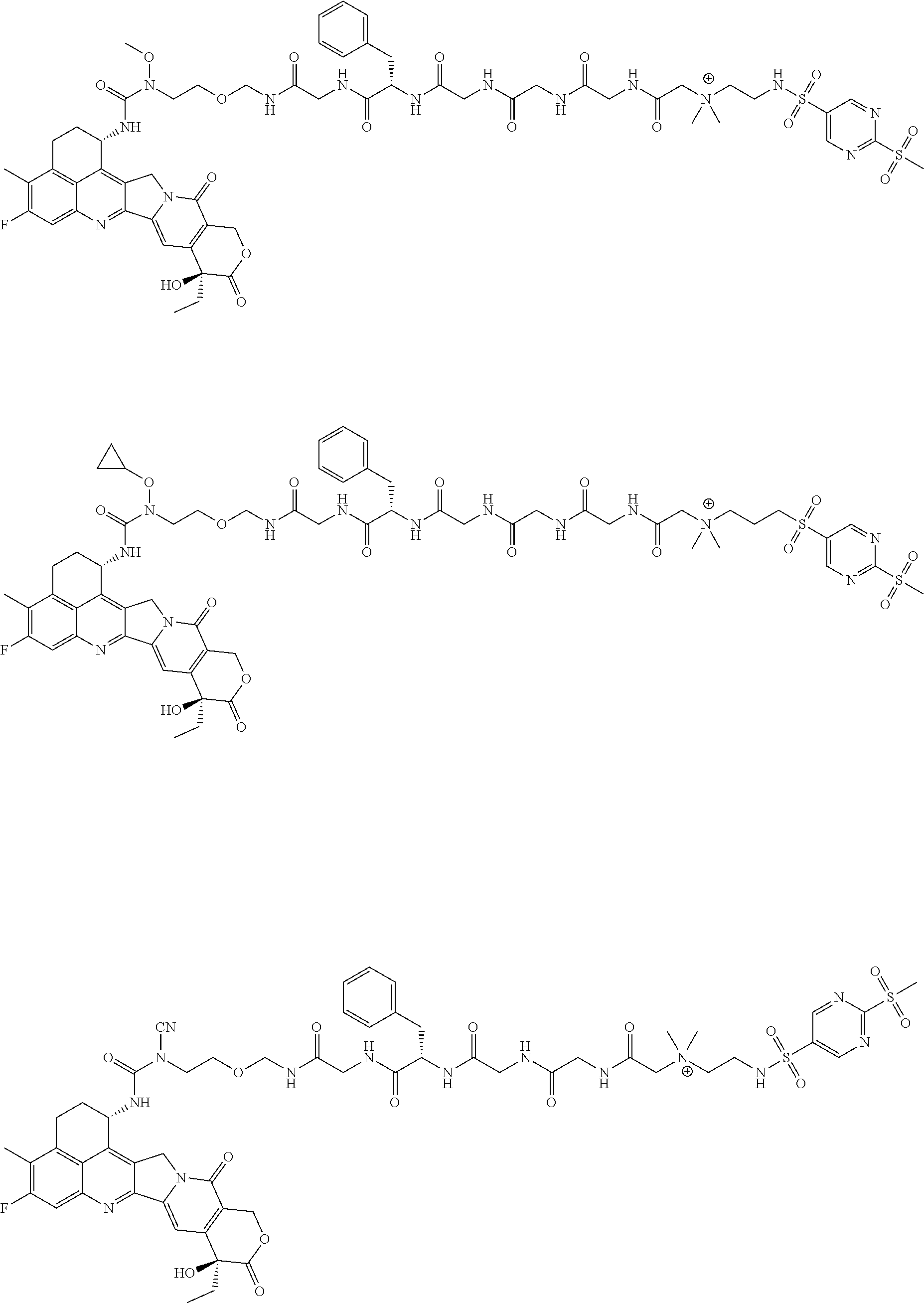

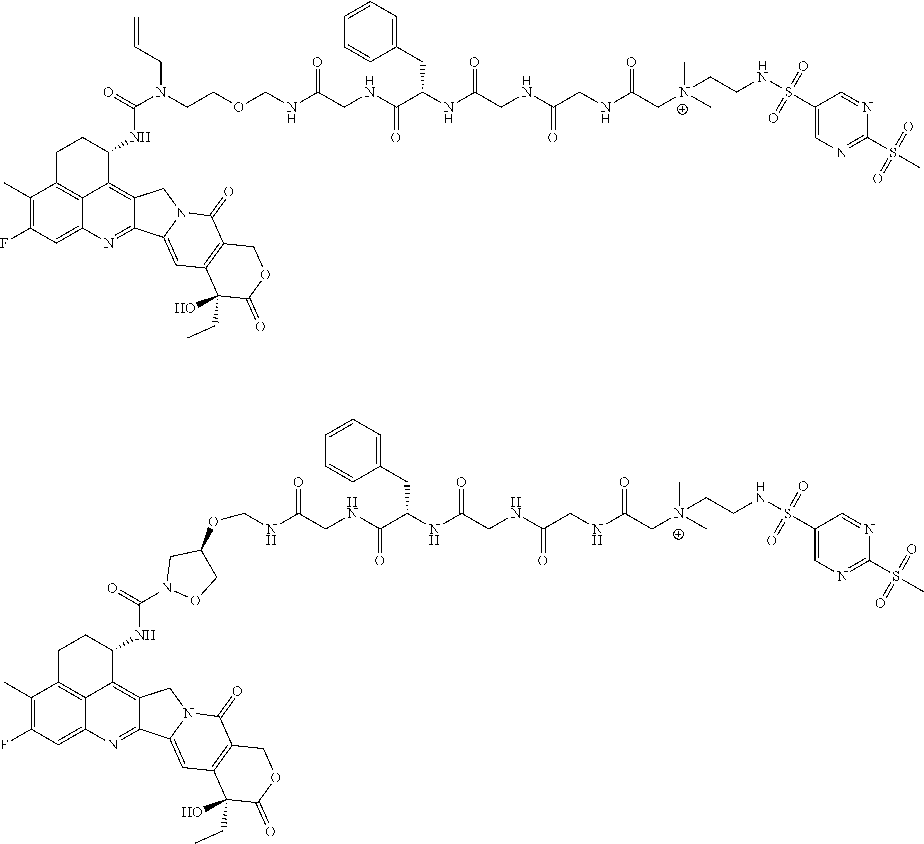

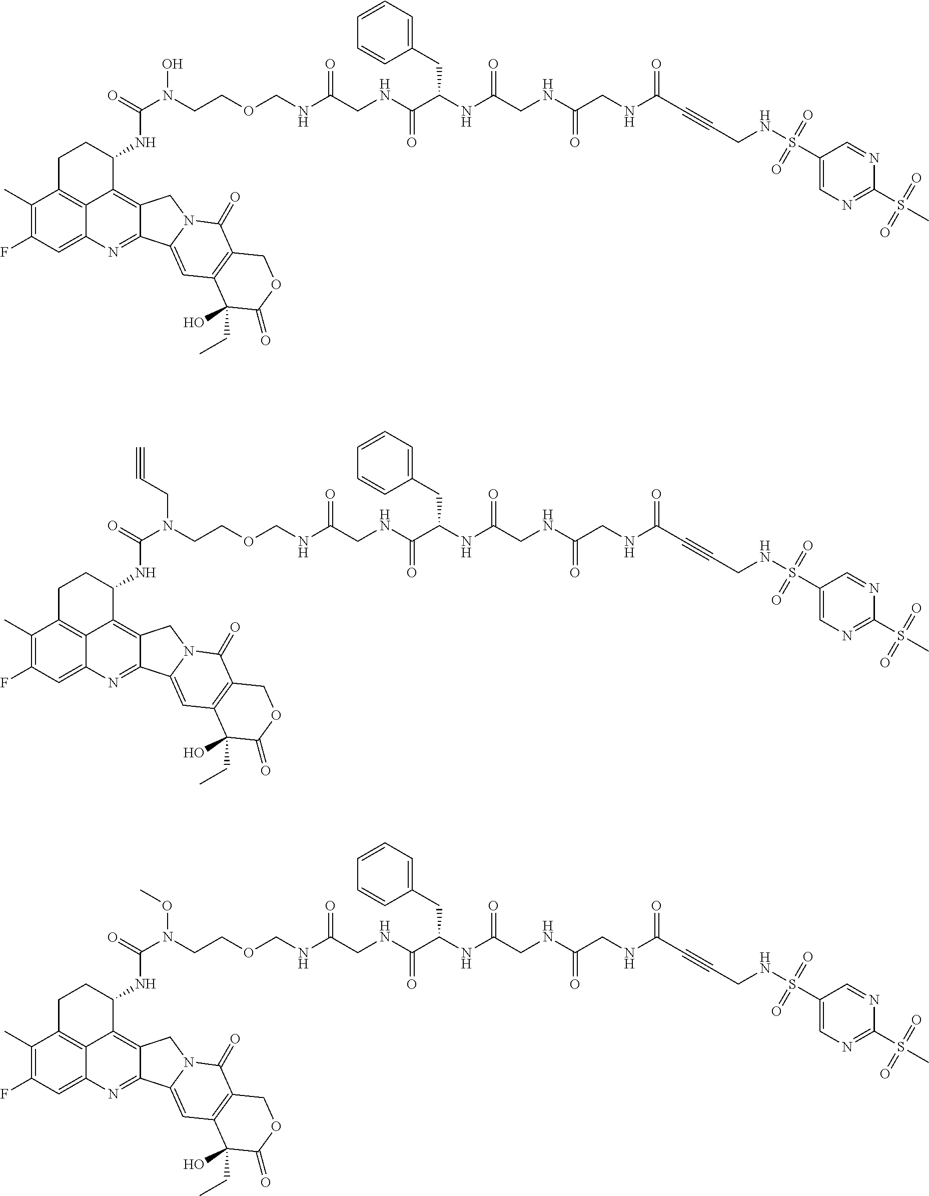 [SMILES: CC[C@@]1(O)C(=O)OCc2c1cc1n(c2=O)Cc2c-1nc1cc(F)c(C)c3c1c2[C@@H](NC(=O)N(O)CCOCNC(=O)CNC(=O)[C@H](Cc1ccccc1)NC(=O)CNC(=O)CNC(=O)C#CCNS(=O)(=O)c1cnc(S(C)(=O)=O)nc1)CC3 ; C#CCN(CCOCNC(=O)CNC(=O)[C@H](Cc1ccccc1)NC(=O)CNC(=O)CNC(=O)C#CCNS(=O)(=O)c1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)C#CCNS(=O)(=O)c1cnc(S(C)(=O)=O)nc1)OC)CC3]

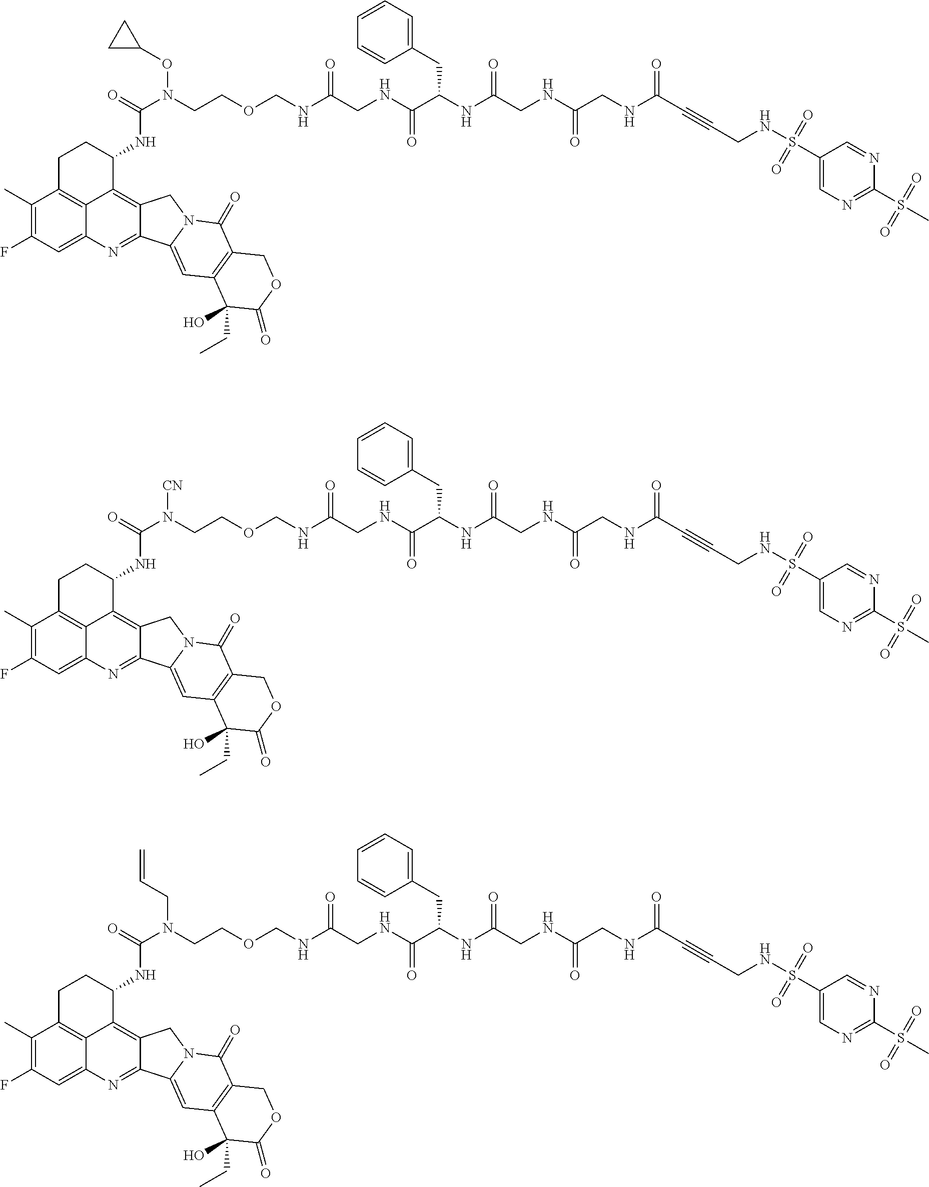

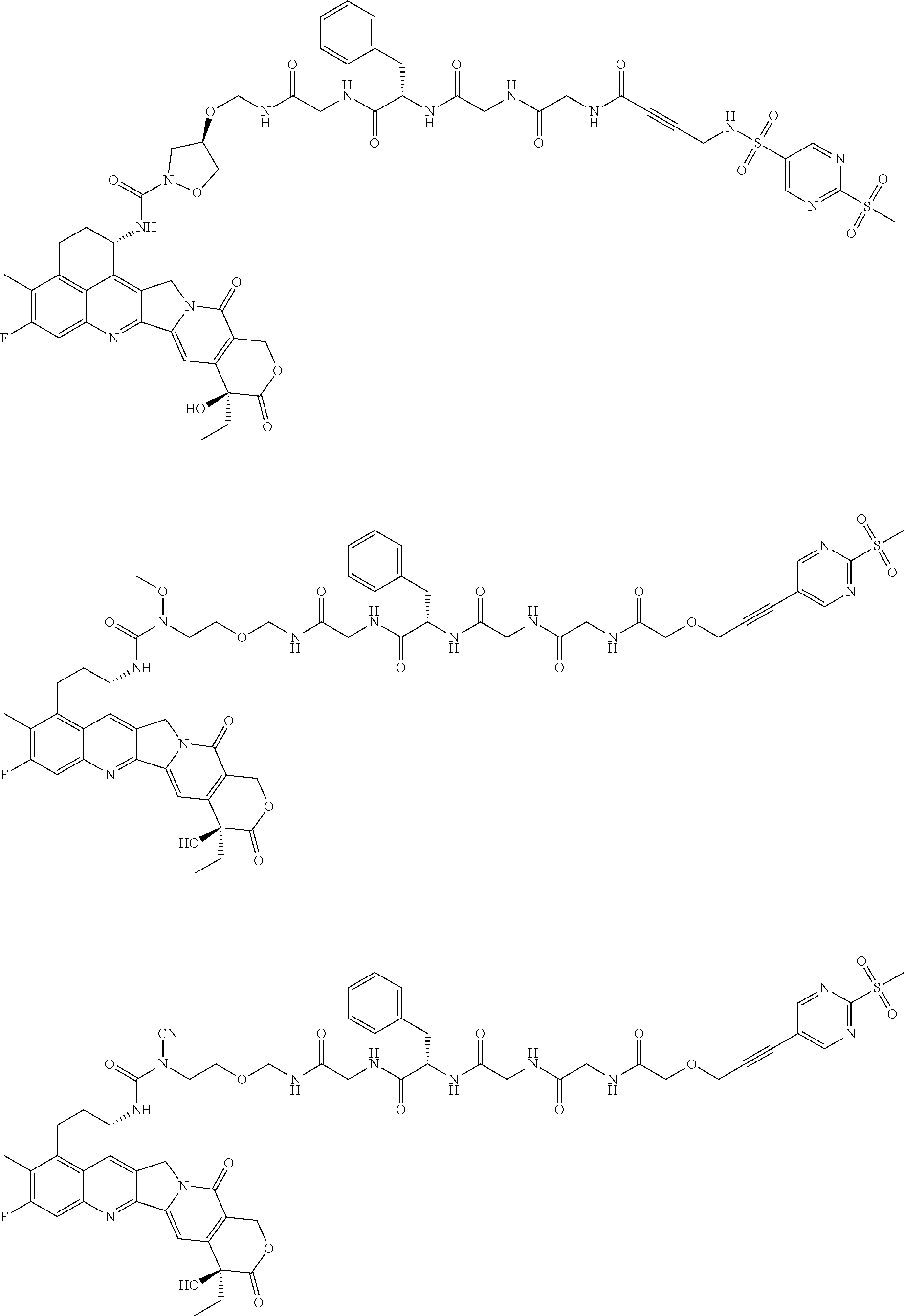 [SMILES: CC[C@@]1(O)C(=O)OCc2c1cc1n(c2=O)Cc2c-1nc1cc(F)c(C)c3c1c2[C@@H](NC(=O)N1C[C@@H](OCNC(=O)CNC(=O)[C@H](Cc2ccccc2)NC(=O)CNC(=O)CNC(=O)C#CCNS(=O)(=O)c2cnc(S(C)(=O)=O)nc2)CO1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)COCC#Cc1cnc(S(C)(=O)=O)nc1)OC)CC3 ; CC[C@@]1(O)C(=O)OCc2c1cc1n(c2=O)Cc2c-1nc1cc(F)c(C)c3c1c2[C@@H](NC(=O)N(C#N)CCOCNC(=O)CNC(=O)[C@H](Cc1ccccc1)NC(=O)CNC(=O)CNC(=O)COCC#Cc1cnc(S(C)(=O)=O)nc1)CC3]

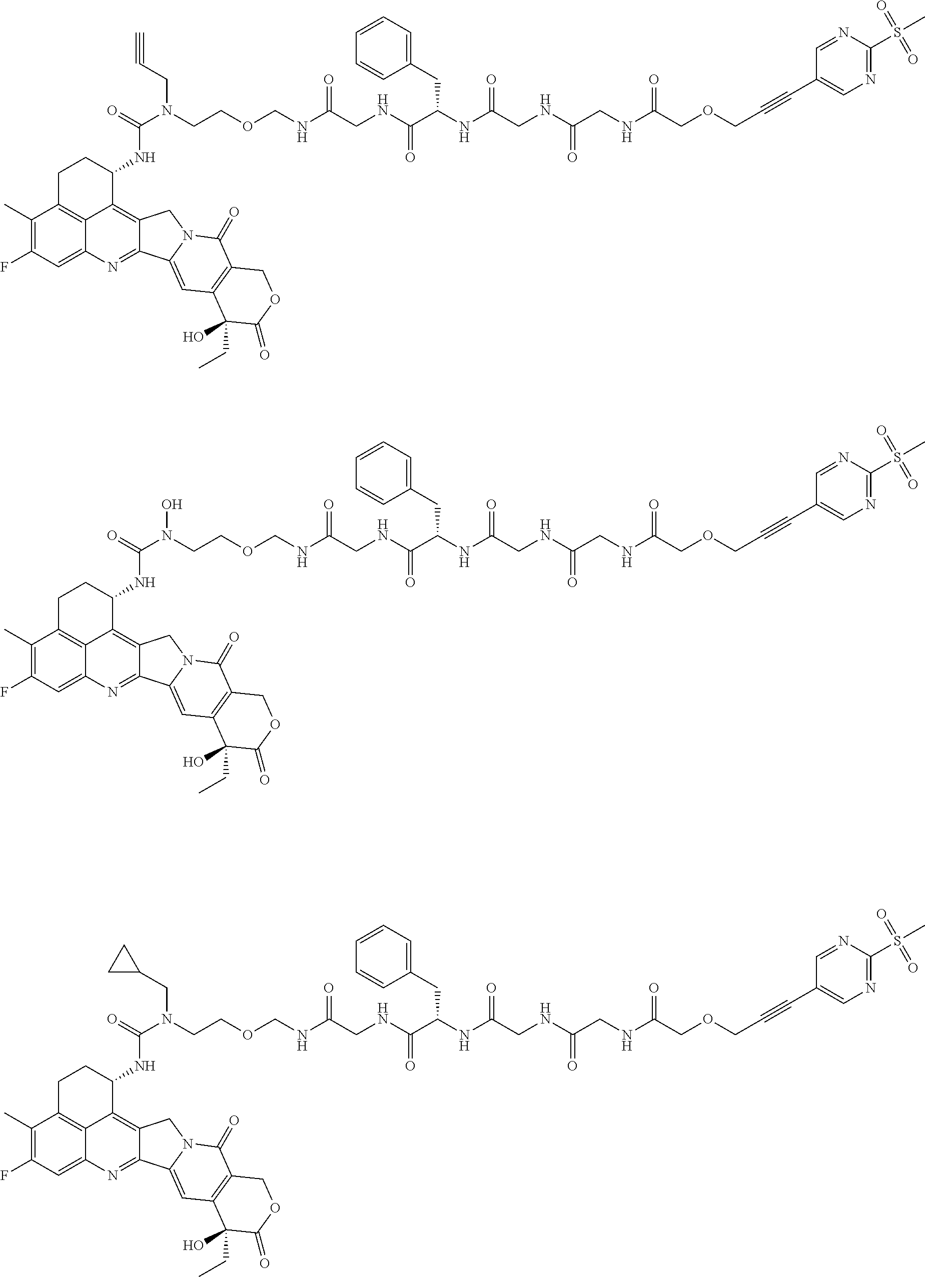 [SMILES: C#CCN(CCOCNC(=O)CNC(=O)[C@H](Cc1ccccc1)NC(=O)CNC(=O)CNC(=O)COCC#Cc1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(O)CCOCNC(=O)CNC(=O)[C@H](Cc1ccccc1)NC(=O)CNC(=O)CNC(=O)COCC#Cc1cnc(S(C)(=O)=O)nc1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)COCC#Cc1cnc(S(C)(=O)=O)nc1)CC1CC1)CC3]

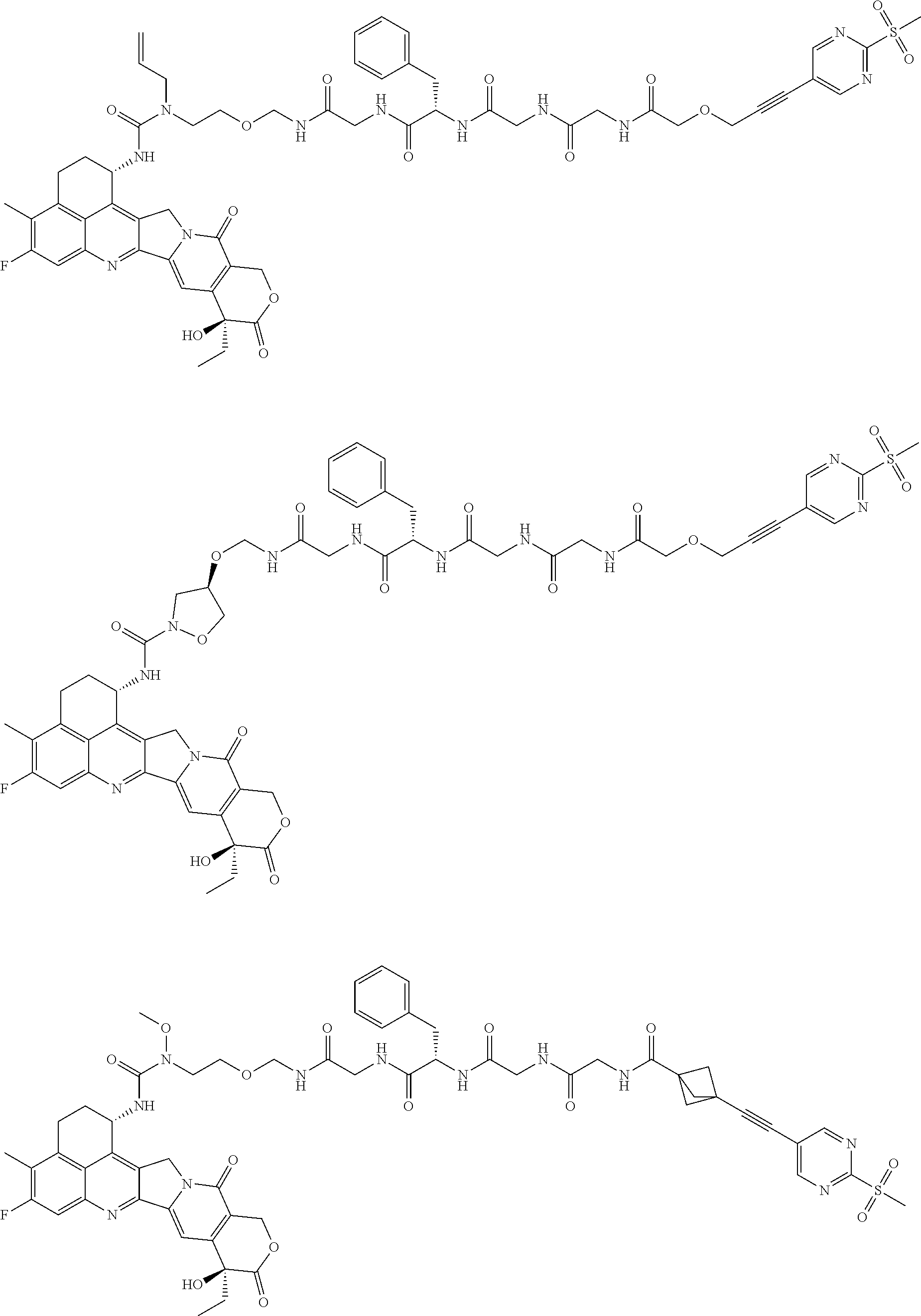 [SMILES: C=CCN(CCOCNC(=O)CNC(=O)[C@H](Cc1ccccc1)NC(=O)CNC(=O)CNC(=O)COCC#Cc1cnc(S(C)(=O)=O)nc1)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N1C[C@@H](OCNC(=O)CNC(=O)[C@H](Cc2ccccc2)NC(=O)CNC(=O)CNC(=O)COCC#Cc2cnc(S(C)(=O)=O)nc2)CO1)CC3 ; CC[C@@]1(O)C(=O)OCc2c1cc1n(c2=O)Cc2c-1nc1cc(F)c(C)c3c1c2[C@@H](NC(=O)N(CCOCNC(=O)CNC(=O)[C@H](Cc1ccccc1)NC(=O)CNC(=O)CNC(=O)C12CC(C#Cc4cnc(S(C)(=O)=O)nc4)(C1)C2)OC)CC3]

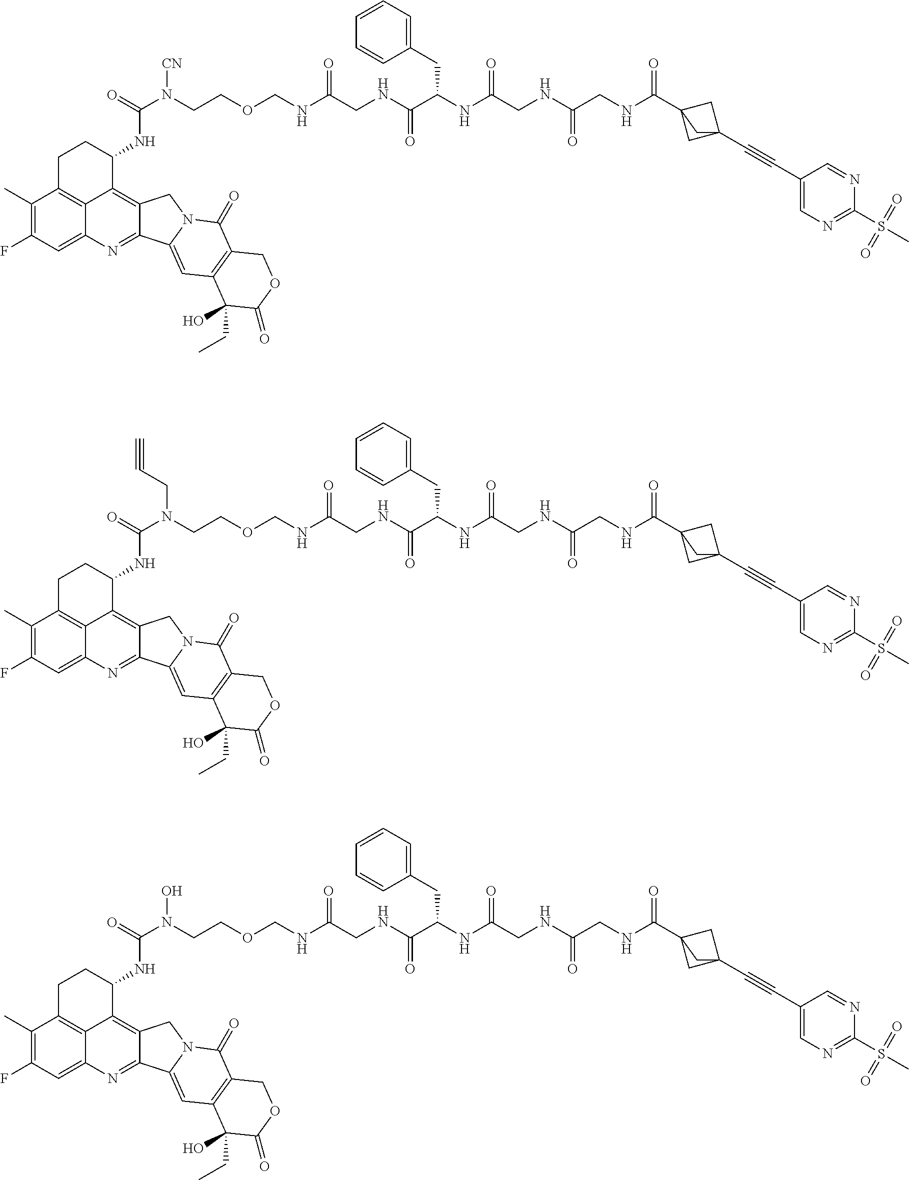

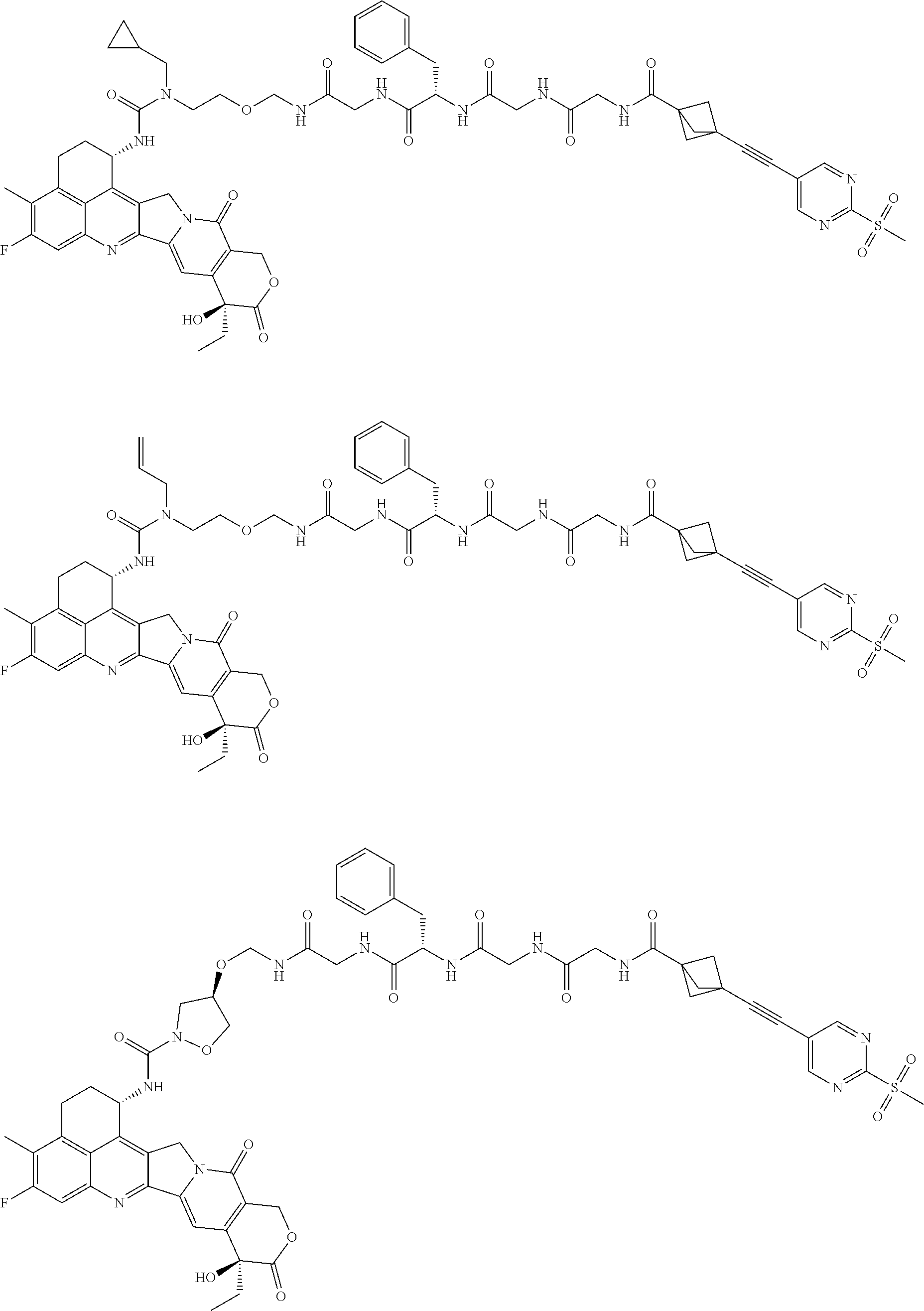

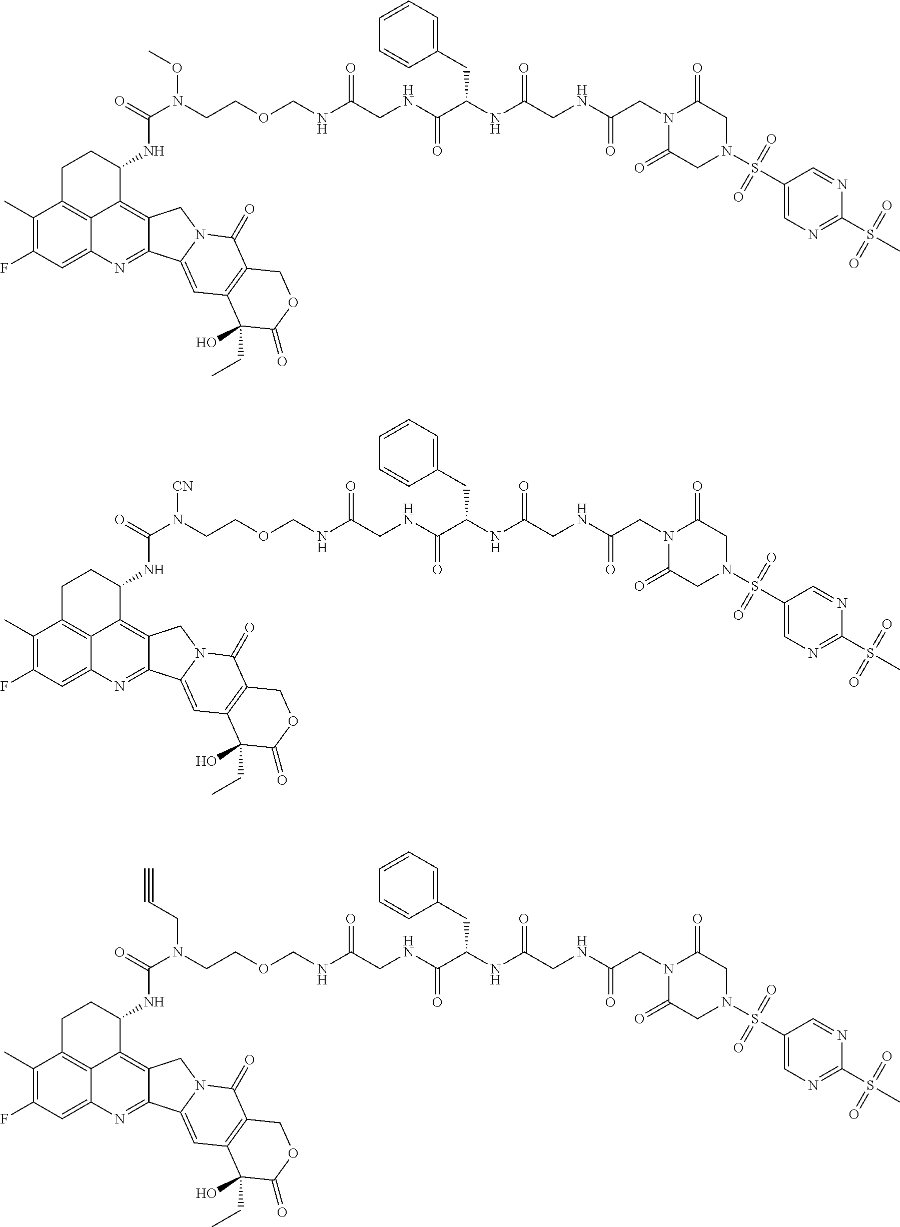

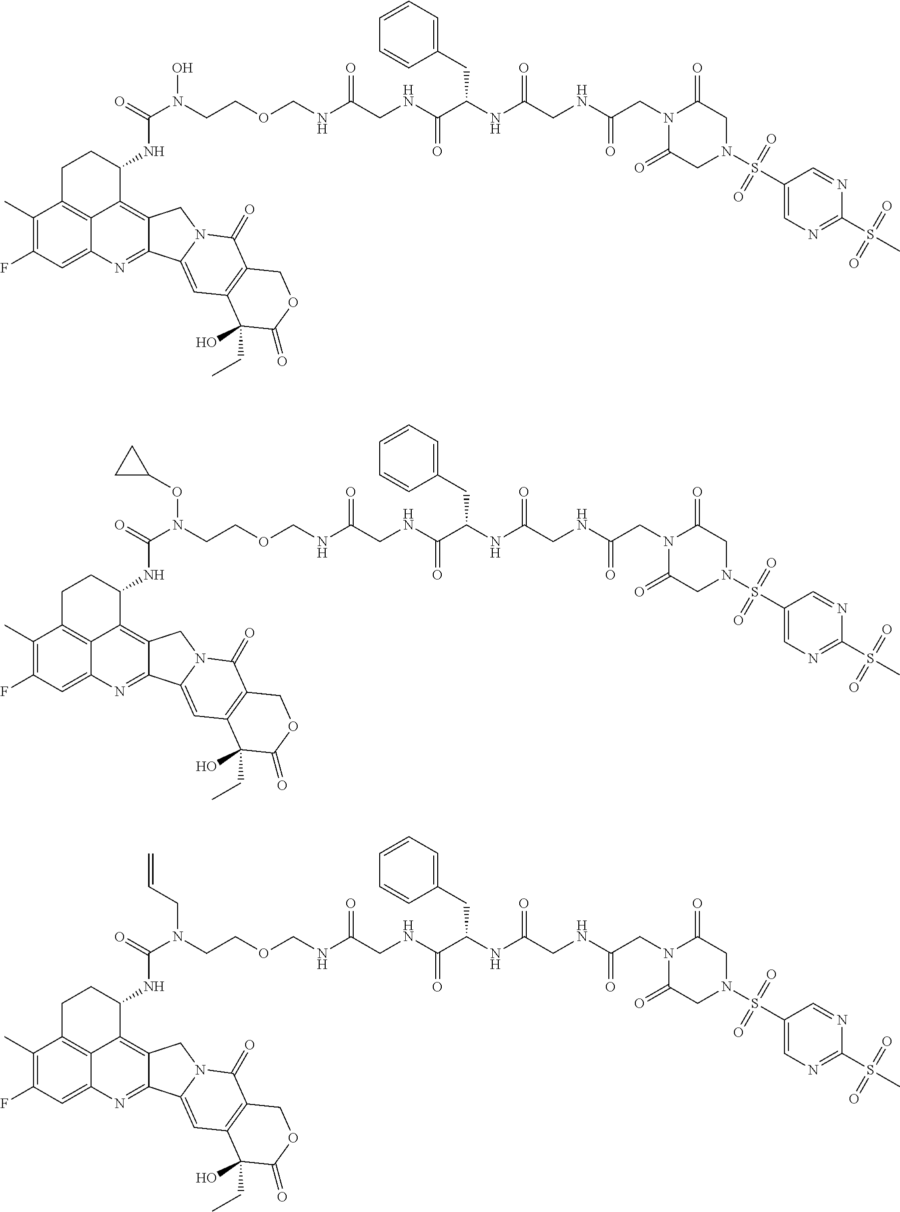

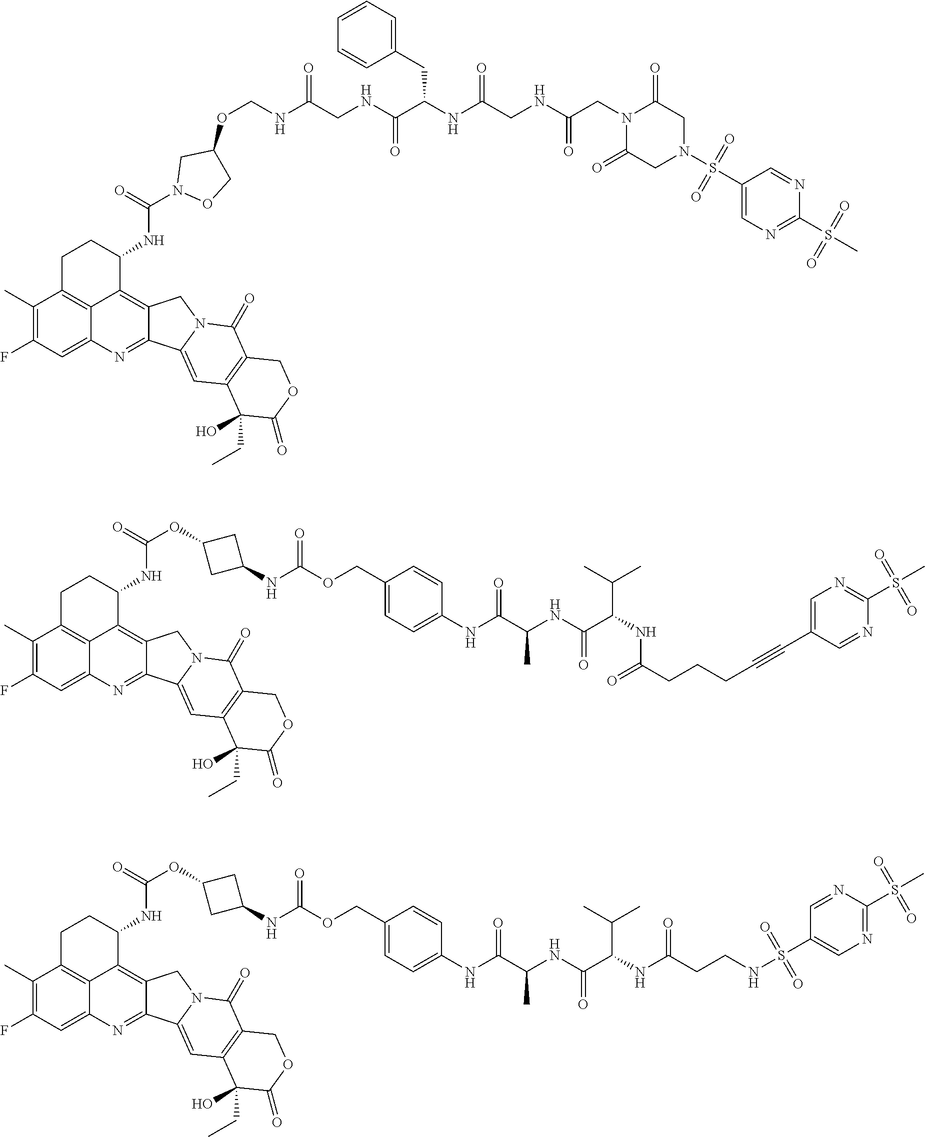

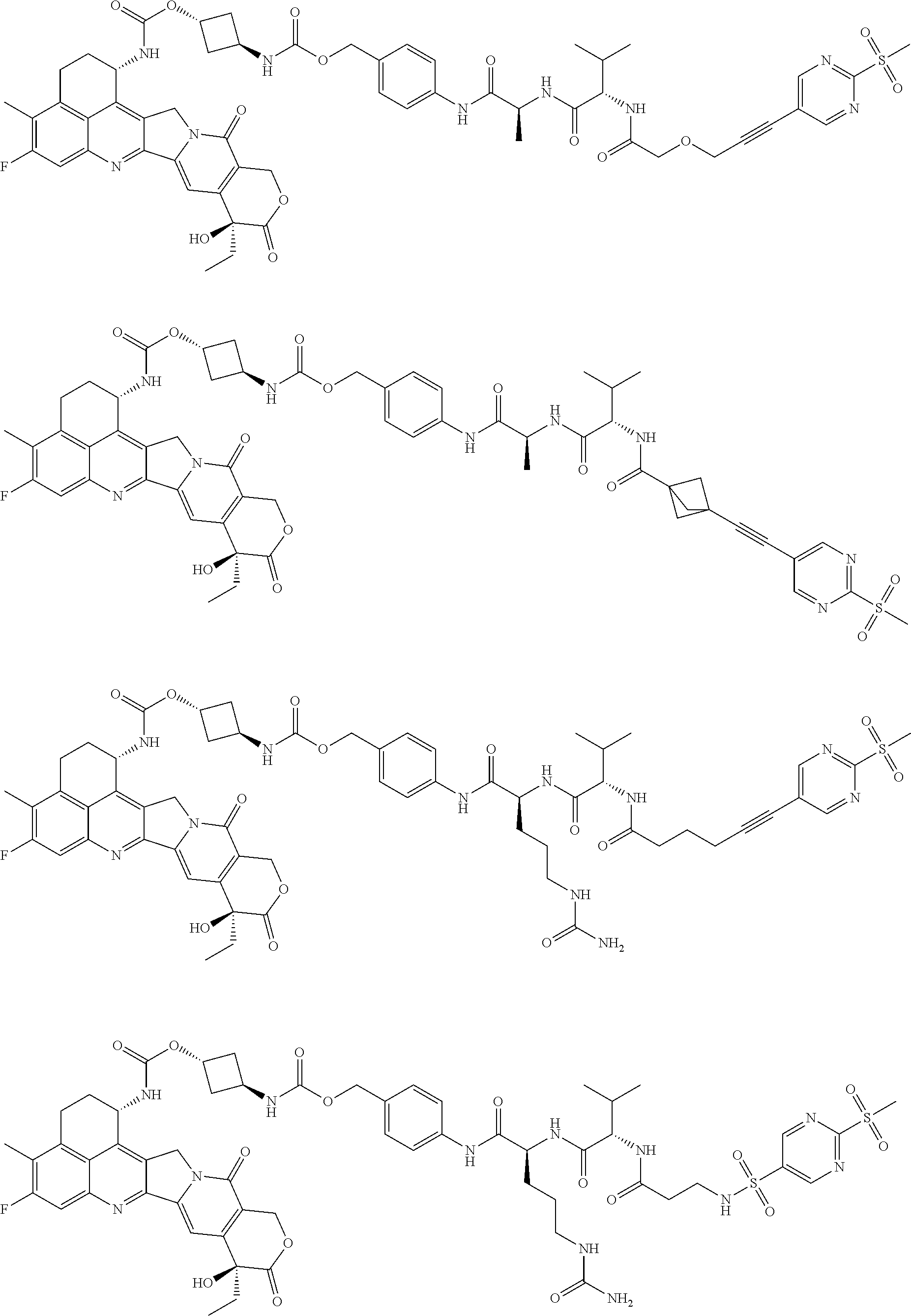

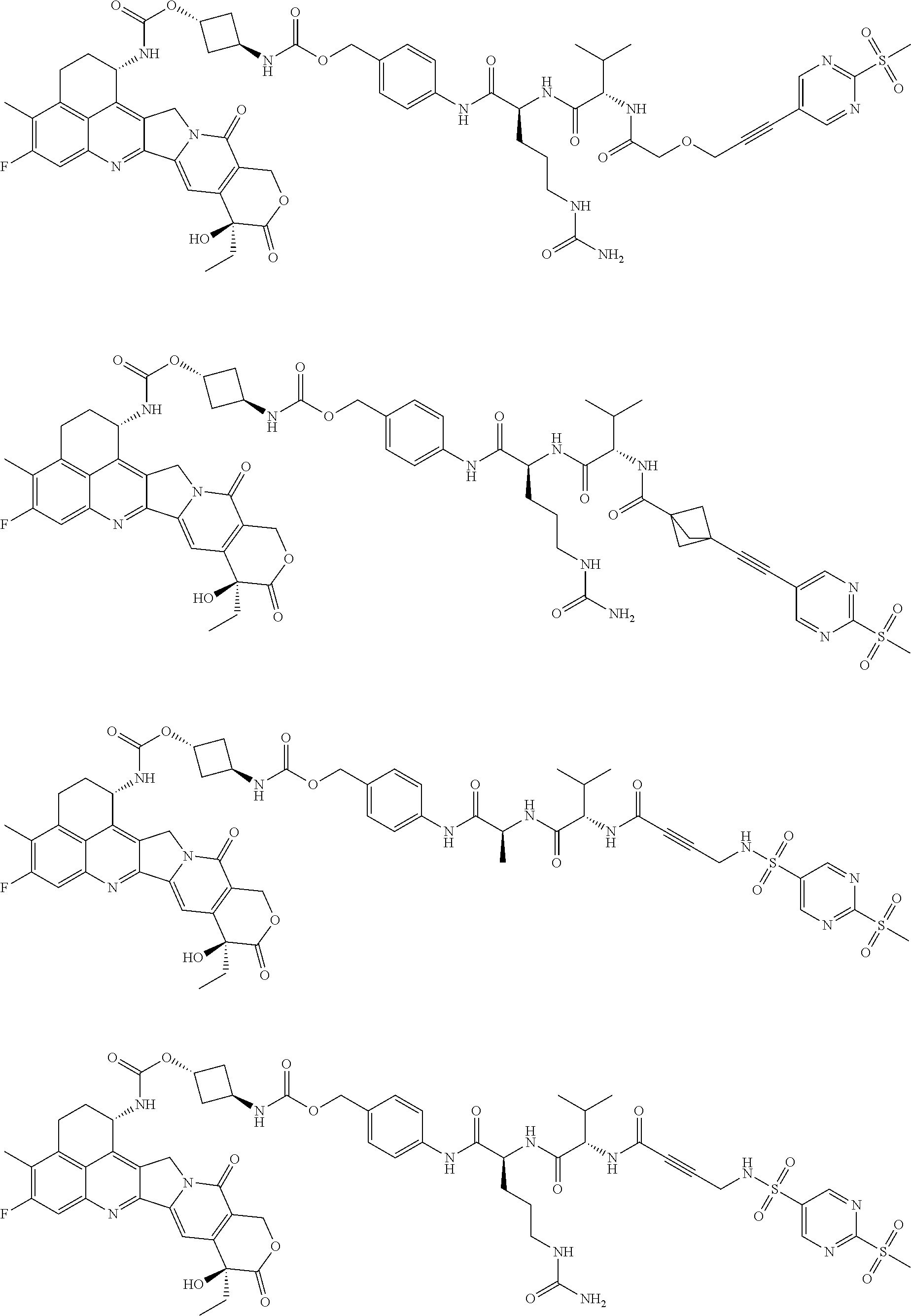

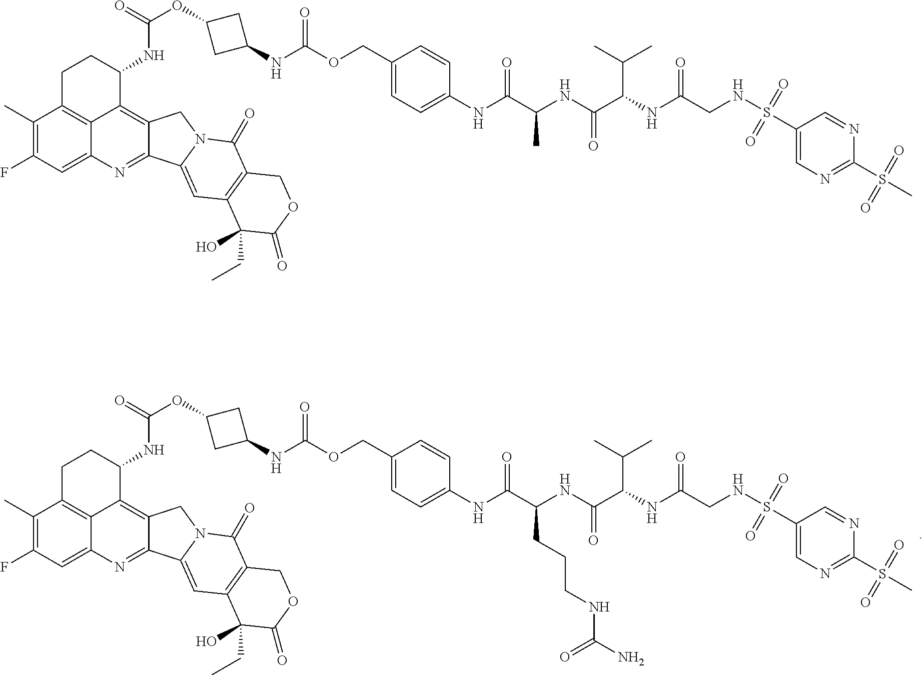 [SMILES: CC[C@@]1(O)C(=O)OCc2c1cc1n(c2=O)Cc2c-1nc1cc(F)c(C)c3c1c2[C@@H](NC(=O)O[C@H]1C[C@H](NC(=O)OCc2ccc(NC(=O)[C@H](C)NC(=O)[C@@H](NC(=O)CNS(=O)(=O)c4cnc(S(C)(=O)=O)nc4)C(C)C)cc2)C1)CC3 ; CC[C@@]1(O)C(=O)OCc2c1cc1n(c2=O)Cc2c-1nc1cc(F)c(C)c3c1c2[C@@H](NC(=O)O[C@H]1C[C@H](NC(=O)OCc2ccc(NC(=O)[C@H](CCCNC(N)=O)NC(=O)[C@@H](NC(=O)CNS(=O)(=O)c4cnc(S(C)(=O)=O)nc4)C(C)C)cc2)C1)CC3]

21. The antibody drug conjugate according to claim 6, which is selected from: 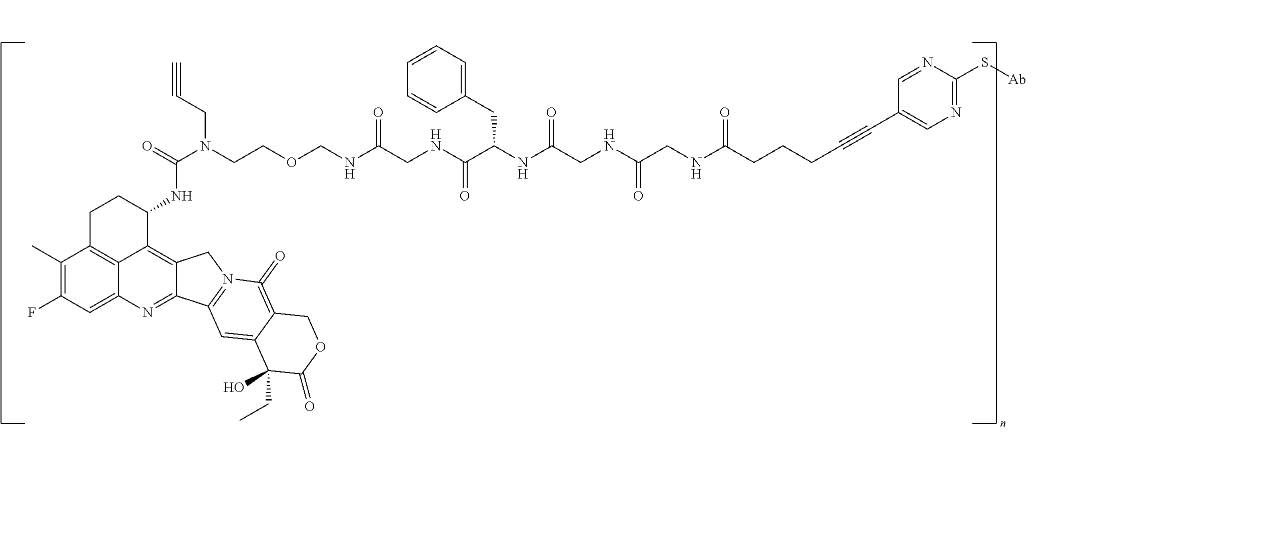 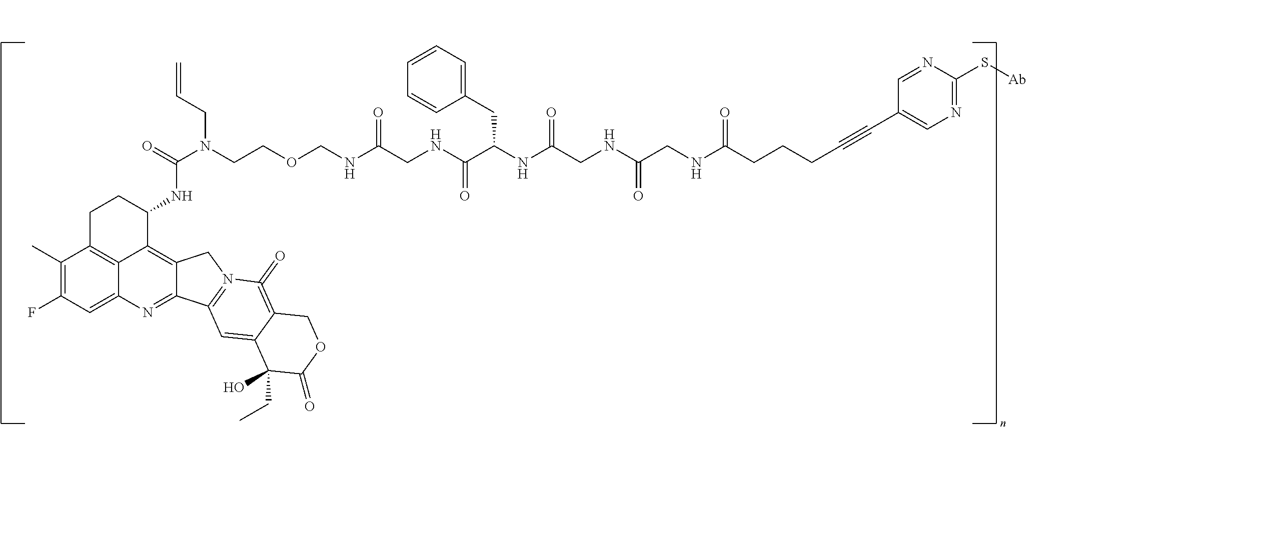 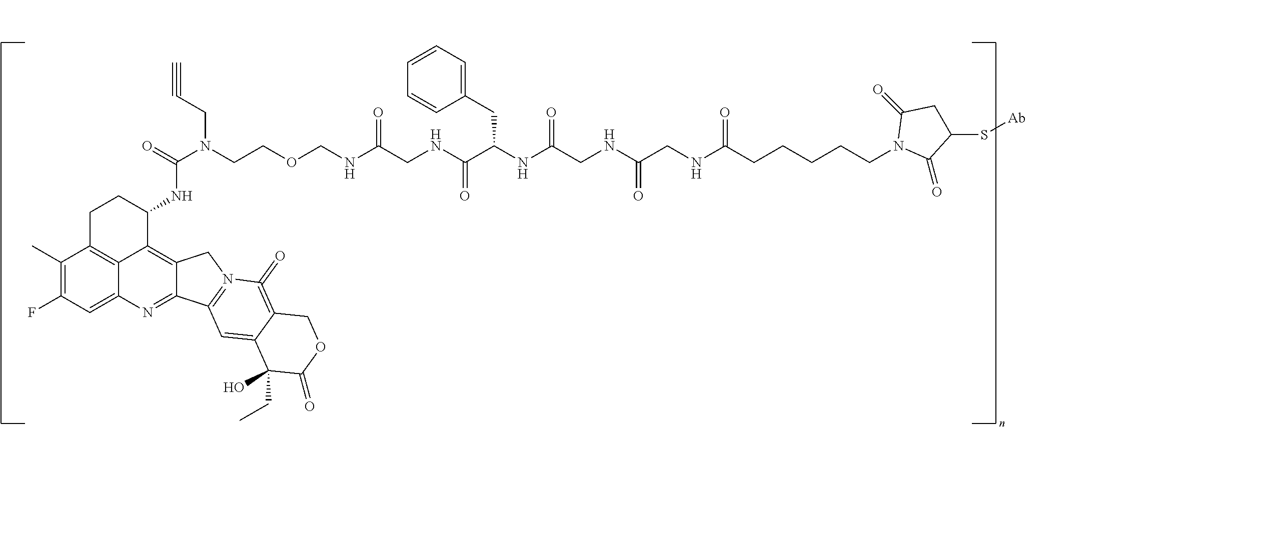 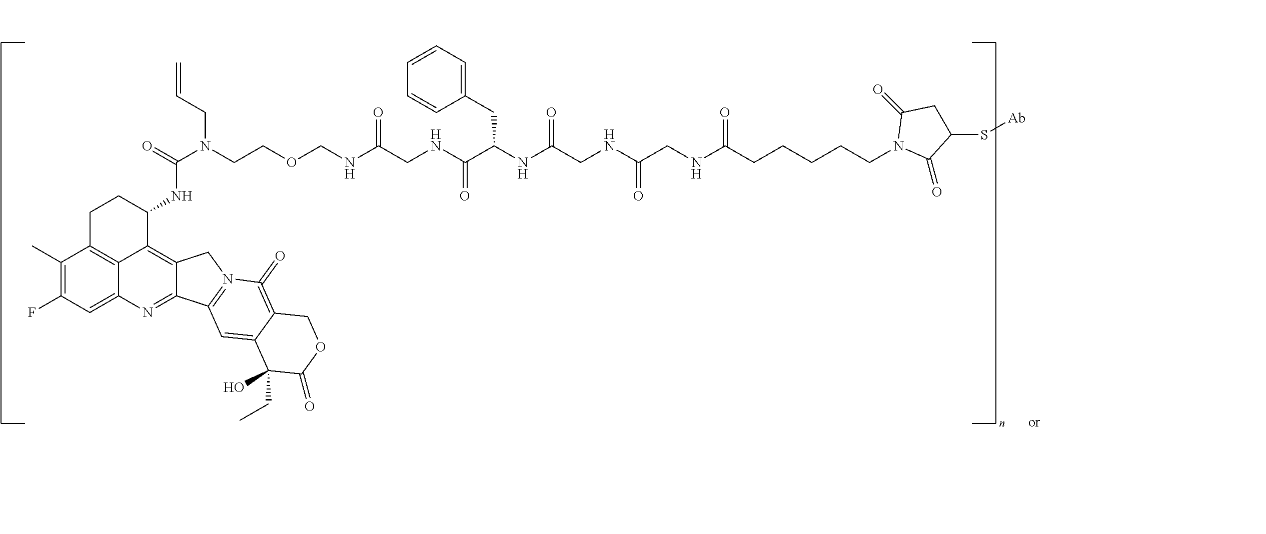 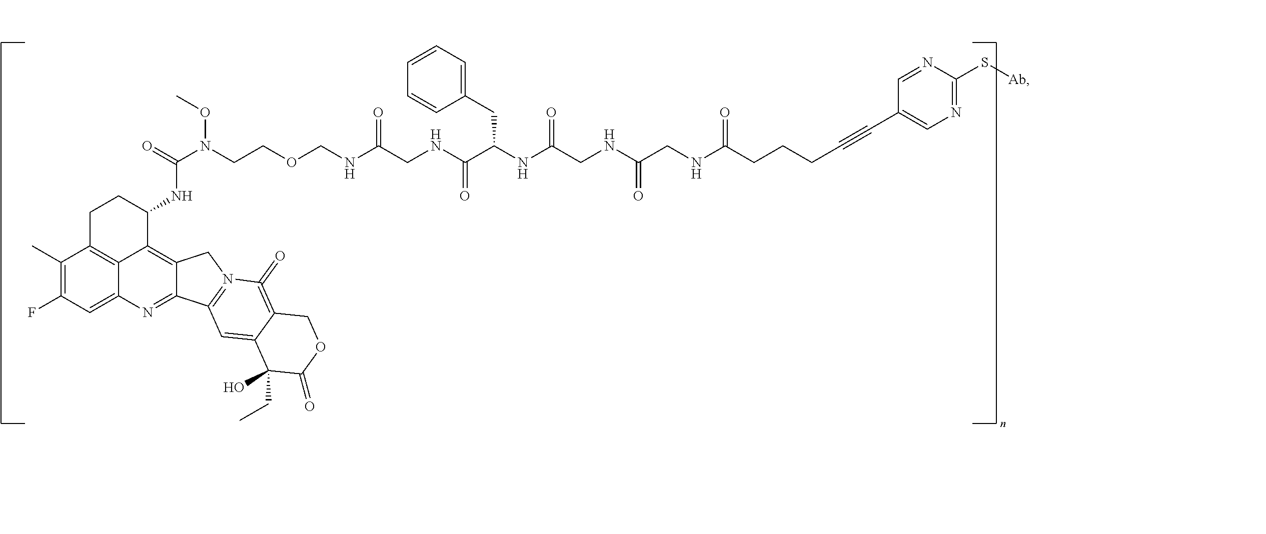
wherein, the Ab and n are as defined in claim 6.

22. The antibody drug conjugate according to claim 6, wherein the Ab is an anti-Her2 antibody; or the Ab is Trastuzumab or Pertuzumab; or the Ab is Pertuzumab.

23. The antibody drug conjugate according to claim 6, which is selected from an antibody drug conjugate as shown by general formula (II): 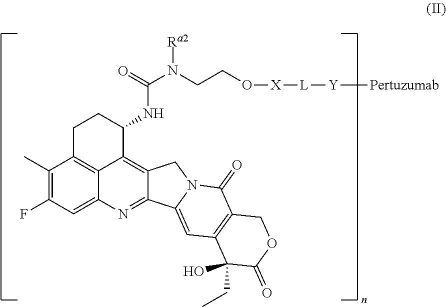
wherein, R^{a2} is selected from 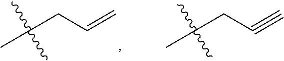
or —OCH₃;
wherein constitutional unit —X-L-Y— is selected from 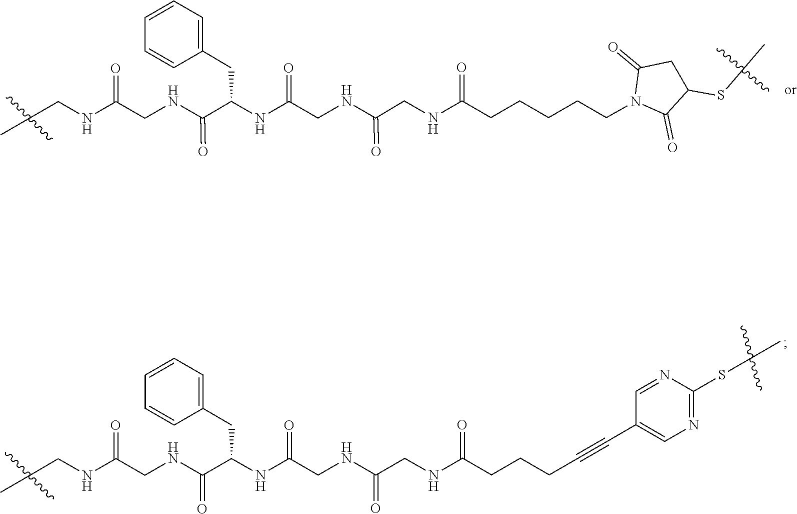 [SMILES: *CNC(=O)CNC(=O)[C@H](Cc1ccccc1)NC(=O)CNC(=O)CNC(=O)CCCCCN1C(=O)CC(S*)C1=O ; *CNC(=O)CNC(=O)[C@H](Cc1ccccc1)NC(=O)CNC(=O)CNC(=O)CCCC#Cc1cnc(S*)nc1]
and
n is selected from numerical values of 4-8.

24. The antibody drug conjugate according to claim 6, which is selected from: 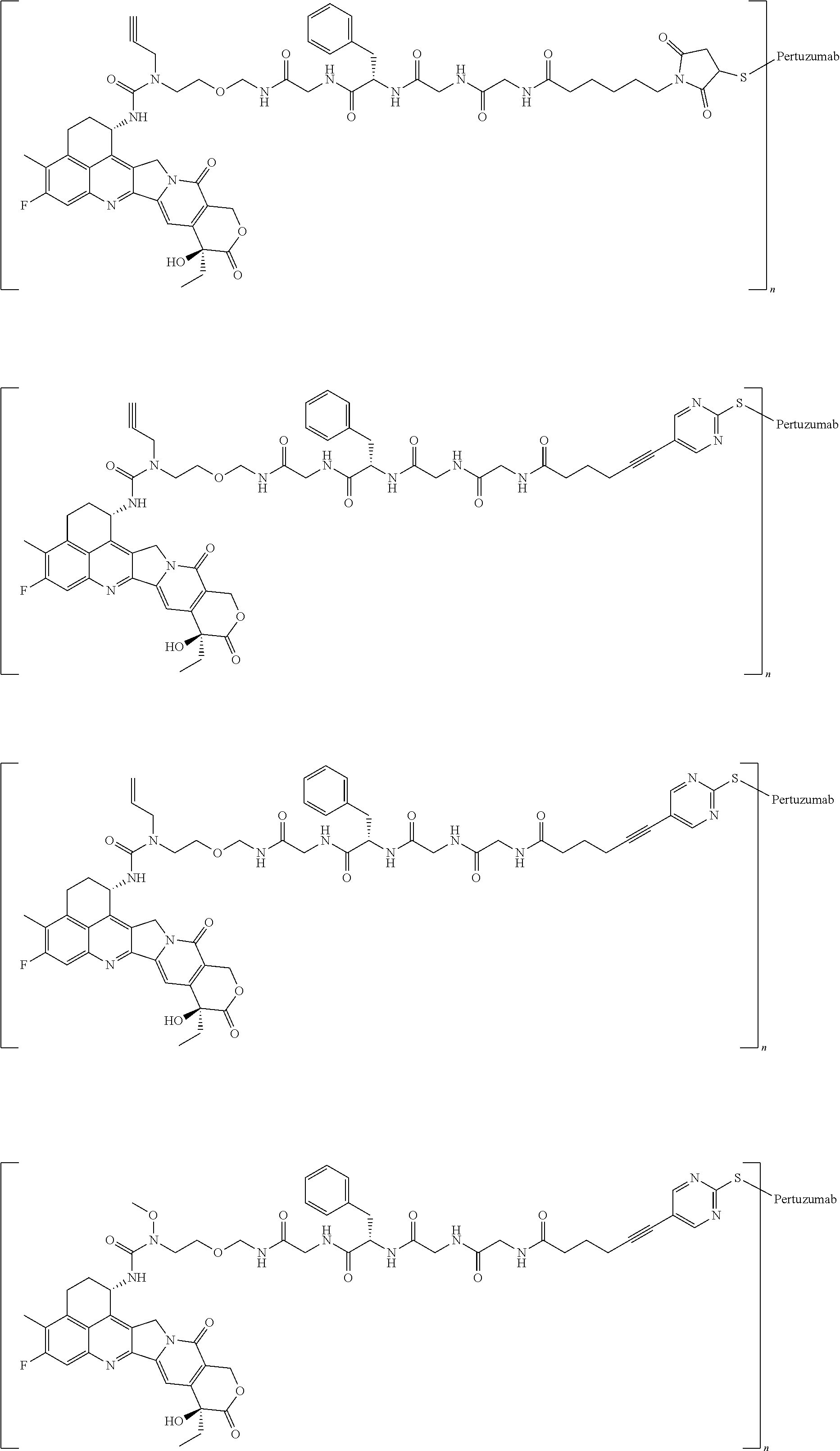
wherein, n is selected from numerical values of 6-8.

25. A pharmaceutical composition, comprising the antibody drug conjugate, the stereoisomer thereof or the pharmaceutically acceptable salt thereof according to claim 6, and a pharmaceutically acceptable carrier.

26. A method for treating cancer, comprising administering a therapeutically effective amount of the cytotoxic drug, the stereoisomer thereof or the pharmaceutically acceptable salt thereof according to claim 1.

27. A preparation method for an antibody drug conjugate, wherein, after treating Ab under reducing conditions, the cytotoxic drug-linker according to claim 20 is linked to the Ab through a thioether bond to form the antibody drug conjugate;
wherein the Ab is an anti-Her2 antibody.

28. (canceled)

29. A compound as shown by the following formula:

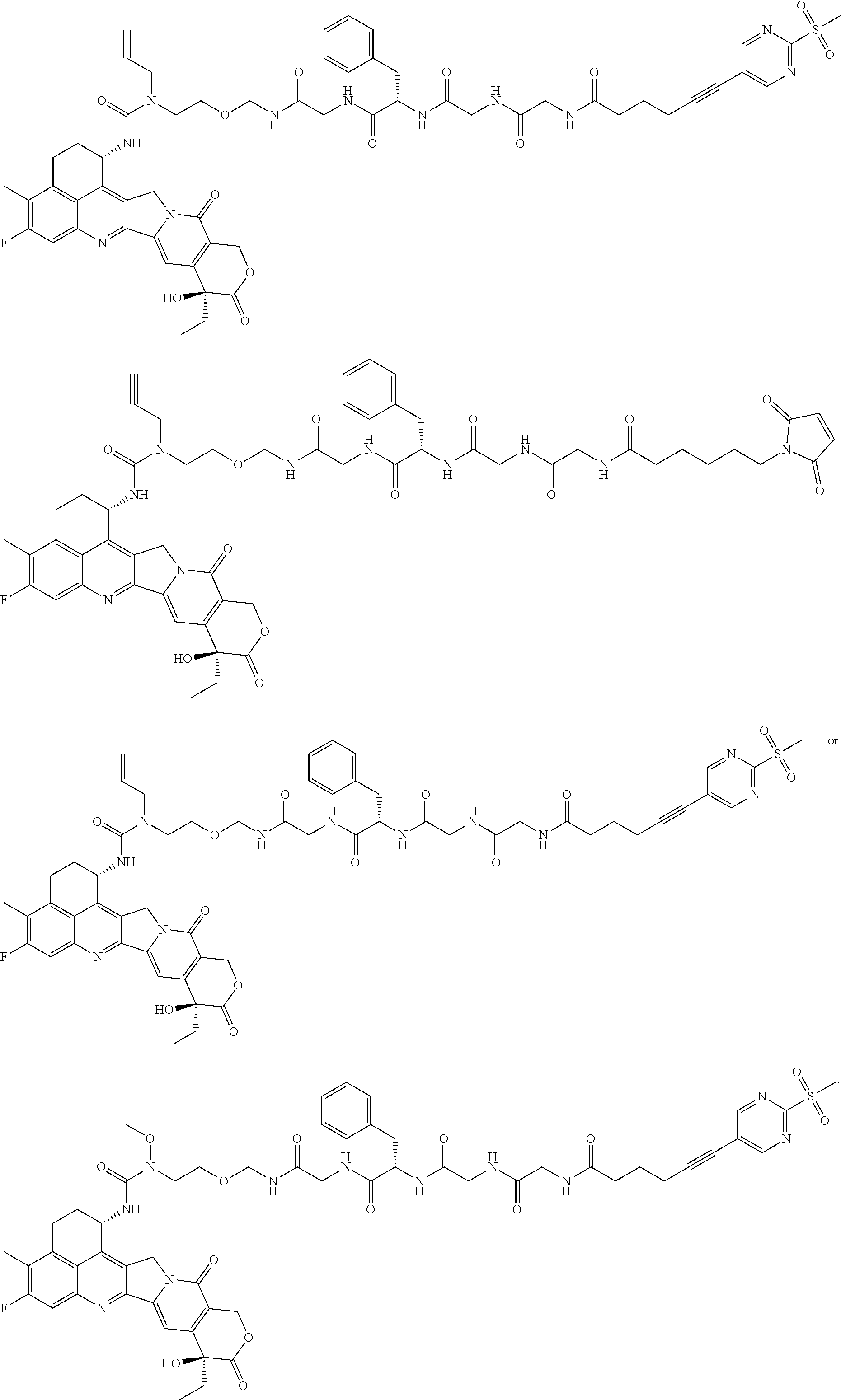

30. A compound as follows:

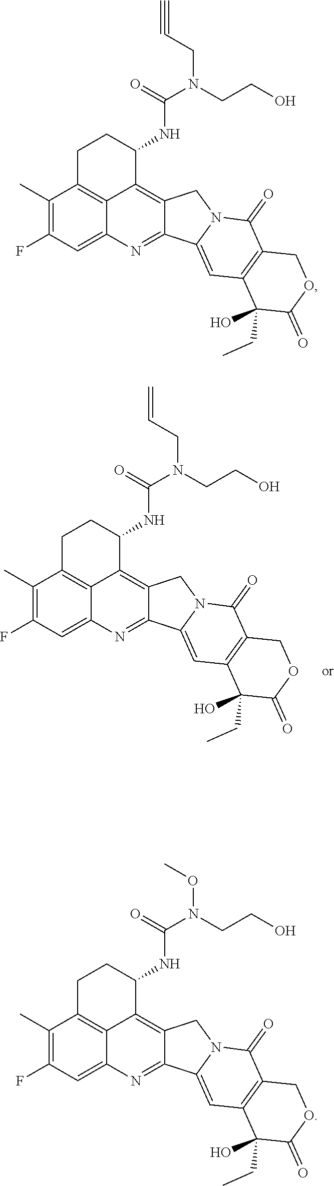 [SMILES: C#CCN(CCO)C(=O)N[C@H]1CCc2c(C)c(F)cc3nc4c(c1c23)Cn1c-4cc2c(c1=O)COC(=O)[C@]2(O)CC ; C=CCN(CCO)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)N(CCO)OC)CC3]

31. A method for treating cancer, comprising administering a therapeutically effective amount of the antibody drug conjugate, the stereoisomer thereof or the pharmaceutically acceptable salt thereof according to claim 6, or the pharmaceutical composition comprising the antibody drug conjugate, the stereoisomer thereof or the pharmaceutically acceptable salt thereof according to claim 6 to a subject in need thereof.

32. A method for treating cancer, comprising administering a therapeutically effective amount of the cytotoxic drug-linker, the stereoisomer thereof or the pharmaceutically acceptable salt thereof according to claim 19 to a subject in need thereof.
